(19) United States
(12) Patent Application Publication
(10) Pub. No.: US 2023/0257453 A1
(43) Pub. Date: Aug. 17, 2023

(54) COLLAGEN-TARGETED FUSION PROTEINS AND ANTIBODIES

(71) Applicant: Proviva Therapeutics (Hong Kong) Limited, Hong Kong (CN)

(72) Inventors: Zijuan LI, Shanghai (CN); Hongxing ZHOU, Bedford, MA (US)

(21) Appl. No.: 18/009,185

(22) PCT Filed: Jun. 11, 2021
(86) PCT No.: PCT/US21/37119

§ 371 Date: Dec. 8, 2022

Related U.S. Application Data
(60) Provisional application No. 63/037,739, filed on Jun. 11, 2020.

(51) Int. Cl. C07K 16/18; C07K 14/55; C07K 14/54; C07K 14/525; C07K 14/56; C12N 15/63

(52) U.S. Cl. CPC C07K 16/18; C07K 14/55; C07K 14/5443; C07K 14/525; C07K 14/5434; C07K 14/5428; C07K 14/56; C07K 14/5418; C12N 15/63; C07K 2317/565; C07K 2317/567

(57) ABSTRACT

Provided are improved antibodies, or antigen-binding fragments thereof, which specifically bind to a denatured human collagen polypeptide at a cryptic collagen epitope (anti-denatured collagen antibodies), and fusion proteins comprising anti-denatured collagen antibodies fused to an effector domain, such as a cytokine or an immunomodulatory antibody.

### CROSS-REFERENCE TO RELATED APPLICATIONS

This application claims the benefit under 35 U.S.C. § 119(e) to U.S. Provisional Application No. 63/037,739, filed Jun. 11, 2020, which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is PRVA_007_01WO_ST25.txt. The text file is about 1,067 KB, created on Jun. 11, 2021, and is being submitted electronically via EFS-Web.

### BACKGROUND

### Technical Field

The present disclosure relates improved antibodies, or antigen-binding fragments thereof, which specifically bind to a denatured human collagen polypeptide at a cryptic collagen epitope (anti-denatured collagen antibodies), and fusion proteins comprising anti-denatured collagen antibodies fused to an effector domain, such as a cytokine or an immunomodulatory antibody.

### Description of the Related Art

Cytokine and antibody therapies have utility in a variety of disease indications. For instance, Interleukin-2 (IL-2) and interleukin-15 (IL-15) immunotherapies have proven utility in the treatment of cancers such as malignant melanoma and renal cell cancer, and chronic infections such as HIV infections.

However, there are certain problems associated with most IL-2 and IL-15 therapies. For example, current forms of IL-2 therapy have a short half-life in circulation and predominantly expand immunosuppressive regulatory T cells, or T_{regs} (see, for example, Arenas-Ramirez et al., Trends in Immunology. 36: 763-777, 2015). Moreover, the effects of IL-2 therapy are predominantly systemic, rather than being localized to target tissues, resulting in many severe side effects such as breathing problems, nausea, low blood pressure, loss of appetite, confusion, serious infections, seizures, allergic reactions, heart problems, renal failure, and vascular leak syndrome.

Similarly, IL-15 has been shown to exhibit a short half-life and high doses can be required to achieve biological responses in vivo, resulting in clinical toxicities and limited anti-tumor responses in patients. IL-15 and IL-15 derivatives are under development to increase therapeutic effectiveness. However, significant drawbacks exist, including high serum Cmax initially causing over-activation of immune system, short PK due to either small molecular size for IL-15 (13-14 kD) or catabolism by the large number of immune cells expressing IL-15 receptors for IL-15 or IL-15 Fc fusion proteins, poor accumulation in the target tumor due to short PK, lack of or ineffective tumor targeting or retention in tumor tissues, and undesirable accumulation and immune activation activities in normal tissues. Nonetheless, IL-2 and IL-15 therapies can be effective, and there is an unmet need in the art to overcome these and other drawbacks.

Activation of cell surface death receptors of the tumor necrosis factor (TNF) receptor superfamily by the appropriate ligands represents an attractive therapeutic strategy to induce cell death by apoptosis in cancer cells (see, e.g., Palacios et al., Curr Pharm Des. 2014; 20(17):2819-33). As one example, TNF-related apoptosis-inducing ligand (TRAIL, also known as Apo2L) possesses the ability to induce apoptosis selectively in cancer cells, and has demonstrated robust anticancer activity in a number of preclinical studies. However, there are similar problems with TRAIL and other cytokine or ligand-based therapies.

Attempts to target effector molecules to tumor tissues have been explored. For example, fusion proteins between a monoclonal antibody with binding specificity for tumor cell surface antigens and an effector molecule have been generated to enhance the drug penetration to tumor tissues. However, at least one drawback to this approach includes internalization of the antigen, which limits the bioavailability of the effector molecule and thus its ability to signal effector cells. Non-cellular target-mediated effector molecule targeting has also been explored, for example, through attachment of effector molecules to tenascin-C, fibronectin splicing domain, EDB, or collagens. The potential issues with these approaches include the variable target levels in tumor tissues and/or lack of tumor tissue specificity, which leads to insufficient accumulation in tumor tissues and/or insufficient selectivity towards tumor tissues relative to normal tissues, resulting in poor efficacy and higher toxicity.

Accordingly, there remains a need to optimize the pharmacokinetics and/or biological activities of these and other agents.

### BRIEF SUMMARY

Embodiments of the present disclosure include fusion protein, comprising

(a) an antibody, or antigen-binding fragment thereof, which specifically binds to a denatured human collagen type I, II, III, IV, and/or V polypeptide at a cryptic collagen epitope, wherein (a) is fused via a linker to;

(b) an effector domain.

In some embodiments, (a) preferentially binds to the denatured human collagen type I, II, III, IV, and/or V polypeptide relative to a corresponding native human collagen polypeptide, optionally wherein (a) has about or at least about 2, 3, 4, 5, 6, 7, 8, 9, 10, 15, 20, 25, 30, 35, 40, 50, 60, 70, 80, 90, or 100-fold higher binding affinity for the denatured human collagen polypeptide than it has for the corresponding native human collagen polypeptide.

In some embodiments, (a) specifically binds to the denatured human collagen type I, II, III, IV, and V polypeptides at the HU177 cryptic collagen epitope, optionally wherein the HU177 cryptic collagen epitope comprises PGXP, LPGXPG (SEQ ID NO: _), and/or GPP′GXP′G (SEQ ID NO: _), wherein X is any amino acid, and wherein P′ is hydroxylproline. In some embodiments, (a) specifically binds to the denatured human collagen type IV polypeptide at the HUIV26 cryptic collagen epitope.

In some embodiments, (a) comprises:

a heavy chain variable (V_{H}) region that comprises complementary determining region V_{H}CDR1, V_{H}CDR2, and V_{H}CDR3 sequences selected from Table A1 and variants thereof which specifically bind to the denatured human collagen polypeptide at the cryptic collagen epitope; and a light chain variable (V_{L}) region that comprises complementary determining region V_{L}CDR1, V_{L}CDR2, and V_{L}CDR3 sequences selected from Table A1 and variants thereof which specifically bind to the denatured human collagen polypeptide at the cryptic collagen epitope.

In some embodiments, (a) comprises:

the V_{H}CDR1, a V_{H}CDR2, and V_{H}CDR3 regions respectively comprise SEQ ID NOs: 1-3; and the V_{L}CDR1, V_{L}CDR2, and V_{L}CDR3 regions respectively comprise SEQ ID NOs: 4-6;

the V_{H}CDR1, a V_{H}CDR2, and V_{H}CDR3 regions respectively comprise SEQ ID NOs: 7-9; and the V_{L}CDR1, V_{L}CDR2, and V_{L}CDR3 regions respectively comprise SEQ ID NOs: 10-12;

the V_{H}CDR1, a V_{H}CDR2, and V_{H}CDR3 regions respectively comprise SEQ ID NOs: 13-15; and the V_{L}CDR1, V_{L}CDR2, and V_{L}CDR3 regions respectively comprise SEQ ID NOs: 16-18;

the V_{H}CDR1, a V_{H}CDR2, and V_{H}CDR3 regions respectively comprise SEQ ID NOs: 19-21; and the V_{L}CDR1, V_{L}CDR2, and V_{L}CDR3 regions respectively comprise SEQ ID NOs: 22-24;

the V_{H}CDR1, a V_{H}CDR2, and V_{H}CDR3 regions respectively comprise SEQ ID NOs: 25-27; and the V_{L}CDR1, V_{L}CDR2, and V_{L}CDR3 regions respectively comprise SEQ ID NOs: 28-30;

the V_{H}CDR1, a V_{H}CDR2, and V_{H}CDR3 regions respectively comprise SEQ ID NOs: 31-33; and the V_{L}CDR1, V_{L}CDR2, and V_{L}CDR3 regions respectively comprise SEQ ID NOs: 34-36;

the V_{H}CDR1, a V_{H}CDR2, and V_{H}CDR3 regions respectively comprise SEQ ID NOs: 37-39; and the V_{L}CDR1, V_{L}CDR2, and V_{L}CDR3 regions respectively comprise SEQ ID NOs: 40-42;

the V_{H}CDR1, a V_{H}CDR2, and V_{H}CDR3 regions respectively comprise SEQ ID NOs: 43-45; and the V_{L}CDR1, V_{L}CDR2, and V_{L}CDR3 regions respectively comprise SEQ ID NOs: 46-48;

the V_{H}CDR1, a V_{H}CDR2, and V_{H}CDR3 regions respectively comprise SEQ ID NOs: 49-51; and the V_{L}CDR1, V_{L}CDR2, and V_{L}CDR3 regions respectively comprise SEQ ID NOs: 52-54;

the V_{H}CDR1, a V_{H}CDR2, and V_{H}CDR3 regions respectively comprise SEQ ID NOs: 55-57; and the V_{L}CDR1, V_{L}CDR2, and V_{L}CDR3 regions respectively comprise SEQ ID NOs: 58-60;

the V_{H}CDR1, a V_{H}CDR2, and V_{H}CDR3 regions respectively comprise SEQ ID NOs: 61-63; and the V_{L}CDR1, V_{L}CDR2, and V_{L}CDR3 regions respectively comprise SEQ ID NOs: 64-66;

the V_{H}CDR1, a V_{H}CDR2, and V_{H}CDR3 regions respectively comprise SEQ ID NOs: 67-69; and the V_{L}CDR1, V_{L}CDR2, and V_{L}CDR3 regions respectively comprise SEQ ID NOs: 70-72;

the V_{H}CDR1, a V_{H}CDR2, and V_{H}CDR3 regions respectively comprise SEQ ID NOs: 73-75; and the V_{L}CDR1, V_{L}CDR2, and V_{L}CDR3 regions respectively comprise SEQ ID NOs: 76-78;

the V_{H}CDR1, a V_{H}CDR2, and V_{H}CDR3 regions respectively comprise SEQ ID NOs: 79-81; and the V_{L}CDR1, V_{L}CDR2, and V_{L}CDR3 regions respectively comprise SEQ ID NOs: 82-84;

the V_{H}CDR1, a V_{H}CDR2, and V_{H}CDR3 regions respectively comprise SEQ ID NOs: 85-87; and the V_{L}CDR1, V_{L}CDR2, and V_{L}CDR3 regions respectively comprise SEQ ID NOs: 88-90;

the V_{H}CDR1, a V_{H}CDR2, and V_{H}CDR3 regions respectively comprise SEQ ID NOs: 91-93; and the V_{L}CDR1, V_{L}CDR2, and V_{L}CDR3 regions respectively comprise SEQ ID NOs: 94-96;

the V_{H}CDR1, a V_{H}CDR2, and V_{H}CDR3 regions respectively comprise SEQ ID NOs: 97-99; and the V_{L}CDR1, V_{L}CDR2, and V_{L}CDR3 regions respectively comprise SEQ ID NOs: 100-102;

the V_{H}CDR1, a V_{H}CDR2, and V_{H}CDR3 regions respectively comprise SEQ ID NOs: 103-105; and the V_{L}CDR1, V_{L}CDR2, and V_{L}CDR3 regions respectively comprise SEQ ID NOs: 106-108;

the V_{H}CDR1, a V_{H}CDR2, and V_{H}CDR3 regions respectively comprise SEQ ID NOs: 109-111; and the V_{L}CDR1, V_{L}CDR2, and V_{L}CDR3 regions respectively comprise SEQ ID NOs: 112-114;

the V_{H}CDR1, a V_{H}CDR2, and V_{H}CDR3 regions respectively comprise SEQ ID NOs: 115-117; and the V_{L}CDR1, V_{L}CDR2, and V_{L}CDR3 regions respectively comprise SEQ ID NOs: 118-120;

the V_{H}CDR1, a V_{H}CDR2, and V_{H}CDR3 regions respectively comprise SEQ ID NOs: 121-123; and the V_{L}CDR1, V_{L}CDR2, and V_{L}CDR3 regions respectively comprise SEQ ID NOs: 124-126;

the V_{H}CDR1, a V_{H}CDR2, and V_{H}CDR3 regions respectively comprise SEQ ID NOs: 127-129; and the V_{L}CDR1, V_{L}CDR2, and V_{L}CDR3 regions respectively comprise SEQ ID NOs: 130-132;

the V_{H}CDR1, a V_{H}CDR2, and V_{H}CDR3 regions respectively comprise SEQ ID NOs: 133-135; and the V_{L}CDR1, V_{L}CDR2, and V_{L}CDR3 regions respectively comprise SEQ ID NOs: 136-138;

the V_{H}CDR1, a V_{H}CDR2, and V_{H}CDR3 regions respectively comprise SEQ ID NOs: 139-141; and the V_{L}CDR1, V_{L}CDR2, and V_{L}CDR3 regions respectively comprise SEQ ID NOs: 142-144;

the V_{H}CDR1, a V_{H}CDR2, and V_{H}CDR3 regions respectively comprise SEQ ID NOs: 145-147; and the V_{L}CDR1, V_{L}CDR2, and V_{L}CDR3 regions respectively comprise SEQ ID NOs: 148-150;

the V_{H}CDR1, a V_{H}CDR2, and V_{H}CDR3 regions respectively comprise SEQ ID NOs: 151-153; and the V_{L}CDR1, V_{L}CDR2, and V_{L}CDR3 regions respectively comprise SEQ ID NOs: 154-156;

the V_{H}CDR1, a V_{H}CDR2, and V_{H}CDR3 regions respectively comprise SEQ ID NOs: 157-159; and the V_{L}CDR1, V_{L}CDR2, and V_{L}CDR3 regions respectively comprise SEQ ID NOs: 160-162;

the V_{H}CDR1, a V_{H}CDR2, and V_{H}CDR3 regions respectively comprise SEQ ID NOs: 163-165; and the V_{L}CDR1, V_{L}CDR2, and V_{L}CDR3 regions respectively comprise SEQ ID NOs: 166-168;

the V_{H}CDR1, a V_{H}CDR2, and V_{H}CDR3 regions respectively comprise SEQ ID NOs: 169-171; and the V_{L}CDR1, V_{L}CDR2, and V_{L}CDR3 regions respectively comprise SEQ ID NOs: 172-174;

the V_{H}CDR1, a V_{H}CDR2, and V_{H}CDR3 regions respectively comprise SEQ ID NOs: 175-177; and the V_{L}CDR1, V_{L}CDR2, and V_{L}CDR3 regions respectively comprise SEQ ID NOs: 178-180;

the V_{H}CDR1, a V_{H}CDR2, and V_{H}CDR3 regions respectively comprise SEQ ID NOs: 181-183; and the V_{L}CDR1, V_{L}CDR2, and V_{L}CDR3 regions respectively comprise SEQ ID NOs: 184-186;

the V_{H}CDR1, a V_{H}CDR2, and V_{H}CDR3 regions respectively comprise SEQ ID NOs: 187-189; and the V_{L}CDR1, V_{L}CDR2, and V_{L}CDR3 regions respectively comprise SEQ ID NOs: 190-192;

the V_{H}CDR1, a V_{H}CDR2, and V_{H}CDR3 regions respectively comprise SEQ ID NOs: 193-195; and the V_{L}CDR1, V_{L}CDR2, and V_{L}CDR3 regions respectively comprise SEQ ID NOs: 196-198;

the V_{H}CDR1, a V_{H}CDR2, and V_{H}CDR3 regions respectively comprise SEQ ID NOs: 199-201; and the V_{L}CDR1, V_{L}CDR2, and V_{L}CDR3 regions respectively comprise SEQ ID NOs: 202-204;

the V_{H}CDR1, a V_{H}CDR2, and V_{H}CDR3 regions respectively comprise SEQ ID NOs: 205-207; and the V_{L}CDR1, V_{L}CDR2, and V_{L}CDR3 regions respectively comprise SEQ ID NOs: 208-210;

the V_{H}CDR1, a V_{H}CDR2, and V_{H}CDR3 regions respectively comprise SEQ ID NOs: 211-213; and the V_{L}CDR1, V_{L}CDR2, and V_{L}CDR3 regions respectively comprise SEQ ID NOs: 214-216;

the V_{H}CDR1, a V_{H}CDR2, and V_{H}CDR3 regions respectively comprise SEQ ID NOs: 217-219; and the V_{L}CDR1, V_{L}CDR2, and V_{L}CDR3 regions respectively comprise SEQ ID NOs: 220-222;

the V_{H}CDR1, a V_{H}CDR2, and V_{H}CDR3 regions respectively comprise SEQ ID NOs: 223-225; and the V_{L}CDR1, V_{L}CDR2, and V_{L}CDR3 regions respectively comprise SEQ ID NOs: 226-228;

the V_{H}CDR1, a V_{H}CDR2, and V_{H}CDR3 regions respectively comprise SEQ ID NOs: 229-231; and the V_{L}CDR1, V_{L}CDR2, and V_{L}CDR3 regions respectively comprise SEQ ID NOs: 232-234;

the V_{H}CDR1, a V_{H}CDR2, and V_{H}CDR3 regions respectively comprise SEQ ID NOs: 235-237; and the V_{L}CDR1, V_{L}CDR2, and V_{L}CDR3 regions respectively comprise SEQ ID NOs: 238-240;

the V_{H}CDR1, a V_{H}CDR2, and V_{H}CDR3 regions respectively comprise SEQ ID NOs: 241-243; and the V_{L}CDR1, V_{L}CDR2, and V_{L}CDR3 regions respectively comprise SEQ ID NOs: 244-246;

the V_{H}CDR1, a V_{H}CDR2, and V_{H}CDR3 regions respectively comprise SEQ ID NOs: 247-249; and the V_{L}CDR1, V_{L}CDR2, and V_{L}CDR3 regions respectively comprise SEQ ID NOs: 250-252;

the V_{H}CDR1, a V_{H}CDR2, and V_{H}CDR3 regions respectively comprise SEQ ID NOs: 253-255; and the V_{L}CDR1, V_{L}CDR2, and V_{L}CDR3 regions respectively comprise SEQ ID NOs: 256-258;

the V_{H}CDR1, a V_{H}CDR2, and V_{H}CDR3 regions respectively comprise SEQ ID NOs: 259-261; and the V_{L}CDR1, V_{L}CDR2, and V_{L}CDR3 regions respectively comprise SEQ ID NOs: 262-264; or

the V_{H}CDR1, a V_{H}CDR2, and V_{H}CDR3 regions respectively comprise SEQ ID NOs: 265-267; and the V_{L}CDR1, V_{L}CDR2, and V_{L}CDR3 regions respectively comprise SEQ ID NOs: 268-270,

including variants thereof that have 1, 2, 3, 4, 5, or 6 total alterations in one or more of the CDR regions and which specifically bind to the denatured human collagen polypeptide at the cryptic collagen epitope.

In some embodiments, for (a):

the heavy chain is at least 80, 85, 90, 95, 97, 98, 99, or 100% identical to a sequence selected from Table A2, including wherein the heavy chain has 1, 2, 3, 4, 5, or 6 alterations in one or more framework regions, and

the light chain is at least 80, 85, 90, 95, 97, 98, 99, or 100% identical to a sequence selected from Table A2, including wherein the light chain has 1, 2, 3, 4, 5, or 6 alterations in one or more framework regions.

In some embodiments, for (a):

the heavy chain comprises a sequence at least 80, 85, 90, 95, 97, 98, 99, or 100% identical to SEQ ID NO: 271, and the light chain comprises a sequence at least 80, 85, 90, 95, 97, 98, 99, or 100% identical to SEQ ID NO: 272;

the heavy chain comprises a sequence at least 80, 85, 90, 95, 97, 98, 99, or 100% identical to SEQ ID NO: 273, and the light chain comprises a sequence at least 80, 85, 90, 95, 97, 98, 99, or 100% identical to SEQ ID NO: 274;

the heavy chain comprises a sequence at least 80, 85, 90, 95, 97, 98, 99, or 100% identical to SEQ ID NO: 275, and the light chain comprises a sequence at least 80, 85, 90, 95, 97, 98, 99, or 100% identical to SEQ ID NO: 276;

the heavy chain comprises a sequence at least 80, 85, 90, 95, 97, 98, 99, or 100% identical to SEQ ID NO: 277, and the light chain comprises a sequence at least 80, 85, 90, 95, 97, 98, 99, or 100% identical to SEQ ID NO: 278;

the heavy chain comprises a sequence at least 80, 85, 90, 95, 97, 98, 99, or 100% identical to SEQ ID NO: 279, and the light chain comprises a sequence at least 80, 85, 90, 95, 97, 98, 99, or 100% identical to SEQ ID NO: 280;

the heavy chain comprises a sequence at least 80, 85, 90, 95, 97, 98, 99, or 100% identical to SEQ ID NO: 281, and the light chain comprises a sequence at least 80, 85, 90, 95, 97, 98, 99, or 100% identical to SEQ ID NO: 282;

the heavy chain comprises a sequence at least 80, 85, 90, 95, 97, 98, 99, or 100% identical to SEQ ID NO: 283, and the light chain comprises a sequence at least 80, 85, 90, 95, 97, 98, 99, or 100% identical to SEQ ID NO: 284;

the heavy chain comprises a sequence at least 80, 85, 90, 95, 97, 98, 99, or 100% identical to SEQ ID NO: 285, and the light chain comprises a sequence at least 80, 85, 90, 95, 97, 98, 99, or 100% identical to SEQ ID NO: 286;

the heavy chain comprises a sequence at least 80, 85, 90, 95, 97, 98, 99, or 100% identical to SEQ ID NO: 287, and the light chain comprises a sequence at least 80, 85, 90, 95, 97, 98, 99, or 100% identical to SEQ ID NO: 288;

the heavy chain comprises a sequence at least 80, 85, 90, 95, 97, 98, 99, or 100% identical to SEQ ID NO: 289, and the light chain comprises a sequence at least 80, 85, 90, 95, 97, 98, 99, or 100% identical to SEQ ID NO: 290;

the heavy chain comprises a sequence at least 80, 85, 90, 95, 97, 98, 99, or 100% identical to SEQ ID NO: 291, and the light chain comprises a sequence at least 80, 85, 90, 95, 97, 98, 99, or 100% identical to SEQ ID NO: 292;

the heavy chain comprises a sequence at least 80, 85, 90, 95, 97, 98, 99, or 100% identical to SEQ ID NO: 293, and the light chain comprises a sequence at least 80, 85, 90, 95, 97, 98, 99, or 100% identical to SEQ ID NO: 294;

the heavy chain comprises a sequence at least 80, 85, 90, 95, 97, 98, 99, or 100% identical to SEQ ID NO: 295, and the light chain comprises a sequence at least 80, 85, 90, 95, 97, 98, 99, or 100% identical to SEQ ID NO: 296;

the heavy chain comprises a sequence at least 80, 85, 90, 95, 97, 98, 99, or 100% identical to SEQ ID NO: 297, and the light chain comprises a sequence at least 80, 85, 90, 95, 97, 98, 99, or 100% identical to SEQ ID NO: 298;

the heavy chain comprises a sequence at least 80, 85, 90, 95, 97, 98, 99, or 100% identical to SEQ ID NO: 299, and the light chain comprises a sequence at least 80, 85, 90, 95, 97, 98, 99, or 100% identical to SEQ ID NO: 300;

the heavy chain comprises a sequence at least 80, 85, 90, 95, 97, 98, 99, or 100% identical to SEQ ID NO: 301, and the light chain comprises a sequence at least 80, 85, 90, 95, 97, 98, 99, or 100% identical to SEQ ID NO: 302;

the heavy chain comprises a sequence at least 80, 85, 90, 95, 97, 98, 99, or 100% identical to SEQ ID NO: 303, and the light chain comprises a sequence at least 80, 85, 90, 95, 97, 98, 99, or 100% identical to SEQ ID NO: 304;

the heavy chain comprises a sequence at least 80, 85, 90, 95, 97, 98, 99, or 100% identical to SEQ ID NO: 305, and the light chain comprises a sequence at least 80, 85, 90, 95, 97, 98, 99, or 100% identical to SEQ ID NO: 306;

the heavy chain comprises a sequence at least 80, 85, 90, 95, 97, 98, 99, or 100% identical to SEQ ID NO: 307, and the light chain comprises a sequence at least 80, 85, 90, 95, 97, 98, 99, or 100% identical to SEQ ID NO: 308;

the heavy chain comprises a sequence at least 80, 85, 90, 95, 97, 98, 99, or 100% identical to SEQ ID NO: 309, and the light chain comprises a sequence at least 80, 85, 90, 95, 97, 98, 99, or 100% identical to SEQ ID NO: 310;

the heavy chain comprises a sequence at least 80, 85, 90, 95, 97, 98, 99, or 100% identical to SEQ ID NO: 311, and the light chain comprises a sequence at least 80, 85, 90, 95, 97, 98, 99, or 100% identical to SEQ ID NO: 312;

the heavy chain comprises a sequence at least 80, 85, 90, 95, 97, 98, 99, or 100% identical to SEQ ID NO: 313, and the light chain comprises a sequence at least 80, 85, 90, 95, 97, 98, 99, or 100% identical to SEQ ID NO: 314;

the heavy chain comprises a sequence at least 80, 85, 90, 95, 97, 98, 99, or 100% identical to SEQ ID NO: 315, and the light chain comprises a sequence at least 80, 85, 90, 95, 97, 98, 99, or 100% identical to SEQ ID NO: 316;

the heavy chain comprises a sequence at least 80, 85, 90, 95, 97, 98, 99, or 100% identical to SEQ ID NO: 317, and the light chain comprises a sequence at least 80, 85, 90, 95, 97, 98, 99, or 100% identical to SEQ ID NO: 318;

the heavy chain comprises a sequence at least 80, 85, 90, 95, 97, 98, 99, or 100% identical to SEQ ID NO: 319, and the light chain comprises a sequence at least 80, 85, 90, 95, 97, 98, 99, or 100% identical to SEQ ID NO: 320;

the heavy chain comprises a sequence at least 80, 85, 90, 95, 97, 98, 99, or 100% identical to SEQ ID NO: 321, and the light chain comprises a sequence at least 80, 85, 90, 95, 97, 98, 99, or 100% identical to SEQ ID NO: 322;

the heavy chain comprises a sequence at least 80, 85, 90, 95, 97, 98, 99, or 100% identical to SEQ ID NO: 323, and the light chain comprises a sequence at least 80, 85, 90, 95, 97, 98, 99, or 100% identical to SEQ ID NO: 324;

the heavy chain comprises a sequence at least 80, 85, 90, 95, 97, 98, 99, or 100% identical to SEQ ID NO: 325, and the light chain comprises a sequence at least 80, 85, 90, 95, 97, 98, 99, or 100% identical to SEQ ID NO: 326;

the heavy chain comprises a sequence at least 80, 85, 90, 95, 97, 98, 99, or 100% identical to SEQ ID NO: 327, and the light chain comprises a sequence at least 80, 85, 90, 95, 97, 98, 99, or 100% identical to SEQ ID NO: 328;

the heavy chain comprises a sequence at least 80, 85, 90, 95, 97, 98, 99, or 100% identical to SEQ ID NO: 329, and the light chain comprises a sequence at least 80, 85, 90, 95, 97, 98, 99, or 100% identical to SEQ ID NO: 330;

the heavy chain comprises a sequence at least 80, 85, 90, 95, 97, 98, 99, or 100% identical to SEQ ID NO: 331, and the light chain comprises a sequence at least 80, 85, 90, 95, 97, 98, 99, or 100% identical to SEQ ID NO: 332;

the heavy chain comprises a sequence at least 80, 85, 90, 95, 97, 98, 99, or 100% identical to SEQ ID NO: 333, and the light chain comprises a sequence at least 80, 85, 90, 95, 97, 98, 99, or 100% identical to SEQ ID NO: 334;

the heavy chain comprises a sequence at least 80, 85, 90, 95, 97, 98, 99, or 100% identical to SEQ ID NO: 335, and the light chain comprises a sequence at least 80, 85, 90, 95, 97, 98, 99, or 100% identical to SEQ ID NO: 336;

the heavy chain comprises a sequence at least 80, 85, 90, 95, 97, 98, 99, or 100% identical to SEQ ID NO: 337, and the light chain comprises a sequence at least 80, 85, 90, 95, 97, 98, 99, or 100% identical to SEQ ID NO: 338;

the heavy chain comprises a sequence at least 80, 85, 90, 95, 97, 98, 99, or 100% identical to SEQ ID NO: 339, and the light chain comprises a sequence at least 80, 85, 90, 95, 97, 98, 99, or 100% identical to SEQ ID NO: 340;

the heavy chain comprises a sequence at least 80, 85, 90, 95, 97, 98, 99, or 100% identical to SEQ ID NO: 341, and the light chain comprises a sequence at least 80, 85, 90, 95, 97, 98, 99, or 100% identical to SEQ ID NO: 342;

the heavy chain comprises a sequence at least 80, 85, 90, 95, 97, 98, 99, or 100% identical to SEQ ID NO: 343, and the light chain comprises a sequence at least 80, 85, 90, 95, 97, 98, 99, or 100% identical to SEQ ID NO: 344;

the heavy chain comprises a sequence at least 80, 85, 90, 95, 97, 98, 99, or 100% identical to SEQ ID NO: 345, and the light chain comprises a sequence at least 80, 85, 90, 95, 97, 98, 99, or 100% identical to SEQ ID NO: 346;

the heavy chain comprises a sequence at least 80, 85, 90, 95, 97, 98, 99, or 100% identical to SEQ ID NO: 347, and the light chain comprises a sequence at least 80, 85, 90, 95, 97, 98, 99, or 100% identical to SEQ ID NO: 348;

the heavy chain comprises a sequence at least 80, 85, 90, 95, 97, 98, 99, or 100% identical to SEQ ID NO: 349, and the light chain comprises a sequence at least 80, 85, 90, 95, 97, 98, 99, or 100% identical to SEQ ID NO: 350;

the heavy chain comprises a sequence at least 80, 85, 90, 95, 97, 98, 99, or 100% identical to SEQ ID NO: 351, and the light chain comprises a sequence at least 80, 85, 90, 95, 97, 98, 99, or 100% identical to SEQ ID NO: 352;

the heavy chain comprises a sequence at least 80, 85, 90, 95, 97, 98, 99, or 100% identical to SEQ ID NO: 353, and the light chain comprises a sequence at least 80, 85, 90, 95, 97, 98, 99, or 100% identical to SEQ ID NO: 354; or

the heavy chain comprises a sequence at least 80, 85, 90, 95, 97, 98, 99, or 100% identical to SEQ ID NO: 355, and the light chain comprises a sequence at least 80, 85, 90, 95, 97, 98, 99, or 100% identical to SEQ ID NO: 356.

In some embodiments, (a) comprises

a V_{H} region or heavy chain comprising:
an HFR1 sequence selected from SEQ ID NOs: 357-361;
an HFR2 sequence selected from SEQ ID NOs: 362-366;
an HFR3 sequence selected from SEQ ID NOs: 367-369;
an HFR4 sequence set forth in SEQ ID NO: 370;
a V_{H}CDR1 sequence selected from SEQ ID NOs: 371-372;
a V_{H}CDR2 sequence selected from SEQ ID NOs: 373-374; and
a V_{H}CDR3 sequence selected from SEQ ID NO: 375,

and/or a V_{L} region or light chain comprising:
an LFR1 sequence set forth in SEQ ID NO: 376;
an LFR2 sequence set forth in SEQ ID NO: 377;
an LFR3 sequence set forth in SEQ ID NO: 378;
an LFR4 sequence set forth in SEQ ID NO: 379;
a V_{L}CDR1 sequence selected from SEQ ID NOs: 380-385;
a V_{L}CDR2 sequence set forth in SEQ ID NO: 386; and
a V_{L}CDR3 sequence selected from SEQ ID NOs: 387-388,

wherein the antibody, or antigen-binding fragment thereof, specifically binds to a human denatured collagen, or wherein (a) comprises
an HFR1 sequence selected from SEQ ID NOs: 389-390;
an HFR2 sequence selected from SEQ ID NOs: 391-392;
an HFR3 sequence set forth in SEQ ID NO: 393;
an HFR4 sequence set forth in SEQ ID NO: 394;
a V_{H}CDR1 sequence selected from SEQ ID NOs: 395-397;
a V_{H}CDR2 sequence selected from SEQ ID NOs: 398-401; and
a V_{H}CDR3 sequence selected from SEQ ID NOs: 402-405,

and/or a VL region or light chain comprising:
an LFR1 sequence set forth in SEQ ID NO: 406;
an LFR2 sequence set forth in SEQ ID NO: 407;
an LFR3 sequence set forth in SEQ ID NO: 408;
an LFR4 sequence set forth in SEQ ID NO: 409;
a V_{L}CDR1 sequence selected from SEQ ID NOs: 410-412;
a V_{L}CDR2 set forth in SEQ ID NO: 413; and
a V_{L}CDR3 set forth in SEQ ID NO: 414,

wherein the antibody, or antigen-binding fragment thereof, specifically binds to a human denatured collagen.

In some embodiments, for (b) the effector domain comprises an immune cell-stimulatory ligand or domain, an immune cell-inhibitory ligand or domain, a cytocidal (e.g., tumor cell cytocidal) ligand or domain, or an immunomodulatory or anti-cancer antibody, or antigen-binding fragment thereof. In some embodiments, the effector domain is an IL-2 polypeptide, optionally an IL-2 polypeptide that comprises, consists, or consists essentially of an amino acid sequence that is at least 80, 85, 90, 95, 98, or 100% identical to a sequence selected from Table S1, and wherein the cell receptor is an IL-2Rβ/γc and/or IL-2Rα/β/γc chain present on the surface of an immune cell.

In some embodiments, the effector domain is an IL-15 polypeptide, optionally an IL-15 polypeptide that comprises, consists, or consists essentially of an amino acid sequence that is at least 80, 85, 90, 95, 98, or 100% identical to a sequence selected from Table S2, and wherein the cell receptor is an IL-15Rβ/γc chain present on the surface of an immune cell. In some embodiments, the effector domain is a hybrid IL-2/IL-15 polypeptide, optionally a hybrid IL-2/IL-15 polypeptide that comprises, consists, or consists essentially of an amino acid sequence that is at least 80, 85, 90, 95, 98, or 100% identical to a sequence selected from Table S3, and wherein the cell receptor is an IL-2Rβ/γc chain, an IL-2Rα/β/γc chain, and/or an IL-15Rβ/γc chain present on the surface of an immune cell.

In some embodiments, the effector domain is a TNF superfamily ligand polypeptide, optionally wherein the TNF superfamily ligand polypeptide and its corresponding cell receptor(s) are selected from Table T1, optionally wherein:

(i) the TNF superfamily ligand polypeptide is TRAIL, including single chain trimeric TRAIL, and the cell receptor is selected from Death receptor 4, Death receptor 5, Decoy receptor 1, and decoy receptor 2 present on an immune cell or cancer cell; or

(ii) the TNF superfamily ligand polypeptide is 4-1BBL, including single chain trimeric 4-1BB, and the cell receptor is 4-1BB (CD137) present on an immune cell or cancer cell. In some embodiments, the TNF superfamily ligand polypeptide comprises, consists, or consists essentially of an amino acid sequence that is at least 80, 85, 90, 95, 98, or 100% identical to a sequence selected from Table S4, and wherein the cell receptor is selected from the corresponding receptor from Table T1.

In some embodiments, the effector domain is an IL-12 polypeptide, optionally an IL-12 polypeptide that comprises, consists, or consists essentially of an amino acid sequence that is at least 80, 85, 90, 95, 98, or 100% identical to a sequence selected from Table S5, and wherein the cell receptor is an IL-12 receptor, optionally an IL-12Rβ1 and IL-12Rβ2 chain present on the surface of an immune cell. In some embodiments, the effector domain is an IL-10 polypeptide, optionally an IL-10 polypeptide that comprises, consists, or consists essentially of an amino acid sequence that is at least 80, 85, 90, 95, 98, or 100% identical to a sequence selected from Table S6, and wherein the cell receptor is an IL-10 receptor complex comprising IL-10α receptor and IL-10β receptor subunits, optionally wherein the cell receptor is an IL-10α receptor subunit.

In some embodiments, the effector domain is an IFN-α polypeptide, optionally an IFN-α polypeptide that comprises, consists, or consists essentially of an amino acid sequence that is at least 80, 85, 90, 95, 98, or 100% identical to a sequence selected from Table S7, and wherein the cell receptor is an interferon α/β receptor. In some embodiments, the effector domain is an interleukin-7 (IL-7) polypeptide, optionally an IL-7 polypeptide that comprises, consists, or consists essentially of an amino acid sequence that is at least 80, 85, 90, 95, 98, or 100% identical to a sequence selected from Table S8, and wherein the cell receptor is an IL-7 receptor (IL-7R); or wherein the effector domain is an interleukin-21 (IL-21) polypeptide, optionally an IL-7 polypeptide that comprises, consists, or consists essentially of an amino acid sequence that is at least 80, 85, 90, 95, 98, or 100% identical to a sequence selected from Table S9, and wherein the cell receptor is an IL-21 receptor (IL-21R).

In some embodiments, the effector domain comprises an immunomodulatory or anti-cancer antibody, or antigen-binding fragment thereof, which specifically binds to a polypeptide selected from human Her2/neu, Her1/EGF receptor (EGFR), EGFR1, EGFR2, EGFR3, Her3, A33 antigen, B7H3, B7H4, CD3, CD4, CD5, CD8, CD16, CD19, CD20, CD30, CD22, CD23 (IgE Receptor), B-cell maturation antigen (BCMA), Trop-2, Claudin 6, claudin 16, MAGE-3, C242 antigen, 5T4, IL-6, IL-13, PD-1, CTLA-4, PD-L1, TIGIT, TIM-3, LAG-3, 4-1BB, vascular endothelial growth factor VEGF (e.g., VEGF-A) VEGFR-1, VEGFR-2, CD27, CD28, CD33, CD37, CD40, CD44, CD51, CD52, CD56, CD74, CD80, CD86, CD137, CD152, CD200, CD221, CCR4, HLA-DR, CTLA-4, NPC-1C, Siglec15, MIC-A, NKG2A, NKG2D, Nkp30, NKp46, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, (b) is fused via the linker to the N-terminus of the V_{H} region of (a). In some embodiments, (b) is fused via the linker to the N-terminus of the V_{L} region of (a). In some embodiments, (b) is fused via the linker to the C-terminus of (a), optionally the C-terminus of an Fc region of (a).

In some embodiments, the linker is a flexible, stable linker, optionally selected from Table L1. In some embodiments, the linker is a flexible, cleavable linker, optionally selected from Table L2. In some embodiments, the cleavable linker comprises a protease cleavage site, or is a low pH-sensitive linker. In some embodiments, the protease cleavage site is cleavable by a protease selected from one or more of a metalloprotease, a serine protease, a cysteine protease, and an aspartic acid protease. In some embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B. In some embodiments, the linker is about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

In some embodiments, binding of (a) to high density denatured collagen in a tissue in vivo, optionally a cancer tissue, increases binding of (b) to its target cell surface receptor or ligand, optionally by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control such as the absence or reduced levels of high density denatured collagen. In some embodiments, the cell surface receptor is on the surface of an immune cell or a cancer cell, optionally wherein the immune cell is selected from one or more of a T cell, a B cell, a natural killer cell, a monocyte, and a macrophage.

In some embodiments, the antibody, or antigen-binding fragment thereof, is a monoclonal antibody and/or a humanized antibody, including wherein the antibody, or antigen-binding fragment thereof, is a whole antibody, a fragment antigen-binding domain (Fab), a F(ab′)2 domain, a single-chain variable fragment (scFv), a dimeric single-chain variable fragment (di-scFv), a single domain antibody (sdAb), or a bi-specific antibody. In some embodiments, the fusion protein, optionally bispecific antibody, comprises, consists, or consists essentially of an amino acid sequence at least 80, 85, 90, 95, 97, 98, 99, or 100% identical to a sequence selected from Table S10.

Also included are recombinant nucleic acid molecules encoding a fusion protein described herein. Some embodiments include a vector comprising the recombinant nucleic acid molecule.

Also included are host cells comprising the recombinant nucleic acid molecules and/or the vectors described herein.

Some embodiments include methods of producing a fusion protein, comprising culturing a host cell described herein under culture conditions suitable for the expression of the fusion protein, and isolating the fusion protein from the culture.

Some embodiments include a pharmaceutical composition, comprising a fusion protein described herein, and a pharmaceutically acceptable carrier.

Also included are methods of treating disease in a subject in need thereof, comprising administering to the subject a therapeutically effective amount of a pharmaceutical composition described herein. In some embodiments, the disease is selected from one or more of a cancer, a viral infection, and an immune disorder.

In some embodiments, the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, the viral infection is selected from one or more of human immunodeficiency virus (HIV), hepatitis A, hepatitis B, hepatitis C, hepatitis E, caliciviruses associated diarrhoea, rotavirus diarrhoea, Haemophilus influenzae B pneumonia and invasive disease, influenza, measles, mumps, rubella, parainfluenza associated pneumonia, respiratory syncytial virus (RSV) pneumonia, severe acute respiratory syndrome (SARS), human papillomavirus, herpes simplex type 2 genital ulcers, dengue fever, Japanese encephalitis, tick-borne encephalitis, West-Nile virus associated disease, yellow fever, Epstein-Barr virus, Lassa fever, Crimean-Congo haemorrhagic fever, Ebola haemorrhagic fever, Marburg haemorrhagic fever, Rabies, Rift Valley fever, smallpox, upper and lower respiratory infections, and poliomyelitis, optionally wherein the subject is HIV-positive.

In some embodiments, the effector domain has immune cell-stimulating activity, and wherein immune disorder is selected from one or more of type 1 diabetes, vasculitis, and an immunodeficiency. In some embodiments, the effector domain has immune cell-inhibitory activity, and wherein the immune disorder is an autoimmune and/or inflammatory disease, optionally multiple sclerosis. In some embodiments, following administration, binding of (a) to high density denatured collagen in a tissue in vivo, optionally a cancer tissue, increases binding of the effector domain to its target cell surface receptor or ligand, optionally by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control such as the absence or reduced levels of high density denatured collagen. In some embodiments, the cell surface receptor is on the surface of an immune cell or a cancer cell, optionally wherein the immune cell is selected from one or more of a T cell, a B cell, a natural killer cell, a monocyte, and a macrophage.

In some embodiments, the effector domain has immune cell-stimulatory activity, and wherein administration of the fusion protein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response. In some embodiments, the effector domain has immune cell-stimulatory activity or cytocidal activity, and wherein administration of the fusion protein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing. In some embodiments, the effector domain has immune cell-inhibitory activity, and wherein administration of the fusion protein reduces an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is associated with an inflammatory and/or autoimmune disease.

In some embodiments, the pharmaceutical composition is administered to the subject by parenteral administration. In some embodiments, the parenteral administration is intravenous administration.

Also included is the use of a pharmaceutical composition described herein in the preparation of a medicament for treating a disease in a subject, optionally wherein the disease is a cancer, a viral infection, or an immune disorder, optionally type 1 diabetes, vasculitis, an immunodeficiency, an inflammatory disease, or an autoimmune disease.

Also included is a pharmaceutical composition described herein for use in treating a disease in a subject, optionally wherein the disease is a cancer, a viral infection, or an immune disorder, optionally type 1 diabetes, vasculitis, an immunodeficiency, an inflammatory disease, or an autoimmune disease.

Certain embodiments include an isolated antibody, or an antigen-binding fragment thereof, which specifically binds to a denatured human collagen type I, II, III, IV, and/or V polypeptide at a cryptic collagen epitope, and comprises,

a heavy chain variable (V_{H}) region that comprises complementary determining region V_{H}CDR1, V_{H}CDR2, and V_{H}CDR3 sequences selected from Table A1 and variants thereof which specifically bind to the human collagen polypeptide at the cryptic collagen epitope; and

a light chain variable (V_{L}) region that comprises complementary determining region V_{L}CDR1, V_{L}CDR2, and V_{L}CDR3 sequences selected from Table A1 and variants thereof which specifically bind to the human collagen polypeptide at the cryptic collagen epitope,

excluding the HU177 and HUIV26.

In some embodiments,

the V_{H}CDR1, a V_{H}CDR2, and V_{H}CDR3 regions respectively comprise SEQ ID NOs: 1-3; and the V_{L}CDR1, V_{L}CDR2, and V_{L}CDR3 regions respectively comprise SEQ ID NOs: 4-6;

the V_{H}CDR1, a V_{H}CDR2, and V_{H}CDR3 regions respectively comprise SEQ ID NOs: 7-9; and the V_{L}CDR1, V_{L}CDR2, and V_{L}CDR3 regions respectively comprise SEQ ID NOs: 10-12;

the V_{H}CDR1, a V_{H}CDR2, and V_{H}CDR3 regions respectively comprise SEQ ID NOs: 13-15; and the V_{L}CDR1, V_{L}CDR2, and V_{L}CDR3 regions respectively comprise SEQ ID NOs: 16-18;

the V_{H}CDR1, a V_{H}CDR2, and V_{H}CDR3 regions respectively comprise SEQ ID NOs: 19-21; and the V_{L}CDR1, V_{L}CDR2, and V_{L}CDR3 regions respectively comprise SEQ ID NOs: 22-24;

the V_{H}CDR1, a V_{H}CDR2, and V_{H}CDR3 regions respectively comprise SEQ ID NOs: 25-27; and the V_{L}CDR1, V_{L}CDR2, and V_{L}CDR3 regions respectively comprise SEQ ID NOs: 28-30;

the V_{H}CDR1, a V_{H}CDR2, and V_{H}CDR3 regions respectively comprise SEQ ID NOs: 31-33; and the V_{L}CDR1, V_{L}CDR2, and V_{L}CDR3 regions respectively comprise SEQ ID NOs: 34-36;

the V_{H}CDR1, a V_{H}CDR2, and V_{H}CDR3 regions respectively comprise SEQ ID NOs: 37-39; and the V_{L}CDR1, V_{L}CDR2, and V_{L}CDR3 regions respectively comprise SEQ ID NOs: 40-42;

the V_{H}CDR1, a V_{H}CDR2, and V_{H}CDR3 regions respectively comprise SEQ ID NOs: 43-45; and the V_{L}CDR1, V_{L}CDR2, and V_{L}CDR3 regions respectively comprise SEQ ID NOs: 46-48;

the V_{H}CDR1, a V_{H}CDR2, and V_{H}CDR3 regions respectively comprise SEQ ID NOs: 49-51; and the V_{L}CDR1, V_{L}CDR2, and V_{L}CDR3 regions respectively comprise SEQ ID NOs: 52-54;

the V_{H}CDR1, a V_{H}CDR2, and V_{H}CDR3 regions respectively comprise SEQ ID NOs: 55-57; and the V_{L}CDR1, V_{L}CDR2, and V_{L}CDR3 regions respectively comprise SEQ ID NOs: 58-60;

the V_{H}CDR1, a V_{H}CDR2, and V_{H}CDR3 regions respectively comprise SEQ ID NOs: 61-63; and the V_{L}CDR1, V_{L}CDR2, and V_{L}CDR3 regions respectively comprise SEQ ID NOs: 64-66;

the V_{H}CDR1, a V_{H}CDR2, and V_{H}CDR3 regions respectively comprise SEQ ID NOs: 67-69; and the V_{L}CDR1, V_{L}CDR2, and V_{L}CDR3 regions respectively comprise SEQ ID NOs: 70-72;

the V_{H}CDR1, a V_{H}CDR2, and V_{H}CDR3 regions respectively comprise SEQ ID NOs: 73-75; and the V_{L}CDR1, V_{L}CDR2, and V_{L}CDR3 regions respectively comprise SEQ ID NOs: 76-78;

the V_{H}CDR1, a V_{H}CDR2, and V_{H}CDR3 regions respectively comprise SEQ ID NOs: 79-81; and the V_{L}CDR1, V_{L}CDR2, and V_{L}CDR3 regions respectively comprise SEQ ID NOs: 82-84;

the V_{H}CDR1, a V_{H}CDR2, and V_{H}CDR3 regions respectively comprise SEQ ID NOs: 85-87; and the V_{L}CDR1, V_{L}CDR2, and V_{L}CDR3 regions respectively comprise SEQ ID NOs: 88-90;

the V_{H}CDR1, a V_{H}CDR2, and V_{H}CDR3 regions respectively comprise SEQ ID NOs: 91-93; and the V_{L}CDR1, V_{L}CDR2, and V_{L}CDR3 regions respectively comprise SEQ ID NOs: 94-96;

the V_{H}CDR1, a V_{H}CDR2, and V_{H}CDR3 regions respectively comprise SEQ ID NOs: 97-99; and the V_{L}CDR1, V_{L}CDR2, and V_{L}CDR3 regions respectively comprise SEQ ID NOs: 100-102;

the V_{H}CDR1, a V_{H}CDR2, and V_{H}CDR3 regions respectively comprise SEQ ID NOs: 103-105; and the V_{L}CDR1, V_{L}CDR2, and V_{L}CDR3 regions respectively comprise SEQ ID NOs: 106-108;

the V_{H}CDR1, a V_{H}CDR2, and V_{H}CDR3 regions respectively comprise SEQ ID NOs: 109-111; and the V_{L}CDR1, V_{L}CDR2, and V_{L}CDR3 regions respectively comprise SEQ ID NOs: 112-114;

the V_{H}CDR1, a V_{H}CDR2, and V_{H}CDR3 regions respectively comprise SEQ ID NOs: 115-117; and the V_{L}CDR1, V_{L}CDR2, and V_{L}CDR3 regions respectively comprise SEQ ID NOs: 118-120;

the V_{H}CDR1, a V_{H}CDR2, and V_{H}CDR3 regions respectively comprise SEQ ID NOs: 121-123; and the V_{L}CDR1, V_{L}CDR2, and V_{L}CDR3 regions respectively comprise SEQ ID NOs: 124-126;

the V_{H}CDR1, a V_{H}CDR2, and V_{H}CDR3 regions respectively comprise SEQ ID NOs: 127-129; and the V_{L}CDR1, V_{L}CDR2, and V_{L}CDR3 regions respectively comprise SEQ ID NOs: 130-132;

the V_{H}CDR1, a V_{H}CDR2, and V_{H}CDR3 regions respectively comprise SEQ ID NOs: 133-135; and the V_{L}CDR1, V_{L}CDR2, and V_{L}CDR3 regions respectively comprise SEQ ID NOs: 136-138;

the V_{H}CDR1, a V_{H}CDR2, and V_{H}CDR3 regions respectively comprise SEQ ID NOs: 139-141; and the V_{L}CDR1, V_{L}CDR2, and V_{L}CDR3 regions respectively comprise SEQ ID NOs: 142-144;

the V_{H}CDR1, a V_{H}CDR2, and V_{H}CDR3 regions respectively comprise SEQ ID NOs: 145-147; and the V_{L}CDR1, V_{L}CDR2, and V_{L}CDR3 regions respectively comprise SEQ ID NOs: 148-150;

the V_{H}CDR1, a V_{H}CDR2, and V_{H}CDR3 regions respectively comprise SEQ ID NOs: 151-153; and the V_{L}CDR1, V_{L}CDR2, and V_{L}CDR3 regions respectively comprise SEQ ID NOs: 154-156;

the V_{H}CDR1, a V_{H}CDR2, and V_{H}CDR3 regions respectively comprise SEQ ID NOs: 157-159; and the V_{L}CDR1, V_{L}CDR2, and V_{L}CDR3 regions respectively comprise SEQ ID NOs: 160-162;

the V_{H}CDR1, a V_{H}CDR2, and V_{H}CDR3 regions respectively comprise SEQ ID NOs: 163-165; and the V_{L}CDR1, V_{L}CDR2, and V_{L}CDR3 regions respectively comprise SEQ ID NOs: 166-168;

the V_{H}CDR1, a V_{H}CDR2, and V_{H}CDR3 regions respectively comprise SEQ ID NOs: 169-171; and the V_{L}CDR1, V_{L}CDR2, and V_{L}CDR3 regions respectively comprise SEQ ID NOs: 172-174;

the V_{H}CDR1, a V_{H}CDR2, and V_{H}CDR3 regions respectively comprise SEQ ID NOs: 175-177; and the V_{L}CDR1, V_{L}CDR2, and V_{L}CDR3 regions respectively comprise SEQ ID NOs: 178-180;

the V_{H}CDR1, a V_{H}CDR2, and V_{H}CDR3 regions respectively comprise SEQ ID NOs: 181-183; and the V_{L}CDR1, V_{L}CDR2, and V_{L}CDR3 regions respectively comprise SEQ ID NOs: 184-186;

the V_{H}CDR1, a V_{H}CDR2, and V_{H}CDR3 regions respectively comprise SEQ ID NOs: 187-189; and the V_{L}CDR1, V_{L}CDR2, and V_{L}CDR3 regions respectively comprise SEQ ID NOs: 190-192;

the V_{H}CDR1, a V_{H}CDR2, and V_{H}CDR3 regions respectively comprise SEQ ID NOs: 193-195; and the V_{L}CDR1, V_{L}CDR2, and V_{L}CDR3 regions respectively comprise SEQ ID NOs: 196-198;

the V_{H}CDR1, a V_{H}CDR2, and V_{H}CDR3 regions respectively comprise SEQ ID NOs: 199-201; and the V_{L}CDR1, V_{L}CDR2, and V_{L}CDR3 regions respectively comprise SEQ ID NOs: 202-204;

the V_{H}CDR1, a V_{H}CDR2, and V_{H}CDR3 regions respectively comprise SEQ ID NOs: 205-207; and the V_{L}CDR1, V_{L}CDR2, and V_{L}CDR3 regions respectively comprise SEQ ID NOs: 208-210;

the V_{H}CDR1, a V_{H}CDR2, and V_{H}CDR3 regions respectively comprise SEQ ID NOs: 211-213; and the V_{L}CDR1, V_{L}CDR2, and V_{L}CDR3 regions respectively comprise SEQ ID NOs: 214-216;

the V_{H}CDR1, a V_{H}CDR2, and V_{H}CDR3 regions respectively comprise SEQ ID NOs: 217-219; and the V_{L}CDR1, V_{L}CDR2, and V_{L}CDR3 regions respectively comprise SEQ ID NOs: 220-222;

the V_{H}CDR1, a V_{H}CDR2, and V_{H}CDR3 regions respectively comprise SEQ ID NOs: 223-225; and the V_{L}CDR1, V_{L}CDR2, and V_{L}CDR3 regions respectively comprise SEQ ID NOs: 226-228;

the V_{H}CDR1, a V_{H}CDR2, and V_{H}CDR3 regions respectively comprise SEQ ID NOs: 229-231; and the V_{L}CDR1, V_{L}CDR2, and V_{L}CDR3 regions respectively comprise SEQ ID NOs: 232-234;

the V_{H}CDR1, a V_{H}CDR2, and V_{H}CDR3 regions respectively comprise SEQ ID NOs: 235-237; and the V_{L}CDR1, V_{L}CDR2, and V_{L}CDR3 regions respectively comprise SEQ ID NOs: 238-240;

the V_{H}CDR1, a V_{H}CDR2, and V_{H}CDR3 regions respectively comprise SEQ ID NOs: 241-243; and the V_{L}CDR1, V_{L}CDR2, and V_{L}CDR3 regions respectively comprise SEQ ID NOs: 244-246;

the V_{H}CDR1, a V_{H}CDR2, and V_{H}CDR3 regions respectively comprise SEQ ID NOs: 247-249; and the V_{L}CDR1, V_{L}CDR2, and V_{L}CDR3 regions respectively comprise SEQ ID NOs: 250-252; or

the V_{H}CDR1, a V_{H}CDR2, and V_{H}CDR3 regions respectively comprise SEQ ID NOs: 253-255; and the V_{L}CDR1, V_{L}CDR2, and V_{L}CDR3 regions respectively comprise SEQ ID NOs: 256-258;

including variants thereof that have 1, 2, 3, 4, 5, or 6 total alterations in one or more of the CDR regions and specifically bind to the human collagen polypeptide at the cryptic collagen epitope.

In some embodiments,

the heavy chain is at least 80, 85, 90, 95, 97, 98, 99, or 100% identical to a sequence selected from Table A2, including wherein the heavy chain has 1, 2, 3, 4, 5, or 6 alterations in one or more framework regions, and

the light chain is at least 80, 85, 90, 95, 97, 98, 99, or 100% identical to a sequence selected from Table A2, including wherein the light chain has 1, 2, 3, 4, 5, or 6 alterations in one or more framework regions,

excluding the HU177 and HUIV26 antibodies.

In some embodiments,

the heavy chain comprises a sequence at least 80, 85, 90, 95, 97, 98, 99, or 100% identical to SEQ ID NO: 271, and the light chain comprises a sequence at least 80, 85, 90, 95, 97, 98, 99, or 100% identical to SEQ ID NO: 272;

the heavy chain comprises a sequence at least 80, 85, 90, 95, 97, 98, 99, or 100% identical to SEQ ID NO: 273, and the light chain comprises a sequence at least 80, 85, 90, 95, 97, 98, 99, or 100% identical to SEQ ID NO: 274;

the heavy chain comprises a sequence at least 80, 85, 90, 95, 97, 98, 99, or 100% identical to SEQ ID NO: 275, and the light chain comprises a sequence at least 80, 85, 90, 95, 97, 98, 99, or 100% identical to SEQ ID NO: 276;

the heavy chain comprises a sequence at least 80, 85, 90, 95, 97, 98, 99, or 100% identical to SEQ ID NO: 277, and the light chain comprises a sequence at least 80, 85, 90, 95, 97, 98, 99, or 100% identical to SEQ ID NO: 278;

the heavy chain comprises a sequence at least 80, 85, 90, 95, 97, 98, 99, or 100% identical to SEQ ID NO: 279, and the light chain comprises a sequence at least 80, 85, 90, 95, 97, 98, 99, or 100% identical to SEQ ID NO: 280;

the heavy chain comprises a sequence at least 80, 85, 90, 95, 97, 98, 99, or 100% identical to SEQ ID NO: 281, and the light chain comprises a sequence at least 80, 85, 90, 95, 97, 98, 99, or 100% identical to SEQ ID NO: 282;

the heavy chain comprises a sequence at least 80, 85, 90, 95, 97, 98, 99, or 100% identical to SEQ ID NO: 283, and the light chain comprises a sequence at least 80, 85, 90, 95, 97, 98, 99, or 100% identical to SEQ ID NO: 284;

the heavy chain comprises a sequence at least 80, 85, 90, 95, 97, 98, 99, or 100% identical to SEQ ID NO: 285, and the light chain comprises a sequence at least 80, 85, 90, 95, 97, 98, 99, or 100% identical to SEQ ID NO: 286;

the heavy chain comprises a sequence at least 80, 85, 90, 95, 97, 98, 99, or 100% identical to SEQ ID NO: 287, and the light chain comprises a sequence at least 80, 85, 90, 95, 97, 98, 99, or 100% identical to SEQ ID NO: 288;

the heavy chain comprises a sequence at least 80, 85, 90, 95, 97, 98, 99, or 100% identical to SEQ ID NO: 289, and the light chain comprises a sequence at least 80, 85, 90, 95, 97, 98, 99, or 100% identical to SEQ ID NO: 290;

the heavy chain comprises a sequence at least 80, 85, 90, 95, 97, 98, 99, or 100% identical to SEQ ID NO: 291, and the light chain comprises a sequence at least 80, 85, 90, 95, 97, 98, 99, or 100% identical to SEQ ID NO: 292;

the heavy chain comprises a sequence at least 80, 85, 90, 95, 97, 98, 99, or 100% identical to SEQ ID NO: 293, and the light chain comprises a sequence at least 80, 85, 90, 95, 97, 98, 99, or 100% identical to SEQ ID NO: 294;

the heavy chain comprises a sequence at least 80, 85, 90, 95, 97, 98, 99, or 100% identical to SEQ ID NO: 295, and the light chain comprises a sequence at least 80, 85, 90, 95, 97, 98, 99, or 100% identical to SEQ ID NO: 296;

the heavy chain comprises a sequence at least 80, 85, 90, 95, 97, 98, 99, or 100% identical to SEQ ID NO: 297, and the light chain comprises a sequence at least 80, 85, 90, 95, 97, 98, 99, or 100% identical to SEQ ID NO: 298;

the heavy chain comprises a sequence at least 80, 85, 90, 95, 97, 98, 99, or 100% identical to SEQ ID NO: 299, and the light chain comprises a sequence at least 80, 85, 90, 95, 97, 98, 99, or 100% identical to SEQ ID NO: 300;

the heavy chain comprises a sequence at least 80, 85, 90, 95, 97, 98, 99, or 100% identical to SEQ ID NO: 301, and the light chain comprises a sequence at least 80, 85, 90, 95, 97, 98, 99, or 100% identical to SEQ ID NO: 302;

the heavy chain comprises a sequence at least 80, 85, 90, 95, 97, 98, 99, or 100% identical to SEQ ID NO: 303, and the light chain comprises a sequence at least 80, 85, 90, 95, 97, 98, 99, or 100% identical to SEQ ID NO: 304;

the heavy chain comprises a sequence at least 80, 85, 90, 95, 97, 98, 99, or 100% identical to SEQ ID NO: 305, and the light chain comprises a sequence at least 80, 85, 90, 95, 97, 98, 99, or 100% identical to SEQ ID NO: 306;

the heavy chain comprises a sequence at least 80, 85, 90, 95, 97, 98, 99, or 100% identical to SEQ ID NO: 307, and the light chain comprises a sequence at least 80, 85, 90, 95, 97, 98, 99, or 100% identical to SEQ ID NO: 308;

the heavy chain comprises a sequence at least 80, 85, 90, 95, 97, 98, 99, or 100% identical to SEQ ID NO: 309, and the light chain comprises a sequence at least 80, 85, 90, 95, 97, 98, 99, or 100% identical to SEQ ID NO: 310;

the heavy chain comprises a sequence at least 80, 85, 90, 95, 97, 98, 99, or 100% identical to SEQ ID NO: 311, and the light chain comprises a sequence at least 80, 85, 90, 95, 97, 98, 99, or 100% identical to SEQ ID NO: 312;

the heavy chain comprises a sequence at least 80, 85, 90, 95, 97, 98, 99, or 100% identical to SEQ ID NO: 313, and the light chain comprises a sequence at least 80, 85, 90, 95, 97, 98, 99, or 100% identical to SEQ ID NO: 314;

the heavy chain comprises a sequence at least 80, 85, 90, 95, 97, 98, 99, or 100% identical to SEQ ID NO: 315, and the light chain comprises a sequence at least 80, 85, 90, 95, 97, 98, 99, or 100% identical to SEQ ID NO: 316;

the heavy chain comprises a sequence at least 80, 85, 90, 95, 97, 98, 99, or 100% identical to SEQ ID NO: 317, and the light chain comprises a sequence at least 80, 85, 90, 95, 97, 98, 99, or 100% identical to SEQ ID NO: 318;

the heavy chain comprises a sequence at least 80, 85, 90, 95, 97, 98, 99, or 100% identical to SEQ ID NO: 319, and the light chain comprises a sequence at least 80, 85, 90, 95, 97, 98, 99, or 100% identical to SEQ ID NO: 320;

the heavy chain comprises a sequence at least 80, 85, 90, 95, 97, 98, 99, or 100% identical to SEQ ID NO: 321, and the light chain comprises a sequence at least 80, 85, 90, 95, 97, 98, 99, or 100% identical to SEQ ID NO: 322;

the heavy chain comprises a sequence at least 80, 85, 90, 95, 97, 98, 99, or 100% identical to SEQ ID NO: 323, and the light chain comprises a sequence at least 80, 85, 90, 95, 97, 98, 99, or 100% identical to SEQ ID NO: 324;

the heavy chain comprises a sequence at least 80, 85, 90, 95, 97, 98, 99, or 100% identical to SEQ ID NO: 325, and the light chain comprises a sequence at least 80, 85, 90, 95, 97, 98, 99, or 100% identical to SEQ ID NO: 326;

the heavy chain comprises a sequence at least 80, 85, 90, 95, 97, 98, 99, or 100% identical to SEQ ID NO: 327, and the light chain comprises a sequence at least 80, 85, 90, 95, 97, 98, 99, or 100% identical to SEQ ID NO: 328;

the heavy chain comprises a sequence at least 80, 85, 90, 95, 97, 98, 99, or 100% identical to SEQ ID NO: 329, and the light chain comprises a sequence at least 80, 85, 90, 95, 97, 98, 99, or 100% identical to SEQ ID NO: 330;

the heavy chain comprises a sequence at least 80, 85, 90, 95, 97, 98, 99, or 100% identical to SEQ ID NO: 331, and the light chain comprises a sequence at least 80, 85, 90, 95, 97, 98, 99, or 100% identical to SEQ ID NO: 332;

the heavy chain comprises a sequence at least 80, 85, 90, 95, 97, 98, 99, or 100% identical to SEQ ID NO: 333, and the light chain comprises a sequence at least 80, 85, 90, 95, 97, 98, 99, or 100% identical to SEQ ID NO: 334;

the heavy chain comprises a sequence at least 80, 85, 90, 95, 97, 98, 99, or 100% identical to SEQ ID NO: 335, and the light chain comprises a sequence at least 80, 85, 90, 95, 97, 98, 99, or 100% identical to SEQ ID NO: 336;

the heavy chain comprises a sequence at least 80, 85, 90, 95, 97, 98, 99, or 100% identical to SEQ ID NO: 337, and the light chain comprises a sequence at least 80, 85, 90, 95, 97, 98, 99, or 100% identical to SEQ ID NO: 338;

the heavy chain comprises a sequence at least 80, 85, 90, 95, 97, 98, 99, or 100% identical to SEQ ID NO: 339, and the light chain comprises a sequence at least 80, 85, 90, 95, 97, 98, 99, or 100% identical to SEQ ID NO: 340;

the heavy chain comprises a sequence at least 80, 85, 90, 95, 97, 98, 99, or 100% identical to SEQ ID NO: 341, and the light chain comprises a sequence at least 80, 85, 90, 95, 97, 98, 99, or 100% identical to SEQ ID NO: 342;

the heavy chain comprises a sequence at least 80, 85, 90, 95, 97, 98, 99, or 100% identical to SEQ ID NO: 343, and the light chain comprises a sequence at least 80, 85, 90, 95, 97, 98, 99, or 100% identical to SEQ ID NO: 344;

the heavy chain comprises a sequence at least 80, 85, 90, 95, 97, 98, 99, or 100% identical to SEQ ID NO: 345, and the light chain comprises a sequence at least 80, 85, 90, 95, 97, 98, 99, or 100% identical to SEQ ID NO: 346;

the heavy chain comprises a sequence at least 80, 85, 90, 95, 97, 98, 99, or 100% identical to SEQ ID NO: 347, and the light chain comprises a sequence at least 80, 85, 90, 95, 97, 98, 99, or 100% identical to SEQ ID NO: 348;

the heavy chain comprises a sequence at least 80, 85, 90, 95, 97, 98, 99, or 100% identical to SEQ ID NO: 349, and the light chain comprises a sequence at least 80, 85, 90, 95, 97, 98, 99, or 100% identical to SEQ ID NO: 350;

the heavy chain comprises a sequence at least 80, 85, 90, 95, 97, 98, 99, or 100% identical to SEQ ID NO: 351, and the light chain comprises a sequence at least 80, 85, 90, 95, 97, 98, 99, or 100% identical to SEQ ID NO: 352;

the heavy chain comprises a sequence at least 80, 85, 90, 95, 97, 98, 99, or 100% identical to SEQ ID NO: 353, and the light chain comprises a sequence at least 80, 85, 90, 95, 97, 98, 99, or 100% identical to SEQ ID NO: 354; or

the heavy chain comprises a sequence at least 80, 85, 90, 95, 97, 98, 99, or 100% identical to SEQ ID NO: 355, and the light chain comprises a sequence at least 80, 85, 90, 95, 97, 98, 99, or 100% identical to SEQ ID NO: 356.

In some embodiments, the isolated antibody, or antigen-binding fragment thereof, comprises a V_{H} region or heavy chain comprising:
an HFR1 sequence selected from SEQ ID NOs: 357-361;
an HFR2 sequence selected from SEQ ID NOs: 362-366;
an HFR3 sequence selected from SEQ ID NOs: 367-369;
an HFR4 sequence set forth in SEQ ID NO: 370;
a V_{H}CDR1 sequence selected from SEQ ID NOs: 371-372;
a V_{H}CDR2 sequence selected from SEQ ID NOs: 373-374; and
a V_{H}CDR3 sequence selected from SEQ ID NO: 375,

and/or a V_{L} region or light chain comprising:
an LFR1 sequence set forth in SEQ ID NO: 376;
an LFR2 sequence set forth in SEQ ID NO: 377;
an LFR3 sequence set forth in SEQ ID NO: 378;
an LFR4 sequence set forth in SEQ ID NO: 379;
a V_{L}CDR1 sequence selected from SEQ ID NOs: 380-385;
a V_{L}CDR2 sequence set forth in SEQ ID NO: 386; and
a V_{L}CDR3 sequence selected from SEQ ID NOs: 387-388,

wherein the antibody, or antigen-binding fragment thereof, specifically binds to a human denatured collagen, or comprising:
an HFR1 sequence selected from SEQ ID NOs: 389-390;
an HFR2 sequence selected from SEQ ID NOs: 391-392;
an HFR3 sequence set forth in SEQ ID NO: 393;
an HFR4 sequence set forth in SEQ ID NO: 394;
a V_{H}CDR1 sequence selected from SEQ ID NOs: 395-397;
a V_{H}CDR2 sequence selected from SEQ ID NOs: 398-401; and
a V_{H}CDR3 sequence selected from SEQ ID NOs: 402-405,

and/or a VL region or light chain comprising:
an LFR1 sequence set forth in SEQ ID NO: 406;
an LFR2 sequence set forth in SEQ ID NO: 407;
an LFR3 sequence set forth in SEQ ID NO: 408;
an LFR4 sequence set forth in SEQ ID NO: 409;
a V_{L}CDR1 sequence selected from SEQ ID NOs: 410-412;
a V_{L}CDR2 set forth in SEQ ID NO: 413; and
a V_{L}CDR3 set forth in SEQ ID NO: 414,

wherein the antibody, or antigen-binding fragment thereof, specifically binds to a human denatured collagen.

In some embodiments, the isolated antibody, or antigen-binding fragment thereof, preferentially binds to the denatured human collagen type I, II, III, IV, and/or V polypeptide relative to a corresponding native human collagen polypeptide, optionally wherein the antibody, or antigen-binding fragment thereof, has about or at least about 2, 3, 4, 5, 6, 7, 8, 9, 10, 15, 20, 25, 30, 35, 40, 50, 60, 70, 80, 90, or 100-fold higher binding affinity for the denatured human collagen polypeptide than it has for the corresponding native human collagen polypeptide.

In some embodiments, the isolated antibody, or antigen-binding fragment thereof, specifically binds to the denatured human collagen type I, II, III, IV, and V polypeptides at the HU177 cryptic collagen epitope, optionally wherein the cryptic collagen epitope comprises PGXP, LPGXPG (SEQ ID NO: _, and/or GPP′GXP′G (SEQ ID NO: _), wherein X is any amino acid, and wherein P′ is hydroxylproline. In some embodiments, the isolated antibody, or antigen-binding fragment thereof, specifically binds to the denatured human collagen type IV polypeptide at the HUIV26 cryptic collagen epitope.

In some embodiments, the isolated antibody, or antigen-binding fragment thereof, is a monoclonal antibody and/or a humanized antibody, or a fragment antigen-binding domain (Fab), a F(ab′)2 domain, or a whole antibody. In some embodiments, the isolated antibody, or antigen-binding fragment thereof, is fused via an optional linker to an effector domain.

Also included are recombinant nucleic acid molecules encoding an antibody, or antigen-binding fragment thereof, described herein, vectors comprising the recombinant nucleic acid molecules, and host cells comprising the recombinant nucleic acid molecules and/or the vectors.

Certain embodiments include methods of producing an antibody, or antigen-binding fragment thereof, comprising culturing a host cell described herein under culture conditions suitable for the expression of the antibody, or antigen-binding fragment thereof, and isolating the antibody, or antigen-binding fragment thereof, from the culture.

Certain embodiments include a pharmaceutical or diagnostic composition, comprising an antibody, or antigen-binding fragment thereof, described herein, and a pharmaceutically-acceptable carrier.

### BRIEF DESCRIPTION OF THE DRAWINGS

FIGS. 1A-1B provide the structural features of an exemplary fusion protein, where the C-terminus of an effector domain is fused to the N-terminus of the light chain (1A) or heavy chain (1B) of an immunoglobulin antigen binding domain (ABD) that binds to human collagen at a cryptic collagen epitope. The N-terminus of an effector domain can also be fused to the C-terminus of the ABD or an Fc region.

FIGS. 2A-2C illustrate how binding of the anti-collagen ABD to high density structures of denatured tissue collagen (e.g., in vivo) in the target environment increases the local concentration and thus the biological activity of the effector domain in a spatially-regulated manner. In FIGS. 2B-2C, the N-terminus of an effector domain is fused to the C-terminus of the Fc region. FIG. 2C further illustrates the fusion of only one effector domain, and an optional knob-hole feature in the Fc regions of the antibody.

FIGS. 3A-3B show non-reduced SDS-PAGE (3A) and reduced SDS-PAGE (3B) of purified collagen antibodies. “M” on the figures represents the protein standard marker.

FIGS. 4A-4D illustrate representative HPLC analysis results of exemplary antibodies purified using protein affinity chromatography.

FIGS. 5A-5P show dose dependent binding activity and specificity of purified collagen antibodies against human collagens; “dn-hu-collagen I” refers to thermally denatured human collagen I.

FIGS. 6A-6F show the binding specificity of purified collagen antibodies against denatured and non-denatured human collagens; “n-hu-collagen” refers to native human collagen.

FIGS. 7A-7H show the binding activity and specificity of purified collagen antibodies against human and mouse collagens; “dn-hu-collagen” refers to thermally denatured human collagen I; “n-hu-collagen” refers to native human collagen.

FIGS. 8A-8B show non-reduced (8A) and reduced (8B) SDS-PAGE of purified collagen antibody proIL-2 fusion proteins. “M” on the figures represents the protein standard marker.

FIGS. 9A-9F illustrate representative HPLC analysis profiles of purified antibodies.

FIGS. 10A-10J show dose dependent binding activity and specificity of purified proIL-2 fusion proteins with respect to collagens.

FIGS. 11A-11B illustrate the biological activity of anti-collagen antibodies and proIL-2 fusion proteins on M-07e proliferation determined by a colorimetric assay (Cell Counting Kit-8 (CCK-8)). Fusion proteins were digested to completion using proteases as indicted.

FIGS. 12A-12B show non-reduced (12A) and reduced (12B) SDS-PAGE analysis of purified collagen targeted proIL-15 fusion proteins. “M” on the figures represents the protein standard marker.

FIGS. 13A-13F illustrate representative HPLC analysis results of purified antibodies.

FIGS. 14A-14L show the binding activity and specificity of purified proIL-15 fusion proteins with respect to collagens.

FIGS. 15A-15B illustrate the biological activity of proIL-15 fusion proteins on M-07e proliferation determined by a colorimetric assay (Cell Counting Kit-8 (CCK-8)).

FIGS. 16A-16E show the amino acid sequences of certain anti-cryptic collagen epitope antibodies described herein. FIGS. 16A-16B and 16D show an alignment of the heavy chains, and FIGS. 16C and 16E show an alignment of the light chains (see also Table E1).

### DETAILED DESCRIPTION

Embodiments of the present disclosure relate, in part, to improved antibodies, and antigen-binding fragments thereof, which specifically bind to a denatured human collagen type I, III, II, IV, and/or V polypeptide, relative to a corresponding native collagen polypeptide, for example, at a cryptic collagen epitope (anti-denatured collagen antibodies). Certain embodiments include antibodies, and antigen-binding fragments thereof, which specifically bind to both denatured and native forms of human collagens. The improved anti-denatured collagen antibodies described herein can find utility as standalone therapeutic, diagnostic, or research agents (see, for example, Wayhudi et al., J. of Controlled Release. 240:323-331, 2016)), or as part of a fusion protein described herein.

Certain embodiments thus include a fusion protein, comprising (a) an antibody, or antigen-binding fragment thereof, which specifically binds to a denatured human collagen type I, II, III, IV, and/or V polypeptide at a cryptic collagen epitope, wherein (a) is fused via a linker to; (b) an effector domain such as a cytokine or other ligand, an immunomodulatory antibody, or antigen-binding fragment thereof (for example, an “antagonist” antibody of an immune-inhibitory molecule such as a receptor, or an “agonist” antibody of an immune-stimulatory molecule such as a receptor), or anti-cancer antibody, or antigen-binding fragment thereof. Certain fusion proteins comprise the improved anti-denatured collagen antibodies described herein, and certain fusion proteins comprise other anti-denatured collagen antibodies, including the HU177, HUIV26, or D93 antibodies (see, for example, U.S. Pat. Nos. 8,025,883; and 7,566,770; and Pernasetti et al., Int J Oncol. 29:1371-9, 2006), and antigen-binding fragments and derivatives thereof.

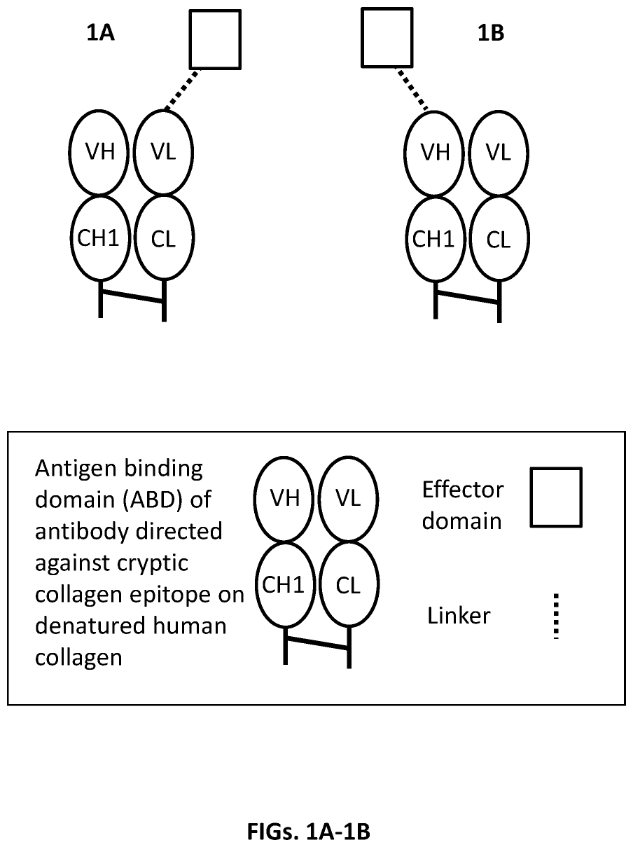

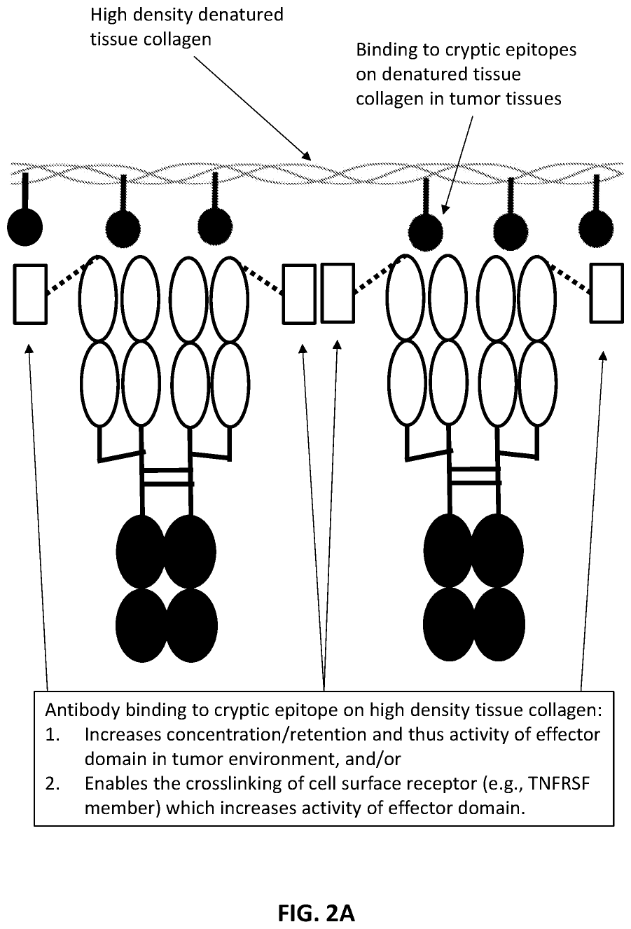

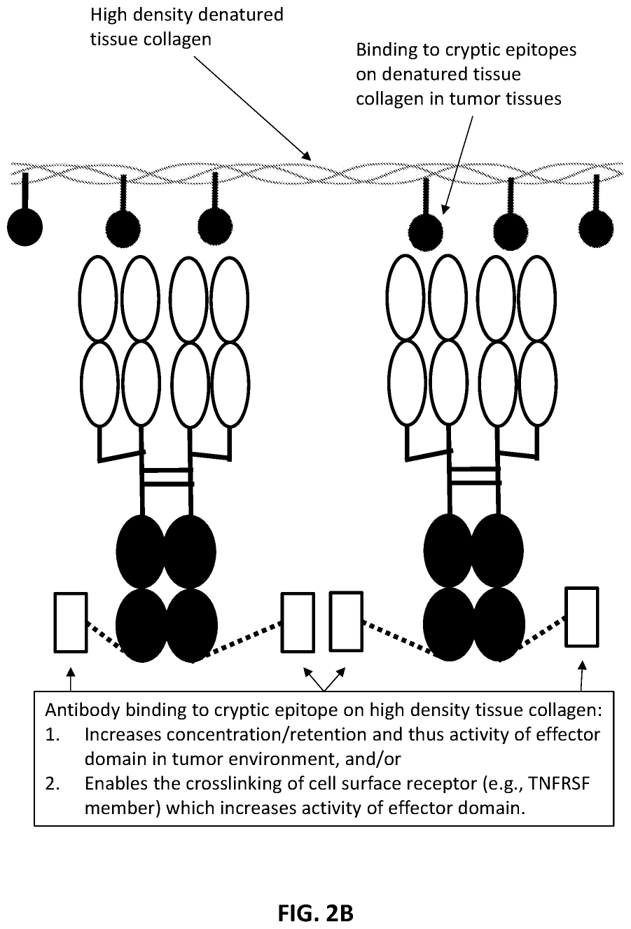

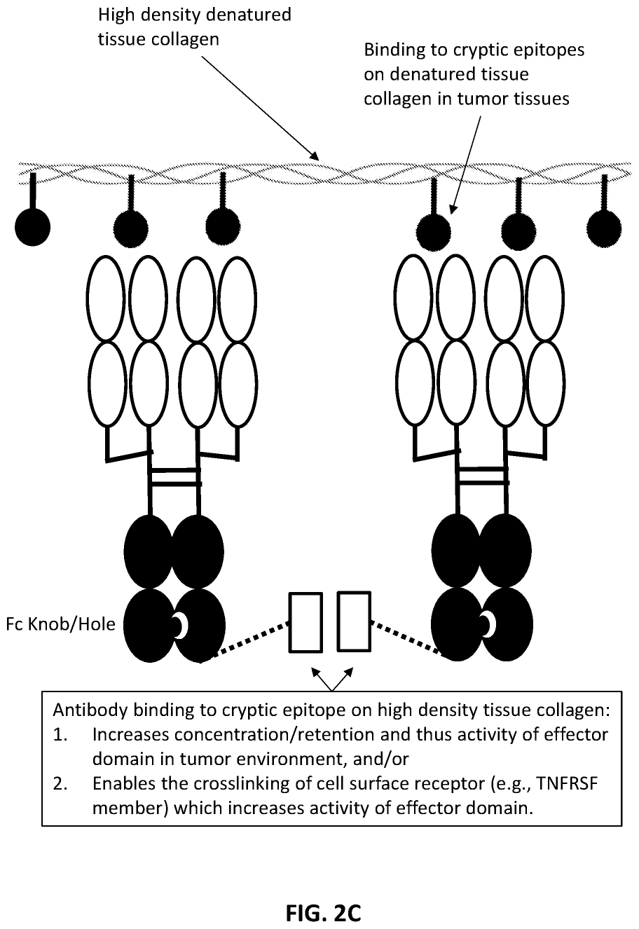

The fusion proteins can be structured in any orientation, and can have one or two or more effector domains (see FIGS. 1A-1B and FIGS. 2A-2C). For instance, in some fusion proteins the effector domain is fused via the linker to the N-terminus of the V_{H} region of (a), and in some fusion proteins the effector domain is fused via the linker to the N-terminus of the V_{L} region of (a). In some instances, the effector domain is fused via the linker to the C-terminus of (a), for example, the C-terminus of the V_{H} or V_{L} region if (a) is a fragment (e.g., Fab, scFv), or the C-terminus of an Fc region if (a) is a whole antibody or comprises only a partial Fc region. In some instances, binding of (a) to high density collagen in a tissue in vivo, for example, a cancer tissue, increases the localized concentration and/or retention of the fusion protein, and thereby increases the binding/activity of the effector domain in relation to its target, for example, its target cell surface receptor or ligand (see FIGS. 2A-2C). In particular embodiments, a fusion protein comprising an effector domain (for example, a TNF superfamily ligand, or an antibody, or antigen-binding fragment thereof, directed against a TNF superfamily receptor) mediates tumor tissue-localized cross-linking between TNF superfamily receptors, thereby increasing anti-tumor immune responses in the tumor microenvironment. These processes spatially-regulate the activity of the effector domain, to increase or optimize its activity at the desired target site, for example, a site that has a relatively high concentration of denatured tissue collagen in relation to the concentration of native tissue collagen. Exemplary anti-denatured collagen antibodies, effector domains, and linkers are described herein.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} region from antibodies that bind to a target molecule.

An “epitope” includes that portion of an antigen or other macromolecule capable of forming a binding interaction that interacts with the variable region binding pocket of an antibody, or antigen-binding fragment thereof. Such binding interaction can be manifested as an intermolecular contact with one or more amino acid residues of a CDR. Antigen binding can involve a CDR3 or a CDR3 pair. An epitope can be a linear peptide sequence (i.e., “continuous”) or can be composed of noncontiguous amino acid sequences (i.e., “conformational” or “discontinuous”). An antibody, or antigen-binding fragment thereof, can recognize one or more amino acid sequences; therefore an epitope can define more than one distinct amino acid sequence. Epitopes can be determined, for example, by peptide mapping and sequence analysis techniques well known to one of skill in the art. In particular embodiments, an epitope comprises, consists, or consists essentially of about, at least about, or no more than about 3, 4, 5, 6, 7, 8, 9, 10, 11, 12, 13, 14, 15, 16, 17, 18, 19, or 20 contiguous amino acids (i.e., a linear epitope) or non-contiguous amino acids (i.e., conformational epitope) of a reference sequence or target molecule described herein.

A “paratope”, also called an antigen-binding site, refers to the part of an immunoglobulin that recognizes and binds to an antigen or antigens. In some instances, a paratope is a small region (e.g., of about 5 to 10 amino acids) within the fragment antigen-binding (Fab) region of an immunoglobulin, and includes a set of six complementarity-determining regions (CDR loops).

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, a cell surface receptor, a cancer or immunomodulatory antigen, or an epitope or complex thereof, with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for a target molecule (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as a polypeptide or antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. In some embodiments, affinity is expressed in the terms of the half maximal effective concentration (EC₅₀), which refers to the concentration of an agent, such as an antibody, as disclosed herein, which induces a response halfway between the baseline and maximum after a specified exposure time. The EC₅₀ is commonly used as a measure of an antibody's potency.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv (scFV) antibodies are contemplated. For example, Kappa bodies (Ill et al., Prot. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (scFv) polypeptide is a covalently linked VH::VL heterodimer which is expressed from a gene fusion including VH- and VL-encoding genes linked by a peptide-encoding linker. Huston et al. (PNAS USA. 85(16):5879-5883, 1988). A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Certain embodiments include “probodies”, or antibodies where the binding site(s) are masked or otherwise inert until activated by proteolytic cleavage in target or disease tissue. Certain of these and related embodiments comprise one or more masking moieties that sterically hinder the antigen-binding site(s) of the antibody, and which are fused to the antibody via one or more proteolytically-cleavable linkers (see, for example, Polu and Lowman, Expert Opin. Biol. Ther. 14:1049-1053, 2014).

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen-binding site: antigen-binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific or multi-specific antibodies are to be used, these may be conventional bispecific antibodies, which can be manufactured in a variety of ways (Holliger and Winter, Current Opinion Biotechnol. 4:446-449, 1993), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction. Bispecific whole antibodies may be made by knobs-into-holes engineering (J. B. B. Ridgeway et al., Protein Eng., 9, 616-621, 1996).

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies of the present disclosure may take the form of a Nanobody®. Nanobodies® are encoded by single genes and are efficiently produced in almost all prokaryotic and eukaryotic hosts e.g. E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). The production process is scalable and multi-kilogram quantities of Nanobodies® have been produced. Nanobodies may be formulated as a ready-to-use solution having a long shelf life. The Nanoclone® method (see, e.g., WO 06/079372) is a proprietary method for generating Nanobodies against a desired target, based on automated high-throughput selection of B-cells.

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (e.g., fusion protein) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₅₀ of an agent (e.g., fusion protein, antibody) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅0 value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as a fusion protein can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7, 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., fusion protein, antibody) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., fusion protein, antibody) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., fusion protein, antibody) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Anti-Denatured Collagen Antibodies

Certain embodiments include an isolated antibody, or antigen-binding fragment thereof, which specifically binds to a denatured human collagen type I, II, III, IV, and/or V polypeptide at a cryptic collagen epitope. In some embodiments, the antibody, or antigen-binding fragment thereof, selectively binds to “denatured” human collagen relative to “native” human collagen. In some embodiments, the antibody, or antigen-binding fragment thereof, specifically binds to both “denatured” and “native” forms of human collagen.

“Native” collagen refers to a collagen molecule in which the three alpha-chains are organized in a triple helical molecule. Native collagen can be of different stages of post-translational processing such as pro collagen and any intermediates in the generation of a mature tissue form of collagen, or collagen molecules isolated by limited proteolysis of tissues under conditions where the triple-helical structure of collagen is not disrupted. Thus, native collagen can be an intact collagen molecule or can contain non-triple-helical sequences flanking triple-helical regions, so long as the triple-helical is not disrupted. “Denatured” collagen refers to a collagen molecule in which the triple helix is completely or partially disrupted such that a cryptic epitope is made accessible. Denaturation of collagen can occur in situ (for example, in a tumor/cancer tissue) by the action of proteinases, for example, matrix metalloproteinases, which cleave collagen within triple helical regions, rendering the resulting fragments of the triple helix unstable. Denaturation of collagen can be induced in vitro by the deal or chemical denaturation of native collagen. Denatured collagen can also be prepared in vitro by treatment of native collagen with proteinases capable of cleaving a triple helical region(s), which are commonly referred to as collagenolytic enzymes, at temperatures where the resulting fragments of the triple helix are thermally unstable. Denatured collagen can be obtained by denaturation of native collagens of different stages of post-translational processing or denaturation of native collagen isolated from tissues by limited proteolysis.

Exemplary human collagen polypeptide sequences are provided in Table C1 below.

TABLE C1
Human Collagen Polypeptides

| | | SEQ ID |
|---|---|---|
| Name | Sequence | NO : |
| Type I, | MFSFVDLRLLLLLAATALLTHGQEEGQVEGQDEDIPPITCVQNGLRYHDRDVWK | 415 |
| alpha 1 | PEPCRICVCDNGKVLCDDVICDETKNCPGAEVPEGECCPVCPDGSESPTDQETT | |
| chain | GVEGPKGDTGPRGPRGPAGPPGRDGIPGQPGLPGPPGPPGPPGPPGLGGNFAPQ | |
| | LSYGYDEKSTGGISVPGPMGPSGPRGLPGPPGAPGPQGFQGPPGEPGEPGASGP | |
| | MGPRGPPGPPGKNGDDGEAGKPGRPGERGPPGPQGARGLPGTAGLPGMKGHRGF | |
| | SGLDGAKGDAGPAGPKGEPGSPGENGAPGQMGPRGLPGERGRPGAPGPAGARGN | |
| | DGATGAAGPPGPTGPAGPPGFPGAVGAKGEAGPQGPRGSEGPQGVRGEPGPPGP | |
| | AGAAGPAGNPGADGQPGAKGANGAPGIAGAPGFPGARGPSGPQGPGGPPGPKGN | |
| | SGEPGAPGSKGDTGAKGEPGPVGVQGPPGPAGEEGKRGARGEPGPTGLPGPPGE | |
| | RGGPGSRGFPGADGVAGPKGPAGERGSPGPAGPKGSPGEAGRPGEAGLPGAKGL | |
| | TGSPGSPGPDGKTGPPGPAGQDGRPGPPGPPGARGQAGVMGFPGPKGAAGEPGK | |
| | AGERGVPGPPGAVGPAGKDGEAGAQGPPGPAGPAGERGEQGPAGSPGFQGLPGP | |
| | AGPPGEAGKPGEQGVPGDLGAPGPSGARGERGFPGERGVQGPPGPAGPRGANGA | |
| | PGNDGAKGDAGAPGAPGSQGAPGLQGMPGERGAAGLPGPKGDRGDAGPKGADGS | |
| | PGKDGVRGLTGPIGPPGPAGAPGDKGESGPSGPAGPTGARGAPGDRGEPGPPGP | |
| | AGFAGPPGADGQPGAKGEPGDAGAKGDAGPPGPAGPAGPPGPIGNVGAPGAKGA | |
| | RGSAGPPGATGFPGAAGRVGPPGPSGNAGPPGPPGPAGKEGGKGPRGETGPAGR | |
| | PGEVGPPGPPGPAGEKGSPGADGPAGAPGTPGPQGIAGQRGVVGLPGQRGERGF | |
| | PGLPGPSGEPGKQGPSGASGERGPPGPMGPPGLAGPPGESGREGAPGAEGSPGR | |
| | DGSPGAKGDRGETGPAGPPGAPGAPGAPGPVGPAGKSGDRGETGPAGPTGPVGP | |
| | VGARGPAGPQGPRGDKGETGEQGDRGIKGHRGFSGLQGPPGPPGSPGEQGPSGA | |
| | SGPAGPRGPPGSAGAPGKDGLNGLPGPIGPPGPRGRTGDAGPVGPPGPPGPPGP | |
| | PGPPSAGFDFSFLPQPPQEKAHDGGRYYRADDANVVRDRDLEVDTTLKSLSQQI | |
| | ENIRSPEGSRKNPARTCRDLKMCHSDWKSGEYWIDPNQGCNLDAIKVFCNMETG | |
| | ETCVYPTOPSVAQKNWYISKNPKDKRHVWFGESMTDGFQFEYGGQGSDPADVAI | |
| | QLTFLRLMSTEASQNITYHCKNSVAYMDQQTGNLKKALLLQGSNEIEIRAEGNS | |
| | RFTYSVTVDGCTSHTGAWGKTVIEYKTTKTSRLPIIDVAPLDVGAPDQEFGFDV | |
| | GPVCFL | |
| Type I, | MLSFVDTRTLLLLAVTLCLATCQSLQEETVRKGPAGDRGPRGERGPPGPPGRDG | 416 |
| alpha 2 | EDGPTGPPGPPGPPGPPGLGGNFAAQYDGKGVGLGPGPMGLMGPRGPPGAAGAP | |
| chain | GPQGFQGPAGEPGEPGQTGPAGARGPAGPPGKAGEDGHPGKPGRPGERGVVGPQ | |
| | GARGFPGTPGLPGFKGIRGHNGLDGLKGQPGAPGVKGEPGAPGENGTPGQTGAR | |
| | GLPGERGRVGAPGPAGARGSDGSVGPVGPAGPIGSAGPPGFPGAPGPKGEIGAV | |
| | GNAGPAGPAGPRGEVGLPGLSGPVGPPGNPGANGLTGAKGAAGLPGVAGAPGLP | |
| | GPRGIPGPVGAAGATGARGLVGEPGPAGSKGESGNKGEPGSAGPQGPPGPSGEE | |
| | GKRGPNGEAGSAGPPGPPGLRGSPGSRGLPGADGRAGVMGPPGSRGASGPAGVR | |
| | GPNGDAGRPGEPGLMGPRGLPGSPGNIGPAGKEGPVGLPGIDGRPGPIGPAGAR | |
| | GEPGNIGFPGPKGPTGDPGKNGDKGHAGLAGARGAPGPDGNNGAQGPPGPQGVQ | |
| | GGKGEQGPPGPPGFQGLPGPSGPAGEVGKPGERGLHGEFGLPGPAGPRGERGPP | |
| | GESGAAGPTGPIGSRGPSGPPGPDGNKGEPGVVGAVGTAGPSGPSGLPGERGAA | |
| | GIPGGKGEKGEPGLRGEIGNPGRDGARGAPGAVGAPGPAGATGDRGEAGAAGPA | |
| | GPAGPRGSPGERGEVGPAGPNGFAGPAGAAGQPGAKGERGAKGPKGENGVVGPT | |
| | GPVGAAGPAGPNGPPGPAGSRGDGGPPGMTGFPGAAGRTGPPGPSGISGPPGPP | |
| | GPAGKEGLRGPRGDQGPVGRTGEVGAVGPPGFAGEKGPSGEAGTAGPPGTPGPQ | |
| | GLLGAPGILGLPGSRGERGLPGVAGAVGEPGPLGIAGPPGARGPPGAVGSPGVN | |
| | GAPGEAGRDGNPGNDGPPGRDGQPGHKGERGYPGNIGPVGAAGAPGPHGPVGPA | |
| | GKHGNRGETGPSGPVGPAGAVGPRGPSGPQGIRGDKGEPGEKGPRGLPGLKGHN | |
| | GLQGLPGIAGHHGDQGAPGSVGPAGPRGPAGPSGPAGKDGRTGHPGTVGPAGIR | |
| | GPQGHQGPAGPPGPPGPPGPPGVSGGGYDFGYDGDFYRADQPRSAPSLRPKDYE | |
| | VDATLKSLNNQIETLLTPEGSRKNPARTCRDLRLSHPEWSSGYYWIDPNQGCTM | |
| | DAIKVYCDFSTGETCIRAQPENIPAKNWYRSSKDKKHVWLGETINAGSQFEYNV | |
| | EGVTSKEMATOLAFMRLLANYASQNITYHCKNSIAYMDEETGNLKKAVILQGSN | |
| | DVELVAEGNSRFTYTVLVDGCSKKTNEWGKTIIEYKTNKPSRLPFLDIAPLDIG | |
| | GADQEFFVDIGPVCFK | |
| Type II, | MIRLGAPQTLVLLTLLVAAVLRCQGQDVQEAGSCVQDGQRYNDKDVWKPEPCRI | 417 |
| alpha 1 | CVCDTGTVLCDDIICEDVKDCLSPEIPFGECCPICPTDLATASGQPGPKGQKGE | |
| chain | PGDIKDIVGPKGPPGPQGPAGEQGPRGDRGDKGEKGAPGPRGRDGEPGTPGNPG | |
| | PPGPPGPPGPPGLGGNFAAQMAGGFDEKAGGAQLGVMQGPMGPMGPRGPPGPAG | |
| | APGPQGFQGNPGEPGEPGVSGPMGPRGPPGPPGKPGDDGEAGKPGKAGERGPPG | |
| | PQGARGFPGTPGLPGVKGHRGYPGLDGAKGEAGAPGVKGESGSPGENGSPGPMG | |
| | PRGLPGERGRTGPAGAAGARGNDGQPGPAGPPGPVGPAGGPGFPGAPGAKGEAG | |
| | PTGARGPEGAQGPRGEPGTPGSPGPAGASGNPGTDGIPGAKGSAGAPGIAGAPG | |
| | FPGPRGPPGPQGATGPLGPKGQTGEPGIAGFKGEQGPKGEPGPAGPQGAPGPAG | |
| | EEGKRGARGEPGGVGPIGPPGERGAPGNRGFPGQDGLAGPKGAPGERGPSGLAG | |
| | PKGANGDPGRPGEPGLPGARGLTGRPGDAGPQGKVGPSGAPGEDGRPGPPGPQG | |
| | ARGQPGVMGFPGPKGANGEPGKAGEKGLPGAPGLRGLPGKDGETGAAGPPGPAG | |
| | PAGERGEQGAPGPSGFQGLPGPPGPPGEGGKPGDQGVPGEAGAPGLVGPRGERG | |
| | FPGERGSPGAQGLQGPRGLPGTPGTDGPKGASGPAGPPGAQGPPGLQGMPGERG | |
| | AAGIAGPKGDRGDVGEKGPEGAPGKDGGRGLTGPIGPPGPAGANGEKGEVGPPG | |
| | PAGSAGARGAPGERGETGPPGPAGFAGPPGADGQPGAKGEQGEAGQKGDAGAPG | |
| | PQGPSGAPGPQGPTGVTGPKGARGAQGPPGATGFPGAAGRVGPPGSNGNPGPPG | |
| | PPGPSGKDGPKGARGDSGPPGRAGEPGLOGPAGPPGEKGEPGDDGPSGAEGPPG | |
| | PQGLAGQRGIVGLPGQRGERGFPGLPGPSGEPGKQGAPGASGDRGPPGPVGPPG | |
| | LTGPAGEPGREGSPGADGPPGRDGAAGVKGDRGETGAVGAPGAPGPPGSPGPAG | |
| | PTGKQGDRGEAGAQGPMGPSGPAGARGIQGPQGPRGDKGEAGEPGERGLKGHRG | |
| | FTGLQGLPGPPGPSGDQGASGPAGPSGPRGPPGPVGPSGKDGANGIPGPIGPPG | |
| | PRGRSGETGPAGPPGNPGPPGPPGPPGPGIDMSAFAGLGPREKGPDPLQYMRAD | |
| | QAAGGLROHDAEVDATLKSLNNQIESIRSPEGSRKNPARTCRDLKLCHPEWKSG | |
| | DYWIDPNQGCTLDAMKVFCNMETGETCVYPNPANVPKKNWWSSKSKEKKHIWFG | |
| | ETINGGFHFSYGDDNLAPNTANVOMTFLRLLSTEGSQNITYHCKNSIAYLDEAA | |
| | GNLKKALLIQGSNDVEIRAEGNSRFTYTALKDGCTKHTGKWGKTVIEYRSQKTS | |
| | RLPIIDIAPMDIGGPEQEFGVDIGPVCFL | |
| Type III, | MMSFVQKGSWLLLALLHPTIILAQQEAVEGGCSHLGQSYADRDVWKPEPCQICV | 418 |
| alpha 1 | CDSGSVLCDDIICDDQELDCPNPEIPFGECCAVCPQPPTAPTRPPNGQGPQGPK | |
| chain | GDPGPPGIPGRNGDPGIPGQPGSPGSPGPPGICESCPTGPQNYSPQYDSYDVKS | |
| | GVAVGGLAGYPGPAGPPGPPGPPGTSGHPGSPGSPGYQGPPGEPGQAGPSGPPG | |
| | PPGAIGPSGPAGKDGESGRPGRPGERGLPGPPGIKGPAGIPGFPGMKGHRGFDG | |
| | RNGEKGETGAPGLKGENGLPGENGAPGPMGPRGAPGERGRPGLPGAAGARGNDG | |
| | ARGSDGQPGPPGPPGTAGFPGSPGAKGEVGPAGSPGSNGAPGORGEPGPQGHAG | |
| | AQGPPGPPGINGSPGGKGEMGPAGIPGAPGLMGARGPPGPAGANGAPGLRGGAG | |
| | EPGKNGAKGEPGPRGERGEAGIPGVPGAKGEDGKDGSPGEPGANGLPGAAGERG | |
| | APGFRGPAGPNGIPGEKGPAGERGAPGPAGPRGAAGEPGRDGVPGGPGMRGMPG | |
| | SPGGPGSDGKPGPPGSQGESGRPGPPGPSGPRGQPGVMGFPGPKGNDGAPGKNG | |
| | ERGGPGGPGPQGPPGKNGETGPQGPPGPTGPGGDKGDTGPPGPQGLQGLPGTGG | |
| | PPGENGKPGEPGPKGDAGAPGAPGGKGDAGAPGERGPPGLAGAPGLRGGAGPPG | |
| | PEGGKGAAGPPGPPGAAGTPGLQGMPGERGGLGSPGPKGDKGEPGGPGADGVPG | |
| | KDGPRGPTGPIGPPGPAGQPGDKGEGGAPGLPGIAGPRGSPGERGETGPPGPAG | |
| | FPGAPGQNGEPGGKGERGAPGEKGEGGPPGVAGPPGGSGPAGPPGPQGVKGERG | |
| | SPGGPGAAGFPGARGLPGPPGSNGNPGPPGPSGSPGKDGPPGPAGNTGAPGSPG | |
| | VSGPKGDAGQPGEKGSPGAQGPPGAPGPLGIAGITGARGLAGPPGMPGPRGSPG | |
| | PQGVKGESGKPGANGLSGERGPPGPQGLPGLAGTAGEPGRDGNPGSDGLPGRDG | |
| | SPGGKGDRGENGSPGAPGAPGHPGPPGPVGPAGKSGDRGESGPAGPAGAPGPAG | |
| | SRGAPGPQGPRGDKGETGERGAAGIKGHRGFPGNPGAPGSPGPAGQQGAIGSPG | |
| | PAGPRGPVGPSGPPGKDGTSGHPGPIGPPGPRGNRGERGSEGSPGHPGQPGPPG | |
| | PPGAPGPCCGGVGAAAIAGIGGEKAGGFAPYYGDEPMDFKINTDEIMTSLKSVN | |
| | GQIESLISPDGSRKNPARNCRDLKFCHPELKSGEYWVDPNQGCKLDAIKVFCNM | |
| | ETGETCISANPLNVPRKHWWTDSSAEKKHVWFGESMDGGFQFSYGNPELPEDVL | |
| | DVHLAFLRLLSSRASQNITYHCKNSIAYMDQASGNVKKALKLMGSNEGEFKAEG | |
| | NSKFTYTVLEDGCTKHTGEWSKTVFEYRTRKAVRLPIVDIAPYDIGGPDQEFGV | |
| | DVGPVCFL | |
| Type IV, | MGPRLSVWLLLLPAALLLHEEHSRAAAKGGCAGSGCGKCDCHGVKGQKGERGLP | 419 |
| alpha 1 | GLOGVIGFPGMOGPEGPQGPPGQKGDTGEPGLPGTKGTRGPPGASGYPGNPGLP | |
| chain | GIPGQDGPPGPPGIPGCNGTKGERGPLGPPGLPGFAGNPGPPGLPGMKGDPGEI | |
| | LGHVPGMLLKGERGFPGIPGTPGPPGLPGLQGPVGPPGFTGPPGPPGPPGPPGE | |
| | KGQMGLSFQGPKGDKGDQGVSGPPGVPGQAQVQEKGDFATKGEKGQKGEPGFQG | |
| | MPGVGEKGEPGKPGPRGKPGKDGDKGEKGSPGFPGEPGYPGLIGRQGPQGEKGE | |
| | AGPPGPPGIVIGTGPLGEKGERGYPGTPGPRGEPGPKGFPGLPGQPGPPGLPVP | |
| | GQAGAPGFPGERGEKGDRGFPGTSLPGPSGRDGLPGPPGSPGPPGQPGYTNGIV | |
| | ECQPGPPGDQGPPGIPGQPGFIGEIGEKGQKGESCLICDIDGYRGPPGPQGPPG | |
| | EIGFPGQPGAKGDRGLPGRDGVAGVPGPQGTPGLIGQPGAKGEPGEFYFDLRLK | |
| | GDKGDPGFPGQPGMPGRAGSPGRDGHPGLPGPKGSPGSVGLKGERGPPGGVGFP | |
| | GSRGDTGPPGPPGYGPAGPIGDKGQAGFPGGPGSPGLPGPKGEPGKIVPLPGPP | |
| | GAEGLPGSPGFPGPQGDRGFPGTPGRPGLPGEKGAVGQPGIGFPGPPGPKGVDG | |
| | LPGDMGPPGTPGRPGFNGLPGNPGVQGQKGEPGVGLPGLKGLPGLPGIPGTPGE | |
| | KGSIGVPGVPGEHGAIGPPGLQGIRGEPGPPGLPGSVGSPGVPGIGPPGARGPP | |
| | GGQGPPGLSGPPGIKGEKGFPGFPGLDMPGPKGDKGAQGLPGITGQSGLPGLPG | |
| | QQGAPGIPGFPGSKGEMGVMGTPGQPGSPGPVGAPGLPGEKGDHGFPGSSGPRG | |
| | DPGLKGDKGDVGLPGKPGSMDKVDMGSMKGQKGDQGEKGQIGPIGEKGSRGDPG | |
| | TPGVPGKDGQAGQPGQPGPKGDPGISGTPGAPGLPGPKGSVGGMGLPGTPGEKG | |
| | VPGIPGPQGSPGLPGDKGAKGEKGOAGPPGIGIPGLRGEKGDQGIAGFPGSPGE | |
| | KGEKGSIGIPGMPGSPGLKGSPGSVGYPGSPGLPGEKGDKGLPGLDGIPGVKGE | |
| | AGLPGTPGPTGPAGQKGEPGSDGIPGSAGEKGEPGLPGRGFPGFPGAKGDKGSK | |
| | GEVGFPGLAGSPGIPGSKGEQGFMGPPGPQGQPGLPGSPGHATEGPKGDRGPQG | |
| | QPGLPGLPGPMGPPGLPGIDGVKGDKGNPGWPGAPGVPGPKGDPGFQGMPGIGG | |
| | SPGITGSKGDMGPPGVPGFQGPKGLPGLQGIKGDQGDQGVPGAKGLPGPPGPPG | |
| | PYDIIKGEPGLPGPEGPPGLKGLQGLPGPKGQQGVTGLVGIPGPPGIPGFDGAP | |
| | GQKGEMGPAGPTGPRGFPGPPGPDGLPGSMGPPGTPSVDHGFLVTRHSQTIDDP | |
| | QCPSGTKILYHGYSLLYVQGNERAHGQDLGTAGSCLRKFSTMPFLFCNINNVCN | |
| | FASRNDYSYWLSTPEPMPMSMAPITGENIRPFISRCAVCEAPAMVMAVHSQTIQ | |
| | IPPCPSGWSSLWIGYSFVMHTSAGAEGSGQALASPGSCLEEFRSAPFIECHGRG | |
| | TCNYYANAYSFWLATIERSEMFKKPTPSTLKAGELRTHVSRCQVCMRRT | |
| Type IV, | MGRDORAVAGPALRRWLLLGTVTVGFLAQSVLAGVKKFDVPCGGRDCSGGCQCY | 420 |
| alpha 2 | PEKGGRGQPGPVGPQGYNGPPGLQGFPGLQGRKGDKGERGAPGVTGPKGDVGAR | |
| chain | GVSGFPGADGIPGHPGQGGPRGRPGYDGCNGTQGDSGPQGPPGSEGFTGPPGPQ | |
| | GPKGQKGEPYALPKEERDRYRGEPGEPGLVGFQGPPGRPGHVGQMGPVGAPGRP | |
| | GPPGPPGPKGQQGNRGLGFYGVKGEKGDVGQPGPNGIPSDTLHPIIAPTGVTFH | |
| | PDQYKGEKGSEGEPGIRGISLKGEEGIMGFPGLRGYPGLSGEKGSPGQKGSRGL | |
| | DGYQGPDGPRGPKGEAGDPGPPGLPAYSPHPSLAKGARGDPGFPGAQGEPGSQG | |
| | EPGDPGLPGPPGLSIGDGDQRRGLPGEMGPKGFIGDPGIPALYGGPPGPDGKRG | |
| | PPGPPGLPGPPGPDGFLFGLKGAKGRAGFPGLPGSPGARGPKGWKGDAGECRCT | |
| | EGDEAIKGLPGLPGPKGFAGINGEPGRKGDRGDPGQHGLPGFPGLKGVPGNIGA | |
| | PGPKGAKGDSRTITTKGERGQPGVPGVPGMKGDDGSPGRDGLDGFPGLPGPPGD | |
| | GIKGPPGDPGYPGIPGTKGTPGEMGPPGLGLPGLKGQRGFPGDAGLPGPPGFLG | |
| | PPGPAGTPGQIDCDTDVKRAVGGDRQEAIQPGCIGGPKGLPGLPGPPGPTGAKG | |
| | LRGIPGFAGADGGPGPRGLPGDAGREGFPGPPGFIGPRGSKGAVGLPGPDGSPG | |
| | PIGLPGPDGPPGERGLPGEVLGAQPGPRGDAGVPGQPGLKGLPGDRGPPGFRGS | |
| | QGMPGMPGLKGQPGLPGPSGQPGLYGPPGLHGFPGAPGQEGPLGLPGIPGREGL | |
| | PGDRGDPGDTGAPGPVGMKGLSGDRGDAGFTGEQGHPGSPGFKGIDGMPGTPGL | |
| | KGDRGSPGMDGFQGMPGLKGRPGFPGSKGEAGFFGIPGLKGLAGEPGFKGSRGD | |
| | PGPPGPPPVILPGMKDIKGEKGDEGPMGLKGYLGAKGIQGMPGIPGLSGIPGLP | |
| | GRPGHIKGVKGDIGVPGIPGLPGFPGVAGPPGITGFPGFIGSRGDKGAPGRAGL | |
| | YGEIGATGDFGDIGDTINLPGRPGLKGERGTTGIPGLKGFFGEKGTEGDIGFPG | |
| | ITGVTGVQGPPGLKGQTGFPGLTGPPGSQGELGRIGLPGGKGDDGWPGAPGLPG | |
| | FPGLRGIRGLHGLPGTKGFPGSPGSDIHGDPGFPGPPGERGDPGEANTLPGPVG | |
| | VPGQKGDQGAPGERGPPGSPGLQGFPGITPPSNISGAPGDKGAPGIFGLKGYRG | |
| | PPGPPGSAALPGSKGDTGNPGAPGTPGTKGWAGDSGPQGRPGVFGLPGEKGPRG | |
| | EQGFMGNTGPTGAVGDRGPKGPKGDPGFPGAPGTVGAPGIAGIPQKIAVQPGTV | |
| | GPQGRRGPPGAPGEMGPQGPPGEPGFRGAPGKAGPQGRGGVSAVPGFRGDEGPI | |
| | GHQGPIGQEGAPGRPGSPGLPGMPGRSVSIGYLLVKHSQTDQEPMCPVGMNKLW | |
| | SGYSLLYFEGQEKAHNQDLGLAGSCLARFSTMPFLYCNPGDVCYYASRNDKSYW | |
| | LSTTAPLPMMPVAEDEIKPYISRCSVCEAPAIAIAVHSQDVSIPHCPAGWRSLW | |
| | IGYSFLMHTAAGDEGGGQSLVSPGSCLEDFRATPFIECNGGRGTCHYYANKYSF | |
| | WLTTIPEQSFOGSPSADTLKAGLIRTHISRCQVCMKNL | |
| Type V, | MDVHTRWKARSALRPGAPLLPPLLLLLLWAPPPSRAAQPADLLKVLDFHNLPDG | 421 |
| alpha 1 | ITKTTGFCATRRSSKGPDVAYRVTKDAQLSAPTKOLYPASAFPEDFSILTTVKA | |
| chain | KKGSQAFLVSIYNEQGIQQIGLELGRSPVFLYEDHTGKPGPEDYPLFRGINLSD | |
| | FEGDIQQLLFVSDHRAAYDYCEHYSPDCDTAVPDTPQSQDPNPDEYYTEGDGEG | |
| | ETYYYEYPYYEDPEDLGKEPTPSKKPVEAAKETTEVPEELTPTPTEAAPMPETS | |
| | EGAGKEEDVGIGDYDYVPSEDYYTPSPYDDLTYGEGEENPDQPTDPGAGAEIPT | |
| | STADTSNSSNPAPPPGEGADDLEGEFTEETIRNLDENYYDPYYDPTSSPSEIGP | |
| | GMPANQDTIYEGIGGPRGEKGQKGEPAIIEPGMLIEGPPGPEGPAGLPGPPGTM | |
| | GPTGQVGDPGERGPPGRPGLPGADGLPGPPGTMLMLPFRFGGGGDAGSKGPMVS | |
| | AQESQAQAILQQARLALRGPAGPMGLTGRPGPVGPPGSGGLKGEPGDVGPQGPR | |
| | GVQGPPGPAGKPGRRGRAGSDGARGMPGQTGPKGDRGFDGLAGLPGEKGHRGDP | |
| | GPSGPPGPPGDDGERGDDGEVGPRGLPGEPGPRGLLGPKGPPGPPGPPGVTGMD | |
| | GQPGPKGNVGPQGEPGPPGQQGNPGAQGLPGPQGAIGPPGEKGPLGKPGLPGMP | |
| | GADGPPGHPGKEGPPGEKGGQGPPGPQGPIGYPGPRGVKGADGIRGLKGTKGEK | |
| | GEDGFPGFKGDMGIKGDRGEIGPPGPRGEDGPEGPKGRGGPNGDPGPLGPPGEK | |
| | GKLGVPGLPGYPGRQGPKGSIGFPGFPGANGEKGGRGTPGKPGPRGQRGPTGPR | |
| | GERGPRGITGKPGPKGNSGGDGPAGPPGERGPNGPQGPTGFPGPKGPPGPPGKD | |
| | GLPGHPGQRGETGFQGKTGPPGPPGVVGPQGPTGETGPMGERGHPGPPGPPGEQ | |
| | GLPGLAGKEGTKGDPGPAGLPGKDGPPGLRGFPGDRGLPGPVGALGLKGNEGPP | |
| | GPPGPAGSPGERGPAGAAGPIGIPGRPGPQGPPGPAGEKGAPGEKGPQGPAGRD | |
| | GLQGPVGLPGPAGPVGPPGEDGDKGEIGEPGQKGSKGDKGEQGPPGPTGPQGPI | |
| | GOPGPSGADGEPGPRGQQGLFGQKGDEGPRGFPGPPGPVGLQGLPGPPGEKGET | |
| | GDVGQMGPPGPPGPRGPSGAPGADGPQGPPGGIGNPGAVGEKGEPGEAGEPGLP | |
| | GEGGPPGPKGERGEKGESGPSGAAGPPGPKGPPGDDGPKGSPGPVGFPGDPGPP | |
| | GEPGPAGQDGPPGDKGDDGEPGQTGSPGPTGEPGPSGPPGKRGPPGPAGPEGRQ | |
| | GEKGAKGEAGLEGPPGKTGPIGPQGAPGKPGPDGLRGIPGPVGEQGLPGSPGPD | |
| | GPPGPMGPPGLPGLKGDSGPKGEKGHPGLIGLIGPPGEQGEKGDRGLPGPQGSS | |
| | GPKGEQGITGPSGPIGPPGPPGLPGPPGPKGAKGSSGPTGPKGEAGHPGPPGPP | |
| | GPPGEVIQPLPIQASRTRRNIDASQLLDDGNGENYVDYADGMEEIFGSLNSLKL | |
| | EIEQMKRPLGTQQNPARTCKDLQLCHPDFPDGEYWVDPNQGCSRDSFKVYCNFT | |
| | AGGSTCVFPDKKSEGARITSWPKENPGSWFSEFKRGKLLSYVDAEGNPVGVVQM | |
| | TFLRLLSASAHQNVTYHCYQSVAWQDAATGSYDKALRFLGSNDEEMSYDNNPYI | |
| | RALVDGCATKKGYQKTVLEIDTPKVEQVPIVDIMFNDFGEASQKFGFEVGPACF | |
| | MG | |

Thus, in certain embodiments, an antibody, or antigen-binding fragment thereof, specifically binds to a denature human collagen type I, II, III, IV, and/or V polypeptide selected from Table C1. In some embodiments, an antibody, or antigen-binding fragment thereof, specifically binds to denatured human collagen type I, II, III, IV, and V polypeptides from Table C1. In some instances, an antibody, or antigen-binding fragment thereof, specifically binds to a denatured human type IV collagen polypeptide from Table C1, and does not substantially bind to the human collagen type I, II, III, or V polypeptides. In some instances, an antibody, or antigen-binding fragment thereof, specifically binds to a denatured human type I collagen polypeptide from Table C1, and does not substantially bind to a denatured human collagen type IV or V polypeptide.

As noted above, an antibody, or antigen-binding fragment thereof, binds to denatured human collagen type I, II, III, IV, and/or V polypeptide at a “cryptic collagen epitope”. A “cryptic collagen epitope” refers to an epitope of a collagen molecule that is less accessible to immunoglobulin binding in a native collagen polypeptide than in a denatured collagen polypeptide. Thus, in certain embodiments, an antibody, or antigen-binding fragment thereof, having binding specificity for a cryptic collagen epitope preferentially binds to a denatured human collagen polypeptide relative to a native human collagen polypeptide, that is, has a higher binding affinity for a denatured human collagen polypeptide relative to the corresponding native human collagen polypeptide. For example, in some embodiments, an antibody, or antigen-binding fragment thereof, has about or at least about 2, 3, 4, 5, 6, 7, 8, 9, 10, 15, 20, 25, 30, 35, 40, 50, 60, 70, 80, 90, or 100-fold or more higher binding affinity for a denatured human collagen polypeptide than it has for the corresponding native human collagen polypeptide. In some instances, a cryptic collagen epitope is a linear epitope. In some instances, a cryptic collagen epitope is a non-linear or conformational epitope. Candidate cryptic collagen epitopes can be identified, for example, by examining the three dimensional structure of a native triple helical collagen (see, for example, Fidler et al., J. Cell Sci. 131, doi:10.1242/jcs.203950, 2018). Peptide sequences that are not solvent exposed or are only partially solvent exposed in the native structure are potential cryptic collagen epitopes.

In certain embodiments, the cryptic collagen epitope is the HU177 cryptic collagen epitope (see, for example, Caron et al., Am. J. of Pathology. 186:1649-1661, 2016; Freimark et al., Molecular Immunology. 44:3741-3750, 2007; U.S. Pat. Nos. 8,025,883; and 7,566,770). In some instances, an antibody, or antigen-binding fragment thereof, specifically binds to denatured human collagen type I, II, III, IV, and/or V polypeptides at the HU177 cryptic collagen epitope. In some instances, an antibody, or antigen-binding fragment thereof, competitively inhibits binding of the HU177 antibody to a denatured human collagen type I, II, III, IV, and/or V polypeptide. In some instances, the HU177 cryptic collagen epitope comprises PGXP (SEQ ID NO:422), LPGXPG (SEQ ID NO: 423), and/or GPP′GXP′G (SEQ ID NO:424), wherein X is any amino acid, and wherein P′ is hydroxylproline.

In certain embodiments, the cryptic collagen epitope is the HUIV26 cryptic collagen epitope (see, for example, Favreau et al., Cancer Med. 3:265-272, 2014; and U.S. Pat. Nos. 8,025,883; and 7,566,770). In some instances, an antibody, or antigen-binding fragment thereof, specifically binds to a denatured human collagen type IV polypeptide at the HUIV26 cryptic collagen epitope. In some instances, an antibody, or antigen-binding fragment thereof, competitively inhibits binding of the HUIV26 antibody to a denatured human collagen type IV polypeptide.

In some instances, the cryptic collagen epitope is the XL313 cryptic collagen epitope (see, for example, Ames et al., J Biol Chem. 291:2731-50, 2016; U.S. Pat. Nos. 8,025,883; and 7,566,770). In some instances, an antibody, or antigen-binding fragment thereof, specifically binds to denatured human collagen type I and/or III polypeptides at the XL313 cryptic collagen epitope, and does not substantially bind to a denatured human collagen type IV or V polypeptide. In some instances, an antibody, or antigen-binding fragment thereof, competitively inhibits binding of the XL313 antibody to denatured human collagen type I and/or III polypeptides. In some instances, the XL313 cryptic collagen epitope comprises RGDKGE (SEQ ID NO: 425).

In certain embodiments, an antibody or antigen-binding fragment thereof is characterized by or comprises a heavy chain variable (V_{H}) region that comprises complementary determining region V_{H}CDR1, V_{H}CDR2, and V_{H}CDR3 sequences, and a light chain variable (V_{L}) region that comprises complementary determining region V_{L}CDR1, V_{L}CDR2, and V_{L}CDR3 sequences. Exemplary V_{H}CDR1, V_{H}CDR2, V_{H}CDR3, V_{L}CDR1, V_{L}CDR2, and V_{L}CDR3 sequences are provided in Table A1 below.

TABLE A1
Exemplary CDR Sequences

| | | |
|---|---|---|
| | | SEQ ID |
| Description | Sequence | NO: |

P27122713

| | | |
|---|---|---|
| V_{H}CDR1 | TSGMGVG | 1 |
| V_{H}CDR2 | DIWWDDNKYANPSLKS | 2 |
| V_{H}CDR3 | RANYGNPYYAQDY | 3 |
| V_{L}CDR1 | RSSQSIVHSNGNTYLE | 4 |
| V_{L}CDR2 | KVSNRFS | 5 |
| V_{L}CDR3 | FQGSHVPWT | 6 |

P27122714

| | | |
|---|---|---|
| V_{H}CDR1 | TSGMGVG | 7 |
| V_{H}CDR2 | DIWWDDNKYANPSLKS | 8 |
| V_{H}CDR3 | RANYGNPYYAQDY | 9 |
| V_{L}CDR1 | RSSQSIVHSQGNTYLE | 10 |
| V_{L}CDR2 | KVSNRFS | 11 |
| V_{L}CDR3 | FQGSHVPWT | 12 |

P27122716

| | | |
|---|---|---|
| V_{H}CDR1 | TSGMGVG | 13 |
| V_{H}CDR2 | DIWWDDNKYANPSLKS | 14 |
| V_{H}CDR3 | RANYGNPYYAQDY | 15 |
| V_{L}CDR1 | RSSQSIVSSWGNTYLE | 16 |
| V_{L}CDR2 | KVSNRFS | 17 |
| V_{L}CDR3 | FQGSHVPWT | 18 |

P27152716

| | | |
|---|---|---|
| V_{H}CDR1 | TPGMGVW | 19 |
| V_{H}CDR2 | DIWWDDNKYTNPSLKS | 20 |
| V_{H}CDR3 | RANYGNPYYAQDY | 21 |
| V_{L}CDR1 | RSSQSIVSSWGNTYLE | 22 |
| V_{L}CDR2 | KVSNRFS | 23 |
| V_{L}CDR3 | FQGSHVPWT | 24 |

P27152714

| | | |
|---|---|---|
| V_{H}CDR1 | TPGMGVW | 25 |
| V_{H}CDR2 | DIWWDDNKYTNPSLKS | 26 |
| V_{H}CDR3 | RANYGNPYYAQDY | 27 |
| V_{L}CDR1 | RSSQSIVHSQGNTYLE | 28 |
| V_{L}CDR2 | KVSNRFS | 29 |
| V_{L}CDR3 | FQGSHVPWT | 30 |

P27522716

| | | |
|---|---|---|
| V_{H}CDR1 | TPGMGVW | 31 |
| V_{H}CDR2 | DIWWDDNKYTNPSLKS | 32 |
| V_{H}CDR3 | RANYGNPYYAQDY | 33 |
| V_{L}CDR1 | RSSQSIVSSWGNTYLE | 34 |
| V_{L}CDR2 | KVSNRFS | 35 |
| V_{L}CDR3 | FQGSHVPWT | 36 |

P27532716

| | | |
|---|---|---|
| V_{H}CDR1 | TPGMGVW | 37 |
| V_{H}CDR2 | DIWWDDNKYTNPSLKS | 38 |
| V_{H}CDR3 | RANYGNPYYAQDY | 39 |
| V_{L}CDR1 | RSSQSIVSSWGNTYLE | 40 |
| V_{L}CDR2 | KVSNRFS | 41 |
| V_{L}CDR3 | FQGSHVPWT | 42 |

P27542716

| | | |
|---|---|---|
| V_{H}CDR1 | TPGMGVW | 43 |
| V_{H}CDR2 | DIWWDDNKYTNPSLKS | 44 |
| V_{H}CDR3 | RANYGNPYYAQDY | 45 |
| V_{L}CDR1 | RSSQSIVSSWGNTYLE | 46 |
| V_{L}CDR2 | KVSNRFS | 47 |
| V_{L}CDR3 | FQGSHVPWT | 48 |

P27552716

| | | |
|---|---|---|
| V_{H}CDR1 | TPGMGVW | 49 |
| V_{H}CDR2 | DIWWDDNKYTNPSLKS | 50 |
| V_{H}CDR3 | RANYGNPYYAQDY | 51 |
| V_{L}CDR1 | RSSQSIVSSWGNTYLE | 52 |
| V_{L}CDR2 | KVSNRFS | 53 |
| V_{L}CDR3 | FQGSHVPWT | 54 |

P27562716

| | | |
|---|---|---|
| V_{H}CDR1 | TPGMGVW | 55 |
| V_{H}CDR2 | DIWWDDNKYTNPSLKS | 56 |
| V_{H}CDR3 | RANYGNPYYAQDY | 57 |
| V_{L}CDR1 | RSSQSIVSSWGNTYLE | 58 |
| V_{L}CDR2 | KVSNRFS | 59 |
| V_{L}CDR3 | FQGSHVPWT | 60 |

P27572716

| | | |
|---|---|---|
| V_{H}CDR1 | TPGMGVW | 61 |
| V_{H}CDR2 | DIWWDDNKYTNPSLKS | 62 |
| V_{H}CDR3 | RANYGNPYYAQDY | 63 |
| V_{L}CDR1 | RSSQSIVSSWGNTYLE | 64 |
| V_{L}CDR2 | KVSNRFS | 65 |
| V_{L}CDR3 | FQGSHVPWT | 66 |

P27582716

| | | |
|---|---|---|
| V_{H}CDR1 | TPGMGVW | 67 |
| V_{H}CDR2 | DIWWDDNKYTNPSLKS | 68 |
| V_{H}CDR3 | RANYGNPYYAQDY | 69 |
| V_{L}CDR1 | RSSQSIVSSWGNTYLE | 70 |
| V_{L}CDR2 | KVSNRFS | 71 |
| V_{L}CDR3 | FQGSHVPWT | 72 |

P27592716

| | | |
|---|---|---|
| V_{H}CDR1 | TPGMGVW | 73 |
| V_{H}CDR2 | DIWWDDNKYTNPSLKS | 74 |
| V_{H}CDR3 | RANYGNPYYAQDY | 75 |
| V_{L}CDR1 | RSSQSIVSSWGNTYLE | 76 |
| V_{L}CDR2 | KVSNRFS | 77 |
| V_{L}CDR3 | FQGSHVPWT | 78 |

P27602716

| | | |
|---|---|---|
| V_{H}CDR1 | TPGMGVW | 79 |
| V_{H}CDR2 | DIWWDDNKYTNPSLKS | 80 |
| V_{H}CDR3 | RANYGNPYYAQDY | 81 |
| V_{L}CDR1 | RSSQSIVSSWGNTYLE | 82 |
| V_{L}CDR2 | KVSNRFS | 83 |
| V_{L}CDR3 | FQGSHVPWT | 84 |

P27612716

| | | |
|---|---|---|
| V_{H}CDR1 | TPGMGVW | 85 |
| V_{H}CDR2 | DIWWDDNKYTNPSLKS | 86 |
| V_{H}CDR3 | RANYGNPYYAQDY | 87 |
| V_{L}CDR1 | RSSQSIVSSWGNTYLE | 88 |
| V_{L}CDR2 | KVSNRFS | 89 |
| V_{L}CDR3 | FQGSHVPWT | 90 |

P27622716

| | | |
|---|---|---|
| V_{H}CDR1 | TPGMGVW | 91 |
| V_{H}CDR2 | DIWWDDNKYTNPSLKS | 92 |
| V_{H}CDR3 | RANYGNPYYAQDY | 93 |
| V_{L}CDR1 | RSSQSIVSSWGNTYLE | 94 |
| V_{L}CDR2 | KVSNRFS | 95 |
| V_{L}CDR3 | FQGSHVPWT | 96 |

P27632716

| | | |
|---|---|---|
| V_{H}CDR1 | TPGMGVW | 97 |
| V_{H}CDR2 | DIWWDDNKYTNPSLKS | 98 |
| V_{H}CDR3 | RANYGNPYYAQDY | 99 |
| V_{L}CDR1 | RSSQSIVSSWGNTYLE | 100 |
| V_{L}CDR2 | KVSNRFS | 101 |
| V_{L}CDR3 | FQGSHVPWT | 102 |

P27642716

| | | |
|---|---|---|
| V_{H}CDR1 | TPGMGVW | 103 |
| V_{H}CDR2 | DIWWDDNKYTNPSLKS | 104 |
| V_{H}CDR3 | RANYGNPYYAQDY | 105 |
| V_{L}CDR1 | RSSQSIVSSWGNTYLE | 106 |
| V_{L}CDR2 | KVSNRFS | 107 |
| V_{L}CDR3 | FQGSHVPWT | 108 |

P27652716

| | | |
|---|---|---|
| V_{H}CDR1 | TPGMGVW | 109 |
| V_{H}CDR2 | DIWWDDNKYTNPSLKS | 110 |
| V_{H}CDR3 | RANYGNPYYAQDY | 111 |
| V_{L}CDR1 | RSSQSIVSSWGNTYLE | 112 |
| V_{L}CDR2 | KVSNRFS | 113 |
| V_{L}CDR3 | FQGSHVPWT | 114 |

P27662716

| | | |
|---|---|---|
| V_{H}CDR1 | TPGMGVW | 115 |
| V_{H}CDR2 | DIWWDDNKYTNPSLKS | 116 |
| V_{H}CDR3 | RANYGNPYYAQDY | 117 |
| V_{L}CDR1 | RSSQSIVSSWGNTYLE | 118 |
| V_{L}CDR2 | KVSNRFS | 119 |
| V_{L}CDR3 | FQGSHVPWT | 120 |

P27122797

| | | |
|---|---|---|
| V_{H}CDR1 | TSGMGVG | 121 |
| V_{H}CDR2 | DIWWDDNKYANPSLKS | 122 |
| V_{H}CDR3 | RANYGNPYYAQDY | 123 |
| V_{L}CDR1 | RSSQSIVHSSGNTYLE | 124 |
| V_{L}CDR2 | KVSNRFS | 125 |
| V_{L}CDR3 | FQGSHVPWT | 126 |

P27122798

| | | |
|---|---|---|
| V_{H}CDR1 | TSGMGVG | 127 |
| V_{H}CDR2 | DIWWDDNKYANPSLKS | 128 |
| V_{H}CDR3 | RANYGNPYYAQDY | 129 |
| V_{L}CDR1 | RSSQSIVHSNANTYLE | 130 |
| V_{L}CDR2 | KVSNRFS | 131 |
| V_{L}CDR3 | FQGSHVPWT | 132 |

P27152797

| | | |
|---|---|---|
| V_{H}CDR1 | TPGMGVW | 133 |
| V_{H}CDR2 | DIWWDDNKYTNPSLKS | 134 |
| V_{H}CDR3 | RANYGNPYYAQDY | 135 |
| V_{L}CDR1 | RSSQSIVHSSGNTYLE | 136 |
| V_{L}CDR2 | KVSNRFS | 137 |
| V_{L}CDR3 | FQGSHVPWT | 138 |

P27152798

| | | |
|---|---|---|
| V_{H}CDR1 | TPGMGVW | 139 |
| V_{H}CDR2 | DIWWDDNKYTNPSLKS | 140 |
| V_{H}CDR3 | RANYGNPYYAQDY | 141 |
| V_{L}CDR1 | RSSQSIVHSNANTYLE | 142 |
| V_{L}CDR2 | KVSNRFS | 143 |
| V_{L}CDR3 | FQGSHVPWT | 144 |

P29342939

| | | |
|---|---|---|
| V_{H}CDR1 | RYWMT | 145 |
| V_{H}CDR2 | EINPDSSTANYTPYLKD | 146 |
| V_{H}CDR3 | PVDGYYDAMDP | 147 |
| V_{L}CDR1 | KSSQSLLNWYNQKNYLA | 148 |
| V_{L}CDR2 | GASTRES | 149 |
| V_{L}CDR3 | QNDHQYPYT | 150 |

P29352939

| | | |
|---|---|---|
| V_{H}CDR1 | RYWMT | 151 |
| V_{H}CDR2 | EINPDSSTANYTPYLKD | 152 |
| V_{H}CDR3 | PVDGYYDAMDP | 153 |
| V_{L}CDR1 | KSSQSLLNWYNQKNYLA | 154 |
| V_{L}CDR2 | GASTRES | 155 |
| V_{L}CDR3 | QNDHQYPYT | 156 |

P29362939

| | | |
|---|---|---|
| V_{H}CDR1 | RYWMT | 157 |
| V_{H}CDR2 | EINPDSSTANYTPYLKD | 158 |
| V_{H}CDR3 | PVDGYYDAMDP | 159 |
| V_{L}CDR1 | KSSQSLLNWYNQKNYLA | 160 |
| V_{L}CDR2 | GASTRES | 161 |
| V_{L}CDR3 | QNDHQYPYT | 162 |

P29372939

| | | |
|---|---|---|
| V_{H}CDR1 | RYWMT | 163 |
| V_{H}CDR2 | EINPDSSTANYTPYLKD | 164 |
| V_{H}CDR3 | PVDGYYDAMDP | 165 |
| V_{L}CDR1 | KSSQSLLNWYNQKNYLA | 166 |
| V_{L}CDR2 | GASTRES | 167 |
| V_{L}CDR3 | QNDHQYPYT | 168 |

P29382939

| | | |
|---|---|---|
| V_{H}CDR1 | RYWMT | 169 |
| V_{H}CDR2 | EINPDSSTANYTPYLKG | 170 |
| V_{H}CDR3 | PVDGYYDAMDP | 171 |
| V_{L}CDR1 | KSSQSLLNWYNQKNYLA | 172 |
| V_{L}CDR2 | GASTRES | 173 |
| V_{L}CDR3 | QNDHQYPYT | 174 |

P30392939

| | | |
|---|---|---|
| V_{H}CDR1 | RYWMT | 175 |
| V_{H}CDR2 | EINPDASTANYTPYLKG | 176 |
| V_{H}CDR3 | PVDGYYDAMDP | 177 |
| V_{L}CDR1 | KSSQSLLNWYNQKNYLA | 178 |
| V_{L}CDR2 | GASTRES | 179 |
| V_{L}CDR3 | QNDHQYPYT | 180 |

P30402939

| | | |
|---|---|---|
| V_{H}CDR1 | RYWMT | 181 |
| V_{H}CDR2 | EINPDSSTANYTPYLKG | 182 |
| V_{H}CDR3 | PVDAYYDAMDP | 183 |
| V_{L}CDR1 | KSSQSLLNWYNQKNYLA | 184 |
| V_{L}CDR2 | GASTRES | 185 |
| V_{L}CDR3 | QNDHQYPYT | 186 |

P30412939

| | | |
|---|---|---|
| V_{H}CDR1 | RYWMT | 187 |
| V_{H}CDR2 | EINPDASTANYTPYLKG | 188 |
| V_{H}CDR3 | PVDAYYDAMDP | 189 |
| V_{L}CDR1 | KSSQSLLNWYNQKNYLA | 190 |
| V_{L}CDR2 | GASTRES | 191 |
| V_{L}CDR3 | QNDHQYPYT | 192 |

P30422939

| | | |
|---|---|---|
| V_{H}CDR1 | RYWMT | 193 |
| V_{H}CDR2 | EINPDSSTANYTPYLKD | 194 |
| V_{H}CDR3 | PVDGYYDAMDY | 195 |
| V_{L}CDR1 | KSSQSLLNWYNQKNYLA | 196 |
| V_{L}CDR2 | GASTRES | 197 |
| V_{L}CDR3 | QNDHQYPYT | 198 |

P30432939

| | | |
|---|---|---|
| V_{H}CDR1 | RYWMT | 199 |
| V_{H}CDR2 | EINPDSSTANYTPYLKD | 200 |
| V_{H}CDR3 | PVDGYYDAMDN | 201 |
| V_{L}CDR1 | KSSQSLLNWYNQKNYLA | 202 |
| V_{L}CDR2 | GASTRES | 203 |
| V_{L}CDR3 | QNDHQYPYT | 204 |

P30442939

| | | |
|---|---|---|
| V_{H}CDR1 | RYWMT | 205 |
| V_{H}CDR2 | EINPDSSTANYTPYLKG | 206 |
| V_{H}CDR3 | PVDGYYDAMDY | 207 |
| V_{L}CDR1 | KSSQSLLNWYNQKNYLA | 208 |
| V_{L}CDR2 | GASTRES | 209 |
| V_{L}CDR3 | QNDHQYPYT | 210 |

P30452939

| | | |
|---|---|---|
| V_{H}CDR1 | RYWMT | 211 |
| V_{H}CDR2 | EINPDSSTANYTPYLKG | 212 |
| V_{H}CDR3 | PVDGYYDAMDN | 213 |
| V_{L}CDR1 | KSSQSLLNWYNQKNYLA | 214 |
| V_{L}CDR2 | GASTRES | 215 |
| V_{L}CDR3 | QNDHQYPYT | 216 |

P30393046

| | | |
|---|---|---|
| V_{H}CDR1 | RYWMT | 217 |
| V_{H}CDR2 | EINPDASTANYTPYLKG | 218 |
| V_{H}CDR3 | PVDGYYDAMDP | 219 |
| V_{L}CDR1 | KSSQSLLNRYNQKNYLA | 220 |
| V_{L}CDR2 | GASTRES | 221 |
| V_{L}CDR3 | QNDHQYPYT | 222 |

P30403046

| | | |
|---|---|---|
| V_{H}CDR1 | RYWMT | 223 |
| V_{H}CDR2 | EINPDSSTANYTPYLKG | 224 |
| V_{H}CDR3 | PVDAYYDAMDP | 225 |
| V_{L}CDR1 | KSSQSLLNRYNQKNYLA | 226 |
| V_{L}CDR2 | GASTRES | 227 |
| V_{L}CDR3 | QNDHQYPYT | 228 |

P30413046

| | | |
|---|---|---|
| V_{H}CDR1 | RYWMT | 229 |
| V_{H}CDR2 | EINPDASTANYTPYLKG | 230 |
| V_{H}CDR3 | PVDAYYDAMDP | 231 |
| V_{L}CDR1 | KSSQSLLNRYNQKNYLA | 232 |
| V_{L}CDR2 | GASTRES | 233 |
| V_{L}CDR3 | QNDHQYPYT | 234 |

P30423046

| | | |
|---|---|---|
| V_{H}CDR1 | RYWMT | 235 |
| V_{H}CDR2 | EINPDSSTANYTPYLKD | 236 |
| V_{H}CDR3 | PVDGYYDAMDY | 237 |
| V_{L}CDR1 | KSSQSLLNRYNQKNYLA | 238 |
| V_{L}CDR2 | GASTRES | 239 |
| V_{L}CDR3 | QNDHQYPYT | 240 |

P30433046

| | | |
|---|---|---|
| V_{H}CDR1 | RYWMT | 241 |
| V_{H}CDR2 | EINPDSSTANYTPYLKD | 242 |
| V_{H}CDR3 | PVDGYYDAMDN | 243 |
| V_{L}CDR1 | KSSQSLLNRYNQKNYLA | 244 |
| V_{L}CDR2 | GASTRES | 245 |
| V_{L}CDR3 | QNDHQYPYT | 246 |

P30443046

| | | |
|---|---|---|
| V_{H}CDR1 | RYWMT | 247 |
| V_{H}CDR2 | EINPDSSTANYTPYLKG | 248 |
| V_{H}CDR3 | PVDGYYDAMDY | 249 |
| V_{L}CDR1 | KSSQSLLNRYNQKNYLA | 250 |
| V_{L}CDR2 | GASTRES | 251 |
| V_{L}CDR3 | QNDHQYPYT | 252 |

P30453046

| | | |
|---|---|---|
| V_{H}CDR1 | RYWMT | 253 |
| V_{H}CDR2 | EINPDSSTANYTPYLKG | 254 |
| V_{H}CDR3 | PVDGYYDAMDN | 255 |
| V_{L}CDR1 | KSSQSLLNRYNQKNYLA | 256 |
| V_{L}CDR2 | GASTRES | 257 |
| V_{L}CDR3 | QNDHQYPYT | 258 |

HU177

| | | |
|---|---|---|
| V_{H}CDR1 | TSGMGVG | 259 |
| V_{H}CDR2 | DIWWDDNKYYNPSLKS | 260 |
| V_{H}CDR3 | RANYGNPYYAMDY | 261 |
| V_{L}CDR1 | RSSQSIVHSNGNTYLE | 262 |
| V_{L}CDR2 | KVSNRFS | 263 |
| V_{L}CDR3 | FQGSHVPWT | 264 |

HUIV26

| | | |
|---|---|---|
| V_{H}CDR1 | RYWMS | 265 |
| V_{H}CDR2 | EINPDSSTINYTPSLKD | 266 |
| V_{H}CDR3 | PVDGYYDAMDY | 267 |
| V_{L}CDR1 | KSSQSLLNSGNQKNYLA | 268 |
| V_{L}CDR2 | GASTRES | 269 |
| V_{L}CDR3 | QNDHSYPYT | 270 |

Thus, in certain embodiments, an antibody or antigen-binding fragment thereof comprises a heavy chain variable (V_{H}) region that comprises complementary determining region V_{H}CDR1, V_{H}CDR2, and V_{H}CDR3 sequences selected from Table A1, and variants thereof which specifically bind to a denatured human collagen polypeptide (selected, for example, from Table C1); and a light chain variable (V_{L}) region that comprises V_{L}CDR1, V_{L}CDR2, and V_{L}CDR3 sequences selected from Table A1, and variants thereof which specifically bind to a denatured human collagen polypeptide (selected, for example, from Table C1).

In certain embodiments, the CDR regions are as follows:

the V_{H}CDR1, a V_{H}CDR2, and V_{H}CDR3 regions respectively comprise SEQ ID NOs: 1-3; and the V_{L}CDR1, V_{L}CDR2, and V_{L}CDR3 regions respectively comprise SEQ ID NOs: 4-6;

the V_{H}CDR1, a V_{H}CDR2, and V_{H}CDR3 regions respectively comprise SEQ ID NOs: 7-9; and the V_{L}CDR1, V_{L}CDR2, and V_{L}CDR3 regions respectively comprise SEQ ID NOs: 10-12;

the V_{H}CDR1, a V_{H}CDR2, and V_{H}CDR3 regions respectively comprise SEQ ID NOs: 13-15; and the V_{L}CDR1, V_{L}CDR2, and V_{L}CDR3 regions respectively comprise SEQ ID NOs: 16-18;

the V_{H}CDR1, a V_{H}CDR2, and V_{H}CDR3 regions respectively comprise SEQ ID NOs: 19-21; and the V_{L}CDR1, V_{L}CDR2, and V_{L}CDR3 regions respectively comprise SEQ ID NOs: 22-24;

the V_{H}CDR1, a V_{H}CDR2, and V_{H}CDR3 regions respectively comprise SEQ ID NOs: 25-27; and the V_{L}CDR1, V_{L}CDR2, and V_{L}CDR3 regions respectively comprise SEQ ID NOs: 28-30;

the V_{H}CDR1, a V_{H}CDR2, and V_{H}CDR3 regions respectively comprise SEQ ID NOs: 31-33; and the V_{L}CDR1, V_{L}CDR2, and V_{L}CDR3 regions respectively comprise SEQ ID NOs: 34-36;

the V_{H}CDR1, a V_{H}CDR2, and V_{H}CDR3 regions respectively comprise SEQ ID NOs: 37-39; and the V_{L}CDR1, V_{L}CDR2, and V_{L}CDR3 regions respectively comprise SEQ ID NOs: 40-42;

the V_{H}CDR1, a V_{H}CDR2, and V_{H}CDR3 regions respectively comprise SEQ ID NOs: 43-45; and the V_{L}CDR1, V_{L}CDR2, and V_{L}CDR3 regions respectively comprise SEQ ID NOs: 46-48;

the V_{H}CDR1, a V_{H}CDR2, and V_{H}CDR3 regions respectively comprise SEQ ID NOs: 49-51; and the V_{L}CDR1, V_{L}CDR2, and V_{L}CDR3 regions respectively comprise SEQ ID NOs: 52-54;

the V_{H}CDR1, a V_{H}CDR2, and V_{H}CDR3 regions respectively comprise SEQ ID NOs: 55-57; and the V_{L}CDR1, V_{L}CDR2, and V_{L}CDR3 regions respectively comprise SEQ ID NOs: 58-60;

the V_{H}CDR1, a V_{H}CDR2, and V_{H}CDR3 regions respectively comprise SEQ ID NOs: 61-63; and the V_{L}CDR1, V_{L}CDR2, and V_{L}CDR3 regions respectively comprise SEQ ID NOs: 64-66;

the V_{H}CDR1, a V_{H}CDR2, and V_{H}CDR3 regions respectively comprise SEQ ID NOs: 67-69; and the V_{L}CDR1, V_{L}CDR2, and V_{L}CDR3 regions respectively comprise SEQ ID NOs: 70-72;

the V_{H}CDR1, a V_{H}CDR2, and V_{H}CDR3 regions respectively comprise SEQ ID NOs: 73-75; and the V_{L}CDR1, V_{L}CDR2, and V_{L}CDR3 regions respectively comprise SEQ ID NOs: 76-78;

the V_{H}CDR1, a V_{H}CDR2, and V_{H}CDR3 regions respectively comprise SEQ ID NOs: 79-81; and the V_{L}CDR1, V_{L}CDR2, and V_{L}CDR3 regions respectively comprise SEQ ID NOs: 82-84;

the V_{H}CDR1, a V_{H}CDR2, and V_{H}CDR3 regions respectively comprise SEQ ID NOs: 85-87; and the V_{L}CDR1, V_{L}CDR2, and V_{L}CDR3 regions respectively comprise SEQ ID NOs: 88-90;

the V_{H}CDR1, a V_{H}CDR2, and V_{H}CDR3 regions respectively comprise SEQ ID NOs: 91-93; and the V_{L}CDR1, V_{L}CDR2, and V_{L}CDR3 regions respectively comprise SEQ ID NOs: 94-96;

the V_{H}CDR1, a V_{H}CDR2, and V_{H}CDR3 regions respectively comprise SEQ ID NOs: 97-99; and the V_{L}CDR1, V_{L}CDR2, and V_{L}CDR3 regions respectively comprise SEQ ID NOs: 100-102;

the V_{H}CDR1, a V_{H}CDR2, and V_{H}CDR3 regions respectively comprise SEQ ID NOs: 103-105; and the V_{L}CDR1, V_{L}CDR2, and V_{L}CDR3 regions respectively comprise SEQ ID NOs: 106-108;

the V_{H}CDR1, a V_{H}CDR2, and V_{H}CDR3 regions respectively comprise SEQ ID NOs: 109-111; and the V_{L}CDR1, V_{L}CDR2, and V_{L}CDR3 regions respectively comprise SEQ ID NOs: 112-114;

the V_{H}CDR1, a V_{H}CDR2, and V_{H}CDR3 regions respectively comprise SEQ ID NOs: 115-117; and the V_{L}CDR1, V_{L}CDR2, and V_{L}CDR3 regions respectively comprise SEQ ID NOs: 118-120;

the V_{H}CDR1, a V_{H}CDR2, and V_{H}CDR3 regions respectively comprise SEQ ID NOs: 121-123; and the V_{L}CDR1, V_{L}CDR2, and V_{L}CDR3 regions respectively comprise SEQ ID NOs: 124-126;

the V_{H}CDR1, a V_{H}CDR2, and V_{H}CDR3 regions respectively comprise SEQ ID NOs: 127-129; and the V_{L}CDR1, V_{L}CDR2, and V_{L}CDR3 regions respectively comprise SEQ ID NOs: 130-132;

the V_{H}CDR1, a V_{H}CDR2, and V_{H}CDR3 regions respectively comprise SEQ ID NOs: 133-135; and the V_{L}CDR1, V_{L}CDR2, and V_{L}CDR3 regions respectively comprise SEQ ID NOs: 136-138;

the V_{H}CDR1, a V_{H}CDR2, and V_{H}CDR3 regions respectively comprise SEQ ID NOs: 139-141; and the V_{L}CDR1, V_{L}CDR2, and V_{L}CDR3 regions respectively comprise SEQ ID NOs: 142-144;

the V_{H}CDR1, a V_{H}CDR2, and V_{H}CDR3 regions respectively comprise SEQ ID NOs: 145-147; and the V_{L}CDR1, V_{L}CDR2, and V_{L}CDR3 regions respectively comprise SEQ ID NOs: 148-150;

the V_{H}CDR1, a V_{H}CDR2, and V_{H}CDR3 regions respectively comprise SEQ ID NOs: 151-153; and the V_{L}CDR1, V_{L}CDR2, and V_{L}CDR3 regions respectively comprise SEQ ID NOs: 154-156;

the V_{H}CDR1, a V_{H}CDR2, and V_{H}CDR3 regions respectively comprise SEQ ID NOs: 157-159; and the V_{L}CDR1, V_{L}CDR2, and V_{L}CDR3 regions respectively comprise SEQ ID NOs: 160-162;

the V_{H}CDR1, a V_{H}CDR2, and V_{H}CDR3 regions respectively comprise SEQ ID NOs: 163-165; and the V_{L}CDR1, V_{L}CDR2, and V_{L}CDR3 regions respectively comprise SEQ ID NOs: 166-168;

the V_{H}CDR1, a V_{H}CDR2, and V_{H}CDR3 regions respectively comprise SEQ ID NOs: 169-171; and the V_{L}CDR1, V_{L}CDR2, and V_{L}CDR3 regions respectively comprise SEQ ID NOs: 172-174;

the V_{H}CDR1, a V_{H}CDR2, and V_{H}CDR3 regions respectively comprise SEQ ID NOs: 175-177; and the V_{L}CDR1, V_{L}CDR2, and V_{L}CDR3 regions respectively comprise SEQ ID NOs: 178-180;

the V_{H}CDR1, a V_{H}CDR2, and V_{H}CDR3 regions respectively comprise SEQ ID NOs: 181-183; and the V_{L}CDR1, V_{L}CDR2, and V_{L}CDR3 regions respectively comprise SEQ ID NOs: 184-186;

the V_{H}CDR1, a V_{H}CDR2, and V_{H}CDR3 regions respectively comprise SEQ ID NOs: 187-189; and the V_{L}CDR1, V_{L}CDR2, and V_{L}CDR3 regions respectively comprise SEQ ID NOs: 190-192;

the V_{H}CDR1, a V_{H}CDR2, and V_{H}CDR3 regions respectively comprise SEQ ID NOs: 193-195; and the V_{L}CDR1, V_{L}CDR2, and V_{L}CDR3 regions respectively comprise SEQ ID NOs: 196-198;

the V_{H}CDR1, a V_{H}CDR2, and V_{H}CDR3 regions respectively comprise SEQ ID NOs: 199-201; and the V_{L}CDR1, V_{L}CDR2, and V_{L}CDR3 regions respectively comprise SEQ ID NOs: 202-204;

the V_{H}CDR1, a V_{H}CDR2, and V_{H}CDR3 regions respectively comprise SEQ ID NOs: 205-207; and the V_{L}CDR1, V_{L}CDR2, and V_{L}CDR3 regions respectively comprise SEQ ID NOs: 208-210;

the V_{H}CDR1, a V_{H}CDR2, and V_{H}CDR3 regions respectively comprise SEQ ID NOs: 211-213; and the V_{L}CDR1, V_{L}CDR2, and V_{L}CDR3 regions respectively comprise SEQ ID NOs: 214-216;

the V_{H}CDR1, a V_{H}CDR2, and V_{H}CDR3 regions respectively comprise SEQ ID NOs: 217-219; and the V_{L}CDR1, V_{L}CDR2, and V_{L}CDR3 regions respectively comprise SEQ ID NOs: 220-222;

the V_{H}CDR1, a V_{H}CDR2, and V_{H}CDR3 regions respectively comprise SEQ ID NOs: 223-225; and the V_{L}CDR1, V_{L}CDR2, and V_{L}CDR3 regions respectively comprise SEQ ID NOs: 226-228;

the V_{H}CDR1, a V_{H}CDR2, and V_{H}CDR3 regions respectively comprise SEQ ID NOs: 229-231; and the V_{L}CDR1, V_{L}CDR2, and V_{L}CDR3 regions respectively comprise SEQ ID NOs: 232-234;

the V_{H}CDR1, a V_{H}CDR2, and V_{H}CDR3 regions respectively comprise SEQ ID NOs: 235-237; and the V_{L}CDR1, V_{L}CDR2, and V_{L}CDR3 regions respectively comprise SEQ ID NOs: 238-240;

the V_{H}CDR1, a V_{H}CDR2, and V_{H}CDR3 regions respectively comprise SEQ ID NOs: 241-243; and the V_{L}CDR1, V_{L}CDR2, and V_{L}CDR3 regions respectively comprise SEQ ID NOs: 244-246;

the V_{H}CDR1, a V_{H}CDR2, and V_{H}CDR3 regions respectively comprise SEQ ID NOs: 247-249; and the V_{L}CDR1, V_{L}CDR2, and V_{L}CDR3 regions respectively comprise SEQ ID NOs: 250-252;

the V_{H}CDR1, a V_{H}CDR2, and V_{H}CDR3 regions respectively comprise SEQ ID NOs: 253-255; and the V_{L}CDR1, V_{L}CDR2, and V_{L}CDR3 regions respectively comprise SEQ ID NOs: 256-258;

the V_{H}CDR1, a V_{H}CDR2, and V_{H}CDR3 regions respectively comprise SEQ ID NOs: 259-261; and the V_{L}CDR1, V_{L}CDR2, and V_{L}CDR3 regions respectively comprise SEQ ID NOs: 262-264; or

the V_{H}CDR1, a V_{H}CDR2, and V_{H}CDR3 regions respectively comprise SEQ ID NOs: 265-267; and the V_{L}CDR1, V_{L}CDR2, and V_{L}CDR3 regions respectively comprise SEQ ID NOs: 268-270.

Also included are variants of the foregoing CDR sequences, including affinity matured variants, which bind to a denatured human collage polypeptide (see Table C1), for example, variants having 1, 2, 3, 4, 5, 6, 7, or 8 total alterations in one or more of the CDR regions, for example, one or more the V_{H}CDR1, V_{H}CDR2, V_{H}CDR3, V_{L}CDR1, V_{L}CDR2, and/or V_{L}CDR3 sequences described herein. Exemplary “alterations” include amino acid substitutions, additions, and deletions.

In certain embodiments, an antibody, or antigen-binding fragment thereof, is characterized by or comprises a heavy chain (V_{H} region, Fc region) and a light chain (V_{L} region). Exemplary heavy and light chain sequences are provided in Table A2 below.

| | | |
|---|---|---|
| | | SEQ ID |
| Description | Sequence | NO: |

P27122713

| | | |
|---|---|---|
| Heavy Chain | QVTLKESGPALVKPTQTLTLTCTFSGFSLSTSGMGVGWIRQPPGKALE | 271 |
| | WLADIWWDDNKYANPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSNGNTYLEWYLQKPGQS | 272 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27122714

| | | |
|---|---|---|
| Heavy Chain | QVTLKESGPALVKPTQTLTLTCTFSGFSLSTSGMGVGWIRQPPGKALE | 273 |
| | WLADIWWDDNKYANPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSQGNTYLEWYLQKPGQS | 274 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27122716

| | | |
|---|---|---|
| Heavy Chain | QVTLKESGPALVKPTQTLTLTCTFSGFSLSTSGMGVGWIRQPPGKALE | 275 |
| | WLADIWWDDNKYANPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 276 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27152716

| | | |
|---|---|---|
| Heavy Chain | QVTLKESGPALVKPTQTLTLTCTFSGFSLSTPGMGVWWIRQPPGKALE | 277 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 278 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27152714

| | | |
|---|---|---|
| Heavy Chain | QVTLKESGPALVKPTQTLTLTCTFSGFSLSTPGMGVWWIRQPPGKALE | 279 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSQGNTYLEWYLQKPGQS | 280 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27522716

| | | |
|---|---|---|
| Heavy Chain | EVQLVESGGGLVQPGGSLRLSCAASGFSFSTPGMGVWWVRQAPGKGLE | 281 |
| | WVADIWWDDNKYTNPSLKSRFTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 282 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27532716

| | | |
|---|---|---|
| Heavy Chain | EVQLVESGGGLVQPGGSLRLSCAASGFTLSTPGMGVWWVRQAPGKGLE | 283 |
| | WVADIWWDDNKYTNPSLKSRFTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 284 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27542716

| | | |
|---|---|---|
| Heavy Chain | EVQLVESGGGLVQPGGSLRLSCAASGFSLSTPGMGVWWVRQAPGKGLE | 285 |
| | WVADIWWDDNKYTNPSLKSRFTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 286 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27552716

| | | |
|---|---|---|
| Heavy Chain | EVQLVESGGGLVQPGGSLRLSCAASGFTFSTPGMGVWWIRQAPGKGLE | 287 |
| | WVADIWWDDNKYTNPSLKSRFTISRDNAKNSLYLOMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 288 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27562716

| | | |
|---|---|---|
| Heavy Chain | EVQLVESGGGLVQPGGSLRLSCAASGFTFSTPGMGVWWVRQAPGKGLE | 289 |
| | WLADIWWDDNKYTNPSLKSRFTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 290 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27572716

| | | |
|---|---|---|
| Heavy Chain | EVQLVESGGGLVQPGGSLRLSCAASGFTFSTPGMGVWWVRQAPGKGLE | 291 |
| | WVADIWWDDNKYTNPSLKSRLTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 292 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27582716

| | | |
|---|---|---|
| Heavy Chain | EVQLVESGGGLVQPGGSLRLSCAASGFSLSTPGMGVWWIRQAPGKGLE | 293 |
| | WVADIWWDDNKYTNPSLKSRLTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 294 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27592716

| | | |
|---|---|---|
| Heavy Chain | EVQLVESGGGLVQPGGSLRLSCAASGFSLSTPGMGVWWIRQAPGKGLE | 295 |
| | WLADIWWDDNKYTNPSLKSRLTISRDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 296 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27602716

| | | |
|---|---|---|
| Heavy Chain | EVQLVESGGGLVQPGGSLRLSCAASGFSLSTPGMGVWWVRQAPGKGLE | 297 |
| | WLADIWWDDNKYTNPSLKSRLTISKDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 298 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27612716

| | | |
|---|---|---|
| Heavy Chain | EVQLVESGGGLVQPGGSLRLSCAASGFSLSTPGMGVWWIRQAPGKGLE | 299 |
| | WLADIWWDDNKYTNPSLKSRLTISKDNAKNSLYLQMNSLRAEDTAVYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 300 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27622716

| | | |
|---|---|---|
| Heavy Chain | QVTLKESGPALVKPTQTLTLTCTFSGFSLSTPGMGVWWIRQPSGKALE | 301 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 302 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27632716

| | | |
|---|---|---|
| Heavy Chain | QVTLKESGPALVKPTQTLTLTCTFSGFSLSTPGMGVWWIRQPPGEALE | 303 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 304 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27642716

| | | |
|---|---|---|
| Heavy Chain | QVTLKESGPALVKPTQTLTLTCTFSGFSLSTPGMGVWWIRQPPGKGLE | 305 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 306 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27652716

| | | |
|---|---|---|
| Heavy Chain | QVTLKESGPALVKPTQTLTLTCTFSGFSLSTPGMGVWWIRQPPGEGLE | 307 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 308 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27662716

| | | |
|---|---|---|
| Heavy Chain | QVTLKESGPALVKPTQTLTLTCTFSGFSLSTPGMGVWWIRQPSGEGLE | 309 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVSSWGNTYLEWYLQKPGQS | 310 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27122797

| | | |
|---|---|---|
| Heavy Chain | QVTLKESGPALVKPTQTLTLTCTFSGFSLSTSGMGVGWIRQPPGKALE | 311 |
| | WLADIWWDDNKYANPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSSGNTYLEWYLQKPGQS | 312 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27122798

| | | |
|---|---|---|
| Heavy Chain | QVTLKESGPALVKPTQTLTLTCTFSGFSLSTSGMGVGWIRQPPGKALE | 313 |
| | WLADIWWDDNKYANPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSNANTYLEWYLQKPGQS | 314 |
| | PQLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27152797

| | | |
|---|---|---|
| Heavy Chain | QVTLKESGPALVKPTQTLTLTCTFSGFSLSTPGMGVWWIRQPPGKALE | 315 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSSGNTYLEWYLQKPGQS | 316 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P27152798

| | | |
|---|---|---|
| Heavy Chain | QVTLKESGPALVKPTQTLTLTCTFSGFSLSTPGMGVWWIRQPPGKALE | 317 |
| | WLADIWWDDNKYTNPSLKSRLTISKDTSKNQVVLTMTNMDPVDTATYY | |
| | CARRANYGNPYYAQDYWGQGTTVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALAA | |
| | PIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCS | |
| | VMHEALHNHYTQKSLSLSPGK | |
| Light Chain | DIVMTQTPLSLPVTPGEPASISCRSSQSIVHSNANTYLEWYLQKPGQS | 318 |
| | POLLIYKVSNRFSGVPDRFSGSGSGTDFTLKISRVEAEDVGVYYCFQG | |
| | SHVPWTFGQGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |

P29342939

| | | |
|---|---|---|
| Heavy Chain | EVQLVESGGGLVQPGGSLRLSCAASGFDFSRYWMTWVRQAPGKGLEWV | 319 |
| | AEINPDSSTANYTPYLKDRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALAAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 320 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P29352939

| | | |
|---|---|---|
| Heavy Chain | EVQLVESGGGLVQPGGSLRLSCAASGFTFSRYWMTWVRQAPGKGLEWV | 321 |
| | AEINPDSSTANYTPYLKDRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALAAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 322 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P29362939

| | | |
|---|---|---|
| Heavy Chain | EVQLVESGGGLVQPGGSLRLSCAASGFTFSRYWMTWVRQAPGKGLEWI | 323 |
| | GEINPDSSTANYTPYLKDRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALAAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 324 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P29372939

| | | |
|---|---|---|
| Heavy Chain | EVQLVESGGGLVQPGGSLRLSCAASGFDFSRYWMTWVRQAPGKGLEWI | 325 |
| | GEINPDSSTANYTPYLKDRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALAAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 326 |
| | PPKLLIYGASTRESGVPDRFSGSGSGTDFTLTISSLQAEDVAVYYCQN | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P29382939

| | | |
|---|---|---|
| Heavy Chain | EVQLVESGGGLVQPGGSLRLSCAASGFTFSRYWMTWVRQAPGKGLEWI | 327 |
| | GEINPDSSTANYTPYLKGRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALAAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 328 |
| | PPKLLIYGASTRESGVPDRFSGSGSGTDFTLTISSLQAEDVAVYYCQN | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30392939

| | | |
|---|---|---|
| Heavy Chain | EVQLVESGGGLVQPGGSLRLSCAASGFTFSRYWMTWVRQAPGKGLEWI | 329 |
| | GEINPDASTANYTPYLKGRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 330 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30402939

| | | |
|---|---|---|
| Heavy Chain | EVQLVESGGGLVQPGGSLRLSCAASGFTFSRYWMTWVRQAPGKGLEWI | 331 |
| | GEINPDSSTANYTPYLKGRFTISRDNAKNSLYLQMNSLRAEDTAVYYC | |
| | ARPVDAYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 332 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30412939

| | | |
|---|---|---|
| Heavy Chain | EVQLVESGGGLVQPGGSLRLSCAASGFTFSRYWMTWVRQAPGKGLEWI | 333 |
| | GEINPDASTANYTPYLKGRFTISRDNAKNSLYLQMNSLRAEDTAVYYC | |
| | ARPVDAYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 334 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30422939

| | | |
|---|---|---|
| Heavy Chain | EVQLVESGGGLVQPGGSLRLSCAASGFTFSRYWMTWVRQAPGKGLEWV | 335 |
| | AEINPDSSTANYTPYLKDRFTISRDNAKNSLYLQMNSLRAEDTAVYYC | |
| | ARPVDGYYDAMDYWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 336 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30432939

| | | |
|---|---|---|
| Heavy Chain | EVQLVESGGGLVQPGGSLRLSCAASGFTFSRYWMTWVRQAPGKGLEWV | 337 |
| | AEINPDSSTANYTPYLKDRFTISRDNAKNSLYLQMNSLRAEDTAVYYC | |
| | ARPVDGYYDAMDNWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 338 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30442939

| | | |
|---|---|---|
| Heavy Chain | EVQLVESGGGLVQPGGSLRLSCAASGFTFSRYWMTWVRQAPGKGLEWI | 339 |
| | GEINPDSSTANYTPYLKGRFTISRDNAKNSLYLQMNSLRAEDTAVYYC | |
| | ARPVDGYYDAMDYWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 340 |
| | PPKLLIYGASTRESGVPDRFSGSGSGTDFTLTISSLQAEDVAVYYCQN | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30452939

| | | |
|---|---|---|
| Heavy Chain | EVQLVESGGGLVQPGGSLRLSCAASGFTFSRYWMTWVRQAPGKGLEWI | 341 |
| | GEINPDSSTANYTPYLKGRFTISRDNAKNSLYLQMNSLRAEDTAVYYC | |
| | ARPVDGYYDAMDNWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNWYNQKNYLAWYQQKPGQ | 342 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30393046

| | | |
|---|---|---|
| Heavy Chain | EVQLVESGGGLVQPGGSLRLSCAASGFTFSRYWMTWVRQAPGKGLEWI | 343 |
| | GEINPDASTANYTPYLKGRFTISRDNAKNSLYLQMNSLRAEDTAVYYC | |
| | ARPVDGYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 344 |
| | PPKLLIYGASTRESGVPDRFSGSGSGTDFTLTISSLQAEDVAVYYCQN | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30403046

| | | |
|---|---|---|
| Heavy Chain | EVQLVESGGGLVQPGGSLRLSCAASGFTFSRYWMTWVRQAPGKGLEWI | 345 |
| | GEINPDSSTANYTPYLKGRFTISRDNAKNSLYLQMNSLRAEDTAVYYC | |
| | ARPVDAYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 346 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30413046

| | | |
|---|---|---|
| Heavy Chain | EVQLVESGGGLVQPGGSLRLSCAASGFTFSRYWMTWVRQAPGKGLEWI | 347 |
| | GEINPDASTANYTPYLKGRFTISRDNAKNSLYLQMNSLRAEDTAVYYC | |
| | ARPVDAYYDAMDPWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 348 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30423046

| | | |
|---|---|---|
| Heavy Chain | EVQLVESGGGLVQPGGSLRLSCAASGFTFSRYWMTWVRQAPGKGLEWV | 349 |
| | AEINPDSSTANYTPYLKDRFTISRDNAKNSLYLQMNSLRAEDTAVYYC | |
| | ARPVDGYYDAMDYWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 350 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30433046

| | | |
|---|---|---|
| Heavy Chain | EVQLVESGGGLVQPGGSLRLSCAASGFTFSRYWMTWVRQAPGKGLEWV | 351 |
| | AEINPDSSTANYTPYLKDRFTISRDNAKNSLYLQMNSLRAEDTAVYYC | |
| | ARPVDGYYDAMDNWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 352 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30443046

| | | |
|---|---|---|
| Heavy Chain | EVQLVESGGGLVQPGGSLRLSCAASGFTFSRYWMTWVRQAPGKGLEWI | 353 |
| | GEINPDSSTANYTPYLKGRFTISRDNAKNSLYLQMNSLRAEDTAVYYC | |
| | ARPVDGYYDAMDYWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 354 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

P30453046

| | | |
|---|---|---|
| Heavy Chain | EVQLVESGGGLVQPGGSLRLSCAASGFTFSRYWMTWVRQAPGKGLEWI | 355 |
| | GEINPDSSTANYTPYLKGRFTISRDNAKNSLYLQMNSLRAEDTAVYYC | |
| | ARPVDGYYDAMDNWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light Chain | DIVMTQSPDSLAVSLGERATINCKSSQSLLNRYNQKNYLAWYQQKPGQ | 356 |
| | PPKLLIYGASTRESGVPDRFSGSGSGTDFTLTISSLQAEDVAVYYCON | |
| | DHQYPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKADY | |
| | EKHKVYACEVTHQGLSSPVTKSFNRGEC | |

Thus, in certain embodiments, an antibody, or antigen-binding fragment thereof, specifically bind to a denatured human collagen polypeptide (selected, for example, from Table C1) and comprises a heavy chain and a corresponding light chain selected from Table A2. In certain embodiments, the heavy chain is at least 80, 85, 90, 95, 97, 98, 99, or 100% identical to a sequence selected from Table A2, including, for example, wherein the heavy chain has 1, 2, 3, 4, 5, or 6 alterations in one or more framework regions. In some embodiments, the light chain is at least 80, 85, 90, 95, 97, 98, 99, or 100% identical to a sequence selected from Table A2, including, for example, wherein the light chain has 1, 2, 3, 4, 5, or 6 alterations in one or more framework regions.

In some embodiments, the heavy chain and light chain sequences of an antibody or antigen-binding fragment are as follows:

the heavy chain comprises a sequence at least 80, 85, 90, 95, 97, 98, 99, or 100% identical to SEQ ID NO: 271, and the light chain comprises a sequence at least 80, 85, 90, 95, 97, 98, 99, or 100% identical to SEQ ID NO: 272;

the heavy chain comprises a sequence at least 80, 85, 90, 95, 97, 98, 99, or 100% identical to SEQ ID NO: 273, and the light chain comprises a sequence at least 80, 85, 90, 95, 97, 98, 99, or 100% identical to SEQ ID NO: 274;

the heavy chain comprises a sequence at least 80, 85, 90, 95, 97, 98, 99, or 100% identical to SEQ ID NO: 275, and the light chain comprises a sequence at least 80, 85, 90, 95, 97, 98, 99, or 100% identical to SEQ ID NO: 276;

the heavy chain comprises a sequence at least 80, 85, 90, 95, 97, 98, 99, or 100% identical to SEQ ID NO: 277, and the light chain comprises a sequence at least 80, 85, 90, 95, 97, 98, 99, or 100% identical to SEQ ID NO: 278;

the heavy chain comprises a sequence at least 80, 85, 90, 95, 97, 98, 99, or 100% identical to SEQ ID NO: 279, and the light chain comprises a sequence at least 80, 85, 90, 95, 97, 98, 99, or 100% identical to SEQ ID NO: 280;

the heavy chain comprises a sequence at least 80, 85, 90, 95, 97, 98, 99, or 100% identical to SEQ ID NO: 281, and the light chain comprises a sequence at least 80, 85, 90, 95, 97, 98, 99, or 100% identical to SEQ ID NO: 282;

the heavy chain comprises a sequence at least 80, 85, 90, 95, 97, 98, 99, or 100% identical to SEQ ID NO: 283, and the light chain comprises a sequence at least 80, 85, 90, 95, 97, 98, 99, or 100% identical to SEQ ID NO: 284;

the heavy chain comprises a sequence at least 80, 85, 90, 95, 97, 98, 99, or 100% identical to SEQ ID NO: 285, and the light chain comprises a sequence at least 80, 85, 90, 95, 97, 98, 99, or 100% identical to SEQ ID NO: 286;

the heavy chain comprises a sequence at least 80, 85, 90, 95, 97, 98, 99, or 100% identical to SEQ ID NO: 287, and the light chain comprises a sequence at least 80, 85, 90, 95, 97, 98, 99, or 100% identical to SEQ ID NO: 288;

the heavy chain comprises a sequence at least 80, 85, 90, 95, 97, 98, 99, or 100% identical to SEQ ID NO: 289, and the light chain comprises a sequence at least 80, 85, 90, 95, 97, 98, 99, or 100% identical to SEQ ID NO: 290;

the heavy chain comprises a sequence at least 80, 85, 90, 95, 97, 98, 99, or 100% identical to SEQ ID NO: 291, and the light chain comprises a sequence at least 80, 85, 90, 95, 97, 98, 99, or 100% identical to SEQ ID NO: 292;

the heavy chain comprises a sequence at least 80, 85, 90, 95, 97, 98, 99, or 100% identical to SEQ ID NO: 293, and the light chain comprises a sequence at least 80, 85, 90, 95, 97, 98, 99, or 100% identical to SEQ ID NO: 294;

the heavy chain comprises a sequence at least 80, 85, 90, 95, 97, 98, 99, or 100% identical to SEQ ID NO: 295, and the light chain comprises a sequence at least 80, 85, 90, 95, 97, 98, 99, or 100% identical to SEQ ID NO: 296;

the heavy chain comprises a sequence at least 80, 85, 90, 95, 97, 98, 99, or 100% identical to SEQ ID NO: 297, and the light chain comprises a sequence at least 80, 85, 90, 95, 97, 98, 99, or 100% identical to SEQ ID NO: 298;

the heavy chain comprises a sequence at least 80, 85, 90, 95, 97, 98, 99, or 100% identical to SEQ ID NO: 299, and the light chain comprises a sequence at least 80, 85, 90, 95, 97, 98, 99, or 100% identical to SEQ ID NO: 300;

the heavy chain comprises a sequence at least 80, 85, 90, 95, 97, 98, 99, or 100% identical to SEQ ID NO: 301, and the light chain comprises a sequence at least 80, 85, 90, 95, 97, 98, 99, or 100% identical to SEQ ID NO: 302;

the heavy chain comprises a sequence at least 80, 85, 90, 95, 97, 98, 99, or 100% identical to SEQ ID NO: 303, and the light chain comprises a sequence at least 80, 85, 90, 95, 97, 98, 99, or 100% identical to SEQ ID NO: 304;

the heavy chain comprises a sequence at least 80, 85, 90, 95, 97, 98, 99, or 100% identical to SEQ ID NO: 305, and the light chain comprises a sequence at least 80, 85, 90, 95, 97, 98, 99, or 100% identical to SEQ ID NO: 306;

the heavy chain comprises a sequence at least 80, 85, 90, 95, 97, 98, 99, or 100% identical to SEQ ID NO: 307, and the light chain comprises a sequence at least 80, 85, 90, 95, 97, 98, 99, or 100% identical to SEQ ID NO: 308;

the heavy chain comprises a sequence at least 80, 85, 90, 95, 97, 98, 99, or 100% identical to SEQ ID NO: 309, and the light chain comprises a sequence at least 80, 85, 90, 95, 97, 98, 99, or 100% identical to SEQ ID NO: 310;

the heavy chain comprises a sequence at least 80, 85, 90, 95, 97, 98, 99, or 100% identical to SEQ ID NO: 311, and the light chain comprises a sequence at least 80, 85, 90, 95, 97, 98, 99, or 100% identical to SEQ ID NO: 312;

the heavy chain comprises a sequence at least 80, 85, 90, 95, 97, 98, 99, or 100% identical to SEQ ID NO: 313, and the light chain comprises a sequence at least 80, 85, 90, 95, 97, 98, 99, or 100% identical to SEQ ID NO: 314;

the heavy chain comprises a sequence at least 80, 85, 90, 95, 97, 98, 99, or 100% identical to SEQ ID NO: 315, and the light chain comprises a sequence at least 80, 85, 90, 95, 97, 98, 99, or 100% identical to SEQ ID NO: 316;

the heavy chain comprises a sequence at least 80, 85, 90, 95, 97, 98, 99, or 100% identical to SEQ ID NO: 317, and the light chain comprises a sequence at least 80, 85, 90, 95, 97, 98, 99, or 100% identical to SEQ ID NO: 318;

the heavy chain comprises a sequence at least 80, 85, 90, 95, 97, 98, 99, or 100% identical to SEQ ID NO: 319, and the light chain comprises a sequence at least 80, 85, 90, 95, 97, 98, 99, or 100% identical to SEQ ID NO: 320;

the heavy chain comprises a sequence at least 80, 85, 90, 95, 97, 98, 99, or 100% identical to SEQ ID NO: 321, and the light chain comprises a sequence at least 80, 85, 90, 95, 97, 98, 99, or 100% identical to SEQ ID NO: 322;

the heavy chain comprises a sequence at least 80, 85, 90, 95, 97, 98, 99, or 100% identical to SEQ ID NO: 323, and the light chain comprises a sequence at least 80, 85, 90, 95, 97, 98, 99, or 100% identical to SEQ ID NO: 324;

the heavy chain comprises a sequence at least 80, 85, 90, 95, 97, 98, 99, or 100% identical to SEQ ID NO: 325, and the light chain comprises a sequence at least 80, 85, 90, 95, 97, 98, 99, or 100% identical to SEQ ID NO: 326;

the heavy chain comprises a sequence at least 80, 85, 90, 95, 97, 98, 99, or 100% identical to SEQ ID NO: 327, and the light chain comprises a sequence at least 80, 85, 90, 95, 97, 98, 99, or 100% identical to SEQ ID NO: 328;

the heavy chain comprises a sequence at least 80, 85, 90, 95, 97, 98, 99, or 100% identical to SEQ ID NO: 329, and the light chain comprises a sequence at least 80, 85, 90, 95, 97, 98, 99, or 100% identical to SEQ ID NO: 330;

the heavy chain comprises a sequence at least 80, 85, 90, 95, 97, 98, 99, or 100% identical to SEQ ID NO: 331, and the light chain comprises a sequence at least 80, 85, 90, 95, 97, 98, 99, or 100% identical to SEQ ID NO: 332;

the heavy chain comprises a sequence at least 80, 85, 90, 95, 97, 98, 99, or 100% identical to SEQ ID NO: 333, and the light chain comprises a sequence at least 80, 85, 90, 95, 97, 98, 99, or 100% identical to SEQ ID NO: 334;

the heavy chain comprises a sequence at least 80, 85, 90, 95, 97, 98, 99, or 100% identical to SEQ ID NO: 335, and the light chain comprises a sequence at least 80, 85, 90, 95, 97, 98, 99, or 100% identical to SEQ ID NO: 336;

the heavy chain comprises a sequence at least 80, 85, 90, 95, 97, 98, 99, or 100% identical to SEQ ID NO: 337, and the light chain comprises a sequence at least 80, 85, 90, 95, 97, 98, 99, or 100% identical to SEQ ID NO: 338;

the heavy chain comprises a sequence at least 80, 85, 90, 95, 97, 98, 99, or 100% identical to SEQ ID NO: 339, and the light chain comprises a sequence at least 80, 85, 90, 95, 97, 98, 99, or 100% identical to SEQ ID NO: 340;

the heavy chain comprises a sequence at least 80, 85, 90, 95, 97, 98, 99, or 100% identical to SEQ ID NO: 341, and the light chain comprises a sequence at least 80, 85, 90, 95, 97, 98, 99, or 100% identical to SEQ ID NO: 342;

the heavy chain comprises a sequence at least 80, 85, 90, 95, 97, 98, 99, or 100% identical to SEQ ID NO: 343, and the light chain comprises a sequence at least 80, 85, 90, 95, 97, 98, 99, or 100% identical to SEQ ID NO: 344;

the heavy chain comprises a sequence at least 80, 85, 90, 95, 97, 98, 99, or 100% identical to SEQ ID NO: 345, and the light chain comprises a sequence at least 80, 85, 90, 95, 97, 98, 99, or 100% identical to SEQ ID NO: 346; or

the heavy chain comprises a sequence at least 80, 85, 90, 95, 97, 98, 99, or 100% identical to SEQ ID NO: 347, and the light chain comprises a sequence at least 80, 85, 90, 95, 97, 98, 99, or 100% identical to SEQ ID NO: 348;

the heavy chain comprises a sequence at least 80, 85, 90, 95, 97, 98, 99, or 100% identical to SEQ ID NO: 349, and the light chain comprises a sequence at least 80, 85, 90, 95, 97, 98, 99, or 100% identical to SEQ ID NO: 350;

the heavy chain comprises a sequence at least 80, 85, 90, 95, 97, 98, 99, or 100% identical to SEQ ID NO: 351, and the light chain comprises a sequence at least 80, 85, 90, 95, 97, 98, 99, or 100% identical to SEQ ID NO: 352;

the heavy chain comprises a sequence at least 80, 85, 90, 95, 97, 98, 99, or 100% identical to SEQ ID NO: 353, and the light chain comprises a sequence at least 80, 85, 90, 95, 97, 98, 99, or 100% identical to SEQ ID NO: 354; or

the heavy chain comprises a sequence at least 80, 85, 90, 95, 97, 98, 99, or 100% identical to SEQ ID NO: 355, and the light chain comprises a sequence at least 80, 85, 90, 95, 97, 98, 99, or 100% identical to SEQ ID NO: 356.

Also included are variants thereof, for example, variants having 1, 2, 3, 4, 5, 6, 7, 8, 9, or 10 alterations in one or more framework regions. Exemplary “alterations” include amino acid substitutions, additions, and deletions.

In some embodiments, an antibody, or antigen-binding fragment thereof, comprises, consists, or consists essentially of a V_{H} region and/or a V_{L} region characterized by a combination of V_{H}CDR1, V_{H}CDR2, V_{H}CDR3, V_{L}CDR1, V_{L}CDR2, and V_{L}CDR3 sequences, and framework region (FR) sequences, including heavy chain framework HFR1, HFR2, HFR2, and HFR4 sequences, and light chain LFR1, LFR2, LFR3, and LFR4 sequences. Table A3 provides a summary of the foregoing sequences.

TABLE A3
Exemplary CDR and Framework Regions

| | | |
|---|---|---|
| | | SEQ |
| | | ID |
| Description | Sequence | NO: |

HU177 Derived Sequences

| | | |
|---|---|---|
| HFR1 | QVTLKESGPALVKPTQTLTLTCTFSGFSLS | 357 |
| HFR1 | EVQLVESGGGLVQPGGSLRLSCAASGFTFS | 358 |
| HFR1 | EVQLVESGGGLVQPGGSLRLSCAASGFSFS | 359 |
| HFR1 | EVQLVESGGGLVQPGGSLRLSCAASGFTLS | 360 |
| HFR1 | EVQLVESGGGLVQPGGSLRLSCAASGFSLS | 361 |
| HFR2 | WIRQPPGKALEWLA | 362 |
| HFR2 | WVRQAPGKGLEWVA | 363 |
| HFR2 | WIRQAPGKGLEWVA | 364 |
| HFR2 | WVRQAPGKGLEWLA | 365 |
| HFR2 | WIRQAPGKGLEWLA | 366 |
| HFR3 | RLTISKDTSKNQVVLTMTNMDPVDTATYYCAR | 367 |
| HFR3 | RLTISRDNAKNSLYLQMNSLRAEDTAVYYCAR | 368 |
| HFR3 | RLTISKDNAKNSLYLQMNSLRAEDTAVYYCAR | 369 |
| HFR4 | WGQGTTVTVSS | 370 |
| VHCDR1 | TSGMGVG | 371 |
| VHCDR1 | TPGMGVW | 372 |
| VHCDR2 | DIWWDDNKYANPSLKS | 373 |
| VHCDR2 | DIWWDDNKYTNPSLKS | 374 |
| VHCDR3 | RANYGNPYYAQDY | 375 |
| LFR1 | DIVMTQTPLSLPVTPGEPASISC | 376 |
| LFR2 | WYLQKPGQSPQLLIY | 377 |
| LFR3 | GVPDRFSGSGSGTDFTLKISRVEAEDVGVYYC | 378 |
| LFR4 | FGQGTKVEIK | 379 |
| VLCDR1 | RSSQSIVHSNGNTYLE | 380 |
| VLCDR1 | RSSQSIVHSQGNTYLE | 381 |
| VLCDR1 | RSSQSIVHSSGNTYLE | 382 |
| VLCDR1 | RSSQSIVHSNANTYLE | 383 |
| VLCDR1 | RSSQSIVHSWGNTYLE | 384 |
| VLCDR1 | RSSQSIVHSWGNTYFE | 385 |
| VLCDR2 | KVSNRFS | 386 |
| VLCDR3 | FQGSHVPWT | 387 |
| VLCDR3 | FQGSHFPWT | 388 |

HUIV26 Derived Sequences

| | | |
|---|---|---|
| HFR1 | EVQLVESGGGLVQPGGSLRLSCAASGFTFS | 389 |
| HFR1 | EVQLVESGGGLVQPGGSLRLSCAASGFDFS | 390 |
| HFR2 | WVRQAPGKGLEWVA | 391 |
| HFR2 | WVRQAPGKGLEWIG | 392 |
| HFR3 | RFTISRDNAKNSLYLQMNSLRAEDTAVYYCAR | 393 |
| HFR4 | WGQGTTVTVSS | 394 |
| VHCDR1 | RYWMS | 395 |
| VHCDR1 | RYWMA | 396 |
| VHCDR1 | RYWMT | 397 |
| VHCDR2 | EINPDSSTANYTPALKG | 398 |
| VHCDR2 | EINPDSSTANYTPSLKG | 399 |
| VHCDR2 | EINPDSSTANYTPYLKG | 400 |
| VHCDR2 | EINPDASTANYTPYLKG | 401 |
| VHCDR3 | PVDGYYDAMDP | 402 |
| VHCDR3 | PVDAYYDAMDP | 403 |
| VHCDR3 | PVDAYYDAMDP | 404 |
| VHCDR3 | PVDGYYDAMDN | 405 |
| LFR1 | DIVMTQSPDSLAVSLGERATINC | 406 |
| LFR2 | WYQQKPGQPPKLLIY | 407 |
| LFR3 | GVPDRFSGSGSGTDFTLTISSLQAEDVAVYYC | 408 |
| LFR4 | FGQGTKLEIK | 409 |
| VLCDR1 | KSSQSLLNWHNQKNYLA | 410 |
| VLCDR1 | KSSQSLLNWYNQKNYLA | 411 |
| VLCDR1 | KSSQSLLNRYNQKNYLA | 412 |
| VLCDR2 | GASTRES | 413 |
| VLCDR3 | QNDHQYPYT | 414 |

Thus, in certain embodiments, an antibody, or antigen-binding fragment thereof, comprises, consists, or consists essentially of a V_{H} region or heavy chain comprising:

an HFR1 sequence selected from SEQ ID NOs: 357-361;

an HFR2 sequence selected from SEQ ID NOs: 362-366;

an HFR3 sequence selected from SEQ ID NOs: 367-369;

an HFR4 sequence set forth in SEQ ID NO: 370;

a V_{H}CDR1 sequence selected from SEQ ID NOs: 371-372;

a V_{H}CDR2 sequence selected from SEQ ID NOs: 373-374; and

a V_{H}CDR3 sequence selected from SEQ ID NO: 375,

and/or a V_{L} region or light chain comprising:

an LFR1 sequence set forth in SEQ ID NO: 376;

an LFR2 sequence set forth in SEQ ID NO: 377;

an LFR3 sequence set forth in SEQ ID NO: 378;

an LFR4 sequence set forth in SEQ ID NO: 379;

a V_{L}CDR1 sequence selected from SEQ ID NOs: 380-385;

a V_{L}CDR2 sequence set forth in SEQ ID NO: 386; and

a V_{L}CDR3 sequence selected from SEQ ID NOs: 387-388,

wherein the antibody, or antigen-binding fragment thereof, specifically binds to a human denatured collagen, as described herein.

In some embodiments, an antibody, or antigen-binding fragment thereof, comprises, consists, or consists essentially of a V_{H} region or heavy chain comprising:

an HFR1 sequence selected from SEQ ID NOs: 389-390;

an HFR2 sequence selected from SEQ ID NOs: 391-392;

an HFR3 sequence set forth in SEQ ID NO: 393;

an HFR4 sequence set forth in SEQ ID NO: 394;

a V_{H}CDR1 sequence selected from SEQ ID NOs: 395-397;

a V_{H}CDR2 sequence selected from SEQ ID NOs: 398-401; and

a V_{H}CDR3 sequence selected from SEQ ID NOs: 402-405,

and/or a VL region or light chain comprising:

an LFR1 sequence set forth in SEQ ID NO: 406;

an LFR2 sequence set forth in SEQ ID NO: 407;

an LFR3 sequence set forth in SEQ ID NO: 408;

an LFR4 sequence set forth in SEQ ID NO: 409;

a V_{L}CDR1 sequence selected from SEQ ID NOs: 410-412;

a V_{L}CDR2 set forth in SEQ ID NO: 413; and

a V_{L}CDR3 set forth in SEQ ID NO: 414,

wherein the antibody, or antigen-binding fragment thereof, specifically binds to a human denatured collagen, as described herein.

In certain embodiments, an antibody, or antigen-binding fragment thereof, specifically binds to human denatured collagen with a binding affinity of about 10 pM to about 500 pM or to about 1 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, or 1 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, or about 100 pM to about 1 nM.

In certain embodiments, an antibody, or antigen-binding fragment thereof, specifically binds to a human denatured collagen polypeptide (see Table C1) with a binding affinity that is about or at least about 1.5, 2, 3, 4, 5, 6, 7, 8, 9, 10, 15, 20, 30, 40, 50, 60, 70, 80, 90, or 100-fold stronger that its binding affinity for a corresponding native human collagen polypeptide.

In certain embodiments, variant antibodies or antigen-binding fragments, or V_{H}, V_{L}, or CDR regions thereof, specifically bind to denatured human collagen at least about 50%, at least about 70%, and in certain embodiments, at least about 90% as well as an antibody sequence specifically set forth herein. In further embodiments, such variant antibodies or antigen-binding fragments, or V_{H}, V_{L}, or CDR regions thereof, bind to denatured human collagen with greater affinity than the antibodies described herein, for example, that bind quantitatively at least about 105%, 106%, 107%, 108%, 109%, or 110% as well as an antibody sequence specifically set forth herein.

Determination of the three-dimensional structures of representative polypeptides (e.g., variant antibody or an antigen-binding fragment thereof) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of antibodies, and antigen-binding domains thereof, as provided herein, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

In some embodiments, an antibody, or antigen-binding fragment thereof, comprises an Fc region, for example, an Fc region selected from an IgA Fc region (including subclasses IgA1 and IgA2), an IgD Fc region, an IgE Fc region, an IgG Fc region (including subclasses IgG1, IgG2, IgG3, and IgG4), and an IgM Fc region. In some instances, the Fc region is a human Fc region. In some embodiments, an Fc region has high effector function in humans, for example, an IgG1 Fc region or an IgG3 Fc region. In some embodiments, an Fc region has low effector function in humans, for example, an IgG2 Fc region or an IgG4 Fc region.

The Fc region of an antibody interacts with a number of Fc receptors and ligands, imparting an array of important functional capabilities referred to as effector functions. For IgG, the Fc region comprises Ig domains CH2 and CH3 and the N-terminal hinge leading into CH2. One family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIlb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and IIIX) (Radaev et al., 2001, J Biol Chem 276:16469-16477.)

The Fc region is also involved in activation of the complement cascade. In the classical complement pathway, C1 binds with its C1q subunits to Fc fragments of IgG or IgM, which has formed a complex with antigen(s). In certain embodiments, modifications to the Fc region comprise modifications that alter (either enhance or decrease) the ability of an antibody, or antigen-binding fragment thereof, as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996)).

In some embodiments, an antibody, or antigen-binding fragment thereof, comprises a modified Fc region, including Fc regions having altered properties or biological activities relative to wild-type Fc region(s). In particular embodiments, a modified Fc region has at least one altered effector function and/or pharmacokinetic (PK) characteristic relative to a wild-type Fc region. Thus in certain embodiments, an antibody, or antigen-binding fragment thereof, has a modified Fc region with altered functional properties, such as reduced or enhanced CDC, ADCC, or ADCP activity, reduced or enhanced binding affinity for a specific FcγR, or increased serum half-life.

Examples of modified Fc regions include those having mutated sequences, for instance, by substitution, insertion, deletion, or truncation of one or more amino acids relative to a wild-type sequence, hybrid Fc polypeptides composed of domains from different immunoglobulin classes/subclasses, Fc polypeptides having altered glycosylation/sialylation patterns, and Fc polypeptides that are modified or derivatized, for example, by biotinylation (see, e.g., US Application No. 2010/0209424), phosphorylation, sulfation, etc., or any combination of the foregoing. Such modifications can be employed to alter (e.g., increase, decrease) the binding properties of the Fc region to one or more particular FcRs (e.g., FcγRI, FcγRIIa, FcγRIIb, FcγRIIc, FcγRIIIa, FcγRIIIb, FcRn), its pharmacokinetic properties (e.g., stability or half-life, bioavailability, tissue distribution, volume of distribution, concentration, elimination rate constant, elimination rate, area under the curve (AUC), clearance, Cmax, tmax, Cmin, fluctuation), its immunogenicity, its complement fixation or activation, and/or the CDC/ADCC/ADCP-related activities of the Fc region, among other properties described herein, relative to a corresponding wild-type Fc sequence of an antibody or antigen-binding fragment thereof. Included are modified Fc regions of human and/or mouse origin.

Certain embodiments include antibodies, or antigen-binding fragments thereof, which comprise hybrid Fc regions, for example, Fc regions that comprise a combination of Fc domains (e.g., hinge, CH2, CH3, CH4) from immunoglobulins of different species (e.g., human, mouse), different Ig classes, and/or different Ig subclasses. General examples include hybrid Fc regions that comprise, consist of, or consist essentially of the following combination of CH2/CH3 domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgE/IgA1, IgE/IgA2, IgE/IgD, IgE/IgE, IgE/IgG1, IgE/IgG2, IgE/IgG3, IgE/IgG4, IgE/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM, IgM/IgA1, IgM/IgA2, IgM/IgD, IgM/IgE, IgM/IgG1, IgM/IgG2, IgM/IgG3, IgM/IgG4, IgM/IgM (or fragments or variants thereof), and optionally include a hinge from one or more of IgA1, IgA2, IgD, IgG1, IgG2, IgG3, or IgG4, and/or a CH4 domain from IgE and/or IgM. In specific embodiments, the hinge, CH2, CH3, and CH4 domains are from human Ig.

Additional examples include hybrid Fc regions that comprise, consist of, or consist essentially of the following combination of CH2/CH4 domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH3 domain from one or more of IgA1, IgA2, IgD, IgE, IgG1, IgG2, IgG3, IgG4, or IgM. In specific embodiments, the hinge, CH2, CH3, and CH4 domains are from human Ig.

Certain examples include hybrid Fc regions that comprise, consist of, or consist essentially of the following combination of CH3/CH4 domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH2 domain from one or more of IgA1, IgA2, IgD, IgE, IgG1, IgG2, IgG3, IgG4, or IgM. In specific embodiments, the hinge, CH2, CH3, and CH4 domains are from human Ig.

Particular examples include hybrid Fc regions that comprise, consist of, or consist essentially of the following combination of hinge/CH2 domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH3 domain from one or more of IgA1, IgA2, IgD, IgE, IgG1, IgG2, IgG3, IgG4, or IgM, and/or a CH4 domain from IgE and/or IgM. In specific embodiments, the hinge, CH2, CH3, and CH4 domains are from human Ig.

Certain examples include hybrid Fc regions that comprise, consist of, or consist essentially of the following combination of hinge/CH3 domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH2 domain from one or more of IgA1, IgA2, IgD, IgE, IgG1, IgG2, IgG3, IgG4, or IgM, and/or a CH4 domain from IgE and/or IgM. In specific embodiments, the hinge, CH2, CH3, and CH4 domains are from human Ig.

Some examples include hybrid Fc regions that comprise, consist of, or consist essentially of the following combination of hinge/CH4 domains: IgA1/IgE, IgA1/IgM, IgA2/IgE, IgA2/IgM, IgD/IgE, IgD/IgM, IgG1/IgE, IgG1/IgM, IgG2/IgE, IgG2/IgM, IgG3/IgE, IgG3/IgM, IgG4/IgE, IgG4/IgM (or fragments or variants thereof), and optionally include a CH2 domain from one or more of IgA1, IgA2, IgD, IgE, IgG1, IgG2, IgG3, IgG4, or IgM, and/or a CH3 domain from one or more of IgA1, IgA2, IgD, IgE, IgG1, IgG2, IgG3, IgG4, or IgM.

Specific examples of hybrid Fc regions can be found, for example, in WO 2008/147143, which are derived from combinations of IgG subclasses or combinations of human IgD and IgG.

Also included are antibodies, or antigen-binding fragments thereof, which have derivatized or otherwise modified Fc regions. In certain aspects, the Fc region may be modified by phosphorylation, sulfation, acrylation, glycosylation, methylation, farnesylation, acetylation, amidation, and the like, for instance, relative to a wild-type or naturally-occurring Fc region. In certain embodiments, the Fc region may comprise wild-type or native glycosylation patterns, or alternatively, it may comprise increased glycosylation relative to a native form, decreased glycosylation relative to a native form, or it may be entirely deglycosylated. As one example of a modified Fc glycoform, decreased glycosylation of an Fc region reduces binding to the C1q region of the first complement component C1, a decrease in ADCC-related activity, and/or a decrease in CDC-related activity. Certain embodiments thus employ a deglycosylated or aglycosylated Fc region. See, e.g., WO 2005/047337 for the production of exemplary aglycosylated Fc regions. Another example of an Fc region glycoform can be generated by substituting the Q295 position with a cysteine residue (see, e.g., U.S. Application No. 2010/0080794), according to the Kabat et al. numbering system. Certain embodiments may include Fc regions where about 80-100% of the glycoprotein in Fc region comprises a mature core carbohydrate structure that lacks fructose (see, e.g., U.S. Application No. 2010/0255013). Some embodiments may include Fc regions that are optimized by substitution or deletion to reduce the level of fucosylation, for instance, to increase affinity for FcγRI, FcγRIa, or FcγRIIIa, and/or to improve phagocytosis by FcγRIIa-expressing cells (see U.S. Application Nos. 2010/0249382 and 2007/0148170).

As another example of a modified Fc glycoform, an Fc region of an antibody or antigen-binding fragment thereof may comprise oligomannose-type N-glycans, and optionally have one or more of the following: increased ADCC effector activity, increased binding affinity for FcγRIIIA (and certain other FcRs), and/or similar or lower binding affinity for mannose receptor, relative to a corresponding Fc region that contains complex-type N-glycans (see, e.g., U.S. Application No. 2007/0092521 and U.S. Pat. No. 7,700,321). As another example, enhanced affinity of Fc regions for FcγRs has been achieved using engineered glycoforms generated by expression of antibodies in engineered or variant cell lines (see, e.g., Umana et al., Nat Biotechnol. 17:176-180, 1999; Davies et al., Biotechnol Bioeng. 74:288-294, 2001; Shields et al., J Biol Chem. 277:26733-26740, 2002; Shinkawa et al., 2003, J Biol Chem. 278:3466-3473, 2003; and U.S. Application No. 2007/0111281). Certain Fc region glycoforms comprise an increased proportion of N-glycoside bond type complex sugar chains, which do not have the 1-position of fucose bound to the 6-position of N-acetylglucosamine at the reducing end of the sugar chain (see, e.g., U.S. Application No. 2010/0092997). Particular embodiments may include IgG Fc region that is glycosylated with at least one galactose moiety connected to a respective terminal sialic acid moiety by an α-2,6 linkage, optionally where the Fc region has a higher anti-inflammatory activity relative to a corresponding, wild-type Fc region (see U.S. Application No. 2008/0206246). Certain of these and related altered glycosylation approaches have generated substantial enhancements of the capacity of Fc regions to selectively bind FcRs such as FcγRIII, to mediate ADCC, and to alter other properties of Fc regions, as described herein.

Certain modified Fc regions of an antibody or antigen-binding fragment thereof may have altered binding to one or more FcRs, and/or corresponding changes to effector function, relative to a corresponding, wild-type Fc sequence (e.g., same species, same Ig class, same Ig subclass). For instance, such Fc regions may have increased binding to one or more of Fcγ receptors, Fcα receptors, Feε receptors, and/or the neonatal Fc receptor, relative to a corresponding, wild-type Fc sequence. In other embodiments, variant, fragment, hybrid, or modified Fc regions may have decreased binding to one or more of Fcγ receptors, Fcα receptors, Feε receptors, and/or the neonatal Fc receptor, relative to a corresponding, wild-type Fc sequence. Specific FcRs are described elsewhere herein.

In some embodiments, an antibody comprises an Fc domain, comprising one or more mutations to increase binding to one or more of Fcγ receptors, Fcα receptors, Feε receptors, and/or the neonatal Fc receptor, relative to a corresponding, wild-type Fc sequence. In some embodiments, an antibody comprises an Fc domain, comprising one or more mutations to decrease binding to one or more of Fcγ receptors, Fcα receptors, Feε receptors, and/or the neonatal Fc receptor, relative to a corresponding, wild-type Fc sequence.

Specific examples of Fc variants having altered (e.g., increased, decreased) effector function/FcR binding can be found, for example, in U.S. Pat. Nos. 5,624,821 and 7,425,619; U.S.

Application Nos. 2009/0017023, 2009/0010921, and 2010/0203046; and WO 2000/42072 and WO 2004/016750. Certain examples include human Fc regions having a one or more substitutions at position 298, 333, and/or 334, for example, S298A, E333A, and/or K334A (based on the numbering of the EU index of Kabat et al.), which have been shown to increase binding to the activating receptor FcγRIIIa and reduce binding to the inhibitory receptor FcγRIIb. These mutations can be combined to obtain double and triple mutation variants that have further improvements in binding to FcRs. Certain embodiments include a S298A/E333A/K334A triple mutant, which has increased binding to FcγRIIIa, decreased binding to FcγRIIb, and increased ADCC (see, e.g., Shields et al., J Biol Chem. 276:6591-6604, 2001; and Presta et al., Biochem Soc Trans. 30:487-490, 2002). See also engineered Fc glycoforms that have increased binding to FcRs, as disclosed in Umana et al., supra; and U.S. Pat. No. 7,662,925. Some embodiments include Fc regions that comprise one or more substitutions selected from 434S, 252Y/428L, 252Y/4345, and 428L/4345 (see U.S. Application Nos. 2009/0163699 and 20060173170), based on the EU index of Kabat et al.

As noted above, certain modified Fc regions may have altered effector functions, relative to a corresponding, wild-type Fc sequence. For example, such Fc regions may have increased complement fixation or activation, increased C1q binding affinity, increased CDC-related activity, increased ADCC-related activity, and/or increased ADCP-related activity, relative to a corresponding, wild-type Fc sequence. In some embodiments, such Fc regions may have decreased complement fixation or activation, decreased C1q binding affinity, decreased CDC-related activity, decreased ADCC-related activity, and/or decreased ADCP-related activity, relative to a corresponding, wild-type Fc sequence. As merely one illustrative example, an Fc region may comprise a deletion or substitution in a complement-binding site, such as a C1q-binding site, and/or a deletion or substitution in an ADCC site. Examples of such deletions/substitutions are described, for example, in U.S. Pat. No. 7,030,226. Many Fc effector functions, such as ADCC, can be assayed according to routine techniques in the art. (see, e.g., Zuckerman et al., CRC Crit Rev Microbiol. 7:1-26, 1978). Useful effector cells for such assays includes, but are not limited to, natural killer (NK) cells, macrophages, and other peripheral blood mononuclear cells (PBMC). Alternatively, or additionally, certain Fc effector functions may be assessed in vivo, for example, by employing an animal model described in Clynes et al. PNAS. 95:652-656, 1998.

Certain modified Fc regions have altered stability or half-life relative to a corresponding, wild-type Fc sequence. In certain embodiments, such Fc regions may have increased half-life relative to a corresponding, wild-type Fc sequence. In some embodiments, modified Fc regions have decreased half-life relative to a corresponding, wild-type Fc sequence. Half-life can be measured in vitro (e.g., under physiological conditions) or in vivo, according to routine techniques in the art, such as radiolabeling, ELISA, or other methods. In vivo measurements of stability or half-life can be measured in one or more bodily fluids, including blood, serum, plasma, urine, or cerebrospinal fluid, or a given tissue, such as the liver, kidneys, muscle, central nervous system tissues, bone, etc. As one example, modifications to an Fc region that alter its ability to bind the FcRn can alter its half-life in vivo. Assays for measuring the in vivo pharmacokinetic properties (e.g., in vivo mean elimination half-life) and non-limiting examples of Fc modifications that alter its binding to the FcRn are described, for example, in U.S. Pat. Nos. 7,217,797 and 7,732,570; and U.S. Application Nos. US 2010/0143254 and 2010/0143254.

Additional non-limiting examples of modifications to alter stability or half-life include substitutions/deletions at one or more of amino acid residues selected from 251-256, 285-290, and 308-314 in the CH2 domain, and 385-389 and 428-436 in the CH3 domain, according to the numbering system of Kabat et al. See U.S. Application No. 2003/0190311. Specific examples include substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, and/or substitution with histidine, tyrosine, arginine or threonine at position 436, including any combination thereof. Such modifications optionally increase affinity of the Fc region for the FcRn and thereby increase half-life, relative to a corresponding, wild-type Fc region.

Certain modified Fc regions have altered solubility relative to a corresponding, wild-type Fc sequence. In certain embodiments, such Fc regions have increased solubility relative to a corresponding, wild-type Fc sequence. In some embodiments, modified Fc regions have decreased solubility relative to a corresponding, wild-type Fc sequence. Solubility can be measured, for example, in vitro (e.g., under physiological conditions) according to routine techniques in the art. Exemplary solubility measurements are described elsewhere herein.

Additional examples of variants include IgG Fc regions having conservative or non-conservative substitutions (as described elsewhere herein) at one or more of positions 250, 314, or 428 of the heavy chain, or in any combination thereof, such as at positions 250 and 428, or at positions 250 and 314, or at positions 314 and 428, or at positions 250, 314, and 428 (see, e.g., U.S. Application No. 2011/0183412). In specific embodiments, the residue at position 250 is substituted with glutamic acid or glutamine, and/or the residue at position 428 is substituted with leucine or phenylalanine. As another illustrative example of an IgG Fc variant, any one or more of the amino acid residues at positions 214 to 238, 297 to 299, 318 to 322, and/or 327 to 331 may be used as a suitable target for modification (e.g., conservative or non-conservative substitution, deletion). In particular embodiments, the IgG Fc variant CH2 domain contains amino acid substitutions at positions 228, 234, 235, and/or 331 (e.g., human IgG4 with Ser228Pro and Leu235Ala mutations) to attenuate the effector functions of the Fc region (see U.S. Pat. No. 7,030,226). Here, the numbering of the residues in the heavy chain is that of the EU index (see Kabat et al., “Sequences of Proteins of Immunological Interest,” 5th Ed., National Institutes of Health, Bethesda, Md. (1991)). Certain of these and related embodiments have altered (e.g., increased, decreased) FcRn binding and/or serum half-life, optionally without reduced effector functions such as ADCC or CDC-related activities.

Additional examples include variant Fc regions that comprise one or more amino acid substitutions at positions 279, 341, 343 or 373 of a wild-type Fc region, or any combination thereof (see, e.g., U.S. Application No. 2007/0224188). The wild-type amino acid residues at these positions for human IgG are valine (279), glycine (341), proline (343) and tyrosine (373). The substation(s) can be conservative or non-conservative, or can include non-naturally occurring amino acids or mimetics, as described herein. Alone or in combination with these substitutions, certain embodiments may also employ a variant Fc region that comprises at least 1, 2, 3, 4, 5, 6, 7, 8, 9, 10 or more amino acid substitutions selected from the following: 235G, 235R, 236F, 236R, 236Y, 237K, 237N, 237R, 238E, 238G, 238H, 238I, 238L, 238V, 238W, 238Y, 244L, 245R, 247A, 247D, 247E, 247F, 247M, 247N, 247Q, 247R, 2475, 247T, 247W, 247Y, 248F, 248P, 248Q, 248W, 249L, 249M, 249N, 249P, 249Y, 251H, 251I, 251W, 254D, 254E, 254F, 254G, 254H, 254I, 254K, 254L, 254M, 254N, 254P, 254Q, 254R, 254V, 254W, 254Y, 255K, 255N, 256H, 256I, 256K, 256L, 256V, 256W, 256Y, 257A, 257I, 257M, 257N, 257S, 258D, 260S, 262L, 264S, 265K, 265S, 267H, 267I, 267K, 268K, 269N, 269Q, 271T, 272H, 272K, 272L, 272R, 279A, 279D, 279F, 279G, 279H, 279I, 279K, 279L, 279M, 279N, 279Q, 279R, 279S, 279T, 279W, 279Y, 280T, 283F, 283G, 283H, 283I, 283K, 283L, 283M, 283P, 283R, 283T, 283W, 283Y, 285N, 286F, 288N, 288P, 292E, 292F, 292G, 292I, 292L, 293S, 293V, 301W, 304E, 307E, 307M, 312P, 315F, 315K, 315L, 315P, 315R, 316F, 316K, 317P, 317T, 318N, 318P, 318T, 332F, 332G, 332L, 332M, 332S, 332V, 332W, 339D, 339E, 339F, 339G, 339H, 339I, 339K, 339L, 339M, 339N, 339Q, 339R, 339S, 339W, 339Y, 341D, 341E, 341F, 341H, 341I, 341K, 341L, 341M, 341N, 341P, 341Q, 341R, 341S, 341T, 341V, 341W, 341Y, 343A, 343D, 343E, 343F, 343G, 343H, 343I, 343K, 343L, 343M, 343N, 343Q, 343R, 343S, 343T, 343V, 343W, 343Y, 373D, 373E, 373F, 373G, 373H, 373I, 373K, 373L, 373M, 373N, 373Q, 373R, 373S, 373T, 373V, 373W, 375R, 376E, 376F, 376G, 376H, 376I, 376L, 376M, 376N, 376P, 376Q, 376R, 376S, 376T, 376V, 376W, 376Y, 377G, 377K, 377P, 378N, 379N, 379Q, 379S, 379T, 380D, 380N, 380S, 380T, 382D, 382F, 382H, 382I, 382K, 382L, 382M, 382N, 382P, 382Q, 382R, 382S, 382T, 382V, 382W, 382Y, 385E, 385P, 386K, 423N, 424H, 424M, 424V, 426D, 426L, 427N, 429A, 429F, 429M, 430A, 430D, 430F, 430G, 430H, 430I, 430K, 430L, 430M, 430N, 430P, 430Q, 430R, 430S, 430T, 430V, 430W, 430Y, 431H, 431K, 431P, 432R, 432S, 438G, 438K, 438L, 438T, 438W, 439E, 439H, 439Q, 440D, 440E, 440F, 440G, 440H, 440I, 440K, 440L, 440M, 440Q, 440T, 440V or 442K. As above, the numbering of the residues in the heavy chain is that of the EU index (see Kabat et al., supra). Such variant Fc regions typically confer an altered effector function or altered serum half-life upon the antibody to which the variant Fc region is operably attached. Preferably the altered effector function is an increase in ADCC, a decrease in ADCC, an increase in CDC, a decrease in CDC, an increase in C1q binding affinity, a decrease in C1q binding affinity, an increase in FcR (preferably FcRn) binding affinity or a decrease in FcR (preferably FcRn) binding affinity as compared to a corresponding Fc region that lacks such amino acid substitution(s).

Additional examples include variant Fc regions that comprise an amino acid substitution at one or more of position(s) 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and/or 428 (see, e.g., U.S. Pat. No. 7,662,925). In specific embodiments, the variant Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, 1332D, I332E, I332N, I332Q, T335D, T335R, and T335Y. In other specific embodiments, the variant Fc region comprises at least one amino acid substitution selected from the group consisting of: V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, In more specific embodiments, the variant Fc region comprises a series of substitutions selected from the group consisting of: N297D/I332E, F241Y/F243Y/V262T/V264T/N297D/I332E, S239D/N297D/I332E, S239E/N297D/I332E, S239D/D265Y/N297D/I332E, S239D/D265H/N297D/I332E, V264E/N297D/I332E, Y296N/N297D/I332E, N297D/A330Y/I332E, S239D/D265V/N297D/I332E, S239D/D265I/N297D/I332E, and N297D/S298A/A330Y/I332E. In specific embodiments, the variant Fc region comprises an amino acid substitution at position 332 (using the numbering of the EU index, Kabat et al., supra). Examples of substitutions include 332A, 332D, 332E, 332F, 332G, 332H, 332K, 332L, 332M, 332N, 332P, 332Q, 332R, 332S, 332T, 332V, 332W and 332Y. The numbering of the residues in the Fc region is that of the EU index of Kabat et al. Among other properties described herein, such variant Fc regions may have increased affinity for an FcγR, increased stability, and/or increased solubility, relative to a corresponding, wild-type Fc region.

Further examples include variant Fc regions that comprise one or more of the following amino acid substitutions: 224N/Y, 225A, 228L, 230S, 239P, 240A, 241L, 2435/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, optionally where the variant has altered recognition of an Fc ligand and/or altered effector function compared with a parent Fc polypeptide, and wherein the numbering of the residues is that of the EU index as in Kabat et al. Specific examples of these and related embodiments include variant Fc regions that comprise or consist of the following sets of substitutions: (1) N276D, R292Q, V305A, 1377V, T394A, V412A and K439E; (2) P244L, K246E, D399G and K409R; (3) S304G, K320R, S324T, K326E and M358T; (4) F243S, P247L, D265V, V266A, S383N and T411I; (5) H224N, F243L, T393A and H433P; (6) V240A, S267G, G341E and E356G; (7) M252T, P291L, P352A, R355W, N390D, S408G, S426F and A431S; (8) P228L, T289A, L365Q, N389S and 5440G; (9) F241L, V273A, K340Q and L441F; (10) F241L, T299A, 1332T and M428T; (11) E269K, Y300H, Q342R, V422I and G446A; (12) T225A, R301c, S304G, D312N, N315D, L351S and N421S; (13) S254T, L306I, K326R and Q362L; (14) H224Y, P230S, V323A, E333D, K338R and S364C; (15) T335I, K414M and P445R; (16) T335I and K414M; (17) P247A, E258K, D280N, K288R, N297D, T299A, K322E, Q342R, S354A and L365P; (18) H268N, V279M, A339T, N361D and S426P; (19) C261Y, K290E, L306F, Q311R, E333G and Q438L; (20) E283G, N315K, E333G, R344Q, L365P and S442T; (21) Q347R, N361Y and K439R; (22) S239P, S254P, S267N, H285R, N315S, F372L, A378T, N390D, Y391C, F404S, E430K, L432P and K447E; and (23) E269G, Y278H, N325S and K370R, wherein the numbering of the residues is that of the EU index as in Kabat et al. (see, e.g., U.S. Application No. 2010/0184959).

Variant Fc regions can also have one or more mutated hinge regions, as described, for example, in U.S. Application No. 2003/0118592. For instance, one or more cysteines in a hinge region can be deleted or substituted with a different amino acid. The mutated hinge region can comprise no cysteine residues, or it can comprise 1, 2, or 3 fewer cysteine residues than a corresponding, wild-type hinge region. In some embodiments, an Fc region having a mutated hinge region of this type exhibits a reduced ability to dimerize, relative to a wild-type Ig hinge region.

As noted above, antibodies having altered Fc regions typically have altered (e.g., improved, increased, decreased) pharmacokinetic properties relative to corresponding wild-type Fc region. Examples of pharmacokinetic properties include stability or half-life, bioavailability (the fraction of a drug that is absorbed), tissue distribution, volume of distribution (apparent volume in which a drug is distributed immediately after it has been injected intravenously and equilibrated between plasma and the surrounding tissues), concentration (initial or steady-state concentration of drug in plasma), elimination rate constant (rate at which drugs are removed from the body), elimination rate (rate of infusion required to balance elimination), area under the curve (AUC or exposure; integral of the concentration-time curve, after a single dose or in steady state), clearance (volume of plasma cleared of the drug per unit time), Cmax (peak plasma concentration of a drug after oral administration), Tmax (time to reach Cmax), Cmin (lowest concentration that a drug reaches before the next dose is administered), and fluctuation (peak trough fluctuation within one dosing interval at steady state).

In particular embodiments, an antibody, or antigen-binding fragment thereof, has a biological half life at about pH 7.4, at about a physiological pH, at about 25° C. or room temperature, and/or at about 37° C. or human body temperature (e.g., in vivo, in serum, in a given tissue, in a given species such as rat, mouse, monkey, or human), of about or at least about 30 minutes, about 1 hour, about 2 hour, about 3 hours, about 4 hours, about 5 hours, about 6 hours, about 12 hours, about 18 hours, about 20 hours, about 24 hours, about 30 hours, about 36 hours, about 40 hours, about 48 hours, about 50 hours, about 60 hours, about 70 hours, about 72 hours, about 80 hours, about 84 hours, about 90 hours, about 96 hours, about 120 hours, or about 144 hours or more, or about 1 week, or about 2 weeks, or about 3 weeks, or about 4 weeks, or about 5 weeks, or about 6 weeks or more, or any intervening half-life, including all ranges in between.

In some embodiments, an antibody, or antigen-binding fragment thereof, has a Tm of about or at least about 60, 62, 64, 66, 68, 70, 72, 74, or 75° C. In some embodiments, an antibody, or antigen-binding fragment, thereof has a Tm of about 60° C. or greater.

Any one or more of the foregoing anti-denatured collagen antibodies (including antigen-binding fragments thereof) can be combined with any one or more of the effector domains and linkers described herein, to generate one or more fusion proteins or larger, multi-chain structures comprising the same.

Effector Domains

The fusion proteins provided herein comprise at least one effector domain (e.g., an effector ligand domain), such as a cytokine or an immunomodulatory or anti-cancer antibody. In some instances, the effector domain comprises an immune cell-stimulatory ligand or domain, an immune cell-inhibitory ligand or domain, and/or a cytocidal (e.g., tumor cell cytocidal) ligand or domain.

Examples of effector domains or effector ligand domains include an interleukin-2 (IL-2) polypeptide, an interleukin-15 (IL-15) polypeptide, a hybrid IL-2/IL-15 polypeptide, a TNF superfamily ligand polypeptide, an interleukin-12 (IL-12) polypeptide, an interleukin-10 (IL-10) polypeptide, an interleukin-7 (IL-7) polypeptide, an interleukin-21 (IL-21) polypeptide, and an interferon-α (IFN-α) polypeptide. Examples of immunomodulatory or anti-cancer antibodies, and antigen-binding fragments thereof, are described herein, including anti-CD40 and anti-4-1BB antibodies, and antigen-binding fragments thereof.

Interleukin-2 (L-2). Certain effector domains comprise one or more “IL-2 polypeptides”, including human IL-2 polypeptides. IL-2 is a cytokine signals through the IL-2 receptor (IL-2R), a complex composed of up to three chains, termed the α (CD25), β (CD122) and γc (CD132) chains. IL-2 is produced by T-cells in response to antigenic or mitogenic stimulation, and is required for T-cell proliferation and other activities crucial to regulation of the immune response. IL-2 can stimulate B-cells, monocytes, lymphokine-activated killer cells, natural killer cells, and glioma cells, among other immune cells.

IL-2 is a 15-16 kDA protein composed of a signal peptide (residues 1-20) and an active mature protein (residues 21-153). Exemplary IL-2 protein sequences are provided in Table S1.

TABLE S1
Exemplary IL-2 polypeptide sequences

| | | SEQ |
|---|---|---|
| Name | Sequence | ID NO: |
| Human IL-2 | MYRMQLLSCIALSLALVTNSAPTSSSTKKTQLQLEHLLLDLQMILNGINNY | 426 |
| FL | KNPKLTRMLTFKFYMPKKATELKHLQCLEEELKPLEEVLNLAQSKNFHLRP | |
| Precursor | RDLISNINVIVLELKGSETTFMCEYADETATIVEFLNRWITFCQSIISTLT | |
| Human IL-2 | APTSSSTKKTQLQLEHLLLDLQMILNGINNYKNPKLTRMLTFKFYMPKKAT | 427 |
| mature form | ELKHLQCLEEELKPLEEVLNLAQSKNFHLRPRDLISNINVIVLELKGSETT | |
| (C125S) | FMCEYADETATIVEFLNRWITFSQSIISTLT | |
| Human IL-2 | APTSSSTKKTQLQLEHLLLDLQMILNGINNYKNPKLTRMLTFKFYMPKKAT | 428 |
| mature form | ELKHLQCLEEELKPLEEVLNLAHSKNFHFDPRDVVSNINVFVLELKGSETT | |
| (D10) | FMCEYADETATIVEFLNRWITFCQSIISTLT | |
| Q74H, L80F, | | |
| R81D, L85V, | | |
| I86V, and | | |
| I92F | | |
| Human IL-2 | APASSSTKKTQLQLEHLLLDLQMILNGINNYKNPKLTRMLTFKFYMPKKAT | 429 |
| mature form | ELKHLQCLEEELKPLEEVLNLAQSKNFHLRPRDLISNINVIVLELKGSETT | |
| with T3A | FMCEYADETATIVEFLNRWITFSQSIISTLT | |
| Human IL-2 | APASSSTKKTQLQLEHLLLDLQMILNGINNYKNPKLTRMLTAKFYMPKKAT | 430 |
| mature form | ELKHLQCLEEELKPLEEVLNLAQSKNFHLRPRDLISNINVIVLELKGSETT | |
| with T3A, | FMCEYADETATIVEFLNRWITFSQSIISTLT | |
| F42A | | |
| Human IL-2 | APASSSTKKTQLQLEHLLLDLQMILNGINNYKNPKLTRMLTFKFYMPKKAT | 431 |
| mature form | ELKHLQCLEEELKPLEEALNLAPSKNFHLRPRDLISNINVIVLELKGSETT | |
| with T3A, | FMCEYADETATIVEFLNRWITFSQSTISTLT | |
| V69A, Q74P, | | |
| I128T | | |
| Human IL-2 | APASSSTKKTQLQLEHLLLTLQMILNGINNYKNPKLTRMLTFKFYMPKKAT | 432 |
| mature form | ELKHLQCLEEELKPLEEVLNLAQSKNFHLRPRDLISNINVIVLELKGSETT | |
| with T3A, | FMCEYADETATIVEFLNRWITFSQSIISTLT | |
| D20T | | |
| Human IL-2 | APASSSTKKTQLQLEHLLLTLQMILNGINNYKNPKLTRMLTAKFYMPKKAT | 433 |
| mature form | ELKHLQCLEEELKPLEEVLNLAQSKNFHLRPRDLISNINVIVLELKGSETT | |
| with T3A, | FMCEYADETATIVEFLNRWITFSQSIISTLT | |
| D20T, F42A | | |
| Human IL-2 | APASSSTKKTQLQLEHLLLDLQMILNGINNYKNPKLTEMLTFKFYMPKKAT | 434 |
| mature form | ELKHLQCLEEELKPLEEVLNLAQSKNFHLRPRDLISNINVIVLELKGSETT | |
| with R38E, | FMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKAT | 435 |
| mature form | ELKHLQCLEKELKPLEEVLNLAQSKNFHLRPRDLISNINVIVLELKGSETT | |
| with E61K, | FMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKAT | 436 |
| mature form | ELKHLQCLEEKLKPLEEVLNLAQSKNFHLRPRDLISNINVIVLELKGSETT | |
| with E62K, | FMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKAT | 437 |
| mature form | ELKHLQCLEKKLKPLEEVLNLAQSKNFHLRPRDLISNINVIVLELKGSETT | |
| with E61K, | FMCEYADETATIVEFLNRWITFSQSIISTLT | |
| E62K, T3A | | |
| Human IL-2 | APASSSTKKTQLQLEHLLLDLQMILNGINNYKNPKLTEMLTFKFYMPKKAT | 438 |
| mature form | ELKHLQCLEKKLKPLEEVLNLAQSKNFHLRPRDLISNINVIVLELKGSETT | |
| with R38E, | FMCEYADETATIVEFLNRWITFSQSIISTLT | |
| E61K, E62K, | | |
| T3A | | |
| Human IL-2 | APASSSTKKTQLQLEHLLLDLQMILNGINNYKNPKLTEMLTAKFYMPKKAT | 439 |
| mature form | ELKHLQCLEEELKPLEEVLNLAQSKNFHLRPRDLISNINVIVLELKGSETT | |
| with F42A, | FMCEYADETATIVEFLNRWITFSQSIISTLT | |
| R38E, T3A | | |
| Human IL-2 | APASSSTKKTQLQLEHLLLDLQMILNGINNYKNPKLTRMLTAKFYMPKKAT | 440 |
| mature form | ELKHLQCLEKELKPLEEVLNLAQSKNFHLRPRDLISNINVIVLELKGSETT | |
| with F42A, | FMCEYADETATIVEFLNRWITFSQSIISTLT | |
| E61K, T3A | | |
| Human IL-2 | APASSSTKKTQLQLEHLLLDLQMILNGINNYKNPKLTRMLTAKFYMPKKAT | 441 |
| mature form | ELKHLQCLEEKLKPLEEVLNLAQSKNFHLRPRDLISNINVIVLELKGSETT | |
| with F42A, | FMCEYADETATIVEFLNRWITFSQSIISTLT | |
| E62K, T3A | | |
| Human IL-2 | APASSSTKKTQLQLEHLLLDLQMILNGINNYKNPKLTRMLTAKFYMPKKAT | 442 |
| mature form | ELKHLQCLEKKLKPLEEVLNLAQSKNFHLRPRDLISNINVIVLELKGSETT | |
| with F42A, | FMCEYADETATIVEFLNRWITFSQSIISTLT | |
| E61K, E62K, | | |
| T3A | | |
| Human IL-2 | APASSSTKKTQLQLEHLLLDLQMILNGINNYKNPKLTEMLTAKFYMPKKAT | 443 |
| mature form | ELKHLQCLEKKLKPLEEVLNLAQSKNFHLRPRDLISNINVIVLELKGSETT | |
| with F42A, | FMCEYADETATIVEFLNRWITFSQSIISTLT | |
| R38E, E61K, | | |
| E62K, T3A | | |

Thus, in certain embodiments, an IL-2 protein comprises, consists, or consists essentially of an amino acid sequence selected from Table S1, or an active variant or fragment thereof that is at least 80, 85, 90, 95, 98, or 100% identical to a sequence selected from Table S1. Additional examples of active polypeptide “variants” and “fragments” are described elsewhere herein.

In some embodiments, an “active” IL-2 protein or fragment or variant is characterized, for example, by its ability to bind to an IL-2Rβ/γc and/or IL-2Rα/β/γc receptor chain present on the surface of an immune cell in vitro or in vivo, and stimulate downstream signaling activities. Examples of downstream signaling activities include IL-2 mediated signaling via one or more of the JAK-STAT, PI3K/Akt/mTOR, and MAPK/ERK pathways, including combinations thereof. Altogether, IL-2 signaling stimulates an array of downstream pathways leading to responses that have a significant role in the development, function, and survival of CD4 T cells, CD8 T cells, NK cells, NKT cells, macrophages, and intestinal intraepithelial lymphocytes, among others. In some embodiments, an IL-2 polypeptide has an anti-cancer activity.

In particular embodiments, the IL-2 protein is a mature form of IL-2, or an active variant or fragment thereof, which comprises, consists, or consists essentially of an amino acid sequence that is at least 80, 85, 90, 95, 98, or 100% identical to amino acids 21-153 of SEQ ID NO: 1. In some embodiments, the IL-2 protein comprises a C145X substitution, as defined by SEQ ID NO: 1, wherein X is any amino acid. In specific embodiments, the IL-2 protein comprises a C145S substitution as defined by SEQ ID NO: 1.

Certain IL-2 polypeptides comprise, consist, or consist essentially of an amino acid sequence that is at least 80, 85, 90, 95, 98, or 100% identical to SEQ ID NO: 2 (mature human IL-2 with C125S substitution). In some embodiments, an active variant or fragment of SEQ ID NO: 2 retains the S125 residue as defined therein. In some embodiments, the IL-2 protein comprises, consists, or consists essentially of an amino acid sequence that is at least 80, 85, 90, 95, 98, or 100% identical to SEQ ID NO: 3 (mature human IL-2 “D10” variant), optionally wherein the IL-2 protein retains any one or more of the Q74H, L80F, R81D, L85V, I86V, and/or I92F substitutions as defined by SEQ ID NO: 3.

Certain IL-2 comprise one or more defined amino acid substitutions relative to the exemplary amino acid sequences in Table S1. For example, some IL-2 polypeptides comprise one or more amino acid substitutions selected from K35C, R38C, T41C, F42C, E61C, and V69C as defined by SEQ ID NO: 2. In some embodiments, the IL-2 protein forms a disulfide bond with the IL-2 binding protein (e.g., IL-2Ra) via one or more of the cysteine substitutions selected from K35C, R38C, T41C, F42C, E61C, and V69C. Certain IL-2 polypeptides comprise one or more amino acid substitutions at position V69, Q74, and/or I128 as defined by SEQ ID NO: 2, including combinations thereof and including, for example, wherein the one or more amino acid substitutions are selected from V69A, Q74P, and I128T as defined by SEQ ID NO: 2. Some IL-2 polypeptides comprise one or more amino acid substitutions at position T3, D20, R38, F42, Y45, E61, E62, E68, and/or L72 as defined by SEQ ID NO: 2, including combinations thereof. Exemplary amino acid substitutions include T3A; D20T; R38A, R38E, and R38K; F42A, F42G, F42S, F42T, F42Q, F42E, F42N, F42D, F42R, F42K, and F42I; Y45A, Y45G, Y45S, Y45T, Y45Q, Y45E, Y45N, Y45D, Y45R, and Y45K; E61S and E61K; E62A, E62L, and E62K; E68A and E68V; and L72A, L72G, L72S, L72T, L72Q, L72E, L72N, L72D, L72R, and L72K, including combinations thereof. Particular examples of combinations of substitutions include T3A and F42A; T3A, V69A, Q74P, and I128T; T3A and D20T; T3A, D20T, and F42A; T3A and R38E; T3A and E62K; T3A, E61K, and E62K; T3A, R38E, E61K, E62K; T3A, R38E, and F42A; T3A, F42A, and E61K; T3A, F42A, and E62K; and T3A, F42A, E61K, and E62K. Thus, an IL-2 protein can comprise any one or more of the foregoing amino acid substitutions, including combinations thereof.

In certain embodiments, a potential O-glycosylation site in IL-2 is substituted with alanine (T3A). In some embodiments, an F42A substitution in IL-2 reduces IL-2 binding affinity towards IL-2Rα. In some embodiments, a triple mutant (V69A, Q74P, and I128T) of IL-2 has higher binding affinity towards IL-2Rα. In certain embodiments, a D20T substitution in IL-2 does not significantly reduce binding affinity to IL-2Ra binding affinity but significantly reduces signaling activity towards intermediate affinity IL-2R receptors.

In some embodiments, the IL-2 protein comprises one or more amino acid substitutions at residues selected from A1, P2, A3, S4, and S5, as defined by SEQ ID NO: 2 or 3, or comprises N-terminal deletion of 1, 2, 3, 4, or 5 amino acids, as defined by SEQ ID NO: 2 or 3.

Any one or more of the foregoing IL-2 polypeptides can be combined with any of the anti-denatured collagen antibodies (including antigen-binding fragments thereof) and linkers described herein, to generate one or more fusion proteins or larger, multi-chain structures comprising the same.

Interleukin-15 (IL-15). Certain effector domains comprise one or more IL-15 polypeptides, including human IL-15 polypeptides. IL-15 is a pleiotropic cytokine that has been shown to induce and regulate a myriad of immune functions. For example, IL-15 is critical for lymphoid development, peripheral maintenance of innate immune cells, and immunological memory of T cells, mainly natural killer (NK) and CD8+ T cell populations. Like IL-2, IL-15 binds to and signals through a complex composed of IL-2/IL-15 receptor beta chain (CD122) and the common gamma chain (gamma-C, CD132).

IL-15 is a 14-15 kDA protein composed of a signal peptide (residues 1-29), a propeptide (residues 30-48), and an active mature protein (residues 49-162). Exemplary IL-15 protein sequences are provided in Table S2.

TABLE S2
Exemplary IL-15 polypeptide sequences

| | | SEQ ID |
|---|---|---|
| Name | Sequence | NO: |
| Human IL-15 | MRISKPHLRSISIQCYLCLLLNSHFLTEAGIHVFILGCFSAGLPKTEANW | 444 |
| FL precursor | VNVISDLKKIEDLIQSMHIDATLYTESDVHPSCKVTAMKCFLLELQVISL | |
| | ESGDASIHDTVENLIILANNSLSSNGNVTESGCKECEELEEKNIKEFLQS | |
| | FVHIVQMFINTS | |
| Human IL-15 | NWVNVISDLKKIEDLIQSMHIDATLYTESDVHPSCKVTAMKCFLLELQVI | 445 |
| mature form | SLESGDASIHDTVENLIILANNSLSSNGNVTESGCKECEELEEKNIKEFL | |
| | QSFVHIVQMFINTS | |
| Human IL-15 | NWVNVISDLKKIEDLIQSMHIDATLYTESDVHPSCKVTAMKCFLLELQVI | 446 |
| mature form | SLESGDASIHDTVENLIILANNSLSSNGNVTESGCKECEELEEKNIKEFL | |
| with S162A | QSFVHIVQMFINTA | |
| Human IL-15 | NWVNVISDLKKIEDLIQSMHIDATLYTESDVHPSCKVTAMKCFLLELQDI | 447 |
| mature form | SLESGDASIHDTVENLIILANNSLSSNGNVTESGCKECEELEEKNIKEFL | |
| with V49D, | QSFVHIVQMFINTA | |
| S162A | | |
| Human IL-15 | NWVNVISDLKKIEDLIQSMHIDATLYTESDVHPSCKVTAMKCFLLELQVD | 448 |
| mature form | SLESGDASIHDTVENLIILANNSLSSNGNVTESGCKECEELEEKNIKEFL | |
| with I50D, | QSFVHIVQMFINTA | |
| S162A | | |
| Human IL-15 | NWVNVISDLKKIEDLIQSMHIDATLYTESDVHPSCKVTAMKCFLLELQVI | 449 |
| mature form | SLESGDASIHDTVENEIILANNSLSSNGNVTESGCKECEELEEKNIKEFL | |
| with L66E, | QSFVHIVQMFINTA | |
| S162A | | |
| Human IL-15 | NWVNVISNLKKIEDLIQSMHIDATLYTESDVHPSCKVTAMKCFLLELQVI | 450 |
| mature form | SLESGDASIHDTVENLIILANNSLSSNGNVTESGCKECEELEEKNIKEFL | |
| with D8N, | QSFVHIVQMFINTA | |
| S162A | | |
| Human IL-15 | NWVNVISDLKKIEDLIQSMHIDATLYTESDVHPSCKVTAMKCFLLKLQVI | 451 |
| mature form | SLESGDASIHDTVENLIILANNSLSSNGNVTESGCKECEELEEKNIKEFL | |
| with E46K, | QSFVHIVQMFINTA | |
| S162A | | |
| Human IL-15 | NWVNVISDLKKIEDLIQSMHIDATLYTESDVHPSCKVTAMKCFLLKLQVI | 452 |
| mature form | SLKSGDASIHDTVENLIILANNSLSSNGNVTESGCKECEELEEKNIKEFL | |
| with E46K, | QSFVHIVQMFINTA | |
| E53K, S162A | | |
| Human IL-15 | NWVNVISDLKKIEDLIQSMHIKATLYTESDVHPSCKVTAMKCFLLKLQVI | 453 |
| mature form | SLESGDASIHDTVENLIILANNSLSSNGNVTESGCKECEELEEKNIKEFL | |
| with D22K, | QSFVHIVQMFINTA | |
| E46K, S162A | | |
| Human IL-15 | NWVNVISDLKKIEDLIQSMHIKATLYTESDVHPSCKVTAMKCFLLKLQVI | 454 |
| mature form | SLKSGDASIHDTVENLIILANNSLSSNGNVTESGCKECEELEEKNIKEFL | |
| with D22K, | QSFVHIVQMFINTA | |
| E46K, E53K, | | |
| S162A | | |

Thus, in certain embodiments, an IL-15 protein comprises, consists, or consists essentially of an amino acid sequence selected from Table S2, or an active variant or fragment thereof that is at least 80, 85, 90, 95, 98, or 100% identical to a sequence selected from Table S2. Additional examples of active polypeptide “variants” and “fragments” are described elsewhere herein.

In some embodiments, an “active” IL-15 protein or fragment or variant is characterized, for example, by its ability to bind to an IL-15Rβ/γc and/or IL-15Rα/β/γc receptor chain present on the surface of an immune cell in vitro or in vivo, and stimulate downstream signaling activities. Examples of downstream signaling activities include IL-15 mediated signaling via Janus kinase 1 (Jak1) and γc subunit Janus kinase 3 (Jak3), which leads to phosphorylation and activation of signal transducer and activator of transcription 3 (STAT3) and STAT5 pathways. Additional examples include activation of Src family kinases including Lck and Fyn, and subsequent activation of PI3K and MAPK signaling pathways. Altogether, IL-15 signaling stimulates an array of downstream pathways leading to responses that have a significant role in the regulating the activation and proliferation of T and natural killer (NK) cells, and the survival of memory T cells, among others. In particular embodiments, an activated IL-15 polypeptide elicits potent antitumor responses upon activation in target tissues, that is, a tumor microenvironment.

In particular embodiments, the IL-15 protein is a mature form of IL-15, or an active variant or fragment thereof, which comprises, consists, or consists essentially of an amino acid sequence that is at least 80, 85, 90, 95, 98, or 100% identical to amino acids 49-162 of SEQ ID NO: 27 (Human IL-15 FL precursor). Certain IL-15 polypeptides comprise, consist, or consist essentially of an amino acid sequence that is at least 80, 85, 90, 95, 98, or 100% identical to SEQ ID NO: 28 (mature human IL-15).

Certain IL-15 polypeptides comprise one or more defined amino acid substitutions relative to the exemplary amino acid sequences in Table S2. For example, in certain embodiments an IL-15 protein comprises or retains one or more amino acid substitutions at position D8, D22, E46, V49, 150, L66, and/or S162 as defined by SEQ ID NO: 26 (mature human IL-15). Specific examples of substitutions are selected from one or more of D8N, D22K, E46K, V49D, I50D, L66E, and 162A, including combinations thereof (see Table S3). Exemplar combinations of substitutions are selected from V49D and S162A; I50D and S162A; L66E and S162A; D8N and S162A; V49D and S162A; E46K and S162A; E46K, E53K, and S162A; D22K, E46K, and S162A; and D22K, E46K, E53K, and S162A.

In some embodiments, a D8N substitution in IL-15 does not significantly reduce binding affinity to IL-15Rα significantly reduces or all but eliminates IL-15 signaling activity. In some embodiments a V49D substitution in IL-15 has significantly lower (e.g., about 13 fold lower) binding affinity to IL-15Rα and retains about or at least about 90-100% of IL-15 signaling activity. In some embodiments, an I50D substitution in IL-15 has significantly lower (e.g., about 100 fold lower) binding affinity to IL-15Rα and retains about 10% of IL-15 signaling activity. In some embodiments, a L66E substitution in IL-15 has significantly lower (e.g., about 15 fold lower) binding affinity to IL-15Rα and retains little to no IL-15 signaling activity.

Any one or more of the foregoing IL-15 polypeptides can be combined with any of the anti-denatured collagen antibodies (including antigen-binding fragments thereof) and linkers described herein, to generate one or more fusion proteins or larger, multi-chain structures comprising the same.

Hybrid IL-2/IL-15. Certain effector domains comprise one or more hybrid IL-2/IL-15 polypeptides (see, for example, Silva et al., Nature 565:186-191, 2019). Exemplary hybrid IL-2/IL-15 polypeptides are provided in Table S3.

TABLE S3
Exemplary Hybrid IL-2/IL-15 Polypeptide

| | | SEQ ID |
|---|---|---|
| Name | Sequence | NO. |
| Neoleukin | GSHMPKKKIQLHAEHALYDALMILN | 455 |
| 2/15 | IVKTNSPPAEEKLEDYAFNFELILE | |
| | EIARLFESGDQKDEAEKAKRMKEWM | |
| | KRIKTTASEDEQEEMANAIITILQS | |
| | WIFS | |

Thus, in certain embodiments, a hybrid IL-2/IL-15 protein comprises, consists, or consists essentially of an amino acid sequence selected from Table S3, or an active variant or fragment thereof that is at least 80, 85, 90, 95, 98, or 100% identical to a sequence selected from Table S3. An active variant or fragment has one or more IL-2 activities and/or IL-15 activities described herein. Additional examples of active polypeptide “variants” and “fragments” are described elsewhere herein.

In some embodiments, an “active” IL-2/IL-15 hybrid protein or fragment or variant is characterized, for example, by its ability to bind to an IL-2Rβ/γc, IL-2Rα/β/γc, IL-15Rβ/γc, and/or IL-15Rα/β/γc receptor chain present on the surface of an immune cell in vitro or in vivo, and stimulate downstream signaling activities

Any one or more of the foregoing hybrid IL-2/IL-15 polypeptides can be combined with any of the anti-denatured collagen antibodies (including antigen-binding fragments thereof) and linkers described herein, to generate one or more fusion proteins or larger, multi-chain structures comprising the same.

TNF Superfamily Ligands. Certain effector domains comprise one or more Tumor Necrosis Factor (TNF) superfamily ligands, also referred to as TNF superfamily ligand polypeptides. The Tumor Necrosis Factor receptor superfamily (TNFRSF) is a protein superfamily of cytokine receptors characterized by the ability to bind tumor necrosis factors (TNFs) via an extracellular cysteine-rich domain. With the exception of nerve growth factor (NGF), all TNFs are homologous to the archetypal TNF-α. TNF receptors are primarily involved in apoptosis and inflammation, but also regulate other signal transduction pathways, such as cell proliferation, survival, and differentiation. The term death receptor refers to those members of the TNF receptor superfamily that contain a death domain, examples of which include TNFR1, the Fas receptor, Death Receptor 4 (DR4), and Death Receptor 5 (DR5).

An illustrative list of TNF superfamily receptors and their corresponding ligands is provided in Table T1 below.

TABLE T1
Exemplary TNF Superfamily Members

| Cell Receptor(s) | Synonyms | Ligand(s) |
|---|---|---|
| Death receptor 4 | TRAILR1, Apo-2, | TRAIL |
| | CD261 | |
| Death receptor 5 | TRAILR2, CD262 | |
| Decoy receptor 1 | TRAILR3, LIT, | |
| | TRID, CD263 | |
| Decoy receptor 2 | TRAILR4, TRUNDD, | |
| | CD264 | |
| Tumor necrosis factor | CD120a | TNF-α |
| receptor 1 | | |
| Tumor necrosis factor | CD120b | |
| receptor 2 | | |
| Fas receptor | Apo-1, CD95 | FasL |
| Lymphotoxin beta | CD18 | Lymphotoxin |
| receptor | | beta (TNF-C) |
| OX4 0 | CD134 | OX40L |
| CD40 | Bp50 | CD154 |
| Decoy receptor 3 | TR6, M68 | FasL, LIGHT, TL1A |
| CD27 | S152, Tp55 | CD70, Siva |
| CD30 | Ki-1 | CD153 |
| 4-1BB | CD137 | 4-1BBL |
| RANK | CD265 | RANKL |
| Osteoprotegerin | OCIF, TR1 | |
| TWEAK receptor | Fn14, CD266 | TWEAK |
| TACI | IGAD2, CD267 | APRIL, BAFF, |
| | | CAMLG |
| BAFF receptor | CD268 | BAFF |
| Herpesvirus entry | ATAR, TR2, CD270 | LIGHT |
| mediator | | |
| Nerve growth factor | p75NTR, CD271 | NGF, BDNF, NT-3, |
| receptor | | NT-4 |
| B-cell maturation antigen | TNFRSF13A, CD269 | BAFF |
| Glucocorticoid-induced | AITR, CD357 | GITR ligand |
| TNFR-related | | |
| Death receptor 3 | Apo-3, TRAMP, | TL1A |
| | LARD, WS-1 | |
| Ectodysplasin A2 | XEDAR | EDA-A2 |
| receptor | | |

Thus, in certain embodiments, the TNF superfamily ligand component of the fusion protein is selected from a ligand polypeptide in Table T1. In certain embodiments, the TNF superfamily ligand is a human polypeptide ligand selected from Table T1.

In some embodiments, the TNF superfamily ligand is a trimeric or homotrimeric polypeptide, for example, a single chain trimeric TRAIL or a single chain trimeric 4-1BBL. In certain embodiments, the TNF superfamily ligand is a trimeric or homotrimeric polypeptide ligand selected from Table T1.

In some embodiments, the TNF superfamily ligand induces apoptosis in cancer cells, for example, by binding to a death domain or death receptor of a TNF superfamily receptor. Thus, in some embodiments, TNF superfamily ligand (e.g., trimeric or homotrimeric ligand) binds to at least one TNF death receptor, or a TNF superfamily receptor that contains at least one death domain. Examples of TNF superfamily death receptors include TNFR1, Fas receptor, DR4, and DR5. Particular examples of death receptor ligands include TRAIL, TNF-α, and FasL. Thus, in certain embodiments, the TNF superfamily ligand component of the conjugate is selected from one or more of TRAIL, TNF-α, and FasL, optionally a human TRAIL, human TNF-α, or human FasL.

The amino acid sequences of exemplary TNF superfamily ligands are provided in Table S4 below.

TABLE S4
Exemplary TNF Superfamily Ligand Sequences

| | | SEQ ID |
|---|---|---|
| Name | Sequence | NO. |
| FL TRAIL | MAMMEVQGGPSLGQTCVLIVIFTVLLQSLCVAVTYVYFTNELKQMQDK | 456 |
| (1-281) | YSKSGIACFLKEDDSYWDPNDEESMNSPCWQVKWQLRQLVRKMILRTS | |
| | EETISTVQEKQQNISPLVRERGPQRVAAHITGTRGRSNTLSSPNSKNE | |
| | KALGRKINSWESSRSGHSFLSNLHLRNGELVIHEKGFYYIYSQTYFRF | |
| | QEEIKENTKNDKQMVQYIYKYTSYPDPILLMKSARNSCWSKDAEYGLY | |
| | SIYQGGIFELKENDRIFVSVTNEHLIDMDHEASFFGAFLVG | |
| TRAIL | VRERGPQRVAAHITGTRGRSNTLSSPNSKNEKALGRKINSWESSRSGH | 457 |
| extracellular | SFLSNLHLRNGELVIHEKGFYYIYSQTYFRFQEEIKENTKNDKQMVQY | |
| domain | IYKYTSYPDPILLMKSARNSCWSKDAEYGLYSIYQGGIFELKENDRIF | |
| (114-281) | VSVTNEHLIDMDHEASFFGAFLVG | |
| 4-1BBL | MEYASDASLDPEAPWPPAPRARACRVLPWALVAGLLLLLLLAAACAVF | 458 |
| | LACPWAVSGARASPGSAASPRLREGPELSPDDPAGLLDLRQGMFAQLV | |
| | AQNVLLIDGPLSWYSDPGLAGVSLTGGLSYKEDTKELVVAKAGVYYVF | |
| | FQLELRRVVAGEGSGSVSLALHLQPLRSAAGAAALALTVDLPPASSEA | |
| | RNSAFGFQGRLLHLSAGQRLGVHLHTEARARHAWQLTQGATVLGLFRV | |
| | TPEIPAGLPSPRSE | |
| 4-1BBL | ACPWAVSGARASPGSAASPRLREGPELSPDDPAGLLDLRQGMFAQLVA | 459 |
| extracellular | QNVLLIDGPLSWYSDPGLAGVSLTGGLSYKEDTKELVVAKAGVYYVFF | |
| domain | QLELRRVVAGEGSGSVSLALHLQPLRSAAGAAALALTVDLPPASSEAR | |
| (50-254) | NSAFGFQGRLLHLSAGQRLGVHLHTEARARHAWQLTQGATVLGLFRVT | |
| | PEIPAGLPSPRSE | |

In some embodiments, the TNF superfamily ligand component of the fusion protein comprises, consists, or consists essentially of an amino acid sequence selected from Table S4, or an active variant or fragment thereof. Particular examples of variants and fragments comprise, consist, or consist essentially of an amino acid sequence that is at least 80%, 95%, 90%, 95%, 96%, 97%, 98%, or 99% identical to a sequence selected from Table S4. Additional examples of active polypeptide “variants” and “fragments” are described elsewhere herein.

In specific embodiments, the TNF superfamily ligand component of the fusion protein is a human TNF-related apoptosis-inducing ligand (TRAIL) polypeptide, or an active variant or fragment thereof, including single chain trimeric TRAIL. TRAIL is a cytokine that is produced and secreted by most normal tissue cells. It causes apoptosis in tumor cells, for example, by binding to certain death receptors. The predicted 281 amino acid TRAIL protein has the characteristic structure of a type II membrane protein, with a single internal hydrophobic domain and no signal sequence. The extracellular C-terminal domain of TRAIL shares 22 to 28% identity with the C-terminal domains of other TNF family members. Formation of a complex between TRAIL and its signaling receptors, DR4 and DR5, triggers apoptosis by inducing the oligomerization of intracellular death domains. In some instances, a TRAIL polypeptide has an anti-cancer activity.

In certain embodiments, the TRAIL component of the conjugate comprises, consists, or consists essentially of a TRAIL sequence from Table S4, or an active variant or fragment thereof. In some embodiments, an “active” TRAIL polypeptide or fragment or variant is characterized, for example, by its ability to bind to death receptors, as described herein, and induce apoptosis, for example, in tumor cells. Specific examples of TRAIL variants include those having any one or more of the following substitutions; S96C, S101C, S111C, R170C, and K179C. In some embodiments, the TRAIL variant has a set of amino acid substitutions at the residue position selected from one or more of Y189Q, R191K, Q193R; H264R, I266L, D267Q; Y189Q, R191K, Q193R; and Y189Q, R191K, Q193R, I266L (see U.S. Application Nos. 2013/0165383; and 2012/0165267, incorporated by reference). Particular examples of TRAIL fragments include residues 114-281 (extracellular domain), residues 95-281, residues 92-281, residues 91-281, residues 41-281, residues 39-281, residues 15-281, residues 119-281, and residues 1-281 of the full-length sequence (SEQ ID NO: _). Additional examples of polypeptide “variants” and “fragments” are described elsewhere herein.

In specific embodiments, the TNF superfamily ligand component of the fusion protein is a human 4-1BBL polypeptide, or an active variant or fragment thereof, including single chain trimeric 4-1BB. 4-1BBL (4-1BB ligand, CD137L) is expressed on antigen presenting cells and binds to 4-1BB (also known as CD137), a type 2 transmembrane glycoprotein receptor of the TNF superfamily that is expressed on activated T Lymphocytes. In certain embodiments, the 4-1BBL component of the conjugate comprises, consists, or consists essentially of a 4-1BBL sequence from Table S4, or an active variant or fragment thereof. In some embodiments, an “active” 4-1BBL polypeptide or fragment or variant is characterized, for example, by its ability to bind to 4-1BB receptor chain present on the surface of an immune cell or cancer cell in vitro or in vivo, and stimulate downstream signaling activities, absent steric hindrance by the binding moieties described herein. Exemplary activities include inducing the proliferation of activated peripheral blood T cells, increasing proliferation, cytokine production, and survival of CD8 T cells, including by enhancing anti-tumor activity in a CD8 T-cell-dependent manner. In certain embodiments, a 4-1BBL polypeptide has an anti-cancer activity.

Any one or more of the foregoing TNF superfamily ligand polypeptides can be combined with any of the anti-denatured collagen antibodies (including antigen-binding fragments thereof) and linkers described herein, to generate one or more fusion proteins or larger, multi-chain structures comprising the same.

Interleukin-12 (IL-12). Certain effector domains comprise one or more IL-12 polypeptides. IL-12 is produced by dendritic cells, macrophages, neutrophils, and human B-lymphoblastoid cells (NC-37) in response to antigenic stimulation. IL-12 binds to the IL-12 receptor, which is a heterodimeric receptor formed by IL-12Rβ1 and IL-12Rβ2.

IL-12 is a heterodimeric cytokine encoded by two separate genes, IL-12A (p35) and IL-12B (p40). The active heterodimer (referred to as “p70”), and a homodimer of p40 are formed following protein synthesis. Exemplary IL-12 polypeptide sequences are provided in Table S5.

TABLE S5
Exemplary IL-12 Polypeptide

| | | SEQ ID |
|---|---|---|
| Name | Sequence | NO. |
| Human IL-12A | MCPARSLLLVATLVLLDHLSLARNLPVATPDPGMFPCLHHSQNLLRAV | 460 |
| Full-length | SNMLQKARQTLEFYPCTSEEIDHEDITKDKTSTVEACLPLELTKNESC | |
| | LNSRETSFITNGSCLASRKTSFMMALCLSSIYEDLKMYQVEFKTMNAK | |
| | LLMDPKRQIFLDQNMLAVIDELMQALNFNSETVPQKSSLEEPDFYKTK | |
| | IKLCILLHAFRIRAVTIDRVMSYLNAS | |
| Human IL-12A | RNLPVATPDPGMFPCLHHSQNLLRAVSNMLQKARQTLEFYPCTSEEID | 461 |
| Mature | HEDITKDKTSTVEACLPLELTKNESCLNSRETSFITNGSCLASRKTSF | |
| | MMALCLSSIYEDLKMYQVEFKTMNAKLLMDPKRQIFLDQNMLAVIDEL | |
| | MQALNFNSETVPQKSSLEEPDFYKTKIKLCILLHAFRIRAVTIDRVMS | |
| | YLNAS | |
| Human IL-12B | MCHQQLVISWFSLVFLASPLVAIWELKKDVYVVELDWYPDAPGEMVVL | 462 |
| Full-length | TCDTPEEDGITWTLDQSSEVLGSGKTLTIQVKEFGDAGQYTCHKGGEV | |
| | LSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVE | |
| | CQEDSACPAAEESLPIEVMVDAVHKLKYENYTSSFFIRDIIKPDPPKN | |
| | LQLKPLKNSRQVEVSWEYPDTWSTPHSYFSLTFCVQVQGKSKREKKDR | |
| | VFTDKTSATVICRKNASISVRAQDRYYSSSWSEWASVPCS | |
| Human IL-12B | IWELKKDVYVVELDWYPDAPGEMVVLTCDTPEEDGITWTLDQSSEVLG | 463 |
| Mature | SGKTLTIQVKEFGDAGQYTCHKGGEVLSHSLLLLHKKEDGIWSTDILK | |
| | DQKEPKNKTFLRCEAKNYSGRFTCWWLTTISTDLTFSVKSSRGSSDPQ | |
| | GVTCGAATLSAERVRGDNKEYEYSVECQEDSACPAAEESLPIEVMVDA | |
| | VHKLKYENYTSSFFIRDIIKPDPPKNLQLKPLKNSRQVEVSWEYPDTW | |
| | STPHSYFSLTFCVQVQGKSKREKKDRVFTDKTSATVICRKNASISVRA | |
| | QDRYYSSSWSEWASVPCS | |
| Human IL-12B | IWELKKDVYVVELDWYPDAPGEMVVLTCDTPEEDGITWTLDQSSEVLG | 464 |
| Ig-like C2- | SGKTLTIQVKEFGDAGQYTCHKGGEVLSHSLLLLHK | |
| type domain | | |
| (23-106) | | |
| Human IL-12B | PPKNLQLKPLKNSRQVEVSWEYPDTWSTPHSYFSLTFCVQVQGKSKRE | 465 |
| Fibronectin | KKDRVFTDKTSATVICRKNASISVRAQDRYYSSSWSEWASVPCS | |
| type-III | | |
| domain | | |
| (237-328) | | |

Thus, in certain embodiments, an IL-12 protein comprises, consists, or consists essentially of one or more amino acid sequences selected from Table S5, or an active variant or fragment thereof that is at least 80, 85, 90, 95, 98, or 100% identical to a sequence selected from Table S5. Additional examples of active polypeptide “variants” and “fragments” are described elsewhere herein.

In some embodiments, an “active” IL-12 protein or fragment or variant is characterized, for example, by its ability to bind to an IL-12Rβ1 and/or IL-12Rβ2 receptor chain present on the surface of an immune cell in vitro or in vivo, and stimulate downstream signaling activities. Certain exemplary downstream activities include differentiation of naive T cells into Th1 cells, stimulating growth and function of T cells generally, activation of the cytotoxic activity of natural killer (NK) cells and cytotoxic T lymphocytes, and anti-angiogenic activities (i.e., reducing formation of new blood vessels), among other activities. Additional examples include stimulating the production of interferon-gamma (IFN-γ) and tumor necrosis factor-alpha (TNF-α) from T cells and NK cells, and reducing IL-4 mediated suppression of IFN-γ.

In some instances, IL-12 binds to the IL-12Rβ2 subunit of IL-12 receptor, which is found on activated T cells and is stimulated by cytokines that promote Th1 cell development and inhibited by those that promote Th2 cell development. Upon binding, IL-12R-β2 becomes tyrosine phosphorylated and provides binding sites for kinases, Tyk2 and Jak2, which in turn activate transcription factor proteins such as STAT4. Thus, in some instances, an active IL-12 polypeptide activates the JAK-STAT pathway. In some instances, an IL-12 polypeptide has an anti-cancer activity.

In particular embodiments, the IL-12 polypeptide is a mature form of IL-12, or an active variant or fragment thereof, which comprises, consists, or consists essentially of an amino acid sequence of the mature forms of human IL-12A and/or IL-12B. Certain IL-12 polypeptides thus comprise, consist, or consist essentially of an amino acid sequence that is at least 80, 85, 90, 95, 98, or 100% identical to SEQ ID NO: _ (mature human IL-12A) and/or_ (mature human IL-12B). Exemplary fragments of IL-12 include the Ig-like C2-type domain (23-106) and the fibronectin type-III domain (237-328) of IL12B. Certain IL-12 polypeptides comprise one or more amino acid substitutions relative to the exemplary amino acid sequences in Table S5.

Any one or more of the foregoing IL-12 polypeptides can be combined with any of the anti-denatured collagen antibodies (including antigen-binding fragments thereof) and linkers described herein, to generate one or more fusion proteins or larger, multi-chain structures comprising the same.

Interleukin-10 (IL-10). Certain effector domains comprise one or more IL-10 polypeptides. IL-10, also known as human cytokine synthesis inhibitory factor (CSIF), is generally considered an anti-inflammatory cytokine, but has immunostimulatory activities in the immuno-oncology context. IL-10 has multiple, pleiotropic, effects in immuno-regulation and inflammation.

IL-10 is a homodimer that signals through a receptor complex consisting of two IL-10α receptor and two IL-10β receptor subunits, such that the functional cell receptor consists of four IL-10 receptor subunits. IL-10 binding induces STAT3 signaling via the phosphorylation of the cytoplasmic tails of IL-10α receptor and IL-10β receptor by JAK1 and Tyk2, respectively. Exemplary IL-10 protein sequences are provided in Table S6.

TABLE S6
Exemplary IL-10 Polypeptides

| | | SEQ ID |
|---|---|---|
| Name | Sequence | NO. |
| Human IL-10 | MHSSALLCCLVLLTGVRASPGQGT | 466 |
| Full-length | QSENSCTHFPGNLPNMLRDLRDAF | |
| | SRVKTFFQMKDQLDNLLLKESLLE | |
| | DFKGYLGCQALSEMIQFYLEEVMP | |
| | QAENQDPDIKAHVNSLGENLKTLR | |
| | LRLRRCHRFLPCENKSKAVEQVKN | |
| | AFNKLQEKGIYKAMSEFDIFINYI | |
| | EAYMTMKIRN | |
| Human IL-10 | SPGQGTQSENSCTHFPGNLPNMLR | 467 |
| Mature | DLRDAFSRVKTFFQMKDQLDNLLL | |
| | KESLLEDFKGYLGCQALSEMIQFY | |
| | LEEVMPQAENQDPDIKAHVNSLGE | |
| | NLKTLRLRLRRCHRFLPCENKSKA | |
| | VEQVKNAFNKLQEKGIYKAMSEFD | |
| | IFINYIEAYMTMKIRN | |

Thus, in certain embodiments, an IL-10 protein comprises, consists, or consists essentially of an amino acid sequence selected from Table S6, or an active variant or fragment thereof that is at least 80, 85, 90, 95, 98, or 100% identical to a sequence selected from Table S6. Additional examples of active polypeptide “variants” and “fragments” are described elsewhere herein.

In some embodiments, an “active” IL-10 polypeptide or fragment or variant is characterized, for example, by its ability to bind to an IL-10α receptor and/or IL-10β receptor protein chain present on the surface of an immune cell in vitro or in vivo, and stimulate downstream signaling activities. Certain exemplary downstream activities include downregulating the expression of Th1 cytokines, MHC class II antigens, and co-stimulatory molecules on macrophages, enhancing B cell survival, proliferation, and antibody production, blocking NF-κB activity, and regulating the JAK-STAT signaling pathway. Certain activities include inhibiting synthesis of pro-inflammatory cytokines such as IFN-γ, IL-2, IL-3, TNFα and GM-CSF made by cells such as macrophages and Th1 T cells, and in some instances inhibiting lipopolysaccharide (LPS) and bacterial product mediated induction of pro-inflammatory cytokines. Thus, in some embodiments, an active IL-10 polypeptide has an anti-inflammatory activity. As noted above, IL-10 can increase anti-cancer immune responses. In some embodiments, an active IL-10 polypeptide has an anti-cancer activity.

In particular embodiments, the IL-10 polypeptide is a mature form of IL-10, or an active variant or fragment thereof, which comprises, consists, or consists essentially of an amino acid sequence of the mature forms of human IL-10. Certain IL-10 polypeptides thus comprise, consist, or consist essentially of an amino acid sequence that is at least 80, 85, 90, 95, 98, or 100% identical to SEQ ID NO: _ (mature human IL-10). Certain IL-10 polypeptides comprise one or more amino acid substitutions relative to the exemplary amino acid sequences in Table S6.

Any one or more of the foregoing IL-10 polypeptides can be combined with any of the anti-denatured collagen antibodies (including antigen-binding fragments thereof) and linkers described herein, to generate one or more fusion proteins or larger, multi-chain structures comprising the same.

Interferon-α (IFN-α). Certain effector domains comprise one or more IFN-α polypeptides. IFN-α is a member of the type I interferon family of immuno-regulatory proteins that bind to the IFN-α cell surface receptor complex. IFN-α polypeptides are produced mainly by plasmacytoid dendritic cells (pDCs), and are primarily involved in regulating innate immunity against viral infection. There are 13 subtypes of IFN-α, encoded by IFNA1, IFNA2, IFNA4, IFNA5, IFNA6, IFNA7, IFNA8, IFNA10, IFNA13, IFNA14, IFNA16, IFNA17, IFNA2. Recombinant IFN-α is used for the treatment of cancer, including hairy cell leukemia, malignant melanoma and AIDS-related Kaposi's sarcoma, venereal or genital warts caused by the Human Papilloma Virus, hepatitis B, and hepatitis C.

Exemplary IFN-α protein sequences are provided in Table S7.

TABLE S7
Exemplary IFN-α Polypeptides

| | | SEQ ID |
|---|---|---|
| Name | Sequence | NO. |
| Human | MALTFALLVALLVLSCKSSCSVGCDLPQTHSLGSRRTLMLLAQMRKIS | 468 |
| Interferon-α2 | LFSCLKDRHDFGFPQEEFGNQFQKAETIPVLHEMIQQIFNLFSTKDSS | |
| Full-length | AAWDETLLDKFYTELYQQLNDLEACVIQGVGVTETPLMKEDSILAVRK | |
| precursor | YFQRITLYLKEKKYSPCAWEVVRAEIMRSFSLSTNLQESLRSKE | |
| Human | CDLPQTHSLGSRRTLMLLAQMRKISLFSCLKDRHDFGFPQEEFGNQFQ | 469 |
| Interferon-α2 | KAETIPVLHEMIQQIFNLFSTKDSSAAWDETLLDKFYTELYQQLNDLE | |
| Mature | ACVIQGVGVTETPLMKEDSILAVRKYFQRITLYLKEKKYSPCAWEVVR | |
| (24-188) | AEIMRSFSLSTNLQESLRSKE | |
| Interferon | MCDLPQTHSLGNRRALILLAQMRRISPFSCLKDRHDFGFPQEEFDGNQ | 470 |
| alfacon-1 | FQKAQAISVLHEMIQQTFNLFSTKDSSAAWDESLLEKFYTELYQQLND | |
| | LEACVIQEVGVEETPLMNVDSILAVKKYFQRITLYLTEKKYSPCAWEV | |
| | VRAEIMRSFSLSTNLQERLRRKE | |
| alpha-2B | CDLPQTHSLGSRRTLMLLAQMRRISLFSCLKDRHDFGFPQEEFGNQFQ | 471 |
| | KAETIPVLHEMIQQIFNLFSTKDSSAAWDETLLDKFYTELYQQLNDLE | |
| | ACVIQGVGVTETPLMNEDSILAVRKYFQRITLYLKEKKYSPCAWEVVR | |
| | AEIMRSFSLSTNLQESLRSKE | |
| alpha-2c | CDLPQTHSLGSRRTLMLLAQMRRISLFSCLKDRRDFGFPQEEFGNQFQ | 472 |
| | KAETIPVLHEMIQQIFNLFSTKDSSAAWDETLLDKFYTELYQQLNDLE | |
| | ACVIQGVGVTETPLMNEDSILAVRKYFQRITLYLKEKKYSPCAWEVVR | |
| | AEIMRSFSLSTNLQESLRSKE | |

Thus, in certain embodiments, an IFN-α polypeptide comprises, consists, or consists essentially of an amino acid sequence selected from Table S7, or an active variant or fragment thereof that is at least 80, 85, 90, 95, 98, or 100% identical to a sequence selected from Table S7. Additional examples of active polypeptide “variants” and “fragments” are described elsewhere herein.

In some embodiments, an “active” IFN-α polypeptide or fragment or variant is characterized, for example, by its ability to bind to an interferon/P receptor present on the surface of an immune cell in vitro or in vivo, and stimulate downstream signaling activities. Certain exemplary downstream activities include upregulating the expression of MHC I proteins, which increases presentation of peptides derived from viral antigens and thereby enhances the activation of CD8+ cytotoxic T cells, inducing antiviral mediators such as 2′-5′ oligoadenylate synthetase (2′-5′ A synthetase) and protein kinase R, and activating Jak (Janus kinase) tyrosine kinases (Jak1 and Tyk2) and Stat1 and Stat2 (signal transducers and activators of transcription). In some embodiments, an IFN-α polypeptide has anti-viral and/or anti-cancer activities. Certain IFNα polypeptides comprise one or more amino acid substitutions relative to the exemplary amino acid sequences in Table S7.

Any one or more of the foregoing IFNα polypeptides can be combined with any of the anti-denatured collagen antibodies (including antigen-binding fragments thereof) and linkers described herein, to generate one or more fusion proteins or larger, multi-chain structures comprising the same.

Interleukin-7 (IL-7). Certain effector domains comprise one or more IL-7 polypeptides. IL-7 is a hematopoietic growth factor secreted by stromal cells in the bone marrow and thymus. It is important for proliferation during certain stages of B-cell maturation, and T cell and NK cell survival, development, and homeostasis.

IL-7 binds to the IL-7 receptor (IL-7R), a heterodimer composed of two subunits, an interleukin-7 receptor-α (CD127) and common-γ chain receptor (CD132). Receptor binding results in a cascade of signals important for T-cell development within the thymus and survival within the periphery, among other activities. IL-7 as an immunotherapy agent has been examined for treatment of various malignancies and during HIV infection. For example, administration of IL-7 in patients with cancer has been shown to transiently disrupt the homeostasis of both CD8+ and CD4+ T cells with a commensurate decrease in the percentage of CD4+CD25+Foxp3+ T regulatory cells. Associated with antiretroviral therapy, IL-7 has been shown to decrease local and systemic inflammations in patients that had incomplete T-cell reconstitution. Exemplary IL-7 protein sequences are provided in Table S8.

TABLE S8
Exemplary IL-7 Polypeptides

| | | SEQ ID |
|---|---|---|
| Name | Sequence | NO. |
| Human IL-7 | MFHVSFRYIFGLPPLILVLLPVASSDCDIEGKDGKQYESVLMVSIDQLL | 473 |
| Full-length | DSMKEIGSNCLNNEFNFFKRHICDANKEGMFLFRAARKLRQFLKMNSTG | |
| | DFDLHLLKVSEGTTILLNCTGQVKGRKPAALGEAQPTKSLEENKSLKEQ | |
| | KKLNDLCFLKRLLQEIKTCWNKILMGTKEH | |
| Human IL-7 | DCDIEGKDGKQYESVLMVSIDQLLDSMKEIGSNCLNNEFNFFKRHICDA | 474 |
| Mature | NKEGMFLFRAARKLRQFLKMNSTGDFDLHLLKVSEGTTILLNCTGQVKG | |
| | RKPAALGEAQPTKSLEENKSLKEQKKLNDLCFLKRLLQEIKTCWNKILM | |
| | GTKEH | |
| Human IL-7 | DCDIEGKDGKQYESVLMVSIDQLLDSMKEIGSNCLNNEFNFFKRHICDA | 475 |
| Mature | NKEGMFLFRAARKLRQFLKMNSTGDFDLHLLKVSEGTTILLNCTGQVKG | |
| E106A | RKPAALGAAQPTKSLEENKSLKEQKKLNDLCFLKRLLQEIKTCWNKILM | |
| | GTKEH | |

Thus, in certain embodiments, an IL-7 polypeptide comprises, consists, or consists essentially of an amino acid sequence selected from Table S8, or an active variant or fragment thereof that is at least 80, 85, 90, 95, 98, or 100% identical to a sequence selected from Table S8. Additional examples of active polypeptide “variants” and “fragments” are described elsewhere herein.

In some embodiments, an “active” IL-7 protein or fragment or variant is characterized, for example, by its ability to bind to an IL-7R receptor-α (CD127) or common-γ chain receptor (CD132) chain present on the surface of an immune cell in vitro or in vivo, and stimulate downstream signaling activities. Exemplary downstream signaling activities include stimulating the differentiation of multipotent (pluripotent) hematopoietic stem cells into lymphoid progenitor cells, and stimulating the proliferation of cells in the lymphoid lineage, including B cells, T cells, and NK cells.

Any one or more of the foregoing IL-7 polypeptides can be combined with any of the anti-denatured collagen antibodies (including antigen-binding fragments thereof) and linkers described herein, to generate one or more fusion proteins or larger, multi-chain structures comprising the same.

Interleukin-21 (IL-21). Certain effector domains comprise one or more IL-21 polypeptides. Interleukin-21 has potent regulatory effects on cells of the immune system, including NK cells and cytotoxic T cells, for example, by inducing cell division/proliferation in its target cells. IL-21 is expressed in activated human CD4+ T cells and NK T cells, and is also up-regulated in Th2 and Th17 subsets of T helper cells and T follicular cells.

IL-21 binds to the IL-21 receptor (IL-21R), which is expressed on the surface of T cells, B cells, and NK cells. IL-21R is similar in structure to the receptors for other type I cytokines like IL-2R and IL-15R and requires dimerization with the common gamma chain (γc) in order to bind IL-21. When bound to IL-21, the IL-21R acts through the Jak/STAT pathway, utilizing Jak1 and Jak3 and a STAT3 homodimer to activate its target genes. IL-21 has shown utility in the treatment of cancers, viral infections, and a variety of inflammatory diseases. Exemplary IL-21 amino acid sequences are provided in Table S9.

TABLE S9
Exemplary IL-21 Polypeptides

| | | SEQ ID |
|---|---|---|
| Name | Sequence | NO. |
| Human IL-21 | MRSSPGNMERIVICLMVIFLGTLVHKSSSQGQDRHMIRMRQLIDIVDQL | 476 |
| Full-length | KNYVNDLVPEFLPAPEDVETNCEWSAFSCFQKAQLKSANTGNNERIINV | |
| | SIKKLKRKPPSTNAGRRQKHRLTCPSCDSYEKKPPKEFLERFKSLLQKM | |
| | IHQHLSSRTHGSEDS | |
| Human IL-21 | HKSSSQGQDRHMIRMRQLIDIVDQLKNYVNDLVPEFLPAPEDVETNCEW | 477 |
| Mature | SAFSCFQKAQLKSANTGNNERIINVSIKKLKRKPPSTNAGRRQKHRLTC | |
| | PSCDSYEKKPPKEFLERFKSLLQKMIHQHLSSRTHGSEDS | |
| Human IL-21 | GQDRHMIRMRQLIDIVDQLKNYVNDLVPEFLPAPEDVETNCEWSAFSCF | 478 |
| (31-162) | QKAQLKSANTGNNERIINVSIKKLKRKPPSTNAGRRQKHRLTCPSCDSY | |
| | EKKPPKEFLERFKSLLQKMIHQHLSSRTHGSEDS | |

Thus, in certain embodiments, an IL-21 polypeptide comprises, consists, or consists essentially of an amino acid sequence selected from Table S9, or an active variant or fragment thereof that is at least 80, 85, 90, 95, 98, or 100% identical to a sequence selected from Table S9. Additional examples of active polypeptide “variants” and “fragments” are described elsewhere herein.

In some embodiments, an “active” IL-21 protein or fragment or variant is characterized, for example, by its ability to bind to an IL-21R chain present on the surface of an immune cell in vitro or in vivo, and stimulate downstream signaling activities. Exemplary downstream signaling activities include inducing cell division/proliferation of NK cells and cytotoxic T cells.

Any one or more of the foregoing IL-21 polypeptides can be combined with any of the anti-denatured collagen antibodies (including antigen-binding fragments thereof) and linkers described herein, to generate one or more fusion proteins or larger, multi-chain structures comprising the same.

Immunomodulatory or Anti-Cancer Antibodies. In certain embodiments, an effector domain comprises an immunomodulatory antibody, or antigen-binding fragment thereof, for example, to create a bi-specific or multi-specific antibody. In some instances, an effector domain comprises an anti-cancer antibody, or antigen-binding fragment thereof. Examples of immunomodulatory antibodies, or antigen-binding fragments thereof, include “antagonists” of an immune-inhibitory receptor, and “agonists” of an immune-stimulatory receptor.

In some instances, the antibody, or antigen-binding fragment thereof, binds to a therapeutically-relevant antigen, such as an immunomodulatory or cancer antigen or receptor.

Exemplary antigens or receptors are selected from one or more of human Her2/neu, Her1/EGF receptor (EGFR), EGFR1, EGFR2, EGFR3, Her3, A33 antigen, B7H3, B7H4, CD3, CD4, CD5, CD8, CD16, CD19, CD20, CD30, CD22, CD23 (IgE Receptor), B-cell maturation antigen (BCMA), Trop-2, Claudin 6, claudin 16, MAGE-3, C242 antigen, 5T4, IL-6, IL-13, PD-1, CTLA-4, PD-L1, TIGIT, TIM-3, LAG-3, 4-1BB, vascular endothelial growth factor VEGF (e.g., VEGF-A) VEGFR-1, VEGFR-2, CD27, CD28, CD33, CD37, CD40, CD44, CD51, CD52, CD56, CD74, CD80, CD86, CD137, CD152, CD200, CD221, CCR4, HLA-DR, CTLA-4, NPC-1C, Siglec15, MIC-A, NKG2A, NKG2D, Nkp30, NKp46, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin. Exemplary antibodies to such antigens or receptors are known and available in the art.

In specific embodiments, the immunomodulatory or anti-cancer antibody, or antigen-binding fragment thereof, specifically binds to human CD40 (see Table S10). In some embodiments, the antibody, or antigen-binding fragment thereof, specifically binds to a TNF superfamily receptor (selected, for example, from Table T1), such as human 4-1BB (see Table S10).

Any one or more of the foregoing immunomodulatory or anti-cancer antibodies can be combined with any of the anti-denatured collagen antibodies (including antigen-binding fragments thereof) and linkers described herein, to generate one or more fusion proteins or larger, multi-chain structures comprising the same.

Linkers. As noted above, in certain embodiments, a fusion protein comprises a linker or a peptide linker, including a flexible linker. In some embodiments, the linker is a cleavable linker, for example, a cleavable linker that comprises a protease cleavage site. In some embodiments, the linker is a non-cleavable linker, that is, a physiologically-stable linker, or a stable linker.

In some embodiments, the linker is about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length. In particular embodiments, the first linker is a cleavable linker, and the second linker is a non-cleavable or stable linker. In some embodiments, the first linker is a non-cleavable or stable linker, and the second linker is a cleavable linker. In some embodiments, the linker facilitates exposure of the effector domain under suitable conditions, for example, in the presence of denatured human collagen in diseased or cancerous tissue, as described herein.

In some embodiments, a cleavable linker comprises at least one protease cleavage site, or is a low pH-sensitive linker. Suitable protease cleavages sites and self-cleaving peptides are known to the skilled person (see, e.g., Ryan et al., J. Gener. Virol. 78:699-722, 1997; and Scymczak et al., Nature Biotech. 5:589-594, 2004). In some embodiments, the protease cleavage site is cleavable by a protease selected from one or more of a metalloprotease, a serine protease, a cysteine protease, and an aspartic acid protease. In particular embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

Exemplary stable linker sequences, including flexible linkers, are provided in Table L1.

TABLE L1
Exemplary stable linkers

| | | SEQ ID |
|---|---|---|
| Name | Sequence | NO: |
| | [G]ₓ | |
| | [S]ₓ | |
| | [N]ₓ | |
| | [GS]ₓ | |
| | [GGS]ₓ | |
| | [GSS]ₓ | |
| | [GSGS]ₓ | 479 |
| | [GGSG]ₓ | 480 |
| | [GGGS]ₓ | 481 |
| | [GSGGG]ₓ | 482 |
| | [GGGGS]ₓ | 483 |
| | [GN]ₓ | |
| | [GGN]ₓ | |
| | [GNN]ₓ | |
| | [GNGN]ₓ | 484 |
| | [GGNG]ₓ | 485 |
| | [GGGN]ₓ | 486 |
| | [GGGGN]ₓ | 487 |
| | DGGGS | 488 |
| | TGEKP | 489 |
| | GGRR | 490 |
| | EGKSSGSGSESKVD | 491 |
| | KESGSVSSEQLAQFRSLD | 492 |
| | GGRRGGGS | 493 |
| | LRQRDGERP | 494 |
| | LRQKDGGGSERP | 495 |
| | LRQKD(GGGS)₂ERP | 496 |

where x is 1, 2, 3, 4, 5, 6, 7, 8, 9, 10, 11, 12, 13, 14, 15, 16, 17, 18, 19, or 20

Thus, in certain embodiment, a stable and/or flexible linker is selected from Table L1. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

Exemplary cleavable linker sequences are provided in Table L2.

TABLE L2
Exemplary cleavable linkers

| | | SEQ ID |
|---|---|---|
| Name | Sequence | NO: |
| | PLGLAG | 497 |
| | SGRSDNR | 498 |
| | PLGLAGSGRSDNR | 499 |
| | GSLSGRSDNHGS | 500 |
| | LGGSGRSANA | 501 |
| | LSGRSANAG | 502 |
| | GPLGLAGRSANA | 503 |
| | PLGLSGRSANAGPA | 504 |
| | PLGLAGRSANAGPA | 505 |
| | GPLGLSGRSANAGPASG | 506 |
| | GPLGLAGRSANAGPASG | 507 |
| | SGPLGLAGRSANAGPAS | 508 |
| | SGPASGRSANAPLGLAG | 509 |
| | GPASGRSANAPLGLAGS | 510 |
| | GPLGLAGRSANPGPASG | 511 |
| | GPLGLAGRSDNHGPASG | 512 |
| | GPLGLAGRSDNPGPASG | 513 |
| | GPLGLAGRSENPGPASG | 514 |
| | GPLGLAGRSDNLGPASG | 515 |
| | GPLGLAGRNAQVGPASG | 516 |
| | LSGRSDNA | 517 |
| | LSGRSDND | 518 |
| | LSGRSDNE | 519 |
| | LSGRSDNF | 520 |
| | LSGRSDNG | 521 |
| | LSGRSDNI | 522 |
| | LSGRSDNK | 523 |
| | LSGRSDNL | 524 |
| | LSGRSDNM | 525 |
| | LSGRSDNN | 526 |
| | LSGRSDNP | 527 |
| | LSGRSDNQ | 528 |
| | LSGRSDNR | 529 |
| | LSGRSDNS | 530 |
| | LSGRSDNT | 531 |
| | LSGRSDNV | 532 |
| | LSGRSDNW | 533 |
| | LSGRSDNY | 534 |
| | LSGRSAND | 535 |
| | LSGRSANE | 536 |
| | LSGRSANF | 537 |
| | LSGRSANG | 538 |
| | LSGRSANH | 539 |
| | LSGRSANI | 540 |
| | LSGRSANK | 541 |
| | LSGRSANL | 542 |
| | LSGRSANM | 543 |
| | LSGRSANN | 544 |
| | LSGRSANP | 545 |
| | LSGRSANQ | 546 |
| | LSGRSANR | 547 |
| | LSGRSANS | 548 |
| | LSGRSANT | 549 |
| | LSGRSANV | 550 |
| | LSGRSANW | 551 |
| | LSGRSANY | 552 |
| | PLGLAGRSDNHS | 553 |
| | PLGLAGSGRSDNRGA | 554 |
| | PLGLAGSGRSDNQGA | 555 |
| | PLGLAGSGRSDNYGA | 556 |
| | GPLGLAGSGRSDNQG | 557 |
| | PLGLAGSGRSDNQ | 558 |
| | PLGLAGSGRSDNH | 559 |
| | PLGLAGSGRSDNT | 560 |
| | SGRSDNH | 561 |

Thus, in certain embodiment, a cleavable linker is selected from Table L2. In particular embodiments, a cleavable linker has a half life at pH 7.4, 25° C., for example, at physiological pH, human body temperature (e.g., in vivo, in serum, in a given tissue), of about or less than about 30 minutes, 1 hour, 2 hours, 3 hours, 4 hours, 5 hours, 6 hours, 12 hours, 18 hours, 24 hours, 36 hours, 48 hours, 72 hours, or 96 hours, or any intervening half-life.

Any one or more of the foregoing linkers can be any of the anti-denatured collagen antibodies (including antigen-binding fragments thereof) and effector domains described herein, to generate one or more fusion proteins or larger, multi-chain structures comprising the same.

Additional Domains. Certain fusion proteins comprise one or more additional domains, for example, binding domains. In some embodiments, each of polypeptides in a fusion protein further comprise one or more protein domains at a free terminus.

In particular embodiments, the protein domains are selected from one or more of cell receptor targeting moieties optionally bi-specific targeting moieties, antigen-binding domains optionally bi-specific antigen-binding domains, cell membrane receptor extracellular domains (ECDs), Fc domains, human serum albumin (HSA), Fc binding domains, HSA binding domains, cytokines, chemokines, and soluble protein ligands.

In some embodiments, the one or more additional protein domains can be used to form complexes of two, three, four, five, or more fusion proteins, which are bound to together via the additional domain(s).

Exemplary fusion proteins, including bi-specific antibodies, are provided in Table S10.

TABLE S10
Exemplary Fusion Proteins and Bi-Specific Antibodies

| | | |
|---|---|---|
| | | SEQ ID |
| Chain Name | Sequence | NO: |

P27992713 - Fusion to proIL-2

| | | |
|---|---|---|
| Chains 1 | QVTLKESGPALVKPTQTLTLTCTFSGFSLSTSGMGVGWIRQPPGKALEWLAD | 562 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_D10- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| PLGLAG- | EEELKPLEEVLNLAHSKNFHFDPRDVVSNINVFVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVHSNGNTYLEWYLQKPGQSPQLL | 563 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28002713 - Fusion to proIL-2

| | | |
|---|---|---|
| Chains 1 | QVTLKESGPALVKPTQTLTLTCTFSGFSLSTSGMGVGWIRQPPGKALEWLAD | 564 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSGGSG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_D10- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| GGSGGS- | EEELKPLEEVLNLAHSKNFHFDPRDVVSNINVFVLELKGSETTFMCEYADET | |
| GGSGGSG- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVHSNGNTYLEWYLQKPGQSPQLL | 565 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28012716 - Fusion to proIL-2

| | | |
|---|---|---|
| Chains 1 | QVTLKESGPALVKPTQTLTLTCTFSGFSLSTPGMGVWWIRQPPGKALEWLAD | 566 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_D10- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| PLGLAG- | EEELKPLEEVLNLAHSKNFHFDPRDVVSNINVFVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVSSWGNTYLEWYLQKPGQSPQLL | 567 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28012798 - Fusion to proIL-2

| | | |
|---|---|---|
| Chains 1 | QVTLKESGPALVKPTQTLTLTCTFSGFSLSTPGMGVWWIRQPPGKALEWLAD | 568 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_D10- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| PLGLAG- | EEELKPLEEVLNLAHSKNFHFDPRDVVSNINVFVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVHSNANTYLEWYLQKPGQSPQLL | 569 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28022716 - Fusion to proIL-2

| | | |
|---|---|---|
| Chains 1 | QVTLKESGPALVKPTQTLTLTCTFSGFSLSTPGMGVWWIRQPPGKALEWLAD | 570 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSGGSG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_D10- | TVDKSRWQQGNVFSCSVMHEALHNHYTQKSLSLSPG [symbol] T | |
| GGSGG- | KKTQLQLEHLLLDLQMILNGINNYKNPELTRMLTAKFYMPKKATELKHLQCL | |
| GGSGGS- | EEELKPLEEVLNLAHSKNFHFDPRDVVSNINVFVLELKGSETTFMCEYADET | |
| GGSGGSG- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVSSWGNTYLEWYLQKPGQSPQLL | 571 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28052797 - Fusion to proIL-2

| | | |
|---|---|---|
| Chains 1 | QVTLKESGPALVKPTQTLTLTCTFSGFSLSTSGMGVGWIRQPPGKALEWLAD | 572 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVHSSGNTYLEWYLQKPGQSPQLL | 573 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28052798 - Fusion to proIL-2

| | | |
|---|---|---|
| Chains 1 | QVTLKESGPALVKPTQTLTLTCTFSGFSLSTSGMGVGWIRQPPGKALEWLAD | 574 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVHSNANTYLEWYLQKPGQSPQLL | 575 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28062798 - Fusion to proIL-2

| | | |
|---|---|---|
| Chains 1 | QVTLKESGPALVKPTQTLTLTCTFSGFSLSTSGMGVGWIRQPPGKALEWLAD | 576 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| PLGLAG- | EEELKPLEEVLNLAQSKNFHLRPRDLISNINVIVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVHSNANTYLEWYLQKPGQSPQLL | 577 |
| and 4 | TYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28072798 - Fusion to proIL-2

| | | |
|---|---|---|
| Chains 1 | QVTLKESGPALVKPTQTLTLTCTFSGFSLSTSGMGVGWIRQPPGKALEWLAD | 578 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_Y45A- | TVDKSRWQQGNVFSCSVMHEALHNHYTQKSLSLSPG [symbol] ST | |
| GGSGG- | KKTQLQLEHLLLDLQMILNGINNYKNPELTRMLTAKFAMPKKATELKHLQCL | |
| PLGLAG- | EEELKPLEEVLNLAQSKNFHLRPRDLISNINVIVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVHSNANTYLEWYLQKPGQSPQLL | 579 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28082714 - Fusion to proIL-2

| | | |
|---|---|---|
| Chains 1 | QVTLKESGPALVKPTQTLTLTCTFSGFSLSTPGMGVWWIRQPPGKALEWLAD | 580 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVHSQGNTYLEWYLQKPGQSPQLL | 581 |
| and 4 | IYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28082716 - Fusion to proIL-2

| | | |
|---|---|---|
| Chains 1 | QVTLKESGPALVKPTQTLTLTCTFSGFSLSTPGMGVWWIRQPPGKALEWLAD | 582 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVSSWGNTYLEWYLQKPGQSPQLL | 583 |
| and 4 | TYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28082797 - Fusion to proIL-2

| | | |
|---|---|---|
| Chains 1 | QVTLKESGPALVKPTQTLTLTCTFSGFSLSTPGMGVWWIRQPPGKALEWLAD | 584 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVHSSGNTYLEWYLQKPGQSPQLL | 585 |
| and 4 | TYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28082798 - Fusion to proIL-2

| | | |
|---|---|---|
| Chains 1 | QVTLKESGPALVKPTQTLTLTCTFSGFSLSTPGMGVWWIRQPPGKALEWLAD | 586 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_F42A_ | TKNQVSLTCLVKGFYPSDIAVEWESNGQPENNYKTTPPVLDSDGSFFLYSKL | |
| K43E_E61K- | TVDKSRWQQGNVFSCSVMHEALHNHYTQKSLSLSPG [symbol] | |
| GGSGG- | STKKTQLQLEHLLLDLQMILNGINNYKNPKLTRMLTAEFYMPKKATELKHLQ | |
| PLGLAG- | CLEKELKPLEEVLNLAQSKNFHLRPRDLISNINVIVLELKGSETTFMCEYAD | |
| SGRSDNR- | ETATIVEFLNRWITFSQSIISTLT [symbol] [symbol] ELCDDDP | |
| GGA- | PEIPHATFKAMAYKEGTMLNCKCKRGFRRIESGSLYMLCTGNSSHSSWDNQC | |
| R_E29K_K38E | QCTSSATRNTTKQVTPQPEEQKERKTTEMQSPMQPVDQASLPGHCREPPPWE | |
| 14/21R | NEATERIYHFVVGQMVYYQCVQGYRALHRGPAESVCKMTHGKTRWTQPQLIC | |
| | TGE | |
| Chains 3 | DIVMTQTPLSLPVTPGEPASISCRSSQSIVHSNANTYLEWYLQKPGQSPQLL | 587 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28092716 - Fusion to proIL-2

| | | |
|---|---|---|
| Chains 1 | QVTLKESGPALVKPTQTLTLTCTFSGFSLSTPGMGVWWIRQPPGKALEWLAD | 588 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_- | TVDKSRWQQGNVFSCSVMHEALHNHYTQKSLSLSPG [symbol] ST | |
| GGSGG- | KKTQLQLEHLLLDLQMILNGINNYKNPELTRMLTAKFYMPKKATELKHLQCL | |
| PLGLAG- | EEELKPLEEVLNLAQSKNFHLRPRDLISNINVIVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVSSWGNTYLEWYLQKPGQSPQLL | 589 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28102716 - Fusion to proIL-2

| | | |
|---|---|---|
| Chains 1 | QVTLKESGPALVKPTQTLTLTCTFSGFSLSTPGMGVWWIRQPPGKALEWLAD | 590 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSPLGLAGG | NHKPSNTKVDKKVEPKSCDKTHTCPPCPAPEAAGGPSVFLFPPKPKDTLMIS | |
| GS-GG- | RTPEVTCVVVDVSHEDPEVKFNWYVDGVEVHNAKTKPREEQYNSTYRVVSVL | |
| L_wt_del_ | TVLHQDWLNGKEYKCKVSNKALAAPIEKTISKAKGQPREPQVYTLPPSRDEL | |
| APASS_K35E_ | TKNQVSLTCLVKGFYPSDIAVEWESNGQPENNYKTTPPVLDSDGSFFLYSKL | |
| F42A_Y45A- | TVDKSRWQQGNVFSCSVMHEALHNHYTQKSLSLSPG [symbol] ST | |
| GGSGG- | KKTQLQLEHLLLDLQMILNGINNYKNPELTRMLTAKFAMPKKATELKHLQCL | |
| PLGLAG- | EEELKPLEEVLNLAQSKNFHLRPRDLISNINVIVLELKGSETTFMCEYADET | |
| SGRSDNR- | ATIVEFLNRWITFSQSIISTLT [symbol] [symbol] ELCDDDPPE | |
| GGA-R | IPHATFKAMAYKEGTMLNCECKRGFRRIKSGSLYMLCTGNSSHSSWDNQCQC | |
| 14/21R | TSSATRNTTKQVTPQPEEQKERKTTEMQSPMQPVDQASLPGHCREPPPWENE | |
| | ATERIYHFVVGQMVYYQCVQGYRALHRGPAESVCKMTHGKTRWTQPQLICTG | |
| | E | |
| Chains 3 | DIVMTQTPLSLPVTPGEPASISCRSSQSIVSSWGNTYLEWYLQKPGQSPQLL | 591 |
| and 4 | TYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28472797 - Fusion to proIL-2

| | | |
|---|---|---|
| Chains 1 | QVTLKESGPALVKPTQTLTLTCTFSGFSLSTSGMGVGWIRQPPGKALEWLAD | 592 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVHSSGNTYLEWYLQKPGQSPQLL | 593 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28472798 - Fusion to proIL-2

| | | |
|---|---|---|
| Chains 1 | QVTLKESGPALVKPTQTLTLTCTFSGFSLSTSGMGVGWIRQPPGKALEWLAD | 594 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVHSNANTYLEWYLQKPGQSPQLL | 595 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28482714 - Fusion to proIL-2

| | | |
|---|---|---|
| Chains 1 | QVTLKESGPALVKPTQTLTLTCTFSGFSLSTPGMGVWWIRQPPGKALEWLAD | 596 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVHSQGNTYLEWYLQKPGQSPQLL | 597 |
| and 4 | TYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28482716 - Fusion to proIL-2

| | | |
|---|---|---|
| Chains 1 | QVTLKESGPALVKPTQTLTLTCTFSGFSLSTPGMGVWWIRQPPGKALEWLAD | 598 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVSSWGNTYLEWYLQKPGQSPQLL | 599 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28482797 - Fusion to proIL-2

| | | |
|---|---|---|
| Chains 1 | QVTLKESGPALVKPTQTLTLTCTFSGFSLSTPGMGVWWIRQPPGKALEWLAD | 600 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVHSSGNTYLEWYLQKPGQSPQLL | 601 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28482798 - Fusion to proIL-2

| | | |
|---|---|---|
| Chains 1 | QVTLKESGPALVKPTQTLTLTCTFSGFSLSTPGMGVWWIRQPPGKALEWLAD | 602 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGSGGSPLGL | NHKPSNTKVDKKVEPKSCDKTHTCPPCPAPEAAGGPSVFLFPPKPKDTLMIS | |
| AGGGSGGS- | RTPEVTCVVVDVSHEDPEVKFNWYVDGVEVHNAKTKPREEQYNSTYRVVSVL | |
| GG- | TVLHQDWLNGKEYKCKVSNKALAAPIEKTISKAKGQPREPQVYTLPPSRDEL | |
| L_wt_del_ | TKNQVSLTCLVKGFYPSDIAVEWESNGQPENNYKTTPPVLDSDGSFFLYSKL | |
| APASS_F42A_ | TVDKSRWQQGNVFSCSVMHEALHNHYTQKSLSLSPG [symbol] | |
| K43E_E61K- | [symbol] GGSTKKTQLQLEHLLLDLQMILNGINNYKNPKLTRMLTAEFYMPKKATEL | |
| GGSGG- | KHLQCLEKELKPLEEVLNLAQSKNFHLRPRDLISNINVIVLELKGSETTFMC | |
| PLGLAG- | EYADETATIVEFLNRWITFSQSIISTLT [symbol] [symbol] ELC | |
| SGRSDNR- | DDDPPEIPHATFKAMAYKEGTMLNCKCKRGFRRIESGSLYMLCTGNSSHSSW | |
| GGA- | DNQCQCTSSATRNTTKQVTPQPEEQKERKTTEMQSPMQPVDQASLPGHCREP | |
| R_E29K_K38E | PPWENEATERIYHFVVGQMVYYQCVQGYRALHRGPAESVCKMTHGKTRWTQP | |
| 20/21R | QLICTGE | |
| Chains 3 | DIVMTQTPLSLPVTPGEPASISCRSSQSIVHSNANTYLEWYLQKPGQSPQLL | 603 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27172713 - Fusion to proIL-15

| | | |
|---|---|---|
| Chains 1 | QVTLKESGPALVKPTQTLTLTCTFSGFSLSTSGMGVGWIRQPPGKALEWLAD | 604 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GS- | TVLHQDWLNGKEYKCKVSNKALAAPIEKTISKAKGQPREPQVYTLPPSRDEL | |
| L_V49D_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] N | |
| GGGSGGGSGG | WVNVISDLKKIEDLIQSMHIDATLYTESDVHPSCKVTAMKCFLLELQDISLE | |
| GSGGGSGGGS | SGDASIHDTVENLIILANNSLSSNGNVTESGCKECEELEEKNIKEFLQSFVH | |
| GGGS-R_T2A | IVQMFINTA [symbol] [symbol] IACPPPMSVEHADIWVKSY | |
| 15R/24 | SLYSRERYICNSGFKRKAGTSSLTECVLNKATNVAHWTTPSLKCIRDPALVH | |
| | QRPAPPS | |
| Chains 3 | DIVMTQTPLSLPVTPGEPASISCRSSQSIVHSNGNTYLEWYLQKPGQSPQLL | 605 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27182713 - Fusion to proIL-15

| | | |
|---|---|---|
| Chains 1 | QVTLKESGPALVKPTQTLTLTCTFSGFSLSTSGMGVGWIRQPPGKALEWLAD | 606 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCKECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSNGNTYLEWYLQKPGQSPQLL | 607 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27192713 - Fusion to proIL-15

| | | |
|---|---|---|
| Chains 1 | QVTLKESGPALVKPTQTLTLTCTFSGFSLSTSGMGVGWIRQPPGKALEWLAD | 608 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86F_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCFECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSNGNTYLEWYLQKPGQSPQLL | 609 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27202713 - Fusion to proIL-15

| | | |
|---|---|---|
| Chains 1 | QVTLKESGPALVKPTQTLTLTCTFSGFSLSTSGMGVGWIRQPPGKALEWLAD | 610 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSNGNTYLEWYLQKPGQSPQLL | 611 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27202714 - Fusion to proIL-15

| | | |
|---|---|---|
| Chains 1 | QVTLKESGPALVKPTQTLTLTCTFSGFSLSTSGMGVGWIRQPPGKALEWLAD | 612 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSQGNTYLEWYLQKPGQSPQLL | 613 |
| and 4 | IYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27202716 - Fusion to proIL-15

| | | |
|---|---|---|
| Chains 1 | QVTLKESGPALVKPTQTLTLTCTFSGFSLSTSGMGVGWIRQPPGKALEWLAD | 614 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVSSWGNTYLEWYLQKPGQSPQLL | 615 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27202797 - Fusion to proIL-15

| | | |
|---|---|---|
| Chains 1 | QVTLKESGPALVKPTQTLTLTCTFSGFSLSTSGMGVGWIRQPPGKALEWLAD | 616 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSSGNTYLEWYLQKPGQSPQLL | 617 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27202798 - Fusion to proIL-15

| | | |
|---|---|---|
| Chains 1 | QVTLKESGPALVKPTQTLTLTCTFSGFSLSTSGMGVGWIRQPPGKALEWLAD | 618 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVHSNANTYLEWYLQKPGQSPQLL | 619 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27212713 - Fusion to proIL-15

| | | |
|---|---|---|
| Chains 1 | QVTLKESGPALVKPTQTLTLTCTFSGFSLSTSGMGVGWIRQPPGKALEWLAD | 620 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GS-PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQD | |
| GSGGGSGGGS | ISLESGDASIHDTVENLIILANNSLSSNGNVTESGCKECEELEEKNIKEFLQ | |
| GGGS-R_T2A | SFVHIVQMFINTA [symbol] [symbol] IACPPPMSVEHADIW | |
| 19R/24 | VKSYSLYSRERYICNSGFKRKAGTSSLTECVLNKATNVAHWTTPSLKCIRDP | |
| | ALVHQRPAPPS | |
| Chains 3 | DIVMTQTPLSLPVTPGEPASISCRSSQSIVHSNGNTYLEWYLQKPGQSPQLL | 621 |
| and 4 | TYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27212714 - Fusion to proIL-15

| | | |
|---|---|---|
| Chains 1 | QVTLKESGPALVKPTQTLTLTCTFSGFSLSTSGMGVGWIRQPPGKALEWLAD | 622 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GS-PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQD | |
| GSGGGSGGGS | ISLESGDASIHDTVENLIILANNSLSSNGNVTESGCKECEELEEKNIKEFLQ | |
| GGGS-R_T2A | SFVHIVQMFINTA [symbol] [symbol] IACPPPMSVEHADIW | |
| 19R/24 | VKSYSLYSRERYICNSGFKRKAGTSSLTECVLNKATNVAHWTTPSLKCIRDP | |
| | ALVHQRPAPPS | |
| Chains 3 | DIVMTQTPLSLPVTPGEPASISCRSSQSIVHSQGNTYLEWYLQKPGQSPQLL | 623 |
| and 4 | TYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27212716 - Fusion to proIL-15

| | | |
|---|---|---|
| Chains 1 | QVTLKESGPALVKPTQTLTLTCTFSGFSLSTSGMGVGWIRQPPGKALEWLAD | 624 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GS-PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQD | |
| GSGGGSGGGS | ISLESGDASIHDTVENLIILANNSLSSNGNVTESGCKECEELEEKNIKEFLQ | |
| GGGS-R_T2A | SFVHIVQMFINTA [symbol] [symbol] IACPPPMSVEHADIW | |
| 19R/24 | VKSYSLYSRERYICNSGFKRKAGTSSLTECVLNKATNVAHWTTPSLKCIRDP | |
| | ALVHQRPAPPS | |
| Chains 3 | DIVMTQTPLSLPVTPGEPASISCRSSQSIVSSWGNTYLEWYLQKPGQSPQLL | 625 |
| and 4 | TYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27902713 - Fusion to proIL-15

| | | |
|---|---|---|
| Chains 1 | QVTLKESGPALVKPTQTLTLTCTFSGFSLSTSGMGVGWIRQPPGKALEWLAD | 626 |
| and 2 | IWWDDNKYANPSLKSRLTISKDTSKNQVVLTMTNMDPVDTATYYCARRANYG | |
| hB7_IgG1 | NPYYAQDYWGQGTTVTVSSASTKGPSVFPLAPSSKSTSGGTAALGCLVKDYF | |
| (AAA)_delK- | PEPVTVSWNSGALTSGVHTFPAVLQSSGLYSLSSVVTVPSSSLGTQTYICNV | |
| GGGGSG- | NHKPSNTKVDKKVEPKSCDKTHTCPPCPAPEAAGGPSVFLFPPKPKDTLMIS | |
| GGSGGGS- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17/24 | SYSLYSRERYICNSGFKRKAGTSSLTECVLNKATNVAHWTTPSLKCIRDPAL | |
| (stable) | VHQRPAPPS | |
| Chains 3 | DIVMTQTPLSLPVTPGEPASISCRSSQSIVHSNGNTYLEWYLQKPGQSPQLL | 627 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27912716 - Fusion to proIL-15

| | | |
|---|---|---|
| Chains 1 | QVTLKESGPALVKPTQTLTLTCTFSGFSLSTPGMGVWWIRQPPGKALEWLAD | 628 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17R/24 | SYSLYSRERYICNSGFKRKAGTSSLTECVLNKATNVAHWTTPSLKCIRDPAL | |
| | VHQRPAPPS | |
| Chains 3 | DIVMTQTPLSLPVTPGEPASISCRSSQSIVSSWGNTYLEWYLQKPGQSPQLL | 629 |
| and 4 | TYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27922716 - Fusion to proIL-15

| | | |
|---|---|---|
| Chains 1 | QVTLKESGPALVKPTQTLTLTCTFSGFSLSTPGMGVWWIRQPPGKALEWLAD | 630 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GGGGSG- | NHKPSNTKVDKKVEPKSCDKTHTCPPCPAPEAAGGPSVFLFPPKPKDTLMIS | |
| GGSGGGS- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] | |
| GGGSGGGSGG | [symbol] NWVNVISDLKKIEDLIQSMHIDATLYTESDVHPSCKVTAMKCFLLELQDIS | |
| GSGGGSGGGS | LESGDASIHDTVENLIILANNSLSSNGNVTESGCGECEELEEKNIKEFLQSF | |
| GGGS-R_T2A | VHIVQMFINTA [symbol] [symbol] IACPPPMSVEHADIWVK | |
| 17/24 | SYSLYSRERYICNSGFKRKAGTSSLTECVLNKATNVAHWTTPSLKCIRDPAL | |
| (stable) | VHQRPAPPS | |
| Chains 3 | DIVMTQTPLSLPVTPGEPASISCRSSQSIVSSWGNTYLEWYLQKPGQSPQLL | 631 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27932713 - Fusion to proIL-15

| | | |
|---|---|---|
| Chains 1 | QVTLKESGPALVKPTQTLTLTCTFSGFSLSTSGMGVGWIRQPPGKALEWLAD | 632 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_K86G_ | TKNQVSLTCLVKGFYPSDIAVEWESNGQPENNYKTTPPVLDSDGSFFLYSKL | |
| (S162A) | TVDKSRWQQGNVFSCSVMHEALHNHYTQKSLSLSPG [symbol] | |
| 17R | [symbol] NWVNVISDLKKIEDLIQSMHIDATLYTESDVHPSCKVTAMKCFLLELQDIS | |
| | LESGDASIHDTVENLIILANNSLSSNGNVTESGCGECEELEEKNIKEFLQSF | |
| | VHIVQMFINTA | |
| Chains 3 | DIVMTQTPLSLPVTPGEPASISCRSSQSIVHSNGNTYLEWYLQKPGQSPQLL | 633 |
| and 4 | IYKVSNRFSGVPDRFSGSGSGTDFTLKISRVEAEDVGVYYCFQGSHVPWTFG | |
| hB7_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27952714 - Fusion to proIL-15

| | | |
|---|---|---|
| Chains 1 | QVTLKESGPALVKPTQTLTLTCTFSGFSLSTSGMGVGWIRQPPGKALEWLAD | 634 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVHSQGNTYLEWYLQKPGQSPQLL | 635 |
| and 4 | IYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27952716 - Fusion to proIL-15

| | | |
|---|---|---|
| Chains 1 | QVTLKESGPALVKPTQTLTLTCTFSGFSLSTSGMGVGWIRQPPGKALEWLAD | 636 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVSSWGNTYLEWYLQKPGQSPQLL | 637 |
| and 4 | TYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27952797 - Fusion to proIL-15

| | | |
|---|---|---|
| Chains 1 | QVTLKESGPALVKPTQTLTLTCTFSGFSLSTSGMGVGWIRQPPGKALEWLAD | 638 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVHSSGNTYLEWYLQKPGQSPQLL | 639 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27952798 - Fusion to proIL-15

| | | |
|---|---|---|
| Chains 1 | QVTLKESGPALVKPTQTLTLTCTFSGFSLSTSGMGVGWIRQPPGKALEWLAD | 640 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVHSNANTYLEWYLQKPGQSPQLL | 641 |
| and 4 | TYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27962716 - Fusion to proIL-15

| | | |
|---|---|---|
| Chains 1 | QVTLKESGPALVKPTQTLTLTCTFSGFSLSTPGMGVWWIRQPPGKALEWLAD | 642 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVSSWGNTYLEWYLQKPGQSPQLL | 643 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27962797 - Fusion to proIL-15

| | | |
|---|---|---|
| Chains 1 | QVTLKESGPALVKPTQTLTLTCTFSGFSLSTPGMGVWWIRQPPGKALEWLAD | 644 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVHSSGNTYLEWYLQKPGQSPQLL | 645 |
| and 4 | IYKVSNRFSGVPDRFSGSGSGTDFTLKISRVEAEDVGVYYCFQGSHVPWTFG | |
| hB7S_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P27962798 - Fusion to proIL-15

| | | |
|---|---|---|
| Chains 1 | QVTLKESGPALVKPTQTLTLTCTFSGFSLSTPGMGVWWIRQPPGKALEWLAD | 646 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)_- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVHSNANTYLEWYLQKPGQSPQLL | 647 |
| and 4 | TYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28492714 - Fusion to proIL-15

| | | |
|---|---|---|
| Chains 1 | QVTLKESGPALVKPTQTLTLTCTFSGFSLSTSGMGVGWIRQPPGKALEWLAD | 648 |
| and 2 | IWWDDNKYANPSLKSRLTISKDTSKNQVVLTMTNMDPVDTATYYCARRANYG | |
| hB7_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GS-PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQD | |
| GGGSGGGSGG | ISLESGDASIHDTVENLIILANNSLSSAGNVTESGCGECEELEEKNIKEFLQ | |
| GSGGGSGGGS | SFVHIVQMFINTA [symbol] [symbol] IACPPPMSVEHADIW | |
| GGGS-R_T2A | VKSYSLYSRERYICNSGFKRKAGTSSLTECVLNKATNVAHWTTPSLKCIRDP | |
| 19R/24 | ALVHQRPAPPS | |
| Chains 3 | DIVMTQTPLSLPVTPGEPASISCRSSQSIVHSQGNTYLEWYLQKPGQSPQLL | 649 |
| and 4 | IYKVSNRFSGVPDRFSGSGSGTDFTLKISRVEAEDVGVYYCFQGSHVPWTFG | |
| hB7Q_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28502716 - Fusion to proIL-15

| | | |
|---|---|---|
| Chains 1 | QVTLKESGPALVKPTQTLTLTCTFSGFSLSTPGMGVWWIRQPPGKALEWLAD | 650 |
| and 2 | IWWDDNKYTNPSLKSRLTISKDTSKNQVVLTMTNMDPVDTATYYCARRANYG | |
| hD3_IgG1(AAA)_ | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| GS-PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQD | |
| GGGSGGGSGG | ISLESGDASIHDTVENLIILANNSLSSAGNVTESGCGECEELEEKNIKEFLQ | |
| GSGGGSGGGS | SFVHIVQMFINTA [symbol] [symbol] IACPPPMSVEHADIW | |
| GGGS-R_T2A | VKSYSLYSRERYICNSGFKRKAGTSSLTECVLNKATNVAHWTTPSLKCIRDP | |
| 19R/24 | ALVHQRPAPPS | |
| Chains 3 | DIVMTQTPLSLPVTPGEPASISCRSSQSIVSSWGNTYLEWYLQKPGQSPQLL | 651 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28612716 - Fusion to proIL-15

| | | |
|---|---|---|
| Chains 1 | QVTLKESGPALVKPTQTLTLTCTFSGFSLSTPGMGVWWIRQPSGKALEWLAD | 652 |
| and 2 | IWWDDNKYTNPSLKSRLTISKDTSKNQVVLTMTNMDPVDTATYYCARRANYG | |
| hD3a_IgG1(AAA) | NPYYAQDYWGQGTTVTVSSASTKGPSVFPLAPSSKSTSGGTAALGCLVKDYF | |
| delK- | PEPVTVSWNSGALTSGVHTFPAVLQSSGLYSLSSVVTVPSSSLGTQTYICNV | |
| PLGLAG- | NHKPSNTKVDKKVEPKSCDKTHTCPPCPAPEAAGGPSVFLFPPKPKDTLMIS | |
| SGRSDNR- | RTPEVTCVVVDVSHEDPEVKFNWYVDGVEVHNAKTKPREEQYNSTYRVVSVL | |
| GGGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| K86G_ | TVDKSRWQQGNVFSCSVMHEALHNHYTQKSLSLSPG [symbol] | |
| (S162A)- | [symbol] NWVNVISDLKKIEDLIQSMHIDATLYTESDVHPSCKVTAMKCFLLELQDIS | |
| GGGSGGGSGG | LESGDASIHDTVENLIILANNSLSSAGNVTESGCGECEELEEKNIKEFLQSF | |
| GSGGGSGGGS | VHIVQMFINTA [symbol] [symbol] IACPPPMSVEHADIWVK | |
| GGGS-R_T2A | SYSLYSRERYICNSGFKRKAGTSSLTECVLNKATNVAHWTTPSLKCIRDPAL | |
| 17R/24 | VHQRPAPPS | |
| Chains 3 | DIVMTQTPLSLPVTPGEPASISCRSSQSIVSSWGNTYLEWYLQKPGQSPQLL | 653 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28672716 - Fusion to proIL-15

| | | |
|---|---|---|
| Chains 1 | QVTLKESGPALVKPTQTLTLTCTFSGFSLSTPGMGVWWIRQPPGKALEWLAD | 654 |
| and 2 | IWWDDNKYTNPSLKSRLTISKDTSKNQVVLTMTNMDPVDTATYYCARRANYG | |
| 15LR- | NPYYAQDYWGQGTTVTVSSASTKGPSVFPLAPSSKSTSGGTAALGCLVKDYF | |
| 273_hD3_ | PEPVTVSWNSGALTSGVHTFPAVLQSSGLYSLSSVVTVPSSSLGTQTYICNV | |
| IgG1(AAA)_ | NHKPSNTKVDKKVEPKSCDKTHTCPPCPAPEAAGGPSVFLFPPKPKDTLMIS | |
| delK-GGS- | RTPEVTCVVVDVSHEDPEVKFNWYVDGVEVHNAKTKPREEQYNSTYRVVSVL | |
| PLGLAG-GGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] NWVN | |
| GGGS- | VISDLKKIEDLIQSMHIDATLYTESDVHPSCKVTAMKCFLLELQDISLESGD | |
| PLGLAG- | ASIHDTVENLIILANNSLSSAGNVTESGCKECEELEEKNIKEFLQSFVHIVQ | |
| SGRSDNR- | MFINTA [symbol] [symbol] IACPPPMSVEHADIWVKSYSLYSRE | |
| SGGG-R_T2A | RYICNSGFKRKAGTSSLTECVLNKATNVAHWTTPSLKCIRDPALVHQRPAPP | |
| 12/21R | S | |
| Chains 3 | DIVMTQTPLSLPVTPGEPASISCRSSQSIVSSWGNTYLEWYLQKPGQSPQLL | 655 |
| and 4 | IYKVSNRFSGVPDRFSGSGSGTDFTLKISRVEAEDVGVYYCFQGSHVPWTFG | |
| hD3_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P28672798 - Fusion to proIL-15

| | | |
|---|---|---|
| Chains 1 | QVTLKESGPALVKPTQTLTLTCTFSGFSLSTPGMGVWWIRQPPGKALEWLAD | 656 |
| and 2 | IWWDDNKYTNPSLKSRLTISKDTSKNQVVLTMTNMDPVDTATYYCARRANYG | |
| 15LR- | NPYYAQDYWGQGTTVTVSSASTKGPSVFPLAPSSKSTSGGTAALGCLVKDYF | |
| 273_hD3_ | PEPVTVSWNSGALTSGVHTFPAVLQSSGLYSLSSVVTVPSSSLGTQTYICNV | |
| IgG1(AAA)_ | NHKPSNTKVDKKVEPKSCDKTHTCPPCPAPEAAGGPSVFLFPPKPKDTLMIS | |
| delK-GGS- | RTPEVTCVVVDVSHEDPEVKFNWYVDGVEVHNAKTKPREEQYNSTYRVVSVL | |
| PLGLAG-GGS- | TVLHQDWLNGKEYKCKVSNKALAAPIEKTISKAKGQPREPQVYTLPPSRDEL | |
| L_V49D_N77A_ | TKNQVSLTCLVKGFYPSDIAVEWESNGQPENNYKTTPPVLDSDGSFFLYSKL | |
| (S162A)- | TVDKSRWQQGNVFSCSVMHEALHNHYTQKSLSLSPG [symbol] NWVN | |
| GGGS- | VISDLKKIEDLIQSMHIDATLYTESDVHPSCKVTAMKCFLLELQDISLESGD | |
| PLGLAG- | ASIHDTVENLIILANNSLSSAGNVTESGCKECEELEEKNIKEFLQSFVHIVQ | |
| SGRSDNR- | MFINTA [symbol] [symbol] IACPPPMSVEHADIWVKSYSLYSRE | |
| SGGG-R_T2A | RYICNSGFKRKAGTSSLTECVLNKATNVAHWTTPSLKCIRDPALVHQRPAPP | |
| 12/21R | S | |
| Chains 3 | DIVMTQTPLSLPVTPGEPASISCRSSQSIVHSNANTYLEWYLQKPGQSPQLL | 657 |
| and 4 | IYKVSNRFSGVPDRFSGSGSGTDFTLKISRVEAEDVGVYYCFQGSHVPWTFG | |
| hB7A_IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P30262939 - Fusion to proIL-15

| | | |
|---|---|---|
| Chains 1 | EVQLVESGGGLVQPGGSLRLSCAASGFTFSRYWMTWVRQAPGKGLEWIGEIN | 658 |
| and 2 | PDSSTANYTPYLKGRFTISRDNAKNSLYLQMNSLRAEDTAVYYCARPVDGYY | |
| 15LR- | DAMDPWGQGTTVTVSSASTKGPSVFPLAPSSKSTSGGTAALGCLVKDYFPEP | |
| 285_hH8VHe_ | VTVSWNSGALTSGVHTFPAVLQSSGLYSLSSVVTVPSSSLGTQTYICNVNHK | |
| IgG1(AAA)_ | PSNTKVDKKVEPKSCDKTHTCPPCPAPEAAGGPSVFLFPPKPKDTLMISRTP | |
| delK-GGS- | EVTCVVVDVSHEDPEVKFNWYVDGVEVHNAKTKPREEQYNSTYRVVSVLTVL | |
| PLGLAG-GGS- | HQDWLNGKEYKCKVSNKALAAPIEKTISKAKGQPREPQVYTLPPSRDELTKN | |
| L_V49D_N77A_ | QVSLTCLVKGFYPSDIAVEWESNGQPENNYKTTPPVLDSDGSFFLYSKLTVD | |
| (S162A)- | KSRWQQGNVFSCSVMHEALHNHYTQKSLSLSPG [symbol] NWVNVIS | |
| GGGS-PLGLAG- | DLKKIEDLIQSMHIDATLYTESDVHPSCKVTAMKCFLLELQDISLESGDASI | |
| SGRSDNR- | HDTVENLIILANNSLSSAGNVTESGCKECEELEEKNIKEFLQSFVHIVQMFI | |
| SGGG-R_T2A | NT [symbol] [symbol] IACPPPMSVEHADIWVKSYSLYSRERYI | |
| | CNSGFKRKAGTSSLTECVLNKATNVAHWTTPSLKCIRDPALVHQRPAPPS | |
| Chains 3 | DIVMTQSPDSLAVSLGERATINCKSSQSLLNWYNQKNYLAWYQQKPGQPPKL | 659 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

P2869362716 - Bi-specific Ab Fusion to anti-CD40 Ab

| | | |
|---|---|---|
| Chains 1 | QVTLKESGPALVKPTQTLTLTCTFSGFSLSTPGMGVWWIRQPPGKALEWLAD | 660 |
| and 2 D3- | IWWDDNKYTNPSLKSRLTISKDTSKNQVVLTMTNMDPVDTATYYCARRANYG | |
| huIgG1HC_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| GGGGS- | RTPEVTCVVVDVSHEDPEVKFNWYVDGVEVHNAKTKPREEQYNSTYRVVSVL | |
| CP870893- | TVLHQDWLNGKEYKCKVSNKALAAPIEKTISKAKGQPREPQVYTLPPSRDEL | |
| VL-CL | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPGK [symbol] DIQMTQSPSS | |
| | VSASVGDRVTITCRASQGIYSWLAWYQQKPGKAPNLLIYTASTLQSGVPSRF | |
| | SGSGSGTDFTLTISSLQPEDFATYYCQQANIFPLTFGGGTKVEIKRTVAAPS | |
| | VFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| Chains 3 | QVQLVQSGAEVKKPGASVKVSCKASGYTFTGYYMHWVRQAPGQGLEWMGWIN | 661 |
| and 4 | PDSGGTNYAQKFQGRVTMTRDTSISTAYMELNRLRSDDTAVYYCARDQPLGY | |
| CP870893- | CTNGVCSYFDYWGQGTLVTVSSASTKGPSVFPLAPCSRSTSESTAALGCLVK | |
| G4Fd | DYFPEPVTVSWNSGALTSGVHTFPAVLQSSGLYSLSSVVTVPSSSLGTKTYT | |
| | CNVDHKPSNTKVDKRV | |
| Chains 5 | DIVMTQTPLSLPVTPGEPASISCRSSQSIVSSWGNTYLEWYLQKPGQSPQLL | 662 |
| and 6 D3- | TYKVSNRFSGVPDRFSGSGSGTDFTLKISRVEAEDVGVYYCFQGSHVPWTFG | |
| Ig-kLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P2870362716 - Bi-specific Ab Fusion to anti-CD40 Ab

| | | |
|---|---|---|
| Chains 1 | QVTLKESGPALVKPTQTLTLTCTFSGFSLSTPGMGVWWIRQPPGKALEWLAD | 663 |
| and 2 D3- | IWWDDNKYTNPSLKSRLTISKDTSKNQVVLTMTNMDPVDTATYYCARRANYG | |
| huIgG1HC_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| GGGGSGGGGS- | RTPEVTCVVVDVSHEDPEVKFNWYVDGVEVHNAKTKPREEQYNSTYRVVSVL | |
| CP870893- | TVLHQDWLNGKEYKCKVSNKALAAPIEKTISKAKGQPREPQVYTLPPSRDEL | |
| VL-CL | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPGK [symbol] DIQMT | |
| | QSPSSVSASVGDRVTITCRASQGIYSWLAWYQQKPGKAPNLLIYTASTLQSG | |
| | VPSRFSGSGSGTDFTLTISSLQPEDFATYYCQQANIFPLTFGGGTKVEIKRT | |
| | VAAPSVFIFPPSDEQLKSGTASVVCLLNNFYPREAKVQWKVDNALQSGNSQE | |
| | SVTEQDSKDSTYSLSSTLTLSKADYEKHKVYACEVTHQGLSSPVTKSFNRGE | |
| | C | |
| Chains 3 | QVQLVQSGAEVKKPGASVKVSCKASGYTFTGYYMHWVRQAPGQGLEWMGWIN | 664 |
| and 4 | PDSGGTNYAQKFQGRVTMTRDTSISTAYMELNRLRSDDTAVYYCARDQPLGY | |
| CP870893- | CTNGVCSYFDYWGQGTLVTVSSASTKGPSVFPLAPCSRSTSESTAALGCLVK | |
| G4Fd | DYFPEPVTVSWNSGALTSGVHTFPAVLQSSGLYSLSSVVTVPSSSLGTKTYT | |
| | CNVDHKPSNTKVDKRV | |
| Chains 5 | DIVMTQTPLSLPVTPGEPASISCRSSQSIVSSWGNTYLEWYLQKPGQSPQLL | 665 |
| and 6 D3- | IYKVSNRFSGVPDRFSGSGSGTDFTLKISRVEAEDVGVYYCFQGSHVPWTFG | |
| Ig-kLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P2871362716 - Bi-specific Ab Fusion to anti-CD40 Ab

| | | |
|---|---|---|
| Chains 1 | QVTLKESGPALVKPTQTLTLTCTFSGFSLSTPGMGVWWIRQPPGKALEWLAD | 666 |
| and 2 D3- | IWWDDNKYTNPSLKSRLTISKDTSKNQVVLTMTNMDPVDTATYYCARRANYG | |
| huIgG1HC_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| GGGGSGGGGS | RTPEVTCVVVDVSHEDPEVKFNWYVDGVEVHNAKTKPREEQYNSTYRVVSVL | |
| GGGGS- | TVLHQDWLNGKEYKCKVSNKALAAPIEKTISKAKGQPREPQVYTLPPSRDEL | |
| CP870893- | TKNQVSLTCLVKGFYPSDIAVEWESNGQPENNYKTTPPVLDSDGSFFLYSKL | |
| VL-CL | TVDKSRWQQGNVFSCSVMHEALHNHYTQKSLSLSPGK [symbol] | |
| | DIQMTQSPSSVSASVGDRVTITCRASQGIYSWLAWYQQKPGKAPNLLIYTAS | |
| | TLQSGVPSRFSGSGSGTDFTLTISSLQPEDFATYYCQQANIFPLTFGGGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |
| Chains 3 | QVQLVQSGAEVKKPGASVKVSCKASGYTFTGYYMHWVRQAPGQGLEWMGWIN | 667 |
| and 4 | PDSGGTNYAQKFQGRVTMTRDTSISTAYMELNRLRSDDTAVYYCARDQPLGY | |
| CP870893- | CTNGVCSYFDYWGQGTLVTVSSASTKGPSVFPLAPCSRSTSESTAALGCLVK | |
| G4Fd | DYFPEPVTVSWNSGALTSGVHTFPAVLQSSGLYSLSSVVTVPSSSLGTKTYT | |
| | CNVDHKPSNTKVDKRV | |
| Chains 5 | DIVMTQTPLSLPVTPGEPASISCRSSQSIVSSWGNTYLEWYLQKPGQSPQLL | 668 |
| and 6 D3- | IYKVSNRFSGVPDRFSGSGSGTDFTLKISRVEAEDVGVYYCFQGSHVPWTFG | |
| Ig-kLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P2851522716 - Bi-specific Ab Fusion to anti-41BB Ab

| | | |
|---|---|---|
| Chains 1 | QVTLKESGPALVKPTQTLTLTCTFSGFSLSTPGMGVWWIRQPPGKALEW | 669 |
| and 2 D3- | LADIWWDDNKYTNPSLKSRLTISKDTSKNQVVLTMTNMDPVDTATYYCA | |
| huIgG1HC_ | RRANYGNPYYAQDYWGQGTTVTVSSASTKGPSVFPLAPSSKSTSGGTAA | |
| L234A_L235A_ | LGCLVKDYFPEPVTVSWNSGALTSGVHTFPAVLQSSGLYSLSSVVTVPS | |
| P329A- | SSLGTQTYICNVNHKPSNTKVDKKVEPKSCDKTHTCPPCPAPEAAGGPS | |
| GGGGSGGGGS | VFLFPPKPKDTLMISRTPEVTCVVVDVSHEDPEVKFNWYVDGVEVHNAK | |
| GGGGS-Uto- | TKPREEQYNSTYRVVSVLTVLHQDWLNGKEYKCKVSNKALAAPIEKTIS | |
| VL-CL | KAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK [symbol] SYELTQPPSVSVSPGQTASITC | |
| | SGDNIGDQYAHWYQQKPGQSPVLVIYQDKNRPSGIPERFSGSNSGNTAT | |
| | LTISGTQAMDEADYYCATYTGFGSLAVFGGGTKLTVLGQPKAAPSVTLF | |
| | PPSSEELQANKATLVCLISDFYPGAVTVAWKADSSPVKAGVETTTPSKQ | |
| | SNNKYAASSYLSLTPEQWKSHRSYSCQVTHEGSTVEKTVAPTECS | |
| Chains 3 | EVQLVQSGAEVKKPGESLRISCKGSGYSFSTYWISWVRQMPGKGLEWMG | 670 |
| and 4 Uto- | KIYPGDSYTNYSPSFQGQVTISADKSISTAYLQWSSLKASDTAMYYCAR | |
| Fd | GYGIFDYWGQGTLVTVSSASTKGPSVFPLAPCSRSTSESTAALGCLVKD | |
| | YFPEPVTVSWNSGALTSGVHTFPAVLQSSGLYSLSSVVTVPSSSLGTKT | |
| | YTCNVDHKPSNTKVDKRV | |
| Chains 5 | DIVMTQTPLSLPVTPGEPASISCRSSQSIVSSWGNTYLEWYLQKPGQSPQLL | 671 |
| and 6 D3- | IYKVSNRFSGVPDRFSGSGSGTDFTLKISRVEAEDVGVYYCFQGSHVPWTFG | |
| Ig-kLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P2853332716 - Bi-specific Ab Fusion to anti-41BB Ab

| | | |
|---|---|---|
| Chains 1 | QVTLKESGPALVKPTQTLTLTCTFSGFSLSTPGMGVWWIRQPPGKALEW | 672 |
| and 2 D3- | LADIWWDDNKYTNPSLKSRLTISKDTSKNQVVLTMTNMDPVDTATYYCA | |
| huIgG1HC_ | RRANYGNPYYAQDYWGQGTTVTVSSASTKGPSVFPLAPSSKSTSGGTAA | |
| L234A_L235A_ | LGCLVKDYFPEPVTVSWNSGALTSGVHTFPAVLQSSGLYSLSSVVTVPS | |
| P329A- | SSLGTQTYICNVNHKPSNTKVDKKVEPKSCDKTHTCPPCPAPEAAGGPS | |
| GGGGSGGGGS | VFLFPPKPKDTLMISRTPEVTCVVVDVSHEDPEVKFNWYVDGVEVHNAK | |
| GGGGS-Ure- | TKPREEQYNSTYRVVSVLTVLHQDWLNGKEYKCKVSNKALAAPIEKTIS | |
| VL-CL | KAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK [symbol] EIVLTQSPATLSLSPGERATLS | |
| | CRASQSVSSYLAWYQQKPGQAPRLLIYDASNRATGIPARFSGSGSGTDF | |
| | TLTISSLEPEDFAVYYCQQRSNWPPALTFGGGTKVEIKRTVAAPSVFIF | |
| | PPSDEQLKSGTASVVCLLNNFYPREAKVQWKVDNALQSGNSQESVTEQD | |
| | SKDSTYSLSSTLTLSKADYEKHKVYACEVTHQGLSSPVTKSFNRGEC | |
| Chains 3 | QVQLQQWGAGLLKPSETLSLTCAVYGGSFSGYYWSWIRQSPEKGLEWIGEIN | 673 |
| and 4 Ure- | HGGYVTYNPSLESRVTISVDTSKNQFSLKLSSVTAADTAVYYCARDYGPGNY | |
| Fd | DWYFDLWGRGTLVTVSSASTKGPSVFPLAPCSRSTSESTAALGCLVKDYFPE | |
| | PVTVSWNSGALTSGVHTFPAVLQSSGLYSLSSVVTVPSSSLGTKTYTCNVDH | |
| | KPSNTKVDKRV | |
| Chains 5 | DIVMTQTPLSLPVTPGEPASISCRSSQSIVSSWGNTYLEWYLQKPGQSPQLL | 674 |
| and 6 D3- | TYKVSNRFSGVPDRFSGSGSGTDFTLKISRVEAEDVGVYYCFQGSHVPWTFG | |
| Ig-kLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P2854551808 - Bi-specific Ab Fusion to anti-41BB Ab

| | | |
|---|---|---|
| Chains 1 | EVQLVQSGAEVKKPGESLRISCKGSGYSFSTYWISWVRQMPGKGLEWMG | 675 |
| and 2 Uto- | KIYPGDSYTNYSPSFQGQVTISADKSISTAYLQWSSLKASDTAMYYCAR | |
| IgG1HC_L234A_ | GYGIFDYWGQGTLVTVSSASTKGPSVFPLAPSSKSTSGGTAALGCLVKD | |
| L235A_P329A- | YFPEPVTVSWNSGALTSGVHTFPAVLQSSGLYSLSSVVTVPSSSLGTQT | |
| GGGGSGGGGS | YICNVNHKPSNTKVDKKVEPKSCDKTHTCPPCPAPEAAGGPSVFLFPPK | |
| GGGGS-D3- | PKDTLMISRTPEVTCVVVDVSHEDPEVKFNWYVDGVEVHNAKTKPREEQ | |
| VL-CL | YNSTYRVVSVLTVLHQDWLNGKEYKCKVSNKALAAPIEKTISKAKGQPR | |
| | EPQVYTLPPSRDELTKNQVSLTCLVKGFYPSDIAVEWESNGQPENNYKT | |
| | TPPVLDSDGSFFLYSKLTVDKSRWQQGNVFSCSVMHEALHNHYTQKSLS | |
| | LSPGK [symbol] DIVMTQTPLSLPVTPGEPASISCRSSQSI | |
| | VSSWGNTYLEWYLQKPGQSPQLLIYKVSNRFSGVPDRFSGSGSGTDFTL | |
| | KISRVEAEDVGVYYCFQGSHVPWTFGQGTKVEIKRTVAAPSVFIFPPSD | |
| | EQLKSGTASVVCLLNNFYPREAKVQWKVDNALQSGNSQESVTEQDSKDS | |
| | TYSLSSTLTLSKADYEKHKVYACEVTHQGLSSPVTKSFNRGEC | |
| Chains 3 | QVTLKESGPALVKPTQTLTLTCTFSGFSLSTPGMGVWWIRQPPGKALEW | 676 |
| and 4 D3- | LADIWWDDNKYTNPSLKSRLTISKDTSKNQVVLTMTNMDPVDTATYYCA | |
| Fd | RRANYGNPYYAQDYWGQGTTVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKKVEPKSCDK | |
| Chains 5 | SYELTQPPSVSVSPGQTASITCSGDNIGDQYAHWYQQKPGQSPVLVIYQDKN | 677 |
| and 6 Uto- | RPSGIPERFSGSNSGNTATLTISGTQAMDEADYYCATYTGFGSLAVFGGGTK | |
| Ig-1LC | LTVLGQPKAAPSVTLFPPSSEELQANKATLVCLISDFYPGAVTVAWKADSSP | |
| | VKAGVETTTPSKQSNNKYAASSYLSLTPEQWKSHRSYSCQVTHEGSTVEKTV | |
| | APTECS | |

P2856551800 - Bi-specific Ab Fusion to anti-41BB Ab

| | | |
|---|---|---|
| Chains 1 | QVQLQQWGAGLLKPSETLSLTCAVYGGSFSGYYWSWIRQSPEKGLEWIG | 678 |
| and 2 Ure- | EINHGGYVTYNPSLESRVTISVDTSKNQFSLKLSSVTAADTAVYYCARD | |
| IgG1HC_L234A_ | YGPGNYDWYFDLWGRGTLVTVSSASTKGPSVFPLAPSSKSTSGGTAALG | |
| L235A_P329A- | CLVKDYFPEPVTVSWNSGALTSGVHTFPAVLQSSGLYSLSSVVTVPSSS | |
| GGGGSGGGGS | LGTQTYICNVNHKPSNTKVDKKVEPKSCDKTHTCPPCPAPEAAGGPSVF | |
| GGGGS-D3- | LFPPKPKDTLMISRTPEVTCVVVDVSHEDPEVKFNWYVDGVEVHNAKTK | |
| VL-CL | PREEQYNSTYRVVSVLTVLHQDWLNGKEYKCKVSNKALAAPIEKTISKA | |
| | KGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYT | |
| | QKSLSLSPGK [symbol] DIVMTQTPLSLPVTPGEPASISCR | |
| | SSQSIVSSWGNTYLEWYLQKPGQSPQLLIYKVSNRFSGVPDRFSGSGSG | |
| | TDFTLKISRVEAEDVGVYYCFQGSHVPWTFGQGTKVEIKRTVAAPSVFI | |
| | FPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| Chains 3 | QVTLKESGPALVKPTQTLTLTCTFSGFSLSTPGMGVWWIRQPPGKALEW | 679 |
| and 4 D3- | LADIWWDDNKYTNPSLKSRLTISKDTSKNQVVLTMTNMDPVDTATYYCA | |
| Fd | RRANYGNPYYAQDYWGQGTTVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKKVEPKSCDK | |
| Chains 5 | EIVLTQSPATLSLSPGERATLSCRASQSVSSYLAWYQQKPGQAPRLLIYDAS | 680 |
| and 6 Ure- | NRATGIPARFSGSGSGTDFTLTISSLEPEDFAVYYCQQRSNWPPALTFGGGT | |
| Ig-kLC | KVEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPVT | |
| | KSFNRGEC | |

P27152798 - Fusion to muIL-12

| | | |
|---|---|---|
| Chains 1 | QVTLKESGPALVKPTQTLTLTCTFSGFSLSTPGMGVWWIRQPPGKALEWLAD | 681 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| muIL-12- | TVLHQDWLNGKEYKCKVSNKALAAPIEKTISKAKGQPREPQVYTLPPSRDEL | |
| p40- | TKNQVSLTCLVKGFYPSDIAVEWESNGQPENNYKTTPPVLDSDGSFFLYSKL | |
| (GGGGS)3- | TVDKSRWQQGNVFSCSVMHEALHNHYTQKSLSLSPG [symbol] M | |
| p35 | WELEKDVYVVEVDWTPDAPGETVNLTCDTPEEDDITWTSDQRHGVIGSGKTL | |
| | TITVKEFLDAGQYTCHKGGETLSHSHLLLHKKENGIWSTEILKNFKNKTFLK | |
| | CEAPNYSGRFTCSWLVQRNMDLKFNIKSSSSSPDSRAVTCGMASLSAEKVTL | |
| | DQRDYEKYSVSCQEDVTCPTAEETLPIELALEARQQNKYENYSTSFFIRDII | |
| | KPDPPKNLQMKPLKNSQVEVSWEYPDSWSTPHSYFSLKFFVRIQRKKEKMKE | |
| | TEEGCNQKGAFLVEKTSTEVQCKGGNVCVQAQDRYYNSSCSKWACVPCRVRS | |
| | [symbol] RVIPVSGPARCLSQSRNLLKTTDDMVKTAREKLKHYS | |
| | CTAEDIDHEDITRDQTSTLKTCLPLELHKNESCLATRETSSTTRGSCLPPQK | |
| | TSLMMTLCLGSIYEDLKMYQTEFQAINAALQNHNHQQIILDKGMLVAIDELM | |
| | QSLNHNGETLRQKPPVGEADPYRVKMKLCILLHAFSTRVVTINRVMGYLSSA | |
| Chains 3 | DIVMTQTPLSLPVTPGEPASISCRSSQSIVHSNANTYLEWYLQKPGQSPQLL | 682 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to muIL-12

| | | |
|---|---|---|
| Chains 1 | EVQLVESGGGLVQPGGSLRLSCAASGFTFSRYWMTWVRQAPGKGLEWIGEIN | 683 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| muIL-12- | HQDWLNGKEYKCKVSNKALAAPIEKTISKAKGQPREPQVYTLPPSRDELTKN | |
| p40- | QVSLTCLVKGFYPSDIAVEWESNGQPENNYKTTPPVLDSDGSFFLYSKLTVD | |
| (GGGGS)3- | KSRWQQGNVFSCSVMHEALHNHYTQKSLSLSPG [symbol] MWEL | |
| p35 | EKDVYVVEVDWTPDAPGETVNLTCDTPEEDDITWTSDQRHGVIGSGKTLTIT | |
| | VKEFLDAGQYTCHKGGETLSHSHLLLHKKENGIWSTEILKNFKNKTFLKCEA | |
| | PNYSGRFTCSWLVQRNMDLKFNIKSSSSSPDSRAVTCGMASLSAEKVTLDQR | |
| | DYEKYSVSCQEDVTCPTAEETLPIELALEARQQNKYENYSTSFFIRDIIKPD | |
| | PPKNLQMKPLKNSQVEVSWEYPDSWSTPHSYFSLKFFVRIQRKKEKMKETEE | |
| | GCNQKGAFLVEKTSTEVQCKGGNVCVQAQDRYYNSSCSKWACVPCRVRS [symbol] | |
| | [symbol] RVIPVSGPARCLSQSRNLLKTTDDMVKTAREKLKHYSCTA | |
| | EDIDHEDITRDQTSTLKTCLPLELHKNESCLATRETSSTTRGSCLPPQKTSL | |
| | MMTLCLGSIYEDLKMYQTEFQAINAALQNHNHQQIILDKGMLVAIDELMQSL | |
| | NHNGETLRQKPPVGEADPYRVKMKLCILLHAFSTRVVTINRVMGYLSSA | |
| Chains 3 | DIVMTQSPDSLAVSLGERATINCKSSQSLLNWYNQKNYLAWYQQKPGQPPKL | 684 |
| and 4 | LIYGASTRESGVPDRFSGSGSGTDFTLTISSLQAEDVAVYYCQNDHQYPYTF | |
| hH8VLa_ | GQGTKLEIKRTVAAPSVFIFPPSDEQLKSGTASVVCLLNNFYPREAKVQWKV | |
| IgkLC | DNALQSGNSQESVTEQDSKDSTYSLSSTLTLSKADYEKHKVYACEVTHQGLS | |
| | SPVTKSFNRGEC | |

P27152798 - Fusion to huIL-2

| | | |
|---|---|---|
| Chains 1 | QVTLKESGPALVKPTQTLTLTCTFSGFSLSTPGMGVWWIRQPPGKALEWLAD | 685 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_ | PEPVTVSWNSGALTSGVHTFPAVLQSSGLYSLSSVVTVPSSSLGTQTYICNV | |
| L235A_P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| huIL- | TVLHQDWLNGKEYKCKVSNKALAAPIEKTISKAKGQPREPQVYTLPPSRDEL | |
| 2_T3A_K35E | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPG [symbol] A | |
| | PASSSTKKTQLQLEHLLLDLQMILNGINNYKNPELTRMLTFKFYMPKKATEL | |
| | KHLQCLEEELKPLEEVLNLAQSKNFHLRPRDLISNINVIVLELKGSETTFMC | |
| | EYADETATIVEFLNRWITFSQSIISTLT | |
| Chains 3 | DIVMTQTPLSLPVTPGEPASISCRSSQSIVHSNANTYLEWYLQKPGQSPQLL | 686 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to huIL-2

| | | |
|---|---|---|
| Chains 1 | EVQLVESGGGLVQPGGSLRLSCAASGFTFSRYWMTWVRQAPGKGLEWIGEIN | 687 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| huIL- | HQDWLNGKEYKCKVSNKALAAPIEKTISKAKGQPREPQVYTLPPSRDELTKN | |
| 2_T3A_K35E | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] APAS | |
| | SSTKKTQLQLEHLLLDLQMILNGINNYKNPELTRMLTFKFYMPKKATELKHL | |
| | QCLEEELKPLEEVLNLAQSKNFHLRPRDLISNINVIVLELKGSETTFMCEYA | |
| | DETATIVEFLNRWITFSQSIISTLT | |
| Chains 3 | DIVMTQSPDSLAVSLGERATINCKSSQSLLNWYNQKNYLAWYQQKPGQPPKL | 688 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

P27152798 - Fusion to huIL-7

| | | |
|---|---|---|
| Chains 1 | QVTLKESGPALVKPTQTLTLTCTFSGFSLSTPGMGVWWIRQPPGKALEWLAD | 689 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| huIL-7 | TVLHQDWLNGKEYKCKVSNKALAAPIEKTISKAKGQPREPQVYTLPPSRDEL | |
| | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPG [symbol] D | |
| | CDIEGKDGKQYESVLMVSIDQLLDSMKEIGSNCLNNEFNFFKRHICDANKEG | |
| | MFLFRAARKLRQFLKMNSTGDFDLHLLKVSEGTTILLNCTGQVKGRKPAALG | |
| | AAQPTKSLEENKSLKEQKKLNDLCFLKRLLQEIKTCWNKILMGTKEH | |
| Chains 3 | DIVMTQTPLSLPVTPGEPASISCRSSQSIVHSNANTYLEWYLQKPGQSPQLL | 690 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to huIL-7

| | | |
|---|---|---|
| Chains 1 | EVQLVESGGGLVQPGGSLRLSCAASGFTFSRYWMTWVRQAPGKGLEWIGEIN | 691 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| huIL-7 | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] DCDI | |
| | EGKDGKQYESVLMVSIDQLLDSMKEIGSNCLNNEFNFFKRHICDANKEGMFL | |
| | FRAARKLRQFLKMNSTGDFDLHLLKVSEGTTILLNCTGQVKGRKPAALGAAQ | |
| | PTKSLEENKSLKEQKKLNDLCFLKRLLQEIKTCWNKILMGTKEH | |
| Chains 3 | DIVMTQSPDSLAVSLGERATINCKSSQSLLNWYNQKNYLAWYQQKPGQPPKL | 692 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

P27152798 - Fusion to huIL-15

| | | |
|---|---|---|
| Chains 1 | QVTLKESGPALVKPTQTLTLTCTFSGFSLSTPGMGVWWIRQPPGKALEWLAD | 693 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| huIL-15 | TVLHQDWLNGKEYKCKVSNKALAAPIEKTISKAKGQPREPQVYTLPPSRDEL | |
| | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPG [symbol] N | |
| | WVNVISDLKKIEDLIQSMHIDATLYTESDVHPSCKVTAMKCFLLELQVISLE | |
| | SGDASIHDTVENLIILANNSLSSNGNVTESGCKECEELEEKNIKEFLQSFVH | |
| | IVQMFINTS | |
| Chains 3 | DIVMTQTPLSLPVTPGEPASISCRSSQSIVHSNANTYLEWYLQKPGQSPQLL | 694 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to huIL-15

| | | |
|---|---|---|
| Chains 1 | EVQLVESGGGLVQPGGSLRLSCAASGFTFSRYWMTWVRQAPGKGLEWIGEIN | 695 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| huIL-15 | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] NWVN | |
| | VISDLKKIEDLIQSMHIDATLYTESDVHPSCKVTAMKCFLLELQVISLESGD | |
| | ASIHDTVENLIILANNSLSSNGNVTESGCKECEELEEKNIKEFLQSFVHIVQ | |
| | MFINTS | |
| Chains 3 | DIVMTQSPDSLAVSLGERATINCKSSQSLLNWYNQKNYLAWYQQKPGQPPKL | 696 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

P27152798 - Fusion to huIL-21

| | | |
|---|---|---|
| Chains 1 | QVTLKESGPALVKPTQTLTLTCTFSGFSLSTPGMGVWWIRQPPGKALEWLAD | 697 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| huIL-21 | TVLHQDWLNGKEYKCKVSNKALAAPIEKTISKAKGQPREPQVYTLPPSRDEL | |
| | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPG [symbol] Q | |
| | GQDRHMIRMRQLIDIVDQLKNYVNDLVPEFLPAPEDVETNCEWSAFSCFQKA | |
| | QLKSANTGNNERIINVSIKKLKRKPPSTNAGRRQKHRLTCPSCDSYEKKPPK | |
| | EFLERFKSLLQKMIHQHLSSRTHGSEDS | |
| Chains 3 | DIVMTQTPLSLPVTPGEPASISCRSSQSIVHSNANTYLEWYLQKPGQSPQLL | 698 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to huIL-21

| | | |
|---|---|---|
| Chains 1 | EVQLVESGGGLVQPGGSLRLSCAASGFTFSRYWMTWVRQAPGKGLEWIGEIN | 699 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| huIL-21 | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] QGQD | |
| | RHMIRMRQLIDIVDQLKNYVNDLVPEFLPAPEDVETNCEWSAFSCFQKAQLK | |
| | SANTGNNERIINVSIKKLKRKPPSTNAGRRQKHRLTCPSCDSYEKKPPKEFL | |
| | ERFKSLLQKMIHQHLSSRTHGSEDS | |
| Chains 3 | DIVMTQSPDSLAVSLGERATINCKSSQSLLNWYNQKNYLAWYQQKPGQPPKL | 700 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

P27152798 - Fusion to huIFNα

| | | |
|---|---|---|
| Chains 1 | QVTLKESGPALVKPTQTLTLTCTFSGFSLSTPGMGVWWIRQPPGKALEWLAD | 701 |
| and 2 | IWWDDNKYTNPSLKSRLTISKDTSKNQVVLTMTNMDPVDTATYYCARRANYG | |
| hD3_huIgG1_ | NPYYAQDYWGQGTTVTVSSASTKGPSVFPLAPSSKSTSGGTAALGCLVKDYF | |
| L234A_L235A_ | PEPVTVSWNSGALTSGVHTFPAVLQSSGLYSLSSVVTVPSSSLGTQTYICNV | |
| P329A- | NHKPSNTKVDKKVEPKSCDKTHTCPPCPAPEAAGGPSVFLFPPKPKDTLMIS | |
| (GGGGS)3- | RTPEVTCVVVDVSHEDPEVKFNWYVDGVEVHNAKTKPREEQYNSTYRVVSVL | |
| huIFNα | TVLHQDWLNGKEYKCKVSNKALAAPIEKTISKAKGQPREPQVYTLPPSRDEL | |
| | TKNQVSLTCLVKGFYPSDIAVEWESNGQPENNYKTTPPVLDSDGSFFLYSKL | |
| | TVDKSRWQQGNVFSCSVMHEALHNHYTQKSLSLSPG [symbol] C | |
| | DLPQTHSLGSRRTLMLLAQMRRISLFSCLKDRHDFGFPQEEFGNQFQKAETI | |
| | PVLHEMIQQIFNLFSTKDSSAAWDETLLDKFYTELYQQLNDLEACVIQGVGV | |
| | TETPLMKEDSILAVRKYFQRITLYLKEKKYSPCAWEVVRAEIMRSFSLSTNL | |
| | QESLRSKE | |
| Chains 3 | DIVMTQTPLSLPVTPGEPASISCRSSQSIVHSNANTYLEWYLQKPGQSPQLL | 702 |
| and 4 | IYKVSNRFSGVPDRFSGSGSGTDFTLKISRVEAEDVGVYYCFQGSHVPWTFG | |
| hB7A-IgkLC | QGTKVEIKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSS | |
| | PVTKSFNRGEC | |

P29382939 - Fusion to huIFNα

| | | |
|---|---|---|
| Chains 1 | EVQLVESGGGLVQPGGSLRLSCAASGFTFSRYWMTWVRQAPGKGLEWIGEIN | 703 |
| and 2 | PDSSTANYTPYLKGRFTISRDNAKNSLYLQMNSLRAEDTAVYYCARPVDGYY | |
| hH8VHe_ | DAMDPWGQGTTVTVSSASTKGPSVFPLAPSSKSTSGGTAALGCLVKDYFPEP | |
| huIgG1_L234A_ | VTVSWNSGALTSGVHTFPAVLQSSGLYSLSSVVTVPSSSLGTQTYICNVNHK | |
| L235A_P329A- | PSNTKVDKKVEPKSCDKTHTCPPCPAPEAAGGPSVFLFPPKPKDTLMISRTP | |
| (GGGGS)3- | EVTCVVVDVSHEDPEVKFNWYVDGVEVHNAKTKPREEQYNSTYRVVSVLTVL | |
| huIFNα | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] CDLP | |
| | QTHSLGSRRTLMLLAQMRRISLFSCLKDRHDFGFPQEEFGNQFQKAETIPVL | |
| | HEMIQQIFNLFSTKDSSAAWDETLLDKFYTELYQQLNDLEACVIQGVGVTET | |
| | PLMKEDSILAVRKYFQRITLYLKEKKYSPCAWEVVRAEIMRSFSLSTNLQES | |
| | LRSKE | |
| Chains 3 | DIVMTQSPDSLAVSLGERATINCKSSQSLLNWYNQKNYLAWYQQKPGQPPKL | 704 |
| and 4 | LIYGASTRESGVPDRFSGSGSGTDFTLTISSLQAEDVAVYYCQNDHQYPYTF | |
| hH8VLa_IgkLC | GQGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSFNRGEC | |

Thus, in certain embodiments, a fusion protein, including a bi-specific antibody, comprises, consists, or consists essentially of an amino acid sequence that is at least 80, 85, 90, 95, 98, or 100% identical to a sequence selected from Table S10.

Methods of Use and Pharmaceutical Compositions

Certain embodiments include methods of treating, ameliorating the symptoms of, and/or reducing the progression of, a disease or condition in a subject in need thereof, comprising administering to the subject at least one fusion protein or improved antibody, or antigen-binding fragment thereof, as described herein. Also included are methods of enhancing an immune response in a subject, comprising administering to the subject at least one fusion protein, wherein the effector domain of the fusion protein has immune cell-stimulatory activity, as described herein. Certain embodiments include methods of reducing an immune response in a subject, comprising administering to the subject at least one fusion protein, wherein the effector domain of the fusion protein has immune cell-inhibitory activity, as described herein. In particular embodiments, the disease is selected from one or more of a cancer, a viral infection, and an immune disorder, including autoimmune and inflammatory disorders.

In some embodiments, following administration, the fusion protein binds to high density denatured collagen in a target tissue, and accumulates in that target tissue. The accumulation of the fusion protein increases the local concentration and thus the biological activity of the effector domain in a spatially-regulated way. In this regard, even though collagen remodeling is an integral part of normal tissue renewal, excessive amount of remodeling activity is involved in cancers, arthritis, wound healing, fibrosis, and many other pathological conditions (see, for example, Li et al., J Vis Exp. (83):e51052, 2014. doi:10.3791/51052). Thus, denatured tissue collagen is a biomarker of tissue remodeling or damage, for example, of the type that occurs in pathologically-inflamed tissue, infected tissue, or at a tumor site. In particular embodiments, the accumulation and increased concentration of the fusion protein, and thus the increased activity of the effector domain, occurs in a cancer cell or cancer tissue, or a virally-infected cell or virally-infected tissue.

In some instances, the fusion protein has at least one immune-stimulatory activity. In some embodiments, the fusion protein has at least one immune-inhibitory activity. In particular embodiments, the fusion protein has at least one cytocidal activity, for example, against an immune cell or a cancer cell. In particular embodiments, the immune cell is selected from one or more of a T cell, a B cell, a natural killer cell, a monocyte, and a macrophage.

In some embodiments, administration of a fusion protein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some instances, the immune response is an anti-cancer or anti-viral immune response. Any one or more of the effector domains described herein can be used to increase an immune response.

In some embodiments, administration of a fusion protein reduces an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some instances, the immune response is a pathological or disease-associated autoimmune or inflammatory response. In some embodiments, the effector domain for reducing an immune response comprises an IL-10 polypeptide.

In some embodiments, administration of a fusion protein increases cell-killing or cytocidal activity in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, wherein the cell-killing is cancer cell-killing or virally-infected cell-killing. Any one or more of the effector domains described herein can be used to increase cell-killing or cytocidal activity, directly or indirect (e.g., by increasing an anti-cancer or anti-viral immune response).

In some embodiments, the disease is a cancer, that is, the subject in need thereof has or is suspected of having a cancer. Certain embodiments thus include methods of treating, ameliorating the symptoms of, or inhibiting the progression of, a cancer in a subject in need thereof, comprising administering to the subject at least one fusion protein, as described herein. In particular embodiments, the cancer is a primary cancer or a metastatic cancer. In specific embodiments, the cancer is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject at least one fusion protein described herein in combination with at least one additional agent, for example, cancer immunotherapy agent, a chemotherapeutic agent, a hormonal therapeutic agent, and/or a kinase inhibitor. In some embodiments, administering the at least one fusion protein enhances the susceptibility of the cancer to the additional agent (for example, cancer immunotherapy agent, chemotherapeutic agent, hormonal therapeutic agent, and/or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and a cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In particular embodiments, the immune checkpoint modulatory agent is a polypeptide or peptide. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein

Antibodies are also included as polypeptides. Thus, in some embodiments, the immune checkpoint modulatory polypeptide agent is an antibody or “antigen-binding fragment thereof”, as described elsewhere herein.

In some embodiments, the agent is or comprises a “ligand,” for example, a natural ligand, of the immune checkpoint molecule. A “ligand” refers generally to a substance or molecule that forms a complex with a target molecule (e.g., biomolecule) to serve a biological purpose, and includes a “protein ligand,” which generally produces a signal by binding to a site on a target molecule or target protein. Thus, certain agents are protein ligands that, in nature, bind to an immune checkpoint molecule and produce a signal. Also included are “modified ligands,” for example, protein ligands that are fused to a pharmacokinetic modifier, for example, an Fc region derived from an immunoglobulin.

The binding properties of polypeptides can be quantified using methods well known in the art (see Davies et al., Annual Rev. Biochem. 59:439-473, 1990). In some embodiments, a polypeptide specifically binds to a target molecule, for example, an immune checkpoint molecule or an epitope thereof, with an equilibrium dissociation constant that is about or ranges from about ≤10-7 to about 10-8 M. In some embodiments, the equilibrium dissociation constant is about or ranges from about ≤10-9 M to about ≤10-10 M. In certain illustrative embodiments, the polypeptide has an affinity (Kd or EC₅₀) for a target described herein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the agent is a “small molecule,” which refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of about or less than about 1000-2000 Daltons, typically between about 300 and 700 Daltons, and including about or less than about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for herein polypeptides such as antibodies. For instance, in some embodiments a small molecule specifically binds to a target, for example, an immune checkpoint molecule, with a binding affinity (Kd or EC₅₀) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a VISTA antagonist or inhibitor. V-domain Ig suppressor of T cell activation (VISTA) is primarily expressed on hematopoietic cells and is an inhibitory immune checkpoint regulator that suppresses T-cell activation, induces Foxp3 expression, and is highly expressed within the tumor microenvironment where it suppresses anti-tumor T cell responses (see, e.g., Lines et al., Cancer Res. 74:1924-32, 2014). General examples of VISTA antagonists or inhibitors include an antibody or antigen-binding fragment or small molecule that specifically binds to VISTA and reduces one or more of its immune-suppressive activities.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3:e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

The various cancer immunotherapy agents described herein can be combined with any one or more of the antibodies, and antigen-binding fragments thereof, described herein, and used according to any one or more of the methods or compositions described herein.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The various chemotherapeutic agents described herein can be combined with any one or more of the fusion proteins or anti-denatured collagen antibodies described herein, and used according to any one or more of the methods or compositions described herein.

Certain combination therapies employ at least one hormonal therapeutic agent. General examples of hormonal therapeutic agents include hormonal agonists and hormonal antagonists.

Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The various hormonal therapeutic agents described herein can be combined with any one or more of the various fusion proteins or improved anti-denatured collagen antibodies described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The various kinase inhibitors described herein can be combined with any one or more of the various fusion proteins or improved anti-denatured collagen antibodies described herein, and used according to any one or more of the methods or compositions described herein.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and pharmaceutical compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In some embodiments, the disease is a viral disease or viral infection. In certain embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis. In specific embodiments, the subject is HIV-positive. In some embodiments, the methods and pharmaceutical compositions described herein increase an anti-viral immune response by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the immune disorder is selected from one or more of type 1 diabetes, vasculitis, and an immunodeficiency. In some embodiments, the methods and pharmaceutical compositions described herein improve or increase immune function in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the immune disorder is an autoimmune and/or inflammatory disease. In some embodiments, the methods and pharmaceutical compositions described herein decrease pathological or disease-associated immune function in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. Exemplary autoimmune diseases include, but are not limited to, arthritis (including rheumatoid arthritis, reactive arthritis), systemic lupus erythematosus (SLE), psoriasis and inflammatory bowel disease (IBD), encephalomyelitis, uveitis, myasthenia gravis, multiple sclerosis, insulin dependent diabetes, Addison's disease, celiac disease, chronic fatigue syndrome, autoimmune hepatitis, autoimmune alopecia, ankylosing spondylitis, ulcerative colitis, Crohn's disease, fibromyalgia, pemphigus vulgaris, Sjogren's syndrome, Kawasaki's Disease, hyperthyroidism/Graves' disease, hypothyroidism/Hashimoto's disease, endometriosis, scleroderma, pernicious anemia, Goodpasture syndrome, Guillain-Barré syndrome, Wegener's disease, glomerulonephritis, aplastic anemia (including multiply transfused aplastic anemia patients), paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever, autoimmune lymphoproliferative syndrome (ALPS), autoimmune bullous pemphigoid, Parkinson's disease, sarcoidosis, vitiligo, primary biliary cirrhosis, and autoimmune myocarditis.

Exemplary inflammatory diseases include, but are not limited to, Crohn's disease, colitis, dermatitis, psoriasis, diverticulitis, hepatitis, irritable bowel syndrome (IBS), lupus erythematous, nephritis, Parkinson's disease, ulcerative colitis, multiple sclerosis (MS), Alzheimer's disease, arthritis, rheumatoid arthritis, asthma, and various cardiovascular diseases such as atherosclerosis and vasculitis. In certain embodiments, the inflammatory disease is selected from the group consisting of rheumatoid arthritis, diabetes, gout, cryopyrin-associated periodic syndrome, and chronic obstructive pulmonary disorder.

In specific embodiments, and further to above, the effector domain comprises an IL-10 polypeptide and the disease is selected from one or more of cancer, autoimmune or inflammatory diseases, for example, of the gastrointestinal tract (for example, Crohn's disease), rheumatoid arthritis, multiple sclerosis, and others. In specific embodiments, and further to above, the effector domain comprises an IFNα polypeptide and the disease is selected from one or more of hairy cell leukemia, malignant melanoma, follicular lymphoma, Kaposi's sarcoma related to AIDS, genital warts, hepatitis B, or hepatitis C.

In certain embodiments, the methods and therapeutic compositions described herein are sufficient to result in clinically relevant reduction in symptoms of a particular disease indication known to the skilled clinician.

For in vivo use, as noted above, for the treatment of human or non-human mammalian disease or testing, the protein agents described herein (e.g., fusion proteins, improved anti-denatured collagen antibodies, or antigen-binding fragments thereof) are incorporated into one or more therapeutic or pharmaceutical or diagnostic compositions prior to administration.

Thus, certain embodiments relate to pharmaceutical or therapeutic compositions that comprise at least one fusion protein, or at least one anti-denatured collagen antibody (or antigen-binding fragment thereof), as described herein. In some instances, a pharmaceutical or therapeutic composition comprises one or more of the protein agents described herein in combination with a pharmaceutically- or physiologically-acceptable carrier or excipient. Certain pharmaceutical or therapeutic compositions further comprise at least one additional agent, for example, a cancer immunotherapy agents, a chemotherapeutic agent, a hormonal therapeutic agent, and/or a kinase inhibitor as described herein.

Some therapeutic compositions comprise (and certain methods utilize) only one fusion protein, or only one improved anti-denatured collagen antibody (or antigen-binding fragment thereof).

Certain therapeutic compositions comprise (and certain methods utilize) a mixture of at least two, three, four, or five different fusion proteins, or improved anti-denatured collagen antibodies (or antigen-binding fragments thereof).

In particular embodiments, the pharmaceutical or therapeutic compositions comprising at least one fusion protein, or at least one improved anti-denatured collagen antibody (or antigen-binding fragment thereof), is substantially pure on a protein basis or a weight-weight basis, for example, the composition has a purity of at least about 80%, 85%, 90%, 95%, 98%, or 99% on a protein basis or a weight-weight basis.

In some embodiments, the protein agents described herein do not form aggregates, have a desired solubility, and/or have an immunogenicity profile that is suitable for use in humans, as known in the art. Thus, in some embodiments, the therapeutic composition comprising a protein agent is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise a protein agent that is at least about 50%, about 60%, about 70%, about 80%, about 90% or about 95% monodisperse with respect to its apparent molecular mass.

In some embodiments, a protein agent is concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients.

Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains a protein agent described herein (e.g., fusion protein, improved anti-denatured collagen antibody or antigen-binding fragment thereof) and an additional therapeutic agent (e.g., cancer immunotherapy agent, chemotherapeutic agent, hormonal therapeutic agent, kinase inhibitor), as well as administration of compositions comprising a protein agent and an additional therapeutic agent in its own separate pharmaceutical dosage formulation. For example, a protein agent described herein and additional therapeutic agent can be administered to the subject together in a single oral dosage composition such as a tablet or capsule, or each agent administered in separate oral dosage formulations. Similarly, a protein agent described herein and additional therapeutic agent can be administered to the subject together in a single parenteral dosage composition such as in a saline solution or other physiologically acceptable solution, or each agent administered in separate parenteral dosage formulations. As another example, for cell-based therapies, a protein agent described herein can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one protein agent described herein, as described herein; and optionally (b) at least one additional therapeutic agent (e.g., cancer immunotherapy agent, chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a protein agent described herein and optionally at least one additional therapeutic agent. For example, the kit includes a plurality of syringes, ampules, foil packets, or blister packs, each containing a single unit dose of a protein agent described herein and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

Expression and Purification Systems

Certain embodiments include methods and related compositions for expressing and purifying recombinant proteins, such as a fusion protein or an anti-denatured collagen antibody, or an antigen-binding fragment thereof, described herein. Such recombinant proteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, recombinant proteins may be prepared by a procedure including one or more of the steps of: (a) preparing one or more vectors or constructs comprising one or more polynucleotide sequences that encode one or more proteins described herein, which are operably linked to one or more regulatory elements; (b) introducing the one or more vectors or constructs into one or more host cells; (c) culturing the one or more host cell to express the one or more proteins; and (d) isolating the one or more proteins from the host cell.

To express a desired polypeptide, a nucleotide sequence encoding a first and/or second polypeptide chain of a protein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors may be used which direct high level expression of fusion proteins or improved anti-denatured collagen antibodies that are readily purified. Such vectors include but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS•TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15L, 50L, 100L, and 200L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1L and 5L spinners, 5L, 14L, 40L, 100L and 200L stir tank bioreactors, or 20/50L and 100/200L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt- cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif. 40:237-43, 2005; and U.S. Pat. No. 6,210,922).

A protein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating the proteins described herein (e.g., fusion proteins, anti-denatured collagen antibodies and antigen-binding fragments thereof), and composition comprising concentrated soluble proteins. In some aspects, such concentrated solutions comprise protein(s) at a concentration of about or at least about 5 mg/mL, 8 mg/mL, 10 mg/mL, 15 mg/mL, 20 mg/mL, or more.

In some aspects, such compositions may be substantially monodisperse, meaning that a protein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, a protein in a composition has a purity of at least about 90%, as measured according to routine techniques in the art. In certain embodiments, such as diagnostic compositions or certain pharmaceutical or therapeutic compositions, a protein composition has a purity of at least about 95%, or at least about 97% or 98% or 99%. In some embodiments, such as when being used as reference or research reagents, proteins can be of lesser purity, and may have a purity of at least about 50%, 60%, 70%, or 80%. Purity can be measured overall or in relation to selected components, such as other proteins, e.g., purity on a protein basis.

Purified proteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to cell-based assays, including those that utilize at least one IL-2 receptor and/or IL-15 receptor, which is optionally functionally coupled to a readout or indicator, such as a fluorescent or luminescent indicator of biological activity, as described herein.

In certain embodiments, as noted above, a composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, a protein composition is made from a eukaryotic cell such as a mammalian or human cell in substantially serum free media. In certain embodiments, as noted herein, an composition has an endotoxin content of less than about 10 EU/mg of protein, or less than about 5 EU/mg of protein, less than about 3 EU/mg of protein, or less than about 1 EU/mg of protein.

In certain embodiments, a composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of proteins and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Proteins with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains a protein of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Engineering of Antibodies Specific to Denatured Human Collagen

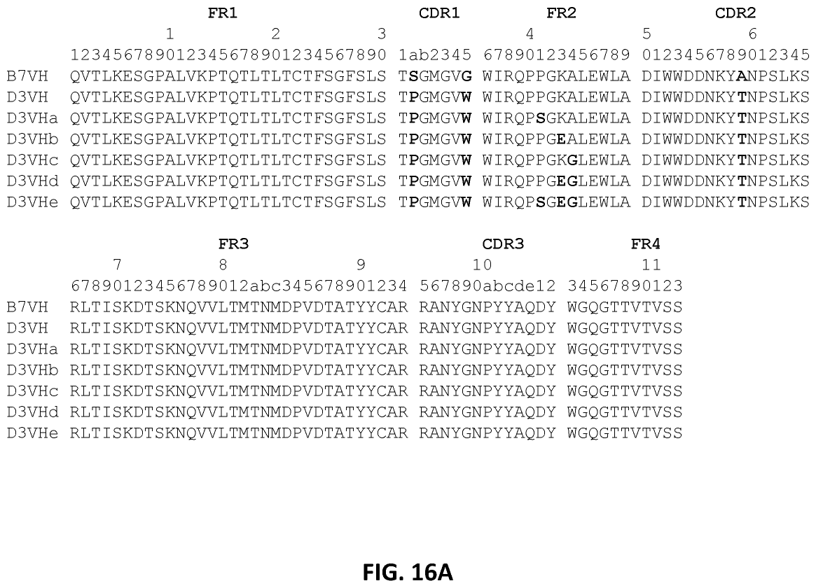

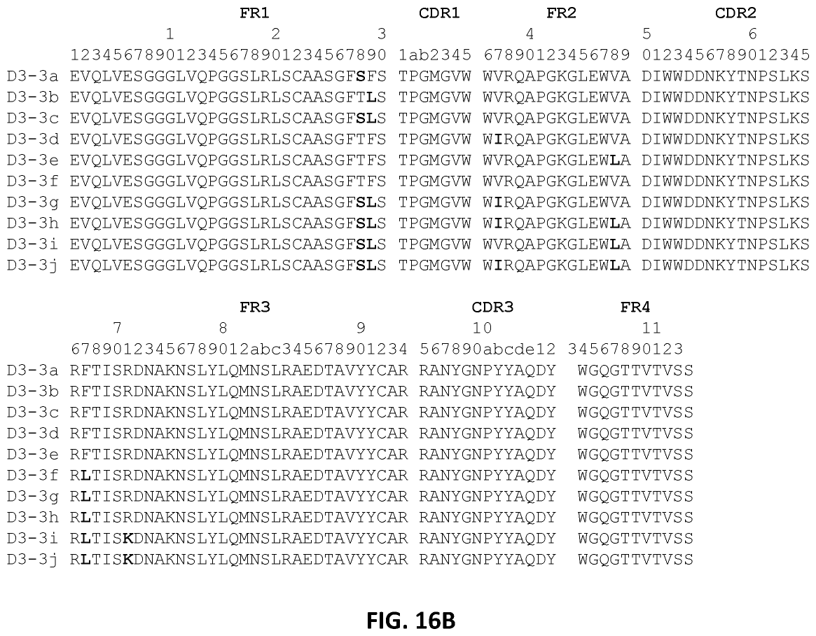

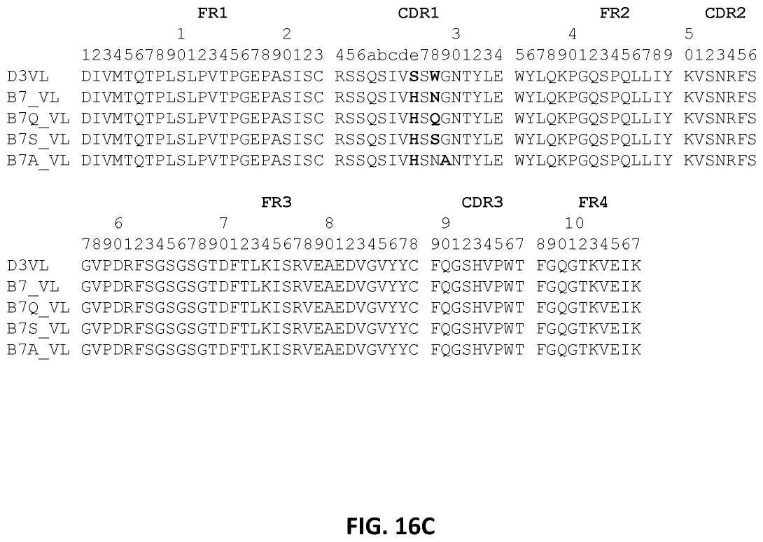

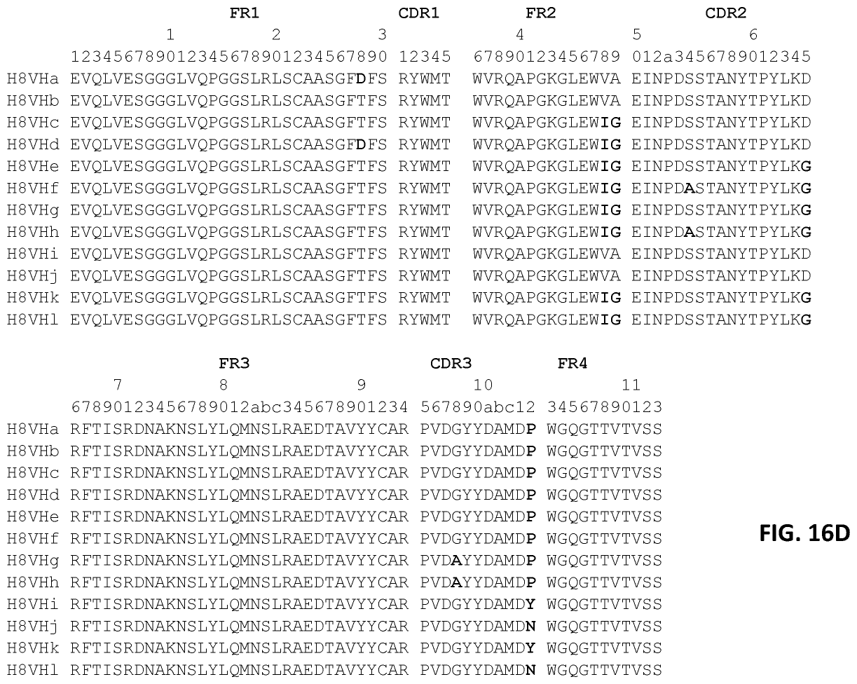

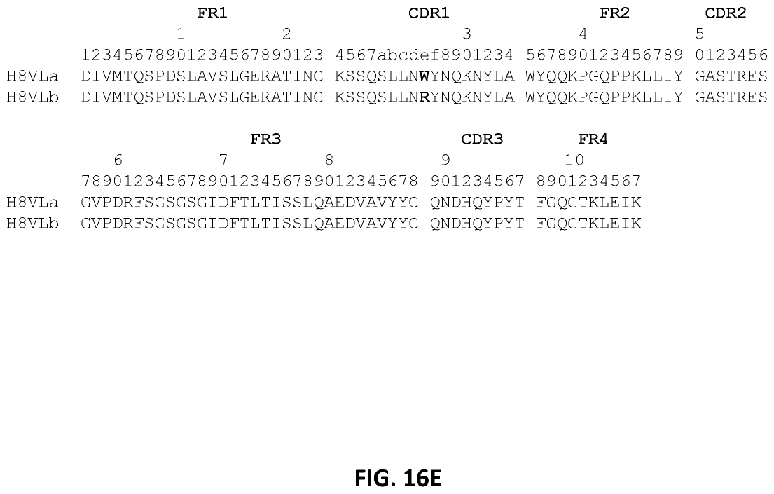

Experiments were performed to engineer antibodies that specifically bind to thermally-denatured human collagen types I, III, IV and V, but not to the corresponding native forms. Experiments were also performed to engineer antibodies that specifically bind to thermally-denatured human collagen types III and IV, and to the native form of human collagen IV. Amino acid sequences of the CDRs and variable regions are provided in Table A1, Table A2, and FIGS. 16A-16E.

Plasmids coding for heavy chain and light chain were constructed by standard gene synthesis, followed by sub-cloning into pTT5 expression vector. The antibody names relative to the heavy and light chain alignments in FIGS. 16A-16E are provided in Table E1 below.

TABLE E1

| Antibody Name | V_{H} Chain | V_{L} Chain |
|---|---|---|
| P27122713 | B7 | B7 |
| P27122714 | B7 | B7Q |
| P27122716 | B7 | D3 |
| P27152716 | D3 | D3 |
| P27152714 | D3 | B7Q |
| P27522716 | D3-3a | D3 |
| P27532716 | D3-3b | D3 |
| P27542716 | D3-3c | D3 |
| P27552716 | D3-3d | D3 |
| P27562716 | D3-3e | D3 |
| P27572716 | D3-3f | D3 |
| P27582716 | D3-3g | D3 |
| P27592716 | D3-3h | D3 |
| P27602716 | D3-3i | D3 |
| P27612716 | D3-3j | D3 |
| P27622716 | D3VHa | D3 |
| P27632716 | D3VHb | D3 |
| P27642716 | D3VHc | D3 |
| P27652716 | D3VHd | D3 |
| P27662716 | D3VHe | D3 |
| P27122797 | B7 | B7S |
| P27122798 | B7 | B7A |
| P27152797 | D3 | B7S |
| P27152798 | D3 | B7A |
| P29342939 | H8VHa | H8VLa |
| P29352939 | H8VHb | H8VLa |
| P29362939 | H8VHc | H8VLa |
| P29372939 | H8VHd | H8VLa |
| P29382939 | H8VHe | H8VLa |
| P30392939 | H8VHf | H8VLa |
| P30402939 | H8VHg | H8VLa |
| P30412939 | H8VHh | H8VLa |
| P30422939 | H8VHi | H8VLa |
| P30432939 | H8VHj | H8VLa |
| P30442939 | H8VHk | H8VLa |
| P30452939 | H8VHl | H8VLa |
| P30393046 | H8VHf | H8VLb |
| P30403046 | H8VHg | H8VLb |
| P30413046 | H8VHh | H8VLb |
| P30423046 | H8VHi | H8VLb |
| P30433046 | H8VHj | H8VLb |
| P30443046 | H8VHk | H8VLb |
| P30453046 | H8VHl | H8VLb |

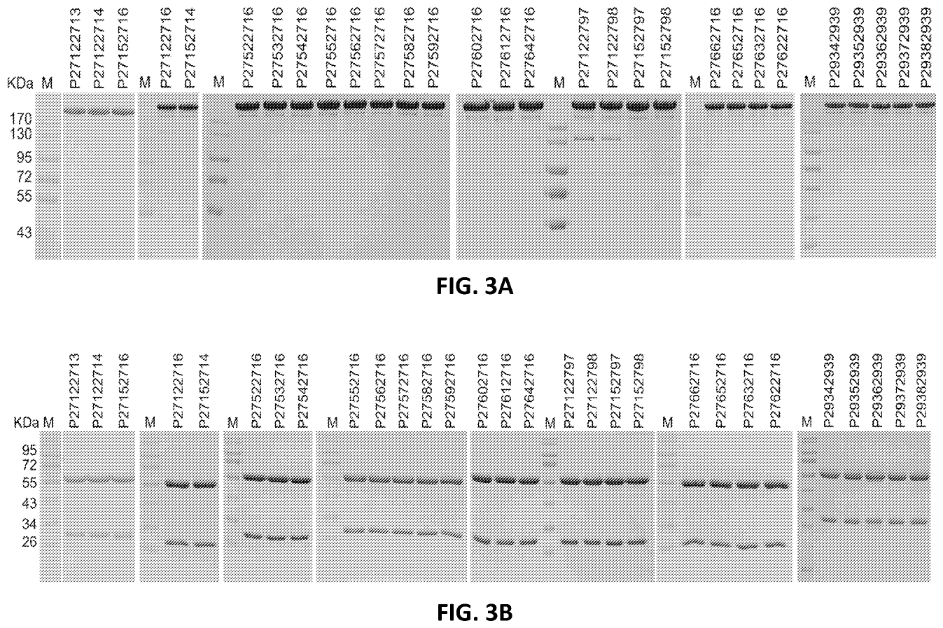

Production, purification and characterization. Antibodies were produced by transient transfection in Expi293 cells and purified by a one-step purification process of MabSelect SuRe chromatography (GE Healthcare). Purified proteins were characterized by SDS-PAGE for purity assessment and showed good purity as shown in FIGS. 3A-3B.

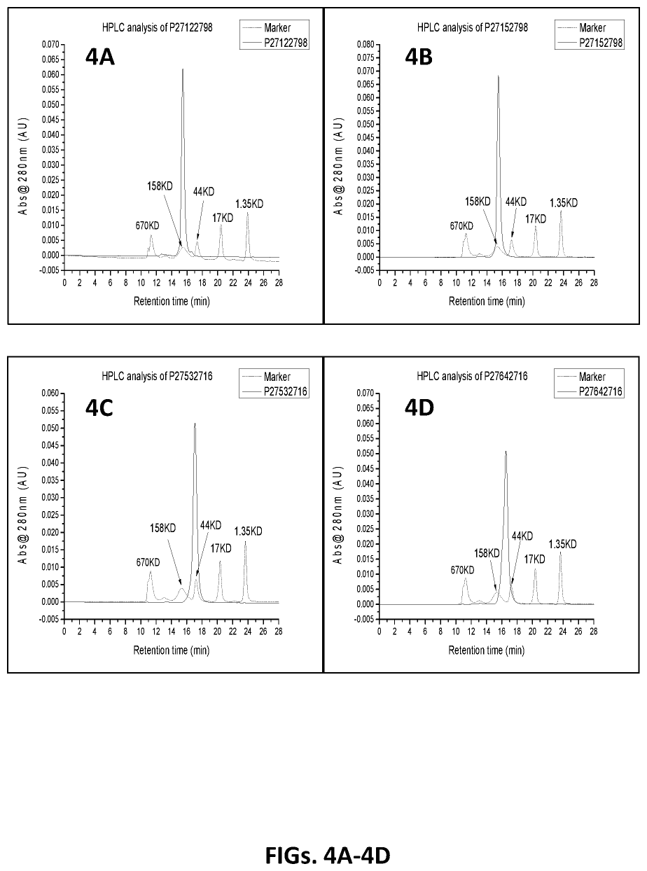

Purified proteins were also characterized by high performance liquid chromatography (HPLC) for homogeneity assessment. HPLC analysis was performed using a Zenix-C SEC-300 or Nanofilm SEC-250 column (Sepax) and Acquity Arc (Agilent) according to the manufacturer's instructions. Representative HPLC results are shown in FIGS. 4A-4D. Most of the proteins showed one single peak, indicating good homogeneity.

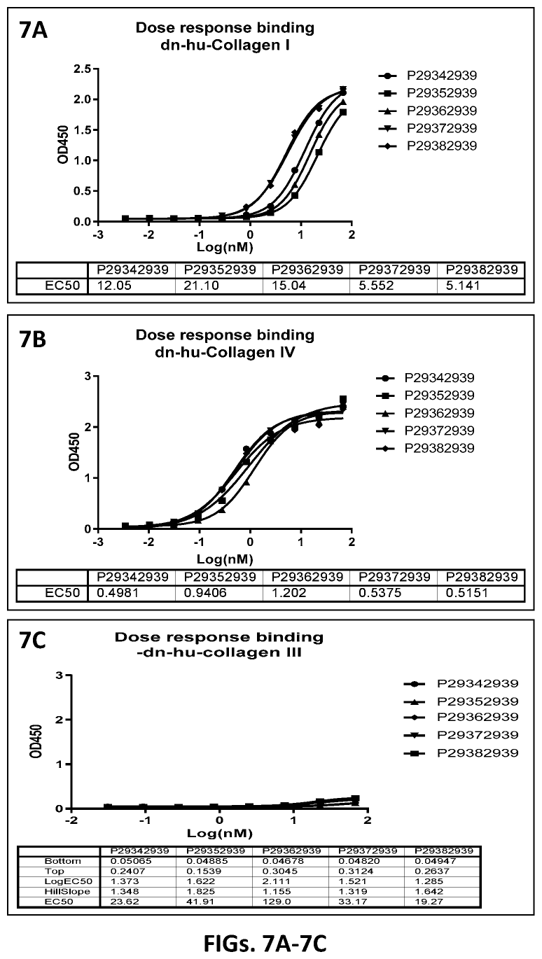

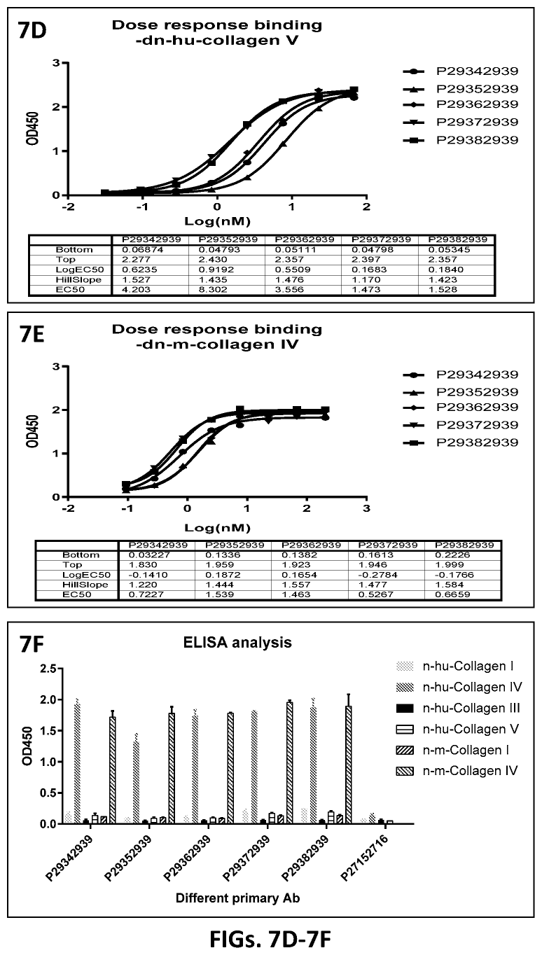

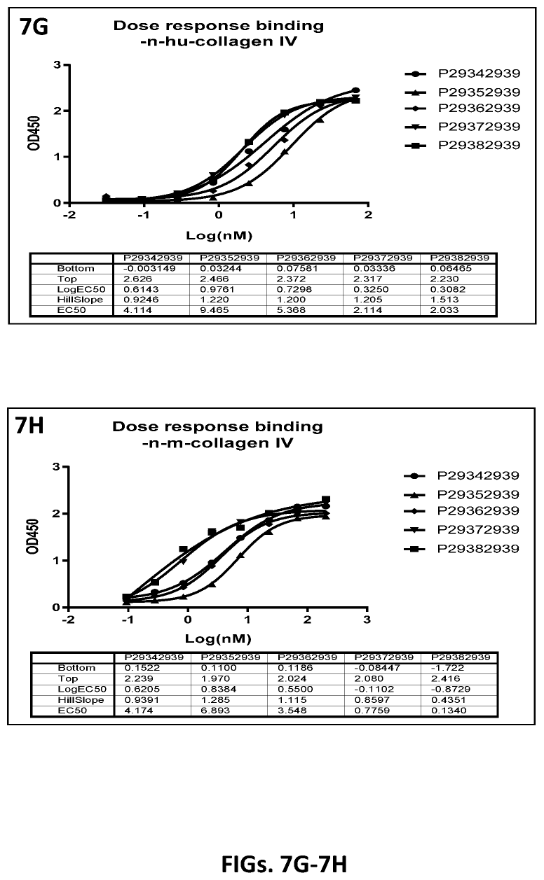

ELISA analysis. The binding activity and specificity of purified antibodies were determined by ELISA. Human collagens I (ab7533, Abcam), III (ab7535, Abcam), IV (ab7536, Abcam), an V (ab7537, Abcam), and mouse collagens I (NBP2-62423, NOVUS) and IV (3410-010-02, R&D) were used in ELISA analysis. “Denatured” collagen samples were prepared by incubation of collagens samples at 99° C. for 15 minutes followed by incubation on ice for 5 minutes Microtiter plates were coated with native or denatured collagen protein overnight at 4° C. The next day, plates were washed with PBS and blocked with 3% non-fat dry milk in PBS or 1% BSA. Serially diluted antibodies were added for binding to the coated antigen. Bound antibodies were detected with peroxidase-conjugated anti-human IgG secondary antibody (Jackson ImmunoResearch). The results were summarized in FIGS. 7, 8 and 9.

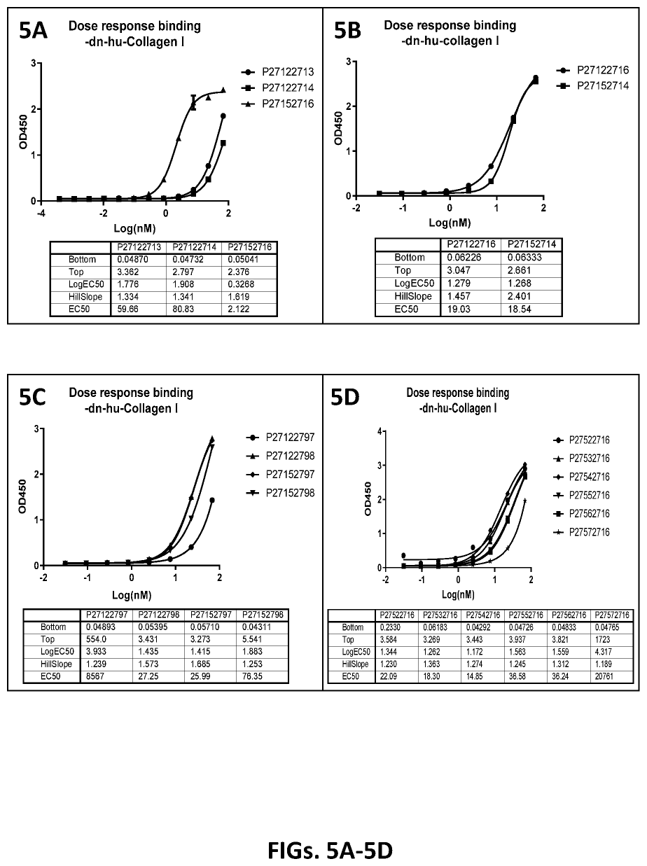

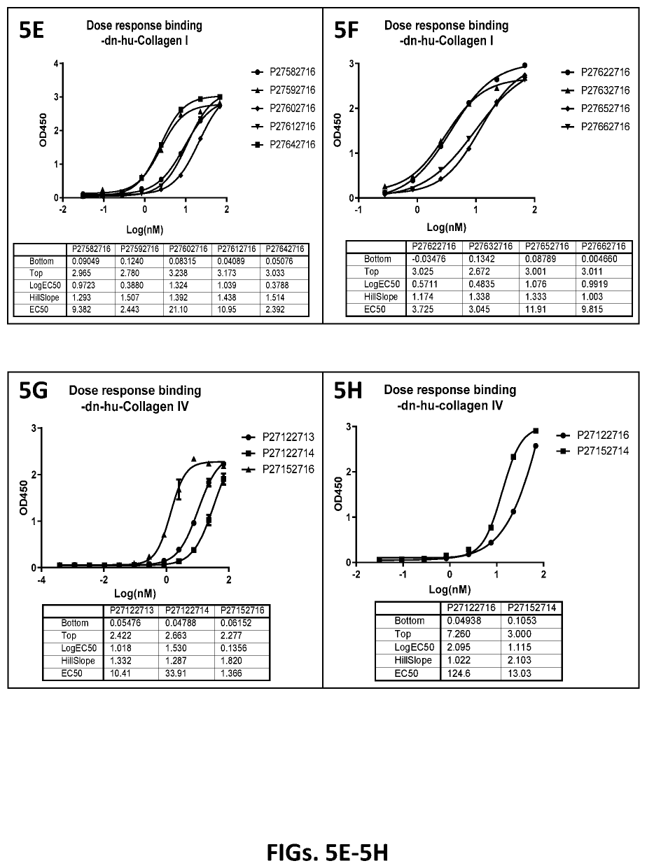

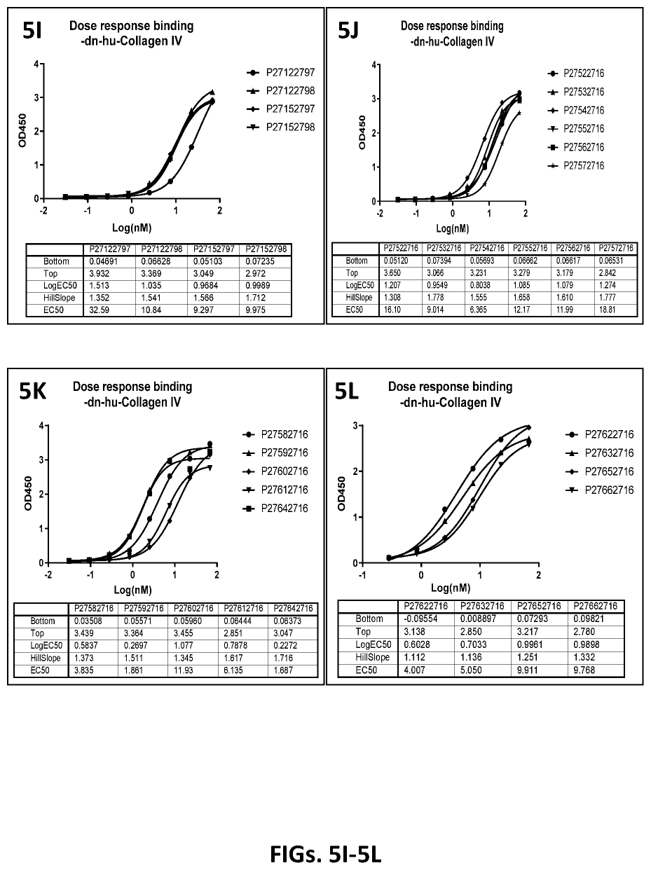

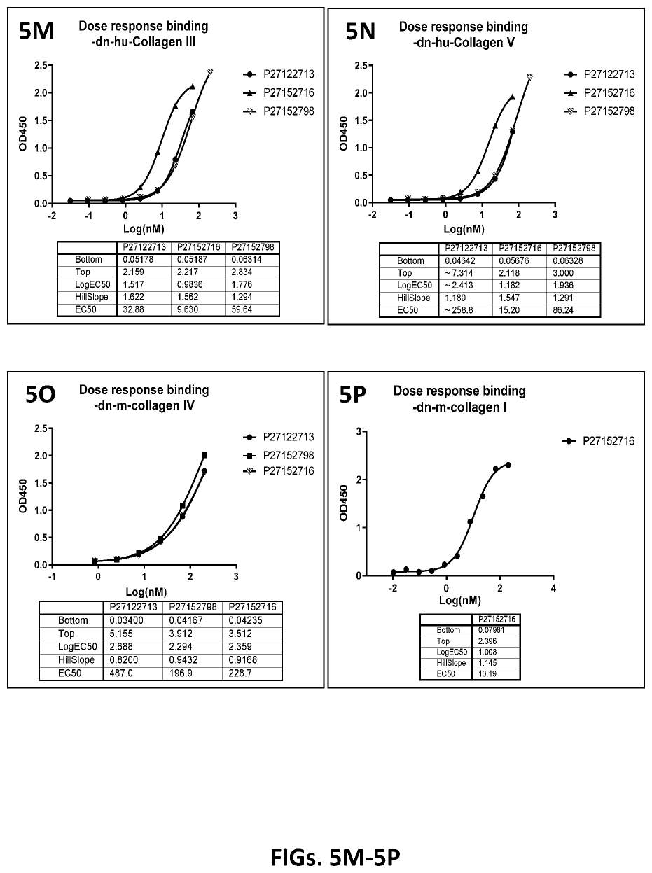

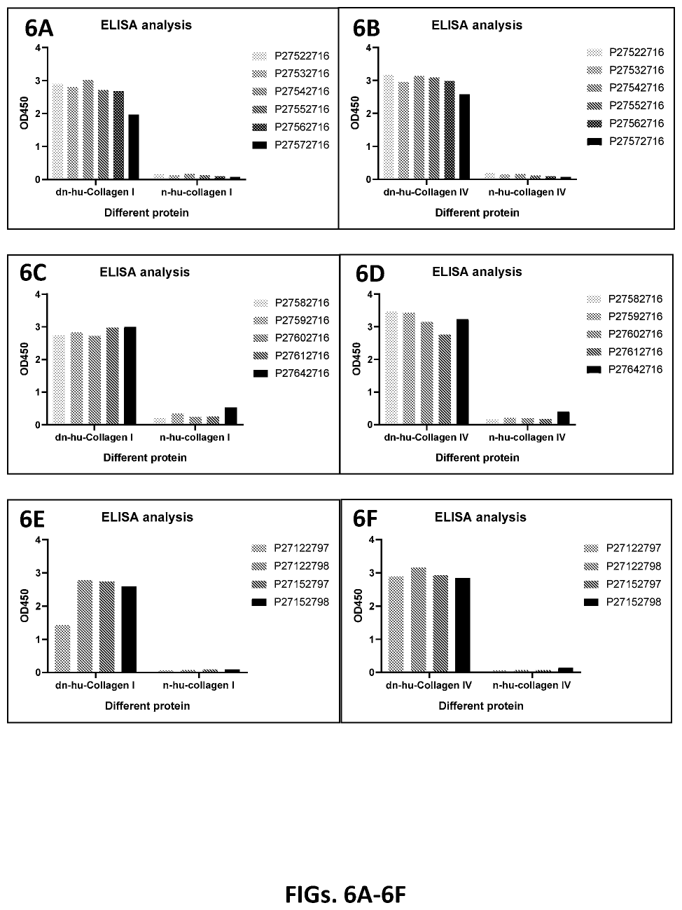

Antibodies exhibited binding activity for denatured human collagen I (FIGS. 5A-5F) and for denatured human collagen type IV (FIGS. 5G-5L). None of the antibodies had significant binding activity for native human collagen types I or IV (FIGS. 6A-6E). The P27122713, P27152716, and P27152798 antibodies showed binding activity for denatured human collagen type III (FIG. 5M) and type V (FIG. 5N), and also for denatured mouse collagen IV (FIG. 5O). P27152716 also exhibited binding activity to denatured mouse collagen I (FIG. 5P).

Antibodies exhibited binding activity for denatured human collagen types I (FIG. 7A), IV (FIG. 7B), and V (FIG. 7D), and also to denatured mouse collagen IV (FIG. 7E). None of the antibodies showed significant binding activity towards denatured human collagen type III (FIG. 7C), or towards native human collagen types I, III, or V, or native mouse collagen I (FIG. 7F). However, all of the antibodies exhibited binding activity for native human and mouse collagen IV (FIGS. 7F, 7G, and 7H).

### Example 2

### Engineering of Collagen-Targeted IL-2 Prodrug

An IL-2 prodrug (activated by protease cleavage) was fused to the C-terminus of a collagen-targeted antibody. Plasmids coding for the fusion proteins were constructed by standard gene synthesis, followed by sub-cloning into pTT5 expression vector.

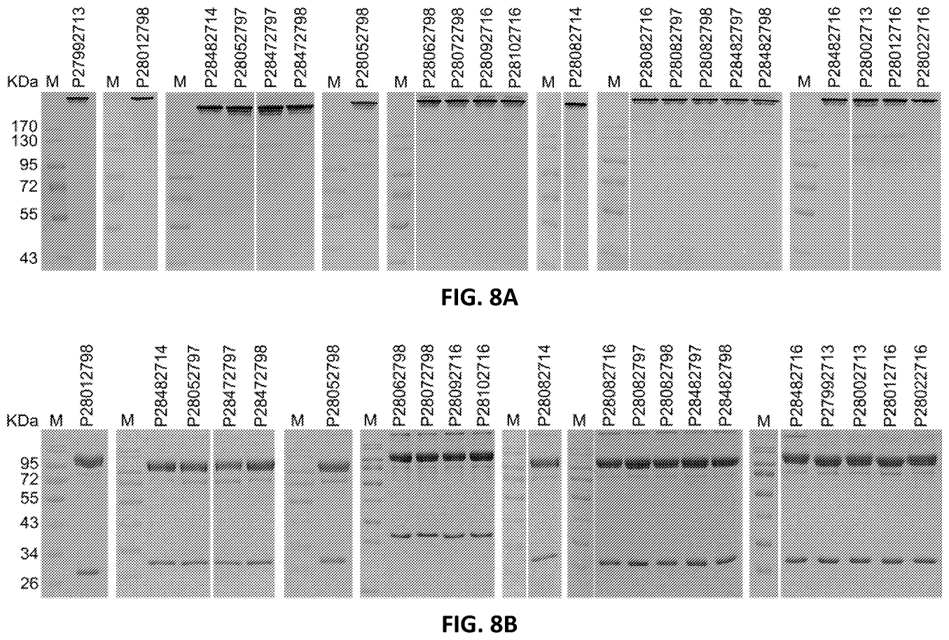

Production, purification and characterization. Fusion proteins were produced by transient transfection in Expi293 cells and purified by a two-step purification process of MabSelect SuRe chromatography (GE Healthcare) and size exclusion chromatography (Superdex 200, GE Healthcare). Purified proteins were characterized by SDS-PAGE for purity assessment and showed good purity as shown in FIGS. 8A-8B.

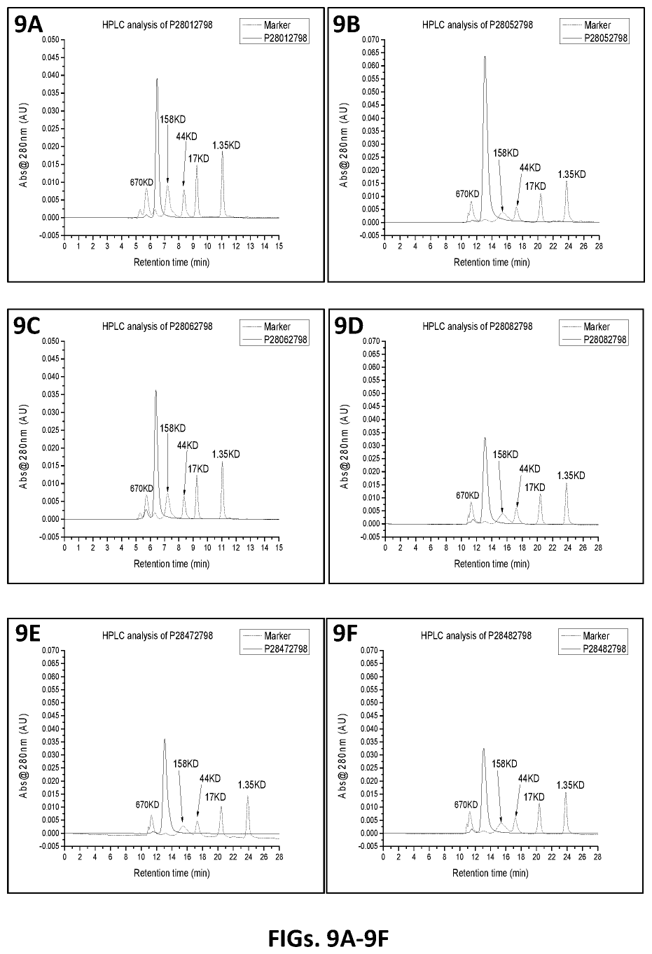

Purified proteins were also characterized by high performance liquid chromatography (HPLC) for homogeneity assessment. HPLC analysis was performed using Zenix-C column (Sepax) and Acquity Arc (Agilent) according to the manufacturer's instructions. Representative HPLC results were shown in FIGS. 9A-9F. Most of the proteins showed one single peak, indicating good homogeneity.

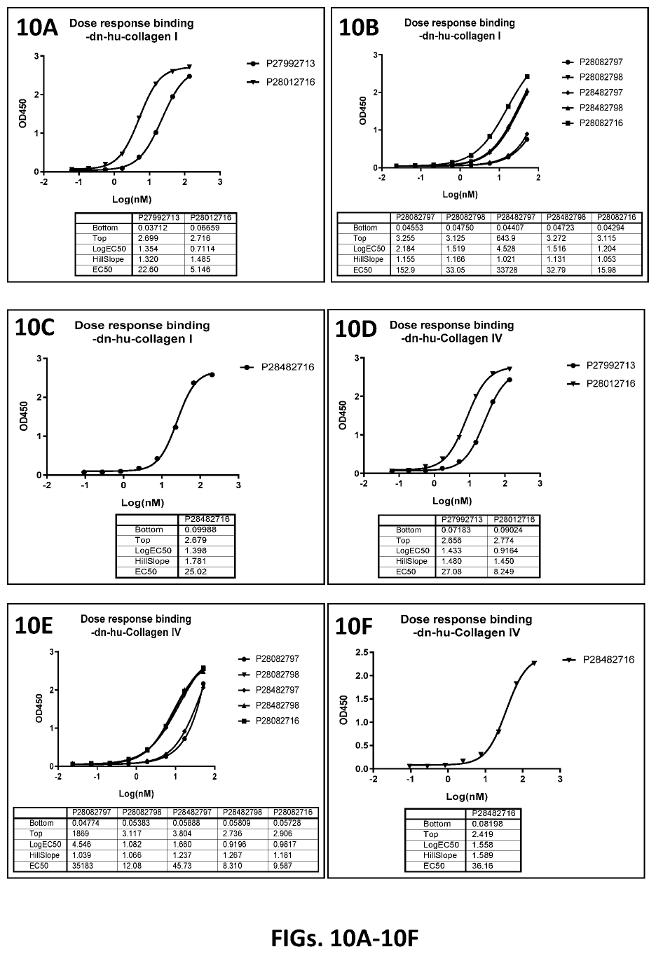

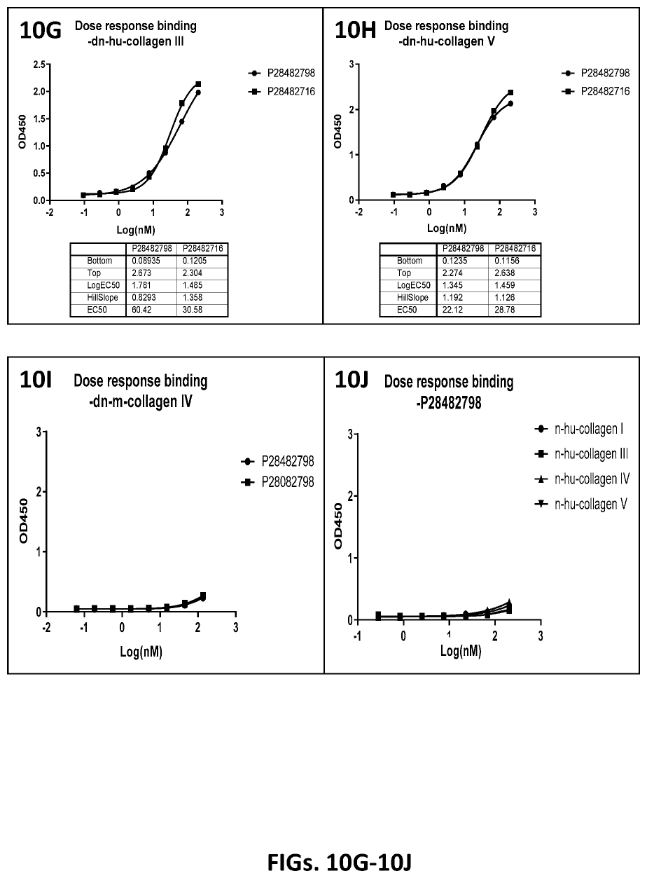

ELISA analysis. The binding activity and specificity of purified antibodies were determined by ELISA. ELISA procedures were the same as described in Example 1. The results are summarized in FIGS. 10A-10J. Purified fusion proteins exhibited binding activity for denatured human collagen type I (FIGS. 10A-10C), IV (FIGS. 10D-10F), III (FIG. 10G), and V (FIG. 10H), and showed almost no binding activity towards denatured mouse collagen IV (FIG. 10I) and native human collagen type I, III, IV, and V (FIG. 10J).

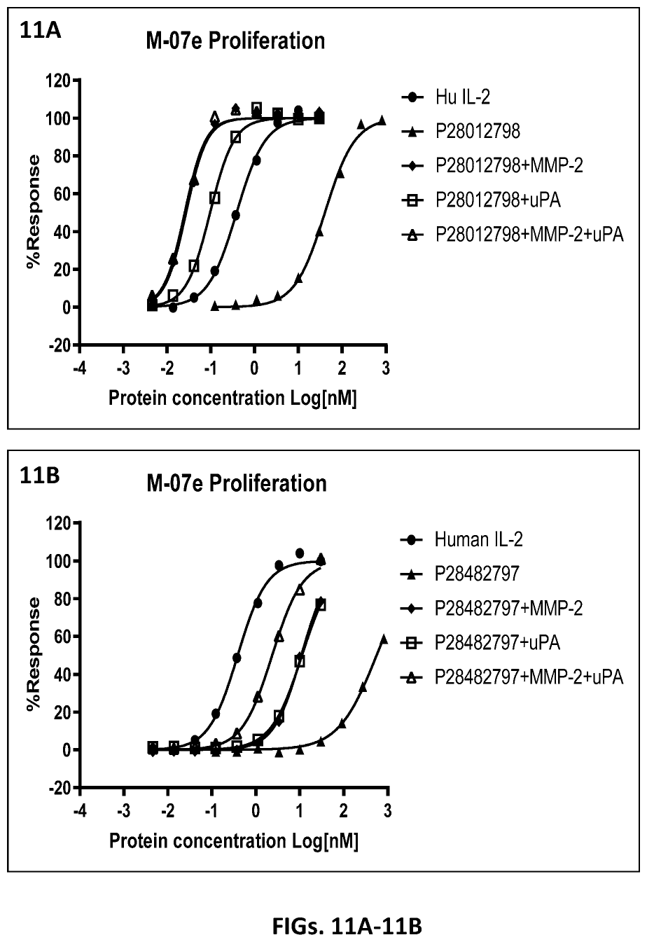

Functional assays—Proliferation. Proliferation assays were performed for purified proteins before and after protease cleavage. M-07e (IL-2Rβ/γc) cells were cultured in RPMI 1640 supplemented with 20% fetal bovine serum (FBS), 1% non-essential amino acids (NEAA), and 10% of 5637 cell culture supernatant. To measure cytokine-dependent cell proliferation, Mo7e cells were harvested in their logarithmic growth phase and washed twice with PBS. 90 μl of cell suspension (2×10⁴ cells/well) was seeded into 96-well plate and incubated for 4 hours in assay medium (RPMI 1640 supplemented with 10% FBS and 1% NEAA) for cytokine starvation at 37° C. and 5% CO2. IL-2 and purified proteins samples used in assays were prepared in assay medium to an initial concentration of 300 nM, followed by 1/3 serial dilutions. 10 μl diluted protein was added into corresponding wells and incubated at 37° C. and 5% CO2 for 72 hours. Colorimetric assays using a Cell Counting Kit-8 (CCK-8, Dojindo, CK04) were performed to measure the amount of live cells. Representative results are presented in FIGS. 11A-11B. Purified proteins before protease cleavage showed low activity and protease cleavage could recover partial or full activity of IL-2.

### Example 3

### Engineering of Collagen-Targeted IL-15 Prodrug

An IL-15 prodrug (activated by protease cleavage) was fused to the C-terminus of a collagen-targeted antibody. Plasmids coding for the fusion proteins were constructed by standard gene synthesis, followed by sub-cloning into pTT5 expression vector.

Production, purification and characterization. Fusion proteins were produced by transient transfection in Expi293 cells and purified by a two-step purification process of MabSelect SuRe chromatography (GE Healthcare) and size exclusion chromatography (Superdex 200, GE Healthcare).

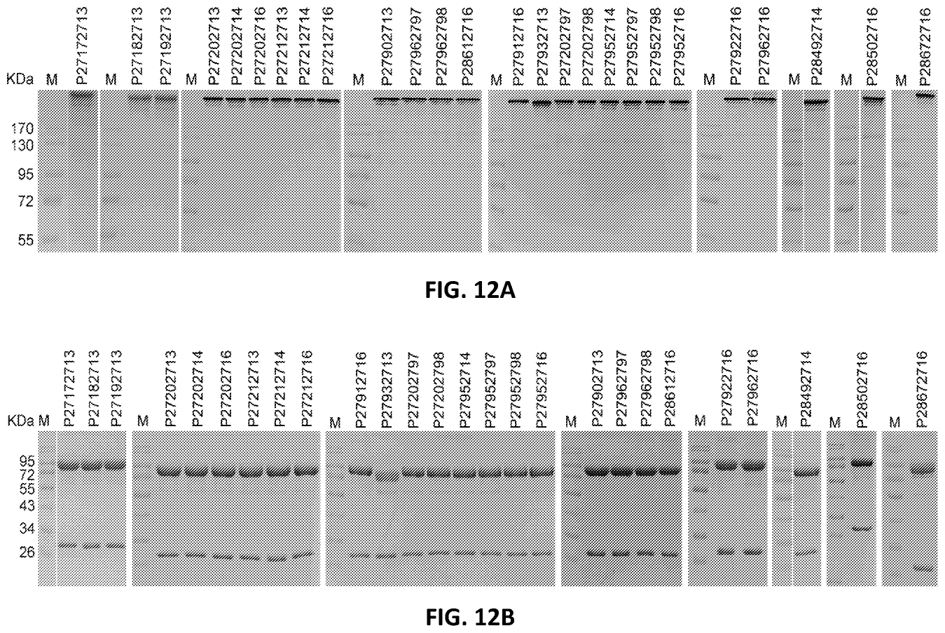

Purified proteins were characterized by SDS-PAGE for purity assessment and showed good purity as shown in FIGS. 12A-12B.

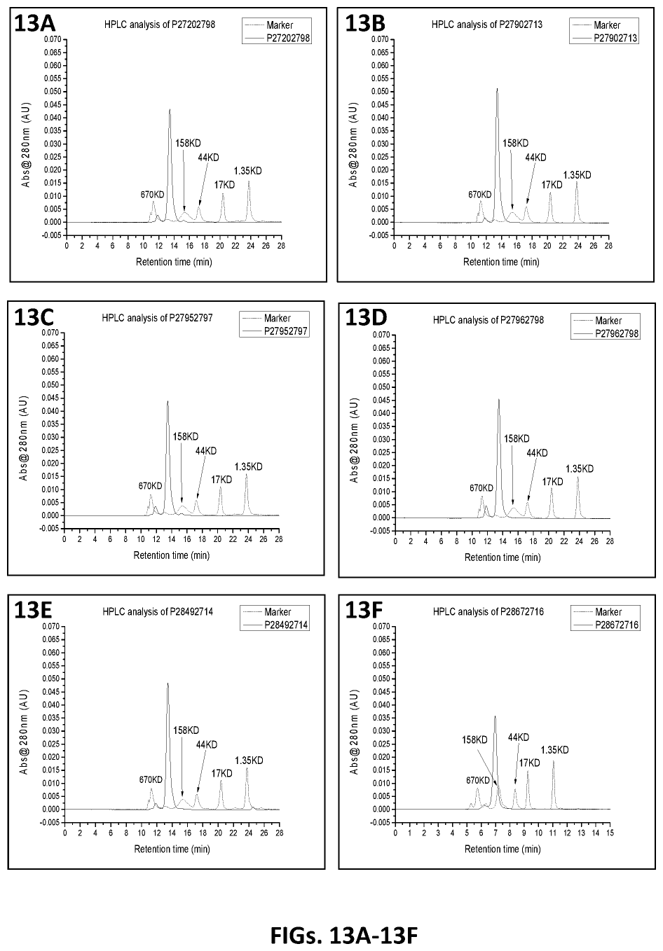

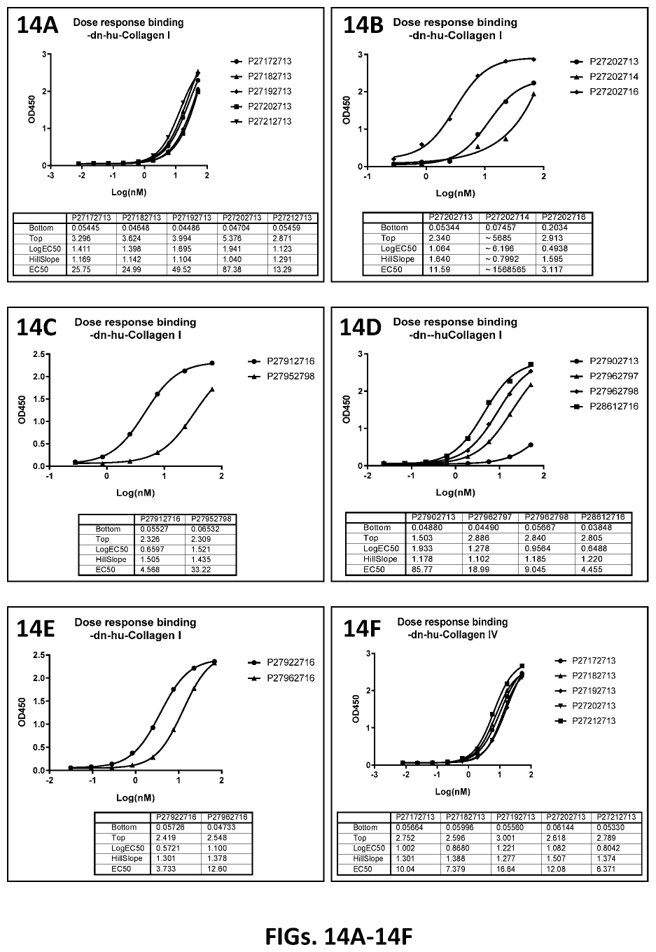

Purified proteins were also characterized by high performance liquid chromatography (HPLC) for homogeneity assessment. HPLC analysis was performed using Zenix-C column (Sepax) and Acquity Arc (Agilent) according to the manufacturer's instructions. Representative HPLC results are shown in FIGS. 13A-13F. Most of the proteins showed one single peak, indicating good homogeneity.

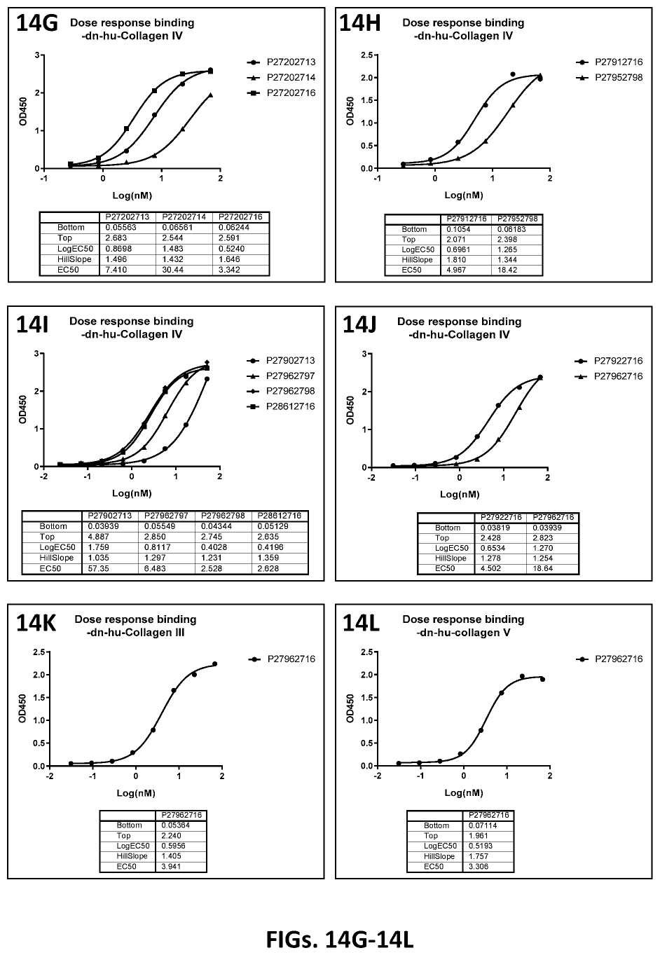

ELISA analysis. The binding activity and specificity of purified antibodies were determined by ELISA. ELISA procedures were the same as described in Example 1. The results are summarized in FIGS. 14A-14L. Purified fusion proteins exhibited binding activity for denatured human collagen type I (FIGS. 14A-14E), IV (FIGS. 14F-14J), III (FIG. 14K), and V (FIG. 14L).

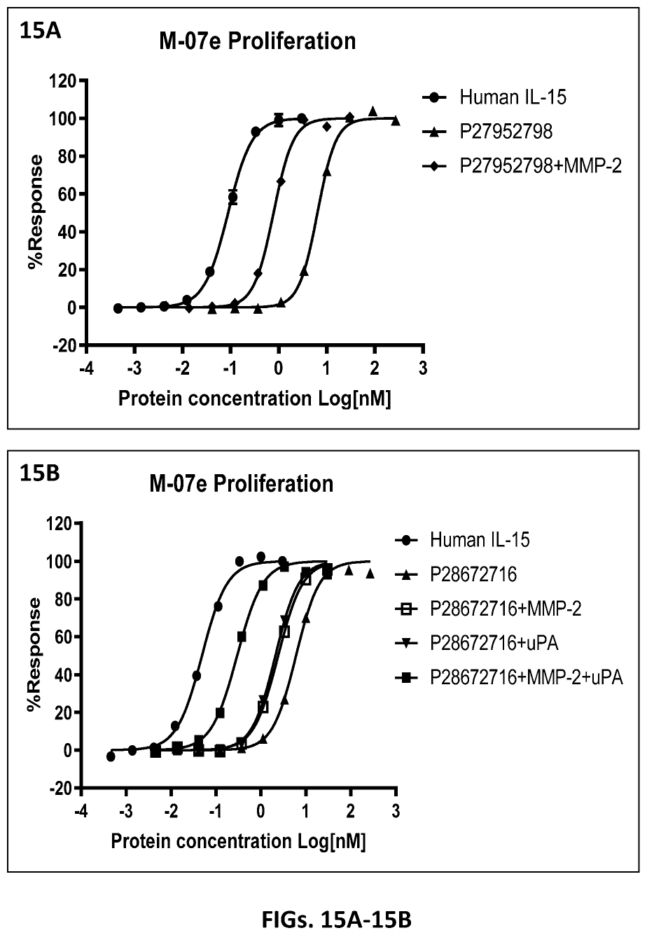

Functional assays—Proliferation. Proliferation assays were performed for purified proteins before and after protease cleavage. Assay procedures were the same as described in Example 2. Representative results are presented in FIGS. 15A-15B. Purified proteins before protease cleavage showed lower activity and protease cleavage could recover the activity of IL-15.

### Example 4

### Engineering of Collagen-Targeted Cytokine Fusion Proteins

To target cytokines to the tumor microenvironment, the cytokines IL-12, IL-7, IL-21, and IFN-α are fused to the C-terminus of a denatured collagen-targeted antibody (see, for example, Table S10). Plasmids coding for the fusion proteins are constructed by standard gene synthesis, followed by sub-cloning into pTT5 expression vector.

### Example 5

### Engineering of Bi-Specific Antibodies Targeted to Collagen and 4-1BB

Plasmids coding for bi-specific antibodies targeted to denatured human collagen and 4-1BB (see, for example, Table S10) are constructed by standard gene synthesis, followed by sub-cloning into pTT5 expression vector.

### Example 6

### Engineering of Bi-Specific Antibodies Targeted to Collagen and CD40

Plasmids coding for the bi-specific antibodies targeted to denatured human collagen and CD40 (see, for example, Table S10) are constructed by standard gene synthesis, followed by sub-cloning into pTT5 expression vector.

1. A fusion protein, comprising
(a) an antibody, or antigen-binding fragment thereof, which specifically binds to a denatured human collagen type I, II, III, IV, and/or V polypeptide at a cryptic collagen epitope, wherein (a) is fused via a linker to;
(b) an effector domain.

2. The fusion protein of claim 1, wherein (a) preferentially binds to the denatured human collagen type I, II, III, IV, and/or V polypeptide relative to a corresponding native human collagen polypeptide, optionally wherein (a) has about or at least about 2, 3, 4, 5, 6, 7, 8, 9, 10, 15, 20, 25, 30, 35, 40, 50, 60, 70, 80, 90, or 100-fold higher binding affinity for the denatured human collagen polypeptide than it has for the corresponding native human collagen polypeptide.

3. The fusion protein of claim 1 or 2, wherein (a) specifically binds to the denatured human collagen type I, II, III, IV, and V polypeptides at the HU177 cryptic collagen epitope, optionally wherein the HU177 cryptic collagen epitope comprises PGXP (SEQ ID NO: 422), LPGXPG (SEQ ID NO: 423), and/or GPP′GXP′G (SEQ ID NO: 424), wherein X is any amino acid, and wherein P′ is hydroxylproline.

4. The fusion protein of claim 1 or 2, wherein (a) specifically binds to the denatured human collagen type IV polypeptide at the HUIV26 cryptic collagen epitope.

5. The fusion protein of any one of claims 1-4, wherein (a) comprises:
a heavy chain variable (V_{H}) region that comprises complementary determining region V_{H}CDR1, V_{H}CDR2, and V_{H}CDR3 sequences selected from Table A1 and variants thereof which specifically bind to the denatured human collagen polypeptide at the cryptic collagen epitope; and
a light chain variable (V_{L}) region that comprises complementary determining region V_{L}CDR1, V_{L}CDR2, and V_{L}CDR3 sequences selected from Table A1 and variants thereof which specifically bind to the denatured human collagen polypeptide at the cryptic collagen epitope.

6. The fusion protein of claim 5, wherein (a) comprises:
the V_{H}CDR1, a V_{H}CDR2, and V_{H}CDR3 regions respectively comprise SEQ ID NOs: 1-3; and the V_{L}CDR1, V_{L}CDR2, and V_{L}CDR3 regions respectively comprise SEQ ID NOs: 4-6;
the V_{H}CDR1, a V_{H}CDR2, and V_{H}CDR3 regions respectively comprise SEQ ID NOs: 7-9; and the V_{L}CDR1, V_{L}CDR2, and V_{L}CDR3 regions respectively comprise SEQ ID NOs: 10-12;
the V_{H}CDR1, a V_{H}CDR2, and V_{H}CDR3 regions respectively comprise SEQ ID NOs: 13-15; and the V_{L}CDR1, V_{L}CDR2, and V_{L}CDR3 regions respectively comprise SEQ ID NOs: 16-18;
the V_{H}CDR1, a V_{H}CDR2, and V_{H}CDR3 regions respectively comprise SEQ ID NOs: 19-21; and the V_{L}CDR1, V_{L}CDR2, and V_{L}CDR3 regions respectively comprise SEQ ID NOs: 22-24;
the V_{H}CDR1, a V_{H}CDR2, and V_{H}CDR3 regions respectively comprise SEQ ID NOs: 25-27; and the V_{L}CDR1, V_{L}CDR2, and V_{L}CDR3 regions respectively comprise SEQ ID NOs: 28-30;
the V_{H}CDR1, a V_{H}CDR2, and V_{H}CDR3 regions respectively comprise SEQ ID NOs: 31-33; and the V_{L}CDR1, V_{L}CDR2, and V_{L}CDR3 regions respectively comprise SEQ ID NOs: 34-36;
the V_{H}CDR1, a V_{H}CDR2, and V_{H}CDR3 regions respectively comprise SEQ ID NOs: 37-39; and the V_{L}CDR1, V_{L}CDR2, and V_{L}CDR3 regions respectively comprise SEQ ID NOs: 40-42;
the V_{H}CDR1, a V_{H}CDR2, and V_{H}CDR3 regions respectively comprise SEQ ID NOs: 43-45; and the V_{L}CDR1, V_{L}CDR2, and V_{L}CDR3 regions respectively comprise SEQ ID NOs: 46-48;
the V_{H}CDR1, a V_{H}CDR2, and V_{H}CDR3 regions respectively comprise SEQ ID NOs: 49-51; and the V_{L}CDR1, V_{L}CDR2, and V_{L}CDR3 regions respectively comprise SEQ ID NOs: 52-54;
the V_{H}CDR1, a V_{H}CDR2, and V_{H}CDR3 regions respectively comprise SEQ ID NOs: 55-57; and the V_{L}CDR1, V_{L}CDR2, and V_{L}CDR3 regions respectively comprise SEQ ID NOs: 58-60;
the V_{H}CDR1, a V_{H}CDR2, and V_{H}CDR3 regions respectively comprise SEQ ID NOs: 61-63; and the V_{L}CDR1, V_{L}CDR2, and V_{L}CDR3 regions respectively comprise SEQ ID NOs: 64-66;
the V_{H}CDR1, a V_{H}CDR2, and V_{H}CDR3 regions respectively comprise SEQ ID NOs: 67-69; and the V_{L}CDR1, V_{L}CDR2, and V_{L}CDR3 regions respectively comprise SEQ ID NOs: 70-72;
the V_{H}CDR1, a V_{H}CDR2, and V_{H}CDR3 regions respectively comprise SEQ ID NOs: 73-75; and the V_{L}CDR1, V_{L}CDR2, and V_{L}CDR3 regions respectively comprise SEQ ID NOs: 76-78;
the V_{H}CDR1, a V_{H}CDR2, and V_{H}CDR3 regions respectively comprise SEQ ID NOs: 79-81; and the V_{L}CDR1, V_{L}CDR2, and V_{L}CDR3 regions respectively comprise SEQ ID NOs: 82-84;
the V_{H}CDR1, a V_{H}CDR2, and V_{H}CDR3 regions respectively comprise SEQ ID NOs: 85-87; and the V_{L}CDR1, V_{L}CDR2, and V_{L}CDR3 regions respectively comprise SEQ ID NOs: 88-90;
the V_{H}CDR1, a V_{H}CDR2, and V_{H}CDR3 regions respectively comprise SEQ ID NOs: 91-93; and the V_{L}CDR1, V_{L}CDR2, and V_{L}CDR3 regions respectively comprise SEQ ID NOs: 94-96;
the V_{H}CDR1, a V_{H}CDR2, and V_{H}CDR3 regions respectively comprise SEQ ID NOs: 97-99; and the V_{L}CDR1, V_{L}CDR2, and V_{L}CDR3 regions respectively comprise SEQ ID NOs: 100-102;
the V_{H}CDR1, a V_{H}CDR2, and V_{H}CDR3 regions respectively comprise SEQ ID NOs: 103-105; and the V_{L}CDR1, V_{L}CDR2, and V_{L}CDR3 regions respectively comprise SEQ ID NOs: 106-108;
the V_{H}CDR1, a V_{H}CDR2, and V_{H}CDR3 regions respectively comprise SEQ ID NOs: 109-111; and the V_{L}CDR1, V_{L}CDR2, and V_{L}CDR3 regions respectively comprise SEQ ID NOs: 112-114;
the V_{H}CDR1, a V_{H}CDR2, and V_{H}CDR3 regions respectively comprise SEQ ID NOs: 115-117; and the V_{L}CDR1, V_{L}CDR2, and V_{L}CDR3 regions respectively comprise SEQ ID NOs: 118-120;
the V_{H}CDR1, a V_{H}CDR2, and V_{H}CDR3 regions respectively comprise SEQ ID NOs: 121-123; and the V_{L}CDR1, V_{L}CDR2, and V_{L}CDR3 regions respectively comprise SEQ ID NOs: 124-126;
the V_{H}CDR1, a V_{H}CDR2, and V_{H}CDR3 regions respectively comprise SEQ ID NOs: 127-129; and the V_{L}CDR1, V_{L}CDR2, and V_{L}CDR3 regions respectively comprise SEQ ID NOs: 130-132;
the V_{H}CDR1, a V_{H}CDR2, and V_{H}CDR3 regions respectively comprise SEQ ID NOs: 133-135; and the V_{L}CDR1, V_{L}CDR2, and V_{L}CDR3 regions respectively comprise SEQ ID NOs: 136-138;
the V_{H}CDR1, a V_{H}CDR2, and V_{H}CDR3 regions respectively comprise SEQ ID NOs: 139-141; and the V_{L}CDR1, V_{L}CDR2, and V_{L}CDR3 regions respectively comprise SEQ ID NOs: 142-144;
the V_{H}CDR1, a V_{H}CDR2, and V_{H}CDR3 regions respectively comprise SEQ ID NOs: 145-147; and the V_{L}CDR1, V_{L}CDR2, and V_{L}CDR3 regions respectively comprise SEQ ID NOs: 148-150;
the V_{H}CDR1, a V_{H}CDR2, and V_{H}CDR3 regions respectively comprise SEQ ID NOs: 151-153; and the V_{L}CDR1, V_{L}CDR2, and V_{L}CDR3 regions respectively comprise SEQ ID NOs: 154-156;
the V_{H}CDR1, a V_{H}CDR2, and V_{H}CDR3 regions respectively comprise SEQ ID NOs: 157-159; and the V_{L}CDR1, V_{L}CDR2, and V_{L}CDR3 regions respectively comprise SEQ ID NOs: 160-162;
the V_{H}CDR1, a V_{H}CDR2, and V_{H}CDR3 regions respectively comprise SEQ ID NOs: 163-165; and the V_{L}CDR1, V_{L}CDR2, and V_{L}CDR3 regions respectively comprise SEQ ID NOs: 166-168;
the V_{H}CDR1, a V_{H}CDR2, and V_{H}CDR3 regions respectively comprise SEQ ID NOs: 169-171; and the V_{L}CDR1, V_{L}CDR2, and V_{L}CDR3 regions respectively comprise SEQ ID NOs: 172-174;
the V_{H}CDR1, a V_{H}CDR2, and V_{H}CDR3 regions respectively comprise SEQ ID NOs: 175-177; and the V_{L}CDR1, V_{L}CDR2, and V_{L}CDR3 regions respectively comprise SEQ ID NOs: 178-180;
the V_{H}CDR1, a V_{H}CDR2, and V_{H}CDR3 regions respectively comprise SEQ ID NOs: 181-183; and the V_{L}CDR1, V_{L}CDR2, and V_{L}CDR3 regions respectively comprise SEQ ID NOs: 184-186;
the V_{H}CDR1, a V_{H}CDR2, and V_{H}CDR3 regions respectively comprise SEQ ID NOs: 187-189; and the V_{L}CDR1, V_{L}CDR2, and V_{L}CDR3 regions respectively comprise SEQ ID NOs: 190-192;
the V_{H}CDR1, a V_{H}CDR2, and V_{H}CDR3 regions respectively comprise SEQ ID NOs: 193-195; and the V_{L}CDR1, V_{L}CDR2, and V_{L}CDR3 regions respectively comprise SEQ ID NOs: 196-198;
the V_{H}CDR1, a V_{H}CDR2, and V_{H}CDR3 regions respectively comprise SEQ ID NOs: 199-201; and the V_{L}CDR1, V_{L}CDR2, and V_{L}CDR3 regions respectively comprise SEQ ID NOs: 202-204;
the V_{H}CDR1, a V_{H}CDR2, and V_{H}CDR3 regions respectively comprise SEQ ID NOs: 205-207; and the V_{L}CDR1, V_{L}CDR2, and V_{L}CDR3 regions respectively comprise SEQ ID NOs: 208-210;
the V_{H}CDR1, a V_{H}CDR2, and V_{H}CDR3 regions respectively comprise SEQ ID NOs: 211-213; and the V_{L}CDR1, V_{L}CDR2, and V_{L}CDR3 regions respectively comprise SEQ ID NOs: 214-216;
the V_{H}CDR1, a V_{H}CDR2, and V_{H}CDR3 regions respectively comprise SEQ ID NOs: 217-219; and the V_{L}CDR1, V_{L}CDR2, and V_{L}CDR3 regions respectively comprise SEQ ID NOs: 220-222;
the V_{H}CDR1, a V_{H}CDR2, and V_{H}CDR3 regions respectively comprise SEQ ID NOs: 223-225; and the V_{L}CDR1, V_{L}CDR2, and V_{L}CDR3 regions respectively comprise SEQ ID NOs: 226-228;
the V_{H}CDR1, a V_{H}CDR2, and V_{H}CDR3 regions respectively comprise SEQ ID NOs: 229-231; and the V_{L}CDR1, V_{L}CDR2, and V_{L}CDR3 regions respectively comprise SEQ ID NOs: 232-234;
the V_{H}CDR1, a V_{H}CDR2, and V_{H}CDR3 regions respectively comprise SEQ ID NOs: 235-237; and the V_{L}CDR1, V_{L}CDR2, and V_{L}CDR3 regions respectively comprise SEQ ID NOs: 238-240;
the V_{H}CDR1, a V_{H}CDR2, and V_{H}CDR3 regions respectively comprise SEQ ID NOs: 241-243; and the V_{L}CDR1, V_{L}CDR2, and V_{L}CDR3 regions respectively comprise SEQ ID NOs: 244-246;
the V_{H}CDR1, a V_{H}CDR2, and V_{H}CDR3 regions respectively comprise SEQ ID NOs: 247-249; and the V_{L}CDR1, V_{L}CDR2, and V_{L}CDR3 regions respectively comprise SEQ ID NOs: 250-252;
the V_{H}CDR1, a V_{H}CDR2, and V_{H}CDR3 regions respectively comprise SEQ ID NOs: 253-255; and the V_{L}CDR1, V_{L}CDR2, and V_{L}CDR3 regions respectively comprise SEQ ID NOs: 256-258;
the V_{H}CDR1, a V_{H}CDR2, and V_{H}CDR3 regions respectively comprise SEQ ID NOs: 259-261; and the V_{L}CDR1, V_{L}CDR2, and V_{L}CDR3 regions respectively comprise SEQ ID NOs: 262-264; or
the V_{H}CDR1, a V_{H}CDR2, and V_{H}CDR3 regions respectively comprise SEQ ID NOs: 265-267; and the V_{L}CDR1, V_{L}CDR2, and V_{L}CDR3 regions respectively comprise SEQ ID NOs: 268-270,
including variants thereof that have 1, 2, 3, 4, 5, or 6 total alterations in one or more of the CDR regions and which specifically bind to the denatured human collagen polypeptide at the cryptic collagen epitope.

7. The fusion protein of claim 5 or 6, wherein for (a):
the heavy chain is at least 80, 85, 90, 95, 97, 98, 99, or 100% identical to a sequence selected from Table A2, including wherein the heavy chain has 1, 2, 3, 4, 5, or 6 alterations in one or more framework regions, and
the light chain is at least 80, 85, 90, 95, 97, 98, 99, or 100% identical to a sequence selected from Table A2, including wherein the light chain has 1, 2, 3, 4, 5, or 6 alterations in one or more framework regions.

8. The fusion protein of claim 7, wherein for (a):
the heavy chain comprises a sequence at least 80, 85, 90, 95, 97, 98, 99, or 100% identical to SEQ ID NO: 271, and the light chain comprises a sequence at least 80, 85, 90, 95, 97, 98, 99, or 100% identical to SEQ ID NO: 272;
the heavy chain comprises a sequence at least 80, 85, 90, 95, 97, 98, 99, or 100% identical to SEQ ID NO: 273, and the light chain comprises a sequence at least 80, 85, 90, 95, 97, 98, 99, or 100% identical to SEQ ID NO: 274;
the heavy chain comprises a sequence at least 80, 85, 90, 95, 97, 98, 99, or 100% identical to SEQ ID NO: 275, and the light chain comprises a sequence at least 80, 85, 90, 95, 97, 98, 99, or 100% identical to SEQ ID NO: 276;
the heavy chain comprises a sequence at least 80, 85, 90, 95, 97, 98, 99, or 100% identical to SEQ ID NO: 277, and the light chain comprises a sequence at least 80, 85, 90, 95, 97, 98, 99, or 100% identical to SEQ ID NO: 278;
the heavy chain comprises a sequence at least 80, 85, 90, 95, 97, 98, 99, or 100% identical to SEQ ID NO: 279, and the light chain comprises a sequence at least 80, 85, 90, 95, 97, 98, 99, or 100% identical to SEQ ID NO: 280;
the heavy chain comprises a sequence at least 80, 85, 90, 95, 97, 98, 99, or 100% identical to SEQ ID NO: 281, and the light chain comprises a sequence at least 80, 85, 90, 95, 97, 98, 99, or 100% identical to SEQ ID NO: 282;
the heavy chain comprises a sequence at least 80, 85, 90, 95, 97, 98, 99, or 100% identical to SEQ ID NO: 283, and the light chain comprises a sequence at least 80, 85, 90, 95, 97, 98, 99, or 100% identical to SEQ ID NO: 284;
the heavy chain comprises a sequence at least 80, 85, 90, 95, 97, 98, 99, or 100% identical to SEQ ID NO: 285, and the light chain comprises a sequence at least 80, 85, 90, 95, 97, 98, 99, or 100% identical to SEQ ID NO: 286;
the heavy chain comprises a sequence at least 80, 85, 90, 95, 97, 98, 99, or 100% identical to SEQ ID NO: 287, and the light chain comprises a sequence at least 80, 85, 90, 95, 97, 98, 99, or 100% identical to SEQ ID NO: 288;
the heavy chain comprises a sequence at least 80, 85, 90, 95, 97, 98, 99, or 100% identical to SEQ ID NO: 289, and the light chain comprises a sequence at least 80, 85, 90, 95, 97, 98, 99, or 100% identical to SEQ ID NO: 290;
the heavy chain comprises a sequence at least 80, 85, 90, 95, 97, 98, 99, or 100% identical to SEQ ID NO: 291, and the light chain comprises a sequence at least 80, 85, 90, 95, 97, 98, 99, or 100% identical to SEQ ID NO: 292;
the heavy chain comprises a sequence at least 80, 85, 90, 95, 97, 98, 99, or 100% identical to SEQ ID NO: 293, and the light chain comprises a sequence at least 80, 85, 90, 95, 97, 98, 99, or 100% identical to SEQ ID NO: 294;
the heavy chain comprises a sequence at least 80, 85, 90, 95, 97, 98, 99, or 100% identical to SEQ ID NO: 295, and the light chain comprises a sequence at least 80, 85, 90, 95, 97, 98, 99, or 100% identical to SEQ ID NO: 296;
the heavy chain comprises a sequence at least 80, 85, 90, 95, 97, 98, 99, or 100% identical to SEQ ID NO: 297, and the light chain comprises a sequence at least 80, 85, 90, 95, 97, 98, 99, or 100% identical to SEQ ID NO: 298;
the heavy chain comprises a sequence at least 80, 85, 90, 95, 97, 98, 99, or 100% identical to SEQ ID NO: 299, and the light chain comprises a sequence at least 80, 85, 90, 95, 97, 98, 99, or 100% identical to SEQ ID NO: 300;
the heavy chain comprises a sequence at least 80, 85, 90, 95, 97, 98, 99, or 100% identical to SEQ ID NO: 301, and the light chain comprises a sequence at least 80, 85, 90, 95, 97, 98, 99, or 100% identical to SEQ ID NO: 302;
the heavy chain comprises a sequence at least 80, 85, 90, 95, 97, 98, 99, or 100% identical to SEQ ID NO: 303, and the light chain comprises a sequence at least 80, 85, 90, 95, 97, 98, 99, or 100% identical to SEQ ID NO: 304;
the heavy chain comprises a sequence at least 80, 85, 90, 95, 97, 98, 99, or 100% identical to SEQ ID NO: 305, and the light chain comprises a sequence at least 80, 85, 90, 95, 97, 98, 99, or 100% identical to SEQ ID NO: 306;
the heavy chain comprises a sequence at least 80, 85, 90, 95, 97, 98, 99, or 100% identical to SEQ ID NO: 307, and the light chain comprises a sequence at least 80, 85, 90, 95, 97, 98, 99, or 100% identical to SEQ ID NO: 308;
the heavy chain comprises a sequence at least 80, 85, 90, 95, 97, 98, 99, or 100% identical to SEQ ID NO: 309, and the light chain comprises a sequence at least 80, 85, 90, 95, 97, 98, 99, or 100% identical to SEQ ID NO: 310;
the heavy chain comprises a sequence at least 80, 85, 90, 95, 97, 98, 99, or 100% identical to SEQ ID NO: 311, and the light chain comprises a sequence at least 80, 85, 90, 95, 97, 98, 99, or 100% identical to SEQ ID NO: 312;
the heavy chain comprises a sequence at least 80, 85, 90, 95, 97, 98, 99, or 100% identical to SEQ ID NO: 313, and the light chain comprises a sequence at least 80, 85, 90, 95, 97, 98, 99, or 100% identical to SEQ ID NO: 314;
the heavy chain comprises a sequence at least 80, 85, 90, 95, 97, 98, 99, or 100% identical to SEQ ID NO: 315, and the light chain comprises a sequence at least 80, 85, 90, 95, 97, 98, 99, or 100% identical to SEQ ID NO: 316;
the heavy chain comprises a sequence at least 80, 85, 90, 95, 97, 98, 99, or 100% identical to SEQ ID NO: 317, and the light chain comprises a sequence at least 80, 85, 90, 95, 97, 98, 99, or 100% identical to SEQ ID NO: 318;
the heavy chain comprises a sequence at least 80, 85, 90, 95, 97, 98, 99, or 100% identical to SEQ ID NO: 319, and the light chain comprises a sequence at least 80, 85, 90, 95, 97, 98, 99, or 100% identical to SEQ ID NO: 320;
the heavy chain comprises a sequence at least 80, 85, 90, 95, 97, 98, 99, or 100% identical to SEQ ID NO: 321, and the light chain comprises a sequence at least 80, 85, 90, 95, 97, 98, 99, or 100% identical to SEQ ID NO: 322;
the heavy chain comprises a sequence at least 80, 85, 90, 95, 97, 98, 99, or 100% identical to SEQ ID NO: 323, and the light chain comprises a sequence at least 80, 85, 90, 95, 97, 98, 99, or 100% identical to SEQ ID NO: 324;
the heavy chain comprises a sequence at least 80, 85, 90, 95, 97, 98, 99, or 100% identical to SEQ ID NO: 325, and the light chain comprises a sequence at least 80, 85, 90, 95, 97, 98, 99, or 100% identical to SEQ ID NO: 326;
the heavy chain comprises a sequence at least 80, 85, 90, 95, 97, 98, 99, or 100% identical to SEQ ID NO: 327, and the light chain comprises a sequence at least 80, 85, 90, 95, 97, 98, 99, or 100% identical to SEQ ID NO: 328;
the heavy chain comprises a sequence at least 80, 85, 90, 95, 97, 98, 99, or 100% identical to SEQ ID NO: 329, and the light chain comprises a sequence at least 80, 85, 90, 95, 97, 98, 99, or 100% identical to SEQ ID NO: 330;
the heavy chain comprises a sequence at least 80, 85, 90, 95, 97, 98, 99, or 100% identical to SEQ ID NO: 331, and the light chain comprises a sequence at least 80, 85, 90, 95, 97, 98, 99, or 100% identical to SEQ ID NO: 332;
the heavy chain comprises a sequence at least 80, 85, 90, 95, 97, 98, 99, or 100% identical to SEQ ID NO: 333, and the light chain comprises a sequence at least 80, 85, 90, 95, 97, 98, 99, or 100% identical to SEQ ID NO: 334;
the heavy chain comprises a sequence at least 80, 85, 90, 95, 97, 98, 99, or 100% identical to SEQ ID NO: 335, and the light chain comprises a sequence at least 80, 85, 90, 95, 97, 98, 99, or 100% identical to SEQ ID NO: 336;
the heavy chain comprises a sequence at least 80, 85, 90, 95, 97, 98, 99, or 100% identical to SEQ ID NO: 337, and the light chain comprises a sequence at least 80, 85, 90, 95, 97, 98, 99, or 100% identical to SEQ ID NO: 338;
the heavy chain comprises a sequence at least 80, 85, 90, 95, 97, 98, 99, or 100% identical to SEQ ID NO: 339, and the light chain comprises a sequence at least 80, 85, 90, 95, 97, 98, 99, or 100% identical to SEQ ID NO: 340;
the heavy chain comprises a sequence at least 80, 85, 90, 95, 97, 98, 99, or 100% identical to SEQ ID NO: 341, and the light chain comprises a sequence at least 80, 85, 90, 95, 97, 98, 99, or 100% identical to SEQ ID NO: 342;
the heavy chain comprises a sequence at least 80, 85, 90, 95, 97, 98, 99, or 100% identical to SEQ ID NO: 343, and the light chain comprises a sequence at least 80, 85, 90, 95, 97, 98, 99, or 100% identical to SEQ ID NO: 344;
the heavy chain comprises a sequence at least 80, 85, 90, 95, 97, 98, 99, or 100% identical to SEQ ID NO: 345, and the light chain comprises a sequence at least 80, 85, 90, 95, 97, 98, 99, or 100% identical to SEQ ID NO: 346;
the heavy chain comprises a sequence at least 80, 85, 90, 95, 97, 98, 99, or 100% identical to SEQ ID NO: 347, and the light chain comprises a sequence at least 80, 85, 90, 95, 97, 98, 99, or 100% identical to SEQ ID NO: 348;
the heavy chain comprises a sequence at least 80, 85, 90, 95, 97, 98, 99, or 100% identical to SEQ ID NO: 349, and the light chain comprises a sequence at least 80, 85, 90, 95, 97, 98, 99, or 100% identical to SEQ ID NO: 350;
the heavy chain comprises a sequence at least 80, 85, 90, 95, 97, 98, 99, or 100% identical to SEQ ID NO: 351, and the light chain comprises a sequence at least 80, 85, 90, 95, 97, 98, 99, or 100% identical to SEQ ID NO: 352;
the heavy chain comprises a sequence at least 80, 85, 90, 95, 97, 98, 99, or 100% identical to SEQ ID NO: 353, and the light chain comprises a sequence at least 80, 85, 90, 95, 97, 98, 99, or 100% identical to SEQ ID NO: 354; or
the heavy chain comprises a sequence at least 80, 85, 90, 95, 97, 98, 99, or 100% identical to SEQ ID NO: 355, and the light chain comprises a sequence at least 80, 85, 90, 95, 97, 98, 99, or 100% identical to SEQ ID NO: 356.

9. The fusion protein of any one of claims 1-4, wherein (a) comprises
a V_{H} region or heavy chain comprising:
an HFR1 sequence selected from SEQ ID NOs: 357-361;
an HFR2 sequence selected from SEQ ID NOs: 362-366;
an HFR3 sequence selected from SEQ ID NOs: 367-369;
an HFR4 sequence set forth in SEQ ID NO: 370;
a V_{H}CDR1 sequence selected from SEQ ID NOs: 371-372;
a V_{H}CDR2 sequence selected from SEQ ID NOs: 373-374; and
a V_{H}CDR3 sequence selected from SEQ ID NO: 375,
and/or a V_{L} region or light chain comprising:
an LFR1 sequence set forth in SEQ ID NO: 376;
an LFR2 sequence set forth in SEQ ID NO: 377;
an LFR3 sequence set forth in SEQ ID NO: 378;
an LFR4 sequence set forth in SEQ ID NO: 379;
a V_{L}CDR1 sequence selected from SEQ ID NOs: 380-385;
a V_{L}CDR2 sequence set forth in SEQ ID NO: 386; and
a V_{L}CDR3 sequence selected from SEQ ID NOs: 387-388,
wherein the antibody, or antigen-binding fragment thereof, specifically binds to a human denatured collagen, or wherein (a) comprises
an HFR1 sequence selected from SEQ ID NOs: 389-390;
an HFR2 sequence selected from SEQ ID NOs: 391-392;
an HFR3 sequence set forth in SEQ ID NO: 393;
an HFR4 sequence set forth in SEQ ID NO: 394;
a V_{H}CDR1 sequence selected from SEQ ID NOs: 395-397;
a V_{H}CDR2 sequence selected from SEQ ID NOs: 398-401; and
a V_{H}CDR3 sequence selected from SEQ ID NOs: 402-405,
and/or a VL region or light chain comprising:
an LFR1 sequence set forth in SEQ ID NO: 406;
an LFR2 sequence set forth in SEQ ID NO: 407;
an LFR3 sequence set forth in SEQ ID NO: 408;
an LFR4 sequence set forth in SEQ ID NO: 409;
a V_{L}CDR1 sequence selected from SEQ ID NOs: 410-412;
a V_{L}CDR2 set forth in SEQ ID NO: 413; and
a V_{L}CDR3 set forth in SEQ ID NO: 414,
wherein the antibody, or antigen-binding fragment thereof, specifically binds to a human denatured collagen.

10. The fusion protein of any one of claims 1-9, wherein (b) the effector domain comprises an immune cell-stimulatory ligand or domain, an immune cell-inhibitory ligand or domain, a cytocidal (e.g., tumor cell cytocidal) ligand or domain, or an immunomodulatory or anti-cancer antibody, or antigen-binding fragment thereof.

11. The fusion protein of claim 10, wherein the effector domain is an IL-2 polypeptide, optionally an IL-2 polypeptide that comprises, consists, or consists essentially of an amino acid sequence that is at least 80, 85, 90, 95, 98, or 100% identical to a sequence selected from Table S1, and wherein the cell receptor is an IL-2Rβ/γc and/or IL-2Rα/β/γc chain present on the surface of an immune cell.

12. The fusion protein of claim 10, wherein the effector domain is an IL-15 polypeptide, optionally an IL-15 polypeptide that comprises, consists, or consists essentially of an amino acid sequence that is at least 80, 85, 90, 95, 98, or 100% identical to a sequence selected from Table S2, and wherein the cell receptor is an IL-15Rβ/γc chain present on the surface of an immune cell.

13. The fusion protein of claim 10, wherein the effector domain is a hybrid IL-2/IL-15 polypeptide, optionally a hybrid IL-2/IL-15 polypeptide that comprises, consists, or consists essentially of an amino acid sequence that is at least 80, 85, 90, 95, 98, or 100% identical to a sequence selected from Table S3, and wherein the cell receptor is an IL-2Rβ/γc chain, an IL-2Rα/β/γc chain, and/or an IL-15Rβ/γc chain present on the surface of an immune cell.

14. The fusion protein of claim 10, wherein the effector domain is a TNF superfamily ligand polypeptide, optionally wherein the TNF superfamily ligand polypeptide and its corresponding cell receptor(s) are selected from Table T1, optionally wherein:
(i) the TNF superfamily ligand polypeptide is TRAIL, including single chain trimeric TRAIL, and the cell receptor is selected from Death receptor 4, Death receptor 5, Decoy receptor 1, and decoy receptor 2 present on an immune cell or cancer cell; or
(ii) the TNF superfamily ligand polypeptide is 4-1BBL, including single chain trimeric 4-1BB, and the cell receptor is 4-1BB (CD137) present on an immune cell or cancer cell.

15. The fusion protein of claim 14, wherein the TNF superfamily ligand polypeptide comprises, consists, or consists essentially of an amino acid sequence that is at least 80, 85, 90, 95, 98, or 100% identical to a sequence selected from Table S4, and wherein the cell receptor is selected from the corresponding receptor from Table T1.

16. The fusion protein of claim 10, wherein the effector domain is an IL-12 polypeptide, optionally an IL-12 polypeptide that comprises, consists, or consists essentially of an amino acid sequence that is at least 80, 85, 90, 95, 98, or 100% identical to a sequence selected from Table S5, and wherein the cell receptor is an IL-12 receptor, optionally an IL-12Rβ1 and IL-12Rβ2 chain present on the surface of an immune cell.

17. The fusion protein of claim 10, wherein the effector domain is an IL-10 polypeptide, optionally an IL-10 polypeptide that comprises, consists, or consists essentially of an amino acid sequence that is at least 80, 85, 90, 95, 98, or 100% identical to a sequence selected from Table S6, and wherein the cell receptor is an IL-10 receptor complex comprising IL-10α receptor and IL-10βreceptor subunits, optionally wherein the cell receptor is an IL-10α receptor subunit.

18. The fusion protein of claim 10, wherein the effector domain is an IFN-α polypeptide, optionally an IFN-α polypeptide that comprises, consists, or consists essentially of an amino acid sequence that is at least 80, 85, 90, 95, 98, or 100% identical to a sequence selected from Table S7, and wherein the cell receptor is an interferon α/β receptor.

19. The fusion protein of claim 10, wherein the effector domain is an interleukin-7 (IL-7) polypeptide, optionally an IL-7 polypeptide that comprises, consists, or consists essentially of an amino acid sequence that is at least 80, 85, 90, 95, 98, or 100% identical to a sequence selected from Table S8, and wherein the cell receptor is an IL-7 receptor (IL-7R); or wherein the effector domain is an interleukin-21 (IL-21) polypeptide, optionally an IL-7 polypeptide that comprises, consists, or consists essentially of an amino acid sequence that is at least 80, 85, 90, 95, 98, or 100% identical to a sequence selected from Table S9, and wherein the cell receptor is an IL-21 receptor (IL-21R).

20. The fusion protein of claim 10, wherein the effector domain comprises an immunomodulatory or anti-cancer antibody, or antigen-binding fragment thereof, which specifically binds to a polypeptide selected from human Her2/neu, Her1/EGF receptor (EGFR), EGFR1, EGFR2, EGFR3, Her3, A33 antigen, B7H3, B7H4, CD3, CD4, CD5, CD8, CD16, CD19, CD20, CD30, CD22, CD23 (IgE Receptor), B-cell maturation antigen (BCMA), Trop-2, Claudin 6, claudin 16, MAGE-3, C242 antigen, 5T4, IL-6, IL-13, PD-1, CTLA-4, PD-L1, TIGIT, TIM-3, LAG-3, 4-1BB, vascular endothelial growth factor VEGF (e.g., VEGF-A) VEGFR-1, VEGFR-2, CD27, CD28, CD33, CD37, CD40, CD44, CD51, CD52, CD56, CD74, CD80, CD86, CD137, CD152, CD200, CD221, CCR4, HLA-DR, CTLA-4, NPC-1C, Siglec15, MIC-A, NKG2A, NKG2D, Nkp30, NKp46, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

21. The fusion protein of any one of claims 1-20, wherein (b) is fused via the linker to the N-terminus of the V_{H} region of (a).

22. The fusion protein of any one of claims 1-20, wherein (b) is fused via the linker to the N-terminus of the V_{L} region of (a).

23. The fusion protein of any one of claims 1-20, wherein (b) is fused via the linker to the C-terminus of (a), optionally the C-terminus of an Fc region of (a).

24. The fusion protein of any one of claims 1-23, wherein the linker is a flexible, stable linker, optionally selected from Table L1.

25. The fusion protein of any one of claims 1-23, wherein the linker is a flexible, cleavable linker, optionally selected from Table L2.

26. The fusion protein of claim 25, wherein the cleavable linker comprises a protease cleavage site, or is a low pH-sensitive linker.

27. The fusion protein of claim 26, wherein the protease cleavage site is cleavable by a protease selected from one or more of a metalloprotease, a serine protease, a cysteine protease, and an aspartic acid protease.

28. The fusion protein of claim 25 or 26, wherein the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

29. The fusion protein of any one of claims 1-28, wherein the linker is about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

30. The fusion protein of any one of claims 1-29, wherein binding of (a) to high density denatured collagen in a tissue in vivo, optionally a cancer tissue, increases binding of (b) to its target cell surface receptor or ligand, optionally by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control such as the absence or reduced levels of high density denatured collagen.

31. The fusion protein of claim 30, wherein the cell surface receptor is on the surface of an immune cell or a cancer cell, optionally wherein the immune cell is selected from one or more of a T cell, a B cell, a natural killer cell, a monocyte, and a macrophage.

32. The fusion protein of any one of claims 1-31, wherein the antibody, or antigen-binding fragment thereof, is a monoclonal antibody and/or a humanized antibody, including wherein the antibody, or antigen-binding fragment thereof, is a whole antibody, a fragment antigen-binding domain (Fab), a F(ab′)2 domain, a single-chain variable fragment (scFv), a dimeric single-chain variable fragment (di-scFv), a single domain antibody (sdAb), or a bi-specific antibody.

33. The fusion protein of any one of claims 1-32, wherein the fusion protein, optionally bispecific antibody, comprises, consists, or consists essentially of an amino acid sequence at least 80, 85, 90, 95, 97, 98, 99, or 100% identical to a sequence selected from Table S10.

34. A recombinant nucleic acid molecule encoding the fusion protein of any one of claims 1-33.

35. A vector comprising the recombinant nucleic acid molecule of claim 34.

36. A host cell comprising the recombinant nucleic acid molecule of claim 34 and/or the vector of claim 35.

37. A method of producing a fusion protein, comprising culturing the host cell of claim 36 under culture conditions suitable for the expression of the fusion protein, and isolating the fusion protein from the culture.

38. A pharmaceutical composition, comprising the fusion protein of any one of claims 1-33, and a pharmaceutically acceptable carrier.

39. A method of treating disease in a subject in need thereof, comprising administering to the subject a therapeutically effective amount of the pharmaceutical composition of claim 38.

40. The method of claim 39, wherein the disease is selected from one or more of a cancer, a viral infection, and an immune disorder.

41. The method of claim 40, wherein the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

42. The method of claim 39, wherein the viral infection is selected from one or more of human immunodeficiency virus (HIV), hepatitis A, hepatitis B, hepatitis C, hepatitis E, caliciviruses associated diarrhoea, rotavirus diarrhoea, Haemophilus influenzae B pneumonia and invasive disease, influenza, measles, mumps, rubella, parainfluenza associated pneumonia, respiratory syncytial virus (RSV) pneumonia, severe acute respiratory syndrome (SARS), human papillomavirus, herpes simplex type 2 genital ulcers, dengue fever, Japanese encephalitis, tick-borne encephalitis, West-Nile virus associated disease, yellow fever, Epstein-Barr virus, Lassa fever, Crimean-Congo haemorrhagic fever, Ebola haemorrhagic fever, Marburg haemorrhagic fever, Rabies, Rift Valley fever, smallpox, upper and lower respiratory infections, and poliomyelitis, optionally wherein the subject is HIV-positive.

43. The method of claim 40, wherein the effector domain has immune cell-stimulating activity, and wherein immune disorder is selected from one or more of type 1 diabetes, vasculitis, and an immunodeficiency.

44. The method of claim 40, wherein the effector domain has immune cell-inhibitory activity, and wherein the immune disorder is an autoimmune and/or inflammatory disease, optionally multiple sclerosis.

45. The method of any one of claims 39-44, wherein following administration, binding of (a) to high density denatured collagen in a tissue in vivo, optionally a cancer tissue, increases binding of the effector domain to its target cell surface receptor or ligand, optionally by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control such as the absence or reduced levels of high density denatured collagen

46. The method of claim 45, wherein the cell surface receptor is on the surface of an immune cell or a cancer cell, optionally wherein the immune cell is selected from one or more of a T cell, a B cell, a natural killer cell, a monocyte, and a macrophage.

47. The method of any one of claims 39-46, wherein the effector domain has immune cell-stimulatory activity, and wherein administration of the fusion protein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response.

48. The method of any one of claims 39-47, wherein the effector domain has immune cell-stimulatory activity or cytocidal activity, and wherein administration of the fusion protein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

49. The method of any one of claims 39-46, wherein the effector domain has immune cell-inhibitory activity, and wherein administration of the fusion protein reduces an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is associated with an inflammatory and/or autoimmune disease.

50. The method of any one of claims 39-49, wherein the pharmaceutical composition is administered to the subject by parenteral administration.

51. The method of claim 50, wherein the parenteral administration is intravenous administration.

52. Use of a pharmaceutical composition of claim 38 in the preparation of a medicament for treating a disease in a subject, optionally wherein the disease is a cancer, a viral infection, or an immune disorder, optionally type 1 diabetes, vasculitis, an immunodeficiency, an inflammatory disease, or an autoimmune disease.

53. A pharmaceutical composition of claim 38 for use in treating a disease in a subject, optionally wherein the disease is a cancer, a viral infection, or an immune disorder, optionally type 1 diabetes, vasculitis, an immunodeficiency, an inflammatory disease, or an autoimmune disease.

54. An isolated antibody, or an antigen-binding fragment thereof, which specifically binds to a denatured human collagen type I, II, III, IV, and/or V polypeptide at a cryptic collagen epitope, and comprises,
a heavy chain variable (V_{H}) region that comprises complementary determining region V_{H}CDR1, V_{H}CDR2, and V_{H}CDR3 sequences selected from Table A1 and variants thereof which specifically bind to the human collagen polypeptide at the cryptic collagen epitope; and
a light chain variable (V_{L}) region that comprises complementary determining region V_{L}CDR1, V_{L}CDR2, and V_{L}CDR3 sequences selected from Table A1 and variants thereof which specifically bind to the human collagen polypeptide at the cryptic collagen epitope,
excluding the HU177 and HUIV26.

55. The isolated antibody, or antigen-binding fragment thereof, of claim 54, wherein
the V_{H}CDR1, a V_{H}CDR2, and V_{H}CDR3 regions respectively comprise SEQ ID NOs: 1-3; and the V_{L}CDR1, V_{L}CDR2, and V_{L}CDR3 regions respectively comprise SEQ ID NOs: 4-6;
the V_{H}CDR1, a V_{H}CDR2, and V_{H}CDR3 regions respectively comprise SEQ ID NOs: 7-9; and the V_{L}CDR1, V_{L}CDR2, and V_{L}CDR3 regions respectively comprise SEQ ID NOs: 10-12;
the V_{H}CDR1, a V_{H}CDR2, and V_{H}CDR3 regions respectively comprise SEQ ID NOs: 13-15; and the V_{L}CDR1, V_{L}CDR2, and V_{L}CDR3 regions respectively comprise SEQ ID NOs: 16-18;
the V_{H}CDR1, a V_{H}CDR2, and V_{H}CDR3 regions respectively comprise SEQ ID NOs: 19-21; and the V_{L}CDR1, V_{L}CDR2, and V_{L}CDR3 regions respectively comprise SEQ ID NOs: 22-24;
the V_{H}CDR1, a V_{H}CDR2, and V_{H}CDR3 regions respectively comprise SEQ ID NOs: 25-27; and the V_{L}CDR1, V_{L}CDR2, and V_{L}CDR3 regions respectively comprise SEQ ID NOs: 28-30;
the V_{H}CDR1, a V_{H}CDR2, and V_{H}CDR3 regions respectively comprise SEQ ID NOs: 31-33; and the V_{L}CDR1, V_{L}CDR2, and V_{L}CDR3 regions respectively comprise SEQ ID NOs: 34-36;
the V_{H}CDR1, a V_{H}CDR2, and V_{H}CDR3 regions respectively comprise SEQ ID NOs: 37-39; and the V_{L}CDR1, V_{L}CDR2, and V_{L}CDR3 regions respectively comprise SEQ ID NOs: 40-42;
the V_{H}CDR1, a V_{H}CDR2, and V_{H}CDR3 regions respectively comprise SEQ ID NOs: 43-45; and the V_{L}CDR1, V_{L}CDR2, and V_{L}CDR3 regions respectively comprise SEQ ID NOs: 46-48;
the V_{H}CDR1, a V_{H}CDR2, and V_{H}CDR3 regions respectively comprise SEQ ID NOs: 49-51; and the V_{L}CDR1, V_{L}CDR2, and V_{L}CDR3 regions respectively comprise SEQ ID NOs: 52-54;
the V_{H}CDR1, a V_{H}CDR2, and V_{H}CDR3 regions respectively comprise SEQ ID NOs: 55-57; and the V_{L}CDR1, V_{L}CDR2, and V_{L}CDR3 regions respectively comprise SEQ ID NOs: 58-60;
the V_{H}CDR1, a V_{H}CDR2, and V_{H}CDR3 regions respectively comprise SEQ ID NOs: 61-63; and the V_{L}CDR1, V_{L}CDR2, and V_{L}CDR3 regions respectively comprise SEQ ID NOs: 64-66;
the V_{H}CDR1, a V_{H}CDR2, and V_{H}CDR3 regions respectively comprise SEQ ID NOs: 67-69; and the V_{L}CDR1, V_{L}CDR2, and V_{L}CDR3 regions respectively comprise SEQ ID NOs: 70-72;
the V_{H}CDR1, a V_{H}CDR2, and V_{H}CDR3 regions respectively comprise SEQ ID NOs: 73-75; and the V_{L}CDR1, V_{L}CDR2, and V_{L}CDR3 regions respectively comprise SEQ ID NOs: 76-78;
the V_{H}CDR1, a V_{H}CDR2, and V_{H}CDR3 regions respectively comprise SEQ ID NOs: 79-81; and the V_{L}CDR1, V_{L}CDR2, and V_{L}CDR3 regions respectively comprise SEQ ID NOs: 82-84;
the V_{H}CDR1, a V_{H}CDR2, and V_{H}CDR3 regions respectively comprise SEQ ID NOs: 85-87; and the V_{L}CDR1, V_{L}CDR2, and V_{L}CDR3 regions respectively comprise SEQ ID NOs: 88-90;
the V_{H}CDR1, a V_{H}CDR2, and V_{H}CDR3 regions respectively comprise SEQ ID NOs: 91-93; and the V_{L}CDR1, V_{L}CDR2, and V_{L}CDR3 regions respectively comprise SEQ ID NOs: 94-96;
the V_{H}CDR1, a V_{H}CDR2, and V_{H}CDR3 regions respectively comprise SEQ ID NOs: 97-99; and the V_{L}CDR1, V_{L}CDR2, and V_{L}CDR3 regions respectively comprise SEQ ID NOs: 100-102;
the V_{H}CDR1, a V_{H}CDR2, and V_{H}CDR3 regions respectively comprise SEQ ID NOs: 103-105; and the V_{L}CDR1, V_{L}CDR2, and V_{L}CDR3 regions respectively comprise SEQ ID NOs: 106-108;
the V_{H}CDR1, a V_{H}CDR2, and V_{H}CDR3 regions respectively comprise SEQ ID NOs: 109-111; and the V_{L}CDR1, V_{L}CDR2, and V_{L}CDR3 regions respectively comprise SEQ ID NOs: 112-114;
the V_{H}CDR1, a V_{H}CDR2, and V_{H}CDR3 regions respectively comprise SEQ ID NOs: 115-117; and the V_{L}CDR1, V_{L}CDR2, and V_{L}CDR3 regions respectively comprise SEQ ID NOs: 118-120;
the V_{H}CDR1, a V_{H}CDR2, and V_{H}CDR3 regions respectively comprise SEQ ID NOs: 121-123; and the V_{L}CDR1, V_{L}CDR2, and V_{L}CDR3 regions respectively comprise SEQ ID NOs: 124-126;
the V_{H}CDR1, a V_{H}CDR2, and V_{H}CDR3 regions respectively comprise SEQ ID NOs: 127-129; and the V_{L}CDR1, V_{L}CDR2, and V_{L}CDR3 regions respectively comprise SEQ ID NOs: 130-132;
the V_{H}CDR1, a V_{H}CDR2, and V_{H}CDR3 regions respectively comprise SEQ ID NOs: 133-135; and the V_{L}CDR1, V_{L}CDR2, and V_{L}CDR3 regions respectively comprise SEQ ID NOs: 136-138;
the V_{H}CDR1, a V_{H}CDR2, and V_{H}CDR3 regions respectively comprise SEQ ID NOs: 139-141; and the V_{L}CDR1, V_{L}CDR2, and V_{L}CDR3 regions respectively comprise SEQ ID NOs: 142-144;
the V_{H}CDR1, a V_{H}CDR2, and V_{H}CDR3 regions respectively comprise SEQ ID NOs: 145-147; and the V_{L}CDR1, V_{L}CDR2, and V_{L}CDR3 regions respectively comprise SEQ ID NOs: 148-150;
the V_{H}CDR1, a V_{H}CDR2, and V_{H}CDR3 regions respectively comprise SEQ ID NOs: 151-153; and the V_{L}CDR1, V_{L}CDR2, and V_{L}CDR3 regions respectively comprise SEQ ID NOs: 154-156;
the V_{H}CDR1, a V_{H}CDR2, and V_{H}CDR3 regions respectively comprise SEQ ID NOs: 157-159; and the V_{L}CDR1, V_{L}CDR2, and V_{L}CDR3 regions respectively comprise SEQ ID NOs: 160-162;
the V_{H}CDR1, a V_{H}CDR2, and V_{H}CDR3 regions respectively comprise SEQ ID NOs: 163-165; and the V_{L}CDR1, V_{L}CDR2, and V_{L}CDR3 regions respectively comprise SEQ ID NOs: 166-168;
the V_{H}CDR1, a V_{H}CDR2, and V_{H}CDR3 regions respectively comprise SEQ ID NOs: 169-171; and the V_{L}CDR1, V_{L}CDR2, and V_{L}CDR3 regions respectively comprise SEQ ID NOs: 172-174;
the V_{H}CDR1, a V_{H}CDR2, and V_{H}CDR3 regions respectively comprise SEQ ID NOs: 175-177; and the V_{L}CDR1, V_{L}CDR2, and V_{L}CDR3 regions respectively comprise SEQ ID NOs: 178-180;
the V_{H}CDR1, a V_{H}CDR2, and V_{H}CDR3 regions respectively comprise SEQ ID NOs: 181-183; and the V_{L}CDR1, V_{L}CDR2, and V_{L}CDR3 regions respectively comprise SEQ ID NOs: 184-186;
the V_{H}CDR1, a V_{H}CDR2, and V_{H}CDR3 regions respectively comprise SEQ ID NOs: 187-189; and the V_{L}CDR1, V_{L}CDR2, and V_{L}CDR3 regions respectively comprise SEQ ID NOs: 190-192;
the V_{H}CDR1, a V_{H}CDR2, and V_{H}CDR3 regions respectively comprise SEQ ID NOs: 193-195; and the V_{L}CDR1, V_{L}CDR2, and V_{L}CDR3 regions respectively comprise SEQ ID NOs: 196-198;
the V_{H}CDR1, a V_{H}CDR2, and V_{H}CDR3 regions respectively comprise SEQ ID NOs: 199-201; and the V_{L}CDR1, V_{L}CDR2, and V_{L}CDR3 regions respectively comprise SEQ ID NOs: 202-204;
the V_{H}CDR1, a V_{H}CDR2, and V_{H}CDR3 regions respectively comprise SEQ ID NOs: 205-207; and the V_{L}CDR1, V_{L}CDR2, and V_{L}CDR3 regions respectively comprise SEQ ID NOs: 208-210;
the V_{H}CDR1, a V_{H}CDR2, and V_{H}CDR3 regions respectively comprise SEQ ID NOs: 211-213; and the V_{L}CDR1, V_{L}CDR2, and V_{L}CDR3 regions respectively comprise SEQ ID NOs: 214-216;
the V_{H}CDR1, a V_{H}CDR2, and V_{H}CDR3 regions respectively comprise SEQ ID NOs: 217-219; and the V_{L}CDR1, V_{L}CDR2, and V_{L}CDR3 regions respectively comprise SEQ ID NOs: 220-222;
the V_{H}CDR1, a V_{H}CDR2, and V_{H}CDR3 regions respectively comprise SEQ ID NOs: 223-225; and the V_{L}CDR1, V_{L}CDR2, and V_{L}CDR3 regions respectively comprise SEQ ID NOs: 226-228;
the V_{H}CDR1, a V_{H}CDR2, and V_{H}CDR3 regions respectively comprise SEQ ID NOs: 229-231; and the V_{L}CDR1, V_{L}CDR2, and V_{L}CDR3 regions respectively comprise SEQ ID NOs: 232-234;
the V_{H}CDR1, a V_{H}CDR2, and V_{H}CDR3 regions respectively comprise SEQ ID NOs: 235-237; and the V_{L}CDR1, V_{L}CDR2, and V_{L}CDR3 regions respectively comprise SEQ ID NOs: 238-240;
the V_{H}CDR1, a V_{H}CDR2, and V_{H}CDR3 regions respectively comprise SEQ ID NOs: 241-243; and the V_{L}CDR1, V_{L}CDR2, and V_{L}CDR3 regions respectively comprise SEQ ID NOs: 244-246;
the V_{H}CDR1, a V_{H}CDR2, and V_{H}CDR3 regions respectively comprise SEQ ID NOs: 247-249; and the V_{L}CDR1, V_{L}CDR2, and V_{L}CDR3 regions respectively comprise SEQ ID NOs: 250-252; or
the V_{H}CDR1, a V_{H}CDR2, and V_{H}CDR3 regions respectively comprise SEQ ID NOs: 253-255; and the V_{L}CDR1, V_{L}CDR2, and V_{L}CDR3 regions respectively comprise SEQ ID NOs: 256-258;
including variants thereof that have 1, 2, 3, 4, 5, or 6 total alterations in one or more of the CDR regions and specifically bind to the human collagen polypeptide at the cryptic collagen epitope.

56. The isolated antibody, or antigen-binding fragment thereof, of claim 55, wherein
the heavy chain is at least 80, 85, 90, 95, 97, 98, 99, or 100% identical to a sequence selected from Table A2, including wherein the heavy chain has 1, 2, 3, 4, 5, or 6 alterations in one or more framework regions, and
the light chain is at least 80, 85, 90, 95, 97, 98, 99, or 100% identical to a sequence selected from Table A2, including wherein the light chain has 1, 2, 3, 4, 5, or 6 alterations in one or more framework regions,
excluding the HU177 and HUIV26 antibodies.

57. The isolated antibody, or antigen-binding fragment thereof, of claim 56, wherein
the heavy chain comprises a sequence at least 80, 85, 90, 95, 97, 98, 99, or 100% identical to SEQ ID NO: 271, and the light chain comprises a sequence at least 80, 85, 90, 95, 97, 98, 99, or 100% identical to SEQ ID NO: 272;
the heavy chain comprises a sequence at least 80, 85, 90, 95, 97, 98, 99, or 100% identical to SEQ ID NO: 273, and the light chain comprises a sequence at least 80, 85, 90, 95, 97, 98, 99, or 100% identical to SEQ ID NO: 274;
the heavy chain comprises a sequence at least 80, 85, 90, 95, 97, 98, 99, or 100% identical to SEQ ID NO: 275, and the light chain comprises a sequence at least 80, 85, 90, 95, 97, 98, 99, or 100% identical to SEQ ID NO: 276;
the heavy chain comprises a sequence at least 80, 85, 90, 95, 97, 98, 99, or 100% identical to SEQ ID NO: 277, and the light chain comprises a sequence at least 80, 85, 90, 95, 97, 98, 99, or 100% identical to SEQ ID NO: 278;
the heavy chain comprises a sequence at least 80, 85, 90, 95, 97, 98, 99, or 100% identical to SEQ ID NO: 279, and the light chain comprises a sequence at least 80, 85, 90, 95, 97, 98, 99, or 100% identical to SEQ ID NO: 280;
the heavy chain comprises a sequence at least 80, 85, 90, 95, 97, 98, 99, or 100% identical to SEQ ID NO: 281, and the light chain comprises a sequence at least 80, 85, 90, 95, 97, 98, 99, or 100% identical to SEQ ID NO: 282;
the heavy chain comprises a sequence at least 80, 85, 90, 95, 97, 98, 99, or 100% identical to SEQ ID NO: 283, and the light chain comprises a sequence at least 80, 85, 90, 95, 97, 98, 99, or 100% identical to SEQ ID NO: 284;
the heavy chain comprises a sequence at least 80, 85, 90, 95, 97, 98, 99, or 100% identical to SEQ ID NO: 285, and the light chain comprises a sequence at least 80, 85, 90, 95, 97, 98, 99, or 100% identical to SEQ ID NO: 286;
the heavy chain comprises a sequence at least 80, 85, 90, 95, 97, 98, 99, or 100% identical to SEQ ID NO: 287, and the light chain comprises a sequence at least 80, 85, 90, 95, 97, 98, 99, or 100% identical to SEQ ID NO: 288;
the heavy chain comprises a sequence at least 80, 85, 90, 95, 97, 98, 99, or 100% identical to SEQ ID NO: 289, and the light chain comprises a sequence at least 80, 85, 90, 95, 97, 98, 99, or 100% identical to SEQ ID NO: 290;
the heavy chain comprises a sequence at least 80, 85, 90, 95, 97, 98, 99, or 100% identical to SEQ ID NO: 291, and the light chain comprises a sequence at least 80, 85, 90, 95, 97, 98, 99, or 100% identical to SEQ ID NO: 292;
the heavy chain comprises a sequence at least 80, 85, 90, 95, 97, 98, 99, or 100% identical to SEQ ID NO: 293, and the light chain comprises a sequence at least 80, 85, 90, 95, 97, 98, 99, or 100% identical to SEQ ID NO: 294;
the heavy chain comprises a sequence at least 80, 85, 90, 95, 97, 98, 99, or 100% identical to SEQ ID NO: 295, and the light chain comprises a sequence at least 80, 85, 90, 95, 97, 98, 99, or 100% identical to SEQ ID NO: 296;
the heavy chain comprises a sequence at least 80, 85, 90, 95, 97, 98, 99, or 100% identical to SEQ ID NO: 297, and the light chain comprises a sequence at least 80, 85, 90, 95, 97, 98, 99, or 100% identical to SEQ ID NO: 298;
the heavy chain comprises a sequence at least 80, 85, 90, 95, 97, 98, 99, or 100% identical to SEQ ID NO: 299, and the light chain comprises a sequence at least 80, 85, 90, 95, 97, 98, 99, or 100% identical to SEQ ID NO: 300;
the heavy chain comprises a sequence at least 80, 85, 90, 95, 97, 98, 99, or 100% identical to SEQ ID NO: 301, and the light chain comprises a sequence at least 80, 85, 90, 95, 97, 98, 99, or 100% identical to SEQ ID NO: 302;
the heavy chain comprises a sequence at least 80, 85, 90, 95, 97, 98, 99, or 100% identical to SEQ ID NO: 303, and the light chain comprises a sequence at least 80, 85, 90, 95, 97, 98, 99, or 100% identical to SEQ ID NO: 304;
the heavy chain comprises a sequence at least 80, 85, 90, 95, 97, 98, 99, or 100% identical to SEQ ID NO: 305, and the light chain comprises a sequence at least 80, 85, 90, 95, 97, 98, 99, or 100% identical to SEQ ID NO: 306;
the heavy chain comprises a sequence at least 80, 85, 90, 95, 97, 98, 99, or 100% identical to SEQ ID NO: 307, and the light chain comprises a sequence at least 80, 85, 90, 95, 97, 98, 99, or 100% identical to SEQ ID NO: 308;
the heavy chain comprises a sequence at least 80, 85, 90, 95, 97, 98, 99, or 100% identical to SEQ ID NO: 309, and the light chain comprises a sequence at least 80, 85, 90, 95, 97, 98, 99, or 100% identical to SEQ ID NO: 310;
the heavy chain comprises a sequence at least 80, 85, 90, 95, 97, 98, 99, or 100% identical to SEQ ID NO: 311, and the light chain comprises a sequence at least 80, 85, 90, 95, 97, 98, 99, or 100% identical to SEQ ID NO: 312;
the heavy chain comprises a sequence at least 80, 85, 90, 95, 97, 98, 99, or 100% identical to SEQ ID NO: 313, and the light chain comprises a sequence at least 80, 85, 90, 95, 97, 98, 99, or 100% identical to SEQ ID NO: 314;
the heavy chain comprises a sequence at least 80, 85, 90, 95, 97, 98, 99, or 100% identical to SEQ ID NO: 315, and the light chain comprises a sequence at least 80, 85, 90, 95, 97, 98, 99, or 100% identical to SEQ ID NO: 316;
the heavy chain comprises a sequence at least 80, 85, 90, 95, 97, 98, 99, or 100% identical to SEQ ID NO: 317, and the light chain comprises a sequence at least 80, 85, 90, 95, 97, 98, 99, or 100% identical to SEQ ID NO: 318;
the heavy chain comprises a sequence at least 80, 85, 90, 95, 97, 98, 99, or 100% identical to SEQ ID NO: 319, and the light chain comprises a sequence at least 80, 85, 90, 95, 97, 98, 99, or 100% identical to SEQ ID NO: 320;
the heavy chain comprises a sequence at least 80, 85, 90, 95, 97, 98, 99, or 100% identical to SEQ ID NO: 321, and the light chain comprises a sequence at least 80, 85, 90, 95, 97, 98, 99, or 100% identical to SEQ ID NO: 322;
the heavy chain comprises a sequence at least 80, 85, 90, 95, 97, 98, 99, or 100% identical to SEQ ID NO: 323, and the light chain comprises a sequence at least 80, 85, 90, 95, 97, 98, 99, or 100% identical to SEQ ID NO: 324;
the heavy chain comprises a sequence at least 80, 85, 90, 95, 97, 98, 99, or 100% identical to SEQ ID NO: 325, and the light chain comprises a sequence at least 80, 85, 90, 95, 97, 98, 99, or 100% identical to SEQ ID NO: 326;
the heavy chain comprises a sequence at least 80, 85, 90, 95, 97, 98, 99, or 100% identical to SEQ ID NO: 327, and the light chain comprises a sequence at least 80, 85, 90, 95, 97, 98, 99, or 100% identical to SEQ ID NO: 328;
the heavy chain comprises a sequence at least 80, 85, 90, 95, 97, 98, 99, or 100% identical to SEQ ID NO: 329, and the light chain comprises a sequence at least 80, 85, 90, 95, 97, 98, 99, or 100% identical to SEQ ID NO: 330;
the heavy chain comprises a sequence at least 80, 85, 90, 95, 97, 98, 99, or 100% identical to SEQ ID NO: 331, and the light chain comprises a sequence at least 80, 85, 90, 95, 97, 98, 99, or 100% identical to SEQ ID NO: 332;
the heavy chain comprises a sequence at least 80, 85, 90, 95, 97, 98, 99, or 100% identical to SEQ ID NO: 333, and the light chain comprises a sequence at least 80, 85, 90, 95, 97, 98, 99, or 100% identical to SEQ ID NO: 334;
the heavy chain comprises a sequence at least 80, 85, 90, 95, 97, 98, 99, or 100% identical to SEQ ID NO: 335, and the light chain comprises a sequence at least 80, 85, 90, 95, 97, 98, 99, or 100% identical to SEQ ID NO: 336;
the heavy chain comprises a sequence at least 80, 85, 90, 95, 97, 98, 99, or 100% identical to SEQ ID NO: 337, and the light chain comprises a sequence at least 80, 85, 90, 95, 97, 98, 99, or 100% identical to SEQ ID NO: 338;
the heavy chain comprises a sequence at least 80, 85, 90, 95, 97, 98, 99, or 100% identical to SEQ ID NO: 339, and the light chain comprises a sequence at least 80, 85, 90, 95, 97, 98, 99, or 100% identical to SEQ ID NO: 340;
the heavy chain comprises a sequence at least 80, 85, 90, 95, 97, 98, 99, or 100% identical to SEQ ID NO: 341, and the light chain comprises a sequence at least 80, 85, 90, 95, 97, 98, 99, or 100% identical to SEQ ID NO: 342;
the heavy chain comprises a sequence at least 80, 85, 90, 95, 97, 98, 99, or 100% identical to SEQ ID NO: 343, and the light chain comprises a sequence at least 80, 85, 90, 95, 97, 98, 99, or 100% identical to SEQ ID NO: 344;
the heavy chain comprises a sequence at least 80, 85, 90, 95, 97, 98, 99, or 100% identical to SEQ ID NO: 345, and the light chain comprises a sequence at least 80, 85, 90, 95, 97, 98, 99, or 100% identical to SEQ ID NO: 346;
the heavy chain comprises a sequence at least 80, 85, 90, 95, 97, 98, 99, or 100% identical to SEQ ID NO: 347, and the light chain comprises a sequence at least 80, 85, 90, 95, 97, 98, 99, or 100% identical to SEQ ID NO: 348;
the heavy chain comprises a sequence at least 80, 85, 90, 95, 97, 98, 99, or 100% identical to SEQ ID NO: 349, and the light chain comprises a sequence at least 80, 85, 90, 95, 97, 98, 99, or 100% identical to SEQ ID NO: 350;
the heavy chain comprises a sequence at least 80, 85, 90, 95, 97, 98, 99, or 100% identical to SEQ ID NO: 351, and the light chain comprises a sequence at least 80, 85, 90, 95, 97, 98, 99, or 100% identical to SEQ ID NO: 352;
the heavy chain comprises a sequence at least 80, 85, 90, 95, 97, 98, 99, or 100% identical to SEQ ID NO: 353, and the light chain comprises a sequence at least 80, 85, 90, 95, 97, 98, 99, or 100% identical to SEQ ID NO: 354; or
the heavy chain comprises a sequence at least 80, 85, 90, 95, 97, 98, 99, or 100% identical to SEQ ID NO: 355, and the light chain comprises a sequence at least 80, 85, 90, 95, 97, 98, 99, or 100% identical to SEQ ID NO: 356.

58. The isolated antibody, or antigen-binding fragment thereof, of any one of claims 54-57, comprising a V_{H} region or heavy chain comprising:
an HFR1 sequence selected from SEQ ID NOs: 357-361;
an HFR2 sequence selected from SEQ ID NOs: 362-366;
an HFR3 sequence selected from SEQ ID NOs: 367-369;
an HFR4 sequence set forth in SEQ ID NO: 370;
a V_{H}CDR1 sequence selected from SEQ ID NOs: 371-372;
a V_{H}CDR2 sequence selected from SEQ ID NOs: 373-374; and
a V_{H}CDR3 sequence selected from SEQ ID NO: 375,
and/or a V_{L} region or light chain comprising:
an LFR1 sequence set forth in SEQ ID NO: 376;
an LFR2 sequence set forth in SEQ ID NO: 377;
an LFR3 sequence set forth in SEQ ID NO: 378;
an LFR4 sequence set forth in SEQ ID NO: 379;
a V_{L}CDR1 sequence selected from SEQ ID NOs: 380-385;
a V_{L}CDR2 sequence set forth in SEQ ID NO: 386; and
a V_{L}CDR3 sequence selected from SEQ ID NOs: 387-388,
wherein the antibody, or antigen-binding fragment thereof, specifically binds to a human denatured collagen, or comprising:
an HFR1 sequence selected from SEQ ID NOs: 389-390;
an HFR2 sequence selected from SEQ ID NOs: 391-392;
an HFR3 sequence set forth in SEQ ID NO: 393;
an HFR4 sequence set forth in SEQ ID NO: 394;
a V_{H}CDR1 sequence selected from SEQ ID NOs: 395-397;
a V_{H}CDR2 sequence selected from SEQ ID NOs: 398-401; and
a V_{H}CDR3 sequence selected from SEQ ID NOs: 402-405,
and/or a VL region or light chain comprising:
an LFR1 sequence set forth in SEQ ID NO: 406;
an LFR2 sequence set forth in SEQ ID NO: 407;
an LFR3 sequence set forth in SEQ ID NO: 408;
an LFR4 sequence set forth in SEQ ID NO: 409;
a V_{L}CDR1 sequence selected from SEQ ID NOs: 410-412;
a V_{L}CDR2 set forth in SEQ ID NO: 413; and
a V_{L}CDR3 set forth in SEQ ID NO: 414,
wherein the antibody, or antigen-binding fragment thereof, specifically binds to a human denatured collagen.

59. The isolated antibody, or antigen-binding fragment thereof, of any one of claims 54-58, which preferentially binds to the denatured human collagen type I, II, III, IV, and/or V polypeptide relative to a corresponding native human collagen polypeptide, optionally wherein the antibody, or antigen-binding fragment thereof, has about or at least about 2, 3, 4, 5, 6, 7, 8, 9, 10, 15, 20, 25, 30, 35, 40, 50, 60, 70, 80, 90, or 100-fold higher binding affinity for the denatured human collagen polypeptide than it has for the corresponding native human collagen polypeptide.

60. The isolated antibody, or antigen-binding fragment thereof, of any one of claims 54-59, which specifically binds to the denatured human collagen type I, II, III, IV, and V polypeptides at the HU177 cryptic collagen epitope, optionally wherein the cryptic collagen epitope comprises PGXP (SEQ ID NO: 422), LPGXPG (SEQ ID NO: 423), and/or GPP′GXP′G (SEQ ID NO: 424), wherein X is any amino acid, and wherein P′ is hydroxylproline.

61. The isolated antibody, or antigen-binding fragment thereof, of any one of claims 54-58, which specifically binds to the denatured human collagen type IV polypeptide at the HUIV26 cryptic collagen epitope.

62. The isolated antibody, or antigen-binding fragment thereof, of any one of claims 54-61, which is a monoclonal antibody and/or a humanized antibody.

63. The isolated antibody, or antigen-binding fragment thereof, of any one of claims 54-62, which is a fragment antigen-binding domain (Fab), a F(ab′)2 domain, or a whole antibody.

64. The isolated antibody, or antigen-binding fragment thereof, of any one of claims 54-63, which is fused via an optional linker to an effector domain.

65. A recombinant nucleic acid molecule encoding the antibody, or antigen-binding fragment thereof, of any one of claims 54-64.

66. A vector comprising the recombinant nucleic acid molecule of claim 65.

67. A host cell comprising the recombinant nucleic acid molecule of claim 65 and/or the vector of claim 66.

68. A method of producing an antibody, or antigen-binding fragment thereof, comprising culturing the host cell of claim 67 under culture conditions suitable for the expression of the antibody, or antigen-binding fragment thereof, and isolating the antibody, or antigen-binding fragment thereof, from the culture.

69. A pharmaceutical or diagnostic composition, comprising the antibody, or antigen-binding fragment thereof, of any one of claims 54-64, and a pharmaceutically-acceptable carrier.
